(19) United States
(12) Patent Application Publication
(10) Pub. No.: US 2019/0382794 A1
(43) Pub. Date: Dec. 19, 2019

(54) MITOCHONDRIAL DELIVERY OF RECOMBINANT NUCLEIC ACIDS

(71) Applicant: NATIONAL UNIVERSITY OF SINGAPORE, Singapore (SG)

(72) Inventors: Volker PATZEL, Singapore (SG); Kaustav CHATTERJEE, Singapore (SG)

(73) Assignee: NATIONAL UNIVERSITY OF SINGAPORE, Singapore (SG)

(21) Appl. No.: 16/099,151

(22) PCT Filed: May 8, 2017
(86) PCT No.: PCT/SG2017/050238

§ 371 Date: Nov. 5, 2018

(30) Foreign Application Priority Data
May 6, 2016 (SG) 10201603628Q

(51) Int. Cl. C12N 15/86; C12N 7/00

(52) U.S. Cl. CPC C12N 15/86; C12N 7/00; C12N 2320/32; A61K 48/00; C12N 2710/16133; C12N 2810/10; C12N 2710/16122

(57) ABSTRACT

The present disclosure describes a nucleic acid delivery construct comprising at least one sense or antisense RNA subdomain of the human cytomegalovirus β2.7 RNA, wherein each subdomain is capable of localization within the mitochondria, for transport into mitochondria. Disclosed herein are also methods of enhancing mitochondrial gene function, or suppressing defective mitochondrial gene function, or both, as well as methods of treating a mitochondrial disorder.

### CROSS-REFERENCE TO RELATED APPLICATIONS

This application claims the benefit of priority of SG provisional application No. 10201603628Q, filed 6 May 2016, the contents of it being hereby incorporated by reference in its entirety for all purposes.

### FIELD OF THE INVENTION

This disclosure relates to the field of molecular biology. In particular, the present invention relates to the use of mitochondria-targeting sequences for the transport of nucleic acid sequences.

### BACKGROUND OF THE INVENTION

Mitochondria are the cellular organelles involved in the terminal part of respiration cycle in almost all living organisms. The genomic organization of the mitochondria is unique and codes for 37 genes, of which 22 are tRNA genes, two are mitochondrial ribosomal RNA (12 s and 16 s) genes and 13 genes for subunits of respiratory enzymes. Defects in these respiratory genes have been associated with a number of neurodegenerative disorders, such as ataxias, optic neuropathies, Parkinson's disease, and also associated with ageing. However, the uptake of nucleic acids by the mitochondria for mitochondrial protection and modulation is poorly investigated, and efficient mitochondrial delivery vectors have not been identified yet. Thus, there is a need for a delivery system capable of delivering payloads (for example, nucleic acid sequences that are carried by the delivery system) into the mitochondria of cells.

### SUMMARY

In one aspect, the present invention refers to a nucleic acid delivery construct comprising at least one sense or antisense RNA subdomain of the human cytomegalovirus β2.7 RNA, wherein each subdomain is capable of localization within the mitochondria.

In another aspect, the present invention refers to a vector, a recombinant cell, or a recombinant organism comprising the nucleic acid sequence as disclosed herein.

In yet another aspect, the present invention refers to a nucleic acid sequence comprising at least one or more sense or antisense RNA sequences of the human cytomegalovirus β2.7 RNA selected from group consisting of domain 1 (D1; SEQ ID NO: 3 or 7), domain 2 (D2; SEQ ID NO: 4 or 8), domain 3 (D3; SEQ ID NO: 5 or 9) and domain 4 (D4; SEQ ID NO: 6 or 10).

In a further aspect, the present invention refers to a method of enhancing mitochondrial gene function, or suppressing defective mitochondrial gene function, or both (provided that in this case the mitochondrial genes are different from each other), the method comprising administering to a subject the nucleic acid delivery construct as disclosed herein, wherein the mitochondrial gene functions are different from each other.

In another aspect, the present invention refers to a method of treating a mitochondrial disorder, the method comprising administering to a subject the nucleic acid delivery construct as disclosed herein.

### BRIEF DESCRIPTION OF THE DRAWINGS

The invention will be better understood with reference to the detailed description when considered in conjunction with the non-limiting examples and the accompanying drawings, in which:

FIG. 1 shows the structural and functional analyses that identify a single structural subdomain that governs the complete mitochondrial localization activity of the full-length β2.7 RNA. a) HepG2 cells were transfected with in vitro transcribed β2.7 RNA. Cytoplasmic and mitochondrial fractions were purified and the relative distribution of the β2.7 RNA in each fraction was determined using real-time RT-PCR. Data represent means±SD of 3 independent experiments (*p<0.05, Student's t-test). b) Thermodynamic profiling of the β2.7 RNA sequence of the CMV Towne strain using the algorithm foldsplit (HUSAR) and a window size of 200 nt and shift increment of 1 nt. The Gibbs free energy (ΔG) values of secondary structure formation for each window was plotted against the position of the sequence window. ΔG minima indicate four thermodynamically conserved and potentially functionally relevant structural subdomains D1 to D4. c) Well-defined secondary structure subdomains D1 to D4 could be assigned to each of the energetic minima within the minimum free energy structure (mfe) of T7 polymerase transcript of the Towne β2.7 RNA as predicted by mfold. d) shows a schematic representation of the δ2.7 RNA domain structure along with the single domain deletion constructs (SDDC) and the single domain constructs (SDCs). e) shows the results of HepG2 cells that were transfected with in vitro transcribed, full-length β2.7 RNA or the SDDCs. Mitochondrial uptake was monitored using rtRT-PCR. Data represent mean values±SD of 3 independent experiments (***p<0.001, ****p<0.0001 one-way ANOVA, Dunnett's multiple comparison test). f) shows the results of HepG2 cells that were transfected with in vitro transcribed full-length β2.7 RNA or the SDCs and mitochondrial uptake was measured using rtRT-PCR and domain-specific primers. Data represents±SD of 3 independent experiments (*p<0.01, ***p<0.001, Student's t-test).

FIG. 2 shows the structural and functional analyses that indicate a remarkable homology between the β2.7 RNA and its antisense RNA. a) Thermodynamic profiling of the Towne β2.7 antisense RNA using foldsplit (HUSAR) identified thermodynamically conserved structural subdomains D1AS to D4AS corresponding to domains D1 to D4 in the complementary strand. b) HepG2 cells were transfected with in vitro transcribed sense (T7) or antisense (SP6) full-length β2.7 RNA and mitochondrial uptake was quantified using rtRT-PCR and domain-specific primers (for D1 to D4) from left to right. Data represent mean values±SD of three independent experiments (***p<0.001, Student's t-test). c) Mitochondrial localization of the full-length β2.7 antisense RNA and antisense SDDCs as detected using rtRT-PCR. Data represent mean values±SD of three independent experiments (*p<0.05, **p<0.01, one-way ANOVA, Dunnett's multiple comparison Test). d) Mitochondrial localization of the full-length δ2.7 RNA, the full-length β2.7 antisense RNA, and antisense SDCs measured using rtRT-PCR and domain-specific primers. Data represent mean values±SD of 3 independent experiments (*p<0.05, ** p<0.01, ***p<0.001, ***p<0.0001, one-way ANOVA, Tukey's multiple comparisons test).

FIG. 3 shows the results of β2.7 RNA-mediated mitochondrial targeting of the GFP mRNA, which triggers mitochondrial GFP expression. a) Design of β2.7-GFP fusion constructs and controls. The β2.7 RNA sequence was fused to either the GFP sequence gene with genomic (gGFP) or mitochondrial (mtGFP) start and stop codons. To preserve the structures of sub-domains D1 to D4 of the β2.7 RNA upon fusion to the GFP sequence, a spacer sequence was designed and inserted. To target any cytoplasmic mtGFP to the nuclei, constructs were designed in which the mtGFP protein was fused to a nuclear localization sequence (NLS). Respective nuclear or mitochondrial translational stop codons (indicated by triangles) were placed downstream of the respective GFP or NLS sequences so that only the GFP protein or a GFP-NLS fusion was translated. b) Example demonstrating the functionality of the spacer sequence using RNA secondary structures predicted by mfold. Insertion of the spacer between the GFP mRNA and the β2.7 RNA restores β2.7 RNA subdomains D2 and D3. c) Mitochondrial targeting activity of β2.7 fusion RNAs relative to the β2.7-negative control or the parental β2.7 RNA. d) HepG2 cells were transfected with in vitro transcribed RNA and mitochondrial localization was monitored using rtRT-PCR. Data represent mean values of three independent experiments (****p<0.0001, one-way ANOVA, Dunnett's multiple comparison test). e) to f) Flow cytometry analyses comprising scatter plots and histogram shifts. HepG2 cells transfected with plasmid DNA vectors expressing the GFP_ β2.7 fusion RNAs and GFP expression was monitored at day 3. h) Summary of flow cytometry data. The Geometric means, percentages of GFP-positive cells, and Medians are indicated. Data represent 3 independent experiments (*p<0.05, **p<0.01, ***p<0.001, ****p<0.0001, one-way ANOVA, Tukey's multiple comparisons Test and Student's t-test). i-k, HepG2 cells were transfected with NLS(+) or NLS(−) GFP-β2.7 RNA fusion constructs and co-localisation of GFP expression and mitochondrial stain (MitoTracker Red) in the confocal microscopy images was analysed and presented in three different ways using ImageJ algorithm JACOP: i, Pearson's coefficient; j, Mander's coefficient; and k, Overlap coefficient. Each five representative images were analysed (*p<0.05, **p<0.01, ***p<0.001, ****p<0.0001, two-way ANOVA, Tukey's multiple comparison's Test).

FIG. 4 shows that β2.7 RNA-mediated delivery of antisense RNA can trigger knockdown of mtATP6 and mtATP8. a) Minimum free energy (MFE) secondary structures of computationally-selected unstructured antisense RNA targeting the mitochondrial genes mtATP6 and mtATP8 as predicted by mfold. b) Design of the β2.7_antisense fusion RNAs and controls. Spacers S6 and S8 ensure the antisense RNA domains remain open upon fusion to the β2.7 RNA. c) MFE structures of the asATP6_ β2.7 and β2.7_asATP8 fusion RNAs predicted by mfold. d) Knockdown of mtATP6 or mtATP8 mRNA levels measured using rtRT-PCR in HEK293T cells transfected with β2.7_antisense fusion RNAs (asATP6 or asATP8) or control RNA. Data represents means±SD of 3 independent experiments (*p<0.05, ***p<0.001, one-way ANOVA, Dunnett's multiple comparisons test). e) Reduction of ATP levels in RNA-transfected HEK293T cells detected using the Cell Titre-Glo Assay. Data represent means+SD of 3 independent experiments (*p<0.05, **p<0.05, ***p<0.001, one-way ANOVA, Dunnett's multiple comparisons test). f, Cell viability of HEK293T cells 24 hours post-transfection of RNA determined by the Alamar Blue cell viability assay. Data represent means±SD of 3 independent experiments (*p<0.05, **p<0.05, ***p<0.001, one-way ANOVA, Dunnett's multiple comparisons test).

FIG. 5 shows results showing that distinct tandem repeats of β2.7 RNA sub-domains 2 and 3 show enhanced mitochondrial uptake and can protect mitochondrial Complex I. a) shows schematics of MFE structures of domain 2 (D2X4) and b) domain 3 (D3X4) tandem repeats as predicted by mfold. Spacers s1 to s4 were used to stabilize the structures of subdomains D2 and D3 in the tandem repeat constructs D2X4 and D3X4. d) shows a schematic, exemplary representation of constructs comprising tetrameric repeats of domain 2 (D2X4) or 3 (D3X4), combinations thereof (D3X4_D2X4 or D2X4_D3X4), or a domain 3/2 repeat ((D3_D2)X4). c) shows line graphs depicting median CT ratios. In vitro transcribed RNAs were serially diluted, reverse transcribed and subjected to rtPCR to determine the CT ratios for each dilution. Each standard curve effectively detected down to 104 molecules. E) shows column graphs depicting relative uptake levels of mitochondrial uptake of domain 2 or 3 monomers or tetramers relative to the parental β2.7 RNA in transfected HepG2 cells as determined by rtRT-PCR. F) shows column graphs depicting relative uptake levels of mitochondrial uptake of different tandem repeat RNAs relative to the parental β2.7 RNA in transfected HepG2 cells as determined by rtRT-PCR. g) and h) show the results of a cell viability staining using Alamar blue cell viability assays, thereby monitoring protection of mitochondrial Complex I against 200 μM rotenone treatment 24 hours (g)) or 48 hours (h)) post-transfection of HEK293T cells with in vitro transcribed RNA or plasmid expression vectors. Data was normalized relative to an untreated control. Data shown in E) to H) is represented as means plus standard deviation of 3 independent experiments (*p<0.05, **p<0.01, ***p<0.001, ****p<0.0001, one-way ANOVA, Tukey's multiple comparisons Test).

FIG. 6 shows schematic examples of using CMV β2.7 RNA-derived sequences/structures (m β2.7) for mitochondrial delivery of recombinant nucleic acids. m β2.7 can be either the full-length β2.7 RNA, a structural sub-domains thereof, a repeat of a β2.7 RNA. a) shows the fusing m β2.7 to the 5′ end of a recombinant no-coding or coding RNA. b) shows the fusing m β2.7 to the 3′ end of a recombinant no-coding or coding RNA. c) shows binding of m β2.7 via complementary base pairing, i.e. a binding domain, towards double-stranded DNA (gene). d) shows linkage of m β2.7 via complementary binding domains (DNA or RNA) to circular single-stranded DNA or a recombinant mitochondrial genome. e) shows the linkage of m β2.7 via complementary binding domains, as shown in d), but the ssDNA enzymatically was converted to dsDNA and ligated. f) shows the linkage of m β2.7 via complementary binding domains, as shown in d),but linking two or more m β2.7 RNAs to a circular ssDNA.

FIG. 7 shows a schematic map of the pVAX1 vector used in the present invention. The NdeI and BcII site were used to clone the construct into the pVAX1 vector.

FIG. 8 shows a schematic of the overlap extension polymerase chain reaction (OE PCR) used to generate the single domain deletion constructs, which was carried out in two steps. In the first step, the region upstream of the deletion domain (ΔD) is PCR amplified using a common forward primer which carries the NheI site and OE reverse primer, which introduces the priming site for the region downstream of the ΔD. Subsequently, the domain downstream of the ΔD is PCR amplified using an OE forward primer, which introduces the priming site for the region upstream of the ΔD, and a common reverse primer which carries the BamHI site. Both PCR products were then gel purified and mixed together in equimolar amounts and then re-amplified with PCR, in which the OE introduced priming sites acted as primers for their respective counterparts thereby giving the full length construct with the desired deletion.

FIG. 9 shows a schematic outlining the synthesis and cloning strategy of the single domain constructs. The forward and the reverse primers were designed to introduce the BspEI and HindIII sites respectively. The PCR products were subsequently digested with BspEI and HindIII, purified and cloned into the pVAX1 vector using the BspEI and HindIII sites.

FIG. 10 shows a schematic representation of the different constructs used to test mitochondrial GFP delivery. To facilitate mitochondria specific GFP expression, the genomic start and stop codons were PCR modified to mitochondria specific start (ATA) and stop codons (respectively).

FIG. 11 shows a schematic representation of the PCR introduction of a nuclear localization sequence (NLS). The NLS sequence was PCR amplified from the pEBFP-NUC plasmid by PCR, using forward and reverse primers and inserted upstream of the stop codon (AGA).

FIG. 12 shows a schematic representation of the cloning strategy used to synthesize the domain 2 and domain 3 tandem repeat sequences. The individual copies were first synthesized in 4 sets using PCR primers. Since all primers for a particular tandem repeat share the same binding region, the Tm is the same for all sets for a particular tandem repeat. Each set has its own set of spacers, as indicated by S1-S4 in the figure. The restriction sites were chosen so that the site at the 3′end of one set matched the one at the 5′ end of the next set within the tandem repeat. The restriction sites used were NheI, EcoRI, KpnI, AgeI and HindIII. After PCR, the products were PCR purified and digested with their respective restriction sites. The digested PCR products were then mixed in equimolar amount of the digested vector backbone and ligated using T4 DNA ligase.

FIG. 13 shows a schematic representation of the strategy for synthesis of D3X4_D2X4, as well as for D2X4_D3X4. The domain 2 tandem repeat (D2X4) was PCR amplified from the pVAX1 vector carrying D2X4. To prevent amplification of shorter fragments, primers were so designed that majority of the binding region of the primer lay on the vector backbone itself. Each of the primers introduced HindIII sites at either end. The PCR product was digested and re-ligated within the HindIII site of the pVAX1 vector carrying the D3X4 sequence. To prevent re-ligation of the vector, the digested vector was dephosphorylated with alkaline phosphatase. The second part of FIG. 13 shows a schematic representation of strategy used for D2X4_D3X4: Similarly The domain 3 tandem repeat (D3X4) was PCR amplified from the pVAX1 vector carrying D3X4.The primer design strategy employed was the same as used for the domain 2 tandem repeat (D2X4) outlined above. The PCR product was digested and re-ligated within the HindIII site of the de-phosphorylated pVAX1 vector carrying the D2X4 sequence.

FIG. 14 shows a schematic representation of the strategy used for synthesizing (D3_D2)x4. In the first step the first D3 of D3x4 was cloned using the same restriction sites into the first repeat position of D2X4. In the second step, the third D3 of D3x4 was cloned using the same restriction sites into the third position of D2X4. The step 2 product thus obtained was then PCR amplified using primers which introduce HindIII sites at either end. The PCR product was then ligated downstream of the step 2 product.

FIG. 15 shows a schematic representation of the strategy used to isolate the sequences targeted by antisense RNA. Mitochondrial RNA was reverse transcribed with gene specific reverse primer sequences (ATP6Rv and ATP8Rv) to obtain first strand ATP6 and ATP8 cDNA pools. Subsequently, these pools were used as templates for PCR to obtain double stranded DNA sequences representing the target elements.

FIG. 16 shows a schematic representation of the strategy used for synthesizing ATP6 β2.7.The ATP6 target sequence was re-amplified by PCR to introduce restriction sites in an opposite orientation to that in β2.7_pVAX1 sequence. The resulting product was digested with NheI and BspeI, and then ligated into the β2.7_pVAX1 vector backbone to obtain the ATP6_No spacer_ β2.7. The products were screened by digestion with NdeI and NheI. Subsequently a spacer sequence was introduced downstream of this construct by a 2 step nested PCR. In the first step, a fragment of the β2.7 ( β2.7F) was PCR amplified with a common forward primer and a reverse primer introducing a part of the spacer sequence (SF1). In the next step, the step 1 product was PCR amplified with the same forward primer as in step I and a reverse primer, which binds to SF1 and simultaneously introduces the remaining spacer sequence (SF2) and the HindIII site for cloning. This reconstituted the spacer sequence. The step 2 PCR product was cloned into the ATP6_No spacer_ β2.7 to obtain the ATP6_ β2.7.

FIG. 17 shows a schematic representation of strategy used to generate β2.7_ATP8 construct. The purified ATP8 target sequence was PCR amplified with the reverse primer, thereby introducing a SpeI site. Simultaneously, a fragment was amplified from β2.7 by PCR so that the resulting product carried a BamHI and HindIII site at the 5′ end and at the 3′ end, respectively. The β2.7 PCR product and the ATP8 PCR product were single digested with HindIII and SpeI, respectively, mixed in equimolar amounts and ligated at 22° C. for 4 hours. SpeI site can be ligated to HindIII site by a 2 base fill in which effectively destroys both sites. Post-ligation, the product of the correct size was purified from the gel, which has the β2.7 fragment fused to the antisense ATP8 sequence. Then, this ligation product was PCR amplified to introduce the HindIII site and cloned back into the β2.7_pVAX1 vector backbone to generate the intact sequence.

FIG. 18 shows data and schematics of MT-ATP6 antisense tandem repeat fusion RNA ATP6_(D3)4_(D2)4 localising in the mitochondria and triggering highly efficient functional MT-ATP6 knockdown. a) shows a schematic representations of the ATP6_(D3)4_(D2)4 fusion RNA (upper panel) and ATP6_(D3)4_(D2)4 mfe structure (lower panel). b) shows the results of agarose gel electrophoresis of in vitro transcribed RNAs ATP6_pVAX, ATP6_ β2.7, and ATP6_(D3)4_(D2)4. c) shows the western blot results of HEK293T cells, which were transfected with antisense fusion RNAs. The levels of ATP6 protein were monitored by western blot analyses 24 hours post-transfection. Mt-COXII (cytochrome c oxidase polypeptide II) was used as a mitochondrial loading control. d) shows a column graph depicting the reduction in ATP levels in HEK293T cells, which were transfected with antisense fusion RNAs along with the control RNAs. Reductions of ATP levels were determined 24 hours post-transfection using Cell Titre-Glo Assay. A cells-only control was used for normalization of the data sets (Dunnett's multiple comparisons test). e) shows images of ethidium bromide-free agarose gel electrophoresis of equimolar amounts of in vitro transcribed fluorescein-12 uracil-labelled RNAs. f) provides column graphs showing the fluorescence intensity to uracil ratios of fluorescein labelled RNAs. g) depicts Mander's overlap coefficient for either HEK293T or HepG2 (h)) cells, which were transfected with fluorescein-12 uracil-labelled RNA. Co-localization of the fluorescein-12 uracil-labelled RNA with the MitoTracker-Orange stained mitochondria was quantified from confocal microscopy images using ImageJ algorithm JACOP. Co-localization represented by Mander's overlap coefficient is shown. RNA labelling intensity-adjusted relative to Mander's overlap coefficient in HEK293T (i)) and HepG2 (j)) are shown. Five images were used for analysis (Tukey's multiple comparison test). Data represent each averages of five representative images±SD. Significance of the data in g) and h) was tested using 1-way ANOVA with Dunnett's multiple comparisons test. Significance of the data in i) and j) was tested using Tukey's multiple comparison test.

FIG. 19 shows HEK293T (a) or HepG2 (b) cells were transfected with fluorescein-12 uracil-labelled in vitro transcribed RNAs (green), nuclei were stained with Hoechst 33342 (blue), and mitochondria were stained with MitoTracker (red). Co-localisation of transfected RNA with mitochondria is indicated by a yellow signal in the overlay images.

FIG. 20 shows minimum free energy (mfe) structures of tandem repeat RNAs, namely (D2)₄, (D3)₄, (D3)₄_(D2)₄_(D2)₄_(D3)₄, and (D3_D2)₄. These structures are as predicted by mfold.

FIG. 21A-21D show Formulas I-IV that are described herein.

### DEFINITION OF TERMS

The term “naked nucleic acid” refers to a nucleic acid (either DNA or RNA) that is, as opposed to non-viral or viral vectors, not complexed with any other compound neither with histones, proteins, lipids, sugars, nanoparticular structures, viral capsids or envelopes nucleic acid that occurs, for example, during cell to cell transfer or transformation of cells with nucleic acid sequences.

The term “coding/non-coding nucleic acid sequences” refers to both coding and non-coding nucleic acid sequences. A non-coding nucleic acid (that is for example RNA or DNA) is a nucleic acid molecule that is not translated into a protein. Conversely, a coding nucleic acid molecule is a nucleic acid molecule that is translated into a protein. For example, when used in regards to RNA, these non-coding RNA are also termed non-protein-coding RNA (npcRNA), non-messenger RNA (nmRNA) and functional RNA (fRNA). For example, a DNA sequence from which a functional, non-coding RNA is transcribed is often known as an RNA gene. Examples of non-coding RNA genes include, but are not limited to, highly abundant and functionally important RNAs such as, for example, transfer RNAs (tRNAs), ribosomal RNAs (rRNAs), as well as RNAs such as small nucleolar RNAs (snoRNAs), microRNAs, small interfering RNAs (siRNAs), antisense RNAs (asRNA), small nuclear RNAs (snRNA or U-RNA), exosomal/extracellular RNAs (exRNAs), Piwi-interacting RNAs (piRNA), small Cajal body RNA genes (scaRNAs) and long non-coding RNAs (ncRNAs or lncRNAs), which include examples such as, but not limited to, X-inactive specific transcript (Xist) and HOX transcript antisense RNA (HOTAIR). The number of non-coding RNAs encoded within the human genome is unknown; however, transcriptomic and bioinformatic studies suggest the existence of thousands of non-coding RNAs. Since many of the newly identified non-coding RNAs have not been validated for their function, it is possible that many are non-functional. It is also likely that many non-coding RNAs are non-functional (often termed “junk RNA”), and are the result of spurious transcription.

The term “sense” and “antisense” refers to concepts used to compare the polarity of nucleic acid molecules, such as DNA or RNA, to other nucleic acid molecules. Depending on the context, these sense and antisense molecules may refer to different molecules compared to the common 5′-3′ naming convention for nucleic acid sequences. For example, in double stranded DNA (dsDNA), a single strand of DNA may be called the sense strand (or positive (+) strand), if the RNA version of the same sequence is translated or translatable into proteins. The complementary strand to this positive DNA strand is called the antisense (or negative (−) strand). This is not to be confused with the concept of coding and non-coding nucleic acid sequences, as defined above. As an example, the two complementary strands of double-stranded DNA (dsDNA) are usually differentiated as the “sense” strand and the “antisense” strand. The DNA sense strand looks like the messenger RNA (mRNA) and can be used to read the expected protein code; for example, ATG in the sense DNA may correspond to an AUG codon in the mRNA, encoding the amino acid methionine. However, the DNA sense strand itself is not used to make protein by the cell. It is the DNA antisense strand which serves as the source for the protein code, because, with bases complementary to the DNA sense strand, it is used as a template for the mRNA. Since transcription results in an RNA product complementary to the DNA template strand, the mRNA is complementary to the DNA antisense strand. The mRNA is what is used for translation (protein synthesis). In an example for RNA, antisense RNA is an RNA sequence (or transcript) that is complementary to endogenous mRNA. In other words, it is a non-coding strand complementary to the coding sequence of RNA. Introducing a transgene coding for antisense RNA is, for example, a technique used to block expression of a gene of interest.

### DETAILED DESCRIPTION OF THE PRESENT INVENTION

Mitochondria, a double membrane-bound organelle found in all eukaryotic organisms, are typically associated with ATP production in all living eukaryotic cells. However, defects in enzymes that form part of the respiratory cycle result in, for example, mitochondria-associated diseases, which can be difficult to treat due to the inaccessibility of the mitochondrial genome. Therefore, in order to treat such diseases associated with, for example, defects in the mitochondrial genome, the present disclosure identifies subdomains and combinations of RNA sequences within, for example, the human cytomegalovirus (CMV) β2.7 RNA for targeted delivery of RNA into mitochondria, using the propensity the human cytomegalovirus β2.7 RNA for targeting and co-localising into mitochondria. Thus, disclosed herein is the mitochondrial delivery of a recombinant coding RNA into the mitochondria, which leads to, for example recombinant mitochondrial gene expression. Also disclosed herein is the mitochondrial delivery of, for example, a non-coding antisense RNA into the mitochondria, which triggers functional knockdown of mitochondrial gene expression. The identified sequences are for use in gene therapy to, for example, suppress mitochondrial malfunction, or to restore mitochondrial gene functions in neurodegenerative, or other mitochondria-associated diseases, or for anti-ageing purposes.

It was shown that the 5′ terminal part of the human cytomegalovirus β2.7 RNA, the so-called p137 RNA, co-localizes with mitochondrial complex I protecting complex I activity, however, functional coding or non-coding RNA has not yet being delivered using the β2.7 RNA sequences. The generation of a vector system that allows delivering recombinant nucleic acids into the mitochondria allow for, for example the genetic therapy of mitochondria-associated, yet incurable, human diseases. The distinct non-coding RNA originating from the human cytomegalovirus, the so called β2.7 RNA, was found to localize to the mitochondria of mammalian cells and bind to mitochondrial complex I. Thus, in one example, the nucleic acid delivery system as disclosed herein comprises RNA, or DNA, or combinations thereof. In another example, the nucleic acid delivery system comprises RNA. In another example, the nucleic acid delivery system comprises DNA.

Thus, in one example, the present invention refers to a nucleic acid delivery construct comprising at least one sense or antisense RNA subdomain of the human cytomegalovirus β2.7 RNA, wherein each subdomain is capable of localisation within the mitochondria. In another example, the nucleic acid delivery construct comprises at least one sense RNA subdomain of the human cytomegalovirus β2.7 RNA. In another example, the nucleic acid delivery construct comprises at least one antisense RNA subdomain of the human cytomegalovirus β2.7 RNA. In yet another example, the nucleic acid delivery construct comprises one or more sense or antisense RNA subdomains of the human cytomegalovirus β2.7 RNA.

The nucleic acid delivery construct, as disclosed herein, can comprise a number of sense or antisense RNA subdomains. In one example, the number of subdomains in the nucleic acid delivery construct is, but is not limited to, between 1 to 10 subdomains, between 5 to 15 subdomains, between 12 to 22 subdomains, or 1, 2, 3, 4, 5, 6, 7, 8, 9, 10, 11, 12, 13, 14, 15 or 16 subdomains. In other words, a nucleic acid delivery construct according to the present disclosure comprises between 1 to 10 RNA sequences, between 5 to 15 RNA sequences, between 12 to 22 RNA sequences, or 1, 2, 3, 4, 5, 6, 7, 8, 9, 10, 11, 12, 13, 14, 15 or 16 RNA sequences.

The present disclosure relates to the identification of RNA sequences within CMV β2.7 RNA for targeted delivery into mitochondria. That is to say that the human cytomegalovirus β2.7 RNA, when introduced to a cell, seeks out and enters the mitochondria, and, as a result, is not found in the cytoplasm of said cell. The same can be said for each of the RNA subdomains of the human cytomegalovirus β2.7 RNA. The identified RNA sequences consist of four thermodynamically conserved structural sub-domains (D1 to D4). From these sub-domains, tandem repeats and combinations of RNA sequences are constructed. Tandem repeats constructed from functionally relevant domains, for example domain 2 (D2X4) and domain 3 (D3X4), among which, for example, domain 3 (D3X4), exhibits enhanced mitochondrial localization potential. Combination of tandem repeats are constructed as, for example, (D3X4_D2X4 or D2X4_D3X4), in which (D3X4_D2X4) exhibits highest mitochondrial targeting potential. Domain 1 and 4 exhibit similar structures on the antisense transcript and the antisense domains AS1 and AS4 exhibit substantial mitochondrial localization potential. Delivery of CMV β2.7 RNA-derived sequences with coding RNA into mitochondria leads to recombinant mitochondrial gene expression. For example, the dual tetrameric of domains D3 and D2 (D3x4_D2x4) protects mitochondrial complex I with higher efficiency than the wild type β2.7 RNA.

Thus, in one example, the nucleic acid sequence is as disclosed herein, wherein each subdomain is capable of localisation within the mitochondria but does not localise into the cytoplasm.

Disclosed herein are isolated RNA sequences of the human cytomegalovirus p137 RNA, which is the 5′ terminal end of the human cytomegalovirus β2.7 RNA. This 5′ terminal end of the human cytomegalovirus β2.7 RNA sequences comprises of four, thermodynamically conserved, structural subdomains, named D1 to D4, respectively, each of which is capable of targeting the mitochondria of a cell. Thus, in one example, the nucleic acid delivery construct, as disclosed herein, comprises RNA sequences from human cytomegalovirus β2.7 RNA, which are, but are not limited to, β2.7 RNA (SEQ ID NO: 1 or SEQ ID NO: 2), domain 1 (D1; SEQ ID NO: 3 or SEQ ID NO: 7) of β2.7 RNA, domain 2 (D2; SEQ ID NO: 4 or SEQ ID NO: 8) of β2.7 RNA, domain 3 (D3; SEQ ID NO: 5 or SEQ ID NO: 9) of β2.7 RNA, domain 4 (D4; SEQ ID NO: 6 or SEQ ID NO: 10) of β2.7 RNA and combinations thereof. In one example, the nucleic acid delivery construct comprises one type of RNA sequence as disclosed herein. Examples of types of RNA sequences are, but are not limited to, sense RNA, antisense RNA, messenger RNA (mRNA), transfer RNA (tRNA), microRNA (miRNA), small interfering RNA (siRNA), small nucleolar RNA (snRNA), Piwi-interacting RNA (piRNA), tRNA-derived RNA (tsRNA), small rDNA-derived RNA (srRNA), ribosomal RNA (rRNA), long non-coding RNA (lncRNA), short hairpin RNA (shRNA) and transfer-messenger RNA (tmRNA). In one example, the nucleic acid delivery construct comprises sense RNA. In another example, the nucleic acid delivery construct comprises antisense RNA. In a further example, the nucleic acid delivery construct comprises a combination of sense and antisense RNA. In another example, the nucleic acid delivery construct comprises a combination of the RNA sequences as disclosed herein. In yet another example, the nucleic acid delivery construct comprises a combination of the RNA sequences as disclosed herein, wherein the nucleic acid delivery construct can comprise multiple repeats of a single RNA sequence. In another example, the nucleic acid delivery construct comprises the full length sequence of β2.7 RNA (SEQ ID NO: 1 (sense)). In another example, the nucleic acid delivery construct comprises the full length sequence of β2.7 RNA (SEQ ID NO: 2 (antisense)). In yet another example, the nucleic acid delivery construct comprises domain 1 of β2.7 RNA (SEQ ID NO: 3 (sense). In yet another example, the nucleic acid delivery construct comprises domain 1 of β2.7 RNA (SEQ ID NO: 7 (antisense)). In a further example, the nucleic acid delivery construct comprises domain 2 of β2.7 RNA (SEQ ID NO: 4 (sense)). In one example, the nucleic acid delivery construct comprises domain 2 of β2.7 RNA (SEQ ID NO: 8 (antisense)). In another example, the nucleic acid delivery construct comprises domain 3 of β2.7 RNA (SEQ ID NO: 5 (sense)). In yet another example, the nucleic acid delivery construct comprises domain 3 of β2.7 RNA (SEQ ID NO: 9 (antisense)). In a further example, the nucleic acid delivery construct comprises domain 4 of β2.7 RNA (SEQ ID NO: 6 (sense)). In one example, the nucleic acid delivery construct comprises domain 4 of β2.7 RNA (SEQ ID NO: 10 (antisense)). In another example, the nucleic acid delivery construct comprises domain 2, domain 3 and domain 4 of β2.7 RNA (SEQ ID NO: 11 (sense) or SEQ ID NO: 39 (antisense)). In a further example, the nucleic acid delivery construct comprises domain 1, domain 3 and domain 4 of β2.7 RNA (SEQ ID NO: 12 (sense) or SEQ ID NO: 40 (antisense)). In a further example, the nucleic acid delivery construct comprises domain 1, domain 2 and domain 4 of β2.7 RNA (SEQ ID NO: 13 (sense) or SEQ ID NO: 41 (antisense)). In another example, the nucleic acid delivery construct comprises domain 1, domain 2 and domain 3 of β2.7 RNA (SEQ ID NO: 14 (sense) or SEQ ID NO: 42 (antisense)).

As used herein, the term “sequence identity” refers to the situation where two polynucleotide or amino acid sequences are identical, or have a number of identical residues (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “sequence identity”, as used herein, denotes a characteristic of a polynucleotide or amino acid sequence, wherein the polynucleotide or amino acid may comprise a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24 to 48 nucleotide (8 to 16 amino acid) positions, wherein the percentage of sequence identity is calculated by comparing the reference sequence to the sequence, which may include deletions or additions, which total 20 percent or less of the reference sequence over the comparison window. The reference sequence may be a subset of a larger sequence. Thus, in another example, the nucleic acid delivery system, as disclosed herein, comprises RNA sequences from human cytomegalovirus β2.7 RNA, wherein the RNA sequences have a sequence identity of between 70% to 99%, of between 75% to 85%, of between 78% to 88%, of between 80% to 89%, of about 90%, of about 91%, of about 92%, of about 93%, of about 94%, of about 95%, of about 96%, of about 97%, of about 98%, or of about 99% of one or more of the RNA sequences disclosed herein. In one example, the nucleic acid delivery construct is as disclosed herein, wherein the RNA sequences from human cytomegalovirus β2.7 RNA has a sequence identity of about 90%, of about 91%, of about 92%, of about 93%, of about 94%, of about 95%, of about 96%, of about 97%, of about 98%, or of about 99% of one or more of the RNA sequences, which are, but are not limited to, β2.7 RNA (SEQ ID NO: 1 or SEQ ID NO: 2), domain 1 (D1; SEQ ID NO: 3 or SEQ ID NO: 7) of β2.7 RNA, domain 2 (D2; SEQ ID NO: 4 or SEQ ID NO: 8) of β2.7 RNA, domain 3 (D3; SEQ ID NO: 5 or SEQ ID NO: 9) β2.7 RNA, domain (D4; SEQ ID NO: 6 or SEQ ID NO: 10) of β2.7 RNA and combinations thereof.

The nucleic acid delivery constructs, as disclosed herein, can also further include one or more changes in the nucleic acid sequence. In one example, such a change is a mutation in the RNA sequence. In another example, the nucleic acid delivery construct comprises one or more non-structural mutations. In yet another example, the nucleic acid delivery construct comprises one or more structure neutral mutations. As used herein, the term “non-structural mutation” or “structure neutral mutation” refers to a mutation in a nucleic acid sequence which changes the sequence of the nucleic acid sequence, but preserves the functional structure of the mutated sequence compared to the unmutated sequence.

From these subdomains, tandem repeats and combinations of RNA sequences are constructed. As used herein, the term “tandem repeats” refers to sections within a nucleic acid sequence where a pattern of one or more nucleotides is repeated and the repetitions are directly adjacent to each other. For example, a sequence of ATGGC repeated 3 times in a row, thus resulting in a sequence comprising ATGGC ATGGC ATGGC, is understood to be a tandem repeat. Based on the invention as disclosed herein, sequences are constructed from functional RNA domains, that is from any of the subdomains D1, D2, D3 or D4. Examples of such constructed domains are, but are not limited to, domain 2 (a four-time repeat of domain 2, in other words D2X4) and domain 3 (a four-time repeat of domain 3, in other words D3X4). Thus, in one example, the nucleic acid delivery system as disclosed herein comprises combinations and/or multiples of the RNA sequences disclosed herein, including, but not limited to, duplicates (2), triplicates (3), quadruplicates (4), quintuplicates (5), sextuplicates (6), septuplicates (7) octuplicates (8) or longer repeats of single domains. In other words, the combinations and/or multiples of the RNA sequences disclosed herein include, but are not limited to, dimers, trimers, tetramers, or polymers of single domains.

As used herein, the term “spacer” refers to a sequence of nucleic acids that are inserted at either the 5′ or 3′ end of a nucleic acid sequence, or at both ends of a nucleic acid sequence, within a construct. The spacer is inserted at defined positions within the nucleic acid sequence in order to ensure that the structural integrity of a nucleic acid sequence, for example in an RNA sequence, remains. This ensures the retention of function or characteristic of the RNA sequence, for example, the mitochondrial targeting capability of the nucleic acid construct. The spacer also functions to prevent any steric effects from occurring and to enable the nucleic acid sequence to attain its natural tertiary structure, thereby also facilitating the retention of its function. Thus, in one example, the nucleic acid delivery construct as disclosed herein comprises between 1 to 10, between 5 to 15, between 8 to 24, at least one, at least two, at least three, at least four, at least 5, about 6, about 7, about 8 or about 9 spacer sequences. In one example, the nucleic acid delivery construct comprises about 6 spacer sequences. In another example, the nucleic acid delivery construct comprises about 7 spacer sequences. In yet another example, the nucleic acid delivery construct comprises about 8 spacer sequences. In a further example, the nucleic acid delivery construct comprises about 9 spacer sequences.

As stated above, the spacers sequences can be placed anywhere within the nucleic acid sequence. For example, spacers can found at the beginning (that is the 5′ end) of an RNA sequence. Spacers can also be found at the end (that is the 3′ end) of an RNA sequence. When more than one spacer is used, these spacers can also be found at both the 5′ and 3′ ends of an RNA sequence. Thus, in one example, when the nucleic acid delivery construct comprises at least two or more spacers, at least one spacer sequence is at the 5′ end and at least one other spacer sequence is at the 3′ end of the RNA sequence of human cytomegalovirus β2.7 RNA.

The length of a spacer is defined, for example, by the specific function that the spacer is intended to fulfil. For example, if the function of a spacer is to prevent steric hindrance between two or more RNA sequences, this spacer could then be between tens to hundreds of nucleotides long, depending on the size of the resulting RNA structure. In other words, the spacer sequence disclosed in the present invention is sufficiently long to prevent any steric hindrance from arising between neighbouring RNA subdomains and/or wherein the length of the spacer sequence is sufficiently long to allow neighbouring RNA subdomains to fold into their thermodynamically preferred structure. Having said that, a person skilled in the art would appreciate that the spacer length is dependent on the length, structure, and combination(s) of the at least one sense or antisense RNA subdomains as disclosed in the nucleic acid delivery system disclosed herein. In one example, the spacer sequence is between 5 to 40 nucleotides, between 5 to 30 nucleotides, between 6 to 10 nucleotides, between 8 to 14 nucleotides, between 15 to 20 nucleotides, between 22 to 28 nucleotides, between 25 to 37, between 28 to 39 nucleotides, about 7 nucleotides, about 9 nucleotides, about 11 nucleotides, about 12 nucleotides, about 13 nucleotides, about 15 nucleotides, about 17 nucleotides, about 21 nucleotides, about 27 nucleotides, about 29 nucleotides, about 30 nucleotides, about 34 nucleotides, about 36 nucleotides in length. In one example, the spacer sequence is 5 nucleotides long. In another example, the spacer sequence is 6 nucleotides long. In yet another example, the spacer sequence is 13 nucleotides long. In a further example, the spacer sequence is 17 nucleotides long. In one example, the spacer sequence is 24 nucleotides long. In yet another example, the spacer sequence is 32 nucleotides long.

In one example, the spacer sequence is, but is not limited to S1a (SEQ ID NO:26), S1b (SEQ ID NO:27), S2a (SEQ ID NO:28), S2b (SEQ ID NO:29), S3a (SEQ ID NO:30), S3b (SEQ ID NO:31), S4a (SEQ ID NO:32), S4b (SEQ ID NO:33), Sha (SEQ ID NO:34), S6b (SEQ ID NO:35), S8a (SEQ ID NO:36), S8b (SEQ ID NO:37) and Spacer F3A (SEQ ID NO: 38), and combinations thereof.

Spacer sequences may also contain functional nucleic acid sequences, or other structural or functional motifs. For example, a spacer sequence can further optionally comprise a stop codon.

As used herein, the term “nuclear localization signal” or “NLS” refers to nucleic acid sequences coding for one or more additional secretory signals or signalling peptides. These nuclear localization sequences can be added to the 5′ or 3′ end of the nucleic acid sequence, thereby resulting in the expression of such a nuclear localization sequence at the C-terminus or N-terminus or both the C- and N-termini of a peptide. One example known in the art is the addition of a nuclear localisation sequence (NLS) which directs the nascent protein for import from the cytoplasm into to the nucleus of the cell. There are many different versions of nuclear localisation sequences and their length and composition is dependent on the cell type from which they have been isolated. For example, the nuclear localisation sequence of nucleoplasmin is “AVKRPAATKKAGQAKKKKLD”, whereas the nuclear localisation sequence of c-myc is “PAAKRVKLD”. Thus, in one example, the nucleic acid delivery construct further optionally comprises a nuclear localization signal (NLS).

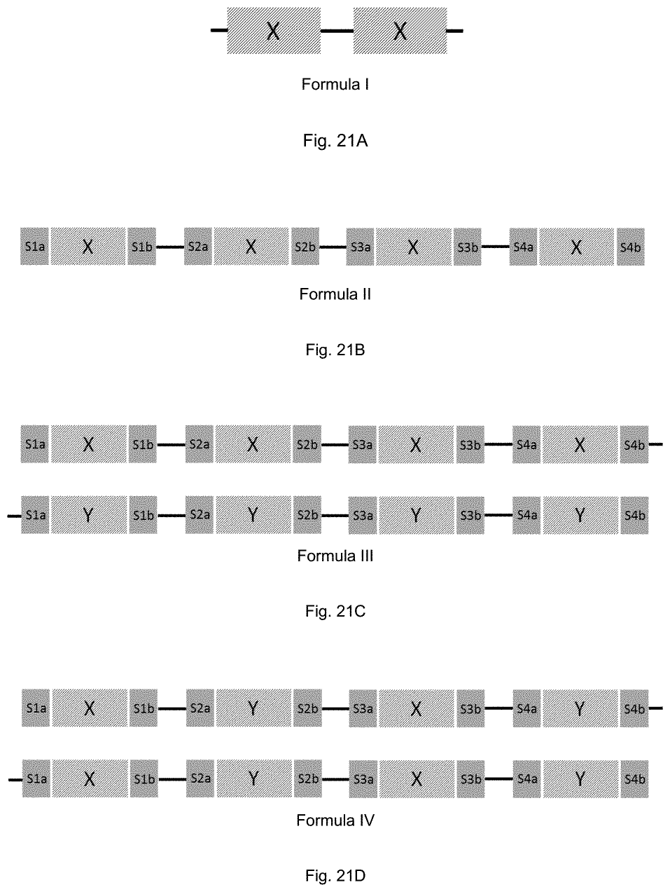

In one example, the nucleic acid delivery system disclosed herein is a RNA sequence according to the formula I shown in FIG. 21A:
wherein each X is independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein each X is optionally preceded or followed or flanked by at least one or more spacer sequences as defined herein. In other words, in one example, the nucleic acid delivery system is a dimer, wherein each X is independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof; and wherein each X is optionally preceded or followed or flanked by at least one or more spacer sequences as defined herein.

In one example, the nucleic acid delivery system disclosed herein is a RNA sequence according to the formula II shown in FIG. 21B:
wherein each X is independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10), or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as disclosed herein. In one example, X is D2 (SEQ ID NO: 4 or SEQ ID NO: 8). In another example, X is D3 (SEQ ID NO: 5 or SEQ ID NO: 9). In yet another example, the nucleic acid delivery construct according to formula II comprises a nucleic acid sequence according to SEQ ID NO: 15 or SEQ ID NO: 51. In a further example, the nucleic acid delivery construct according to formula II comprises a nucleic acid sequence according to SEQ ID NO: 16 or SEQ ID NO: 52. In other words, in one example, the nucleic acid delivery system is a tetramer, wherein each X is independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof; and wherein each X is optionally preceded or followed or flanked by at least one or more spacer sequences as defined herein.

In one example, the nucleic acid delivery system disclosed herein is a RNA sequence according to the formula III shown in FIG. 21C:
wherein X and Y are different from each other, wherein each X and each Y are independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as defined herein. In one example, X is D3 (SEQ ID NO: 5 or SEQ ID NO: 9) and Y is D2 (SEQ ID NO: 4 or SEQ ID NO: 8). In another example, X is D2 (SEQ ID NO: 4 or SEQ ID NO: 8) and Y is D3 (SEQ ID NO: 5 or SEQ ID NO: 9). In a further example, the nucleic acid delivery construct according to formula III comprises a nucleic acid sequence according to SEQ ID NO: 17 or SEQ ID NO: 53. In another example, the nucleic acid delivery construct according to formula III comprises a nucleic acid sequence according to SEQ ID NO: 18. In other words, in one example, the nucleic acid delivery system is a tetramer, wherein each X and Y are independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof; and wherein each X and Y are optionally preceded or followed or flanked by at least one or more spacer sequences as defined herein.

In one example, the nucleic acid delivery system disclosed herein is an octamer of RNA sequences according to the formula IV shown in FIG. 21D:
wherein X and Y are different from each other, wherein each X and each Y are independently, but not limited to, D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as defined herein. In one example, X is D3 (SEQ ID NO: 5 or SEQ ID NO: 9) and Y is D2 (SEQ ID NO: 4 or SEQ ID NO: 8). In another example, the nucleic acid delivery construct according to formula III comprises a nucleic acid sequence according to SEQ ID NO: 19.

In order for the claimed nucleic acid delivery system to function as a delivery system, the nucleic acid sequence needs to further comprise a payload. As used herein, the term “payload” refers to one or more nucleic acid sequences that can be inserted into the sequence of the nucleic acid delivery system, which, as a result of its insertion, then acts on or within the mitochondria of the cell. A payload can be, but is not limited to, a recombinant nucleic acid sequence, RNA, DNA, modified nucleic acids, nucleic acid analogues and nucleic acid mimics including pyranosyl nucleic acids (p-RNA), threose nucleic acids (TNA), glycol nucleic acids (GNA), peptide nucleic acids (PNA), alanyl nucleic acids (ANA), locked nucleic acids (LNA), morpholinophosphoramidates (MF), non-nucleic acid-based molecules including peptides, proteins, lipids, carbohydrates, synthetic polymers, small molecular weight compounds, and the like. In one example, the recombinant nucleic acid sequence is, but is not limited to, non-coding nucleic acid sequence, coding nucleic acid sequence, single-stranded nucleic acid sequence, linear double-stranded nucleic acid sequence, antisense nucleic acid sequences, sense nucleic acid sequence circular single-stranded nucleic acid sequence and circular double-stranded nucleic acid sequence. In another example, the recombinant nucleic acid sequence is a complete, natural or recombinant mitochondrial genome. In yet another example, the nucleic acid delivery construct comprises a sequence according to any one of SEQ ID NO: 20 to SEQ ID NO: 82.

Furthermore, said payload needs to be attached to the nucleic acid delivery system in order to be able to be transported. In one example, the payload is covalently linked to the nucleic acid delivery system. In another example, the payload is non-covalently linked to the nucleic acid delivery system. Non-covalent linkage can be achieved via electrostatic interactions including ionic interactions, hydrogen bonding, or halogen bonding, via Van der Waals forces including dipole-dipole interactions, induced dipole interactions, or London dispersion forces, via π-effects including π-π interactions, cation- or anion-π interactions, or polar-π interactions, or via hydrophobic effects. A covalent linkage, on the other hand, is a linkage that involves the sharing of electron pairs between atoms. Examples of covalent bonds or linkages include many kinds of interactions including, but not limited to, σ-bonding, π-bonding, metal-to-metal bonding, agostic interactions, bent bonds, and three-centre two-electron bonds.

Akin to the physical concepts governing, for example, the aeronautical concept of payload transportation, a person skilled in the art would appreciate that the size of a payload dictates the size of the carrier (that is the nucleic acid delivery system) required to carry such a payload to its intended destination. Thus, in one example, the total size of nucleic acid delivery construct is proportional to the size of a payload. This means that a nucleic acid delivery system for a payload of, for example, 200 nucleic acids in length, would be four times larger than a nucleic acid delivery system of a payload which is only 50 nucleic acids long. Conversely, a payload, which is only 10 nucleic acids long, can make use of a nucleic acid delivery system that is half the size of a nucleic acid delivery system for a payload which is 20 nucleic acids long. Thus, in one example, the nucleic acid delivery system is scalable. In another example, the nucleic acid delivery system is scalable according to, or proportionally to, the size of the payload.

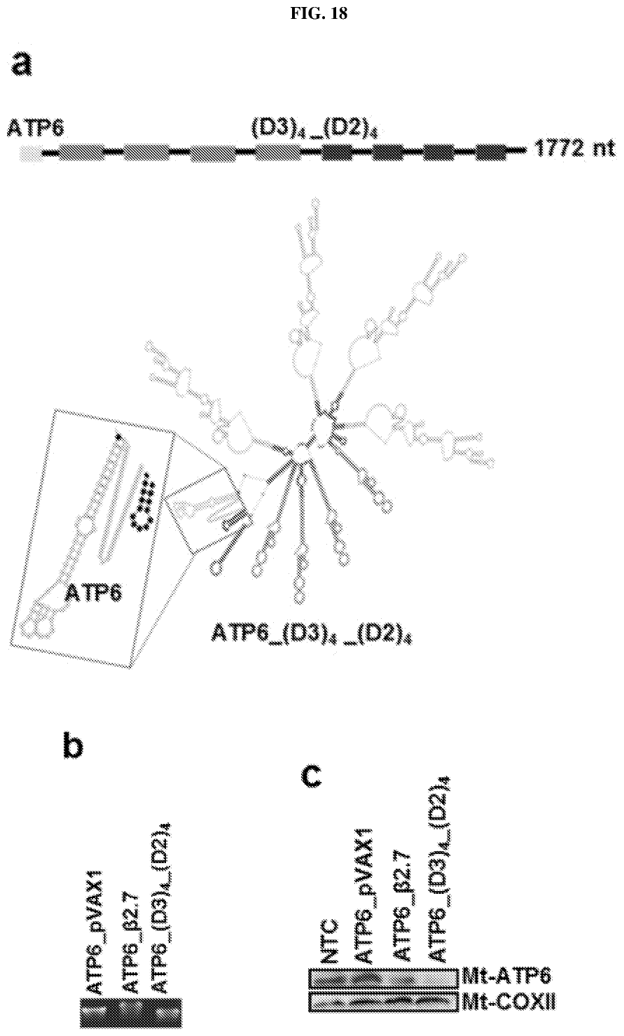

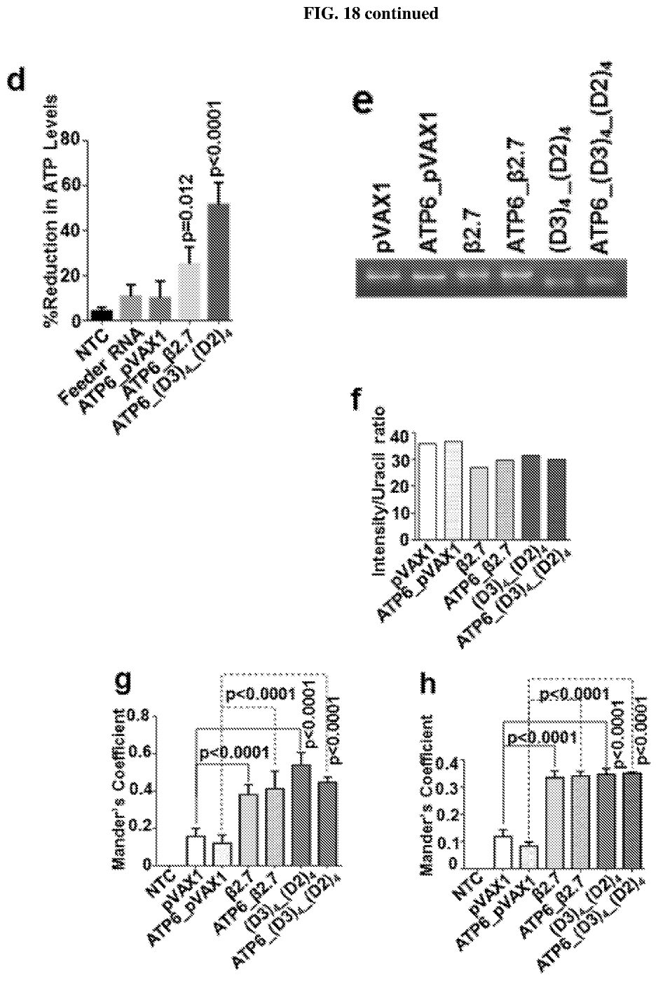

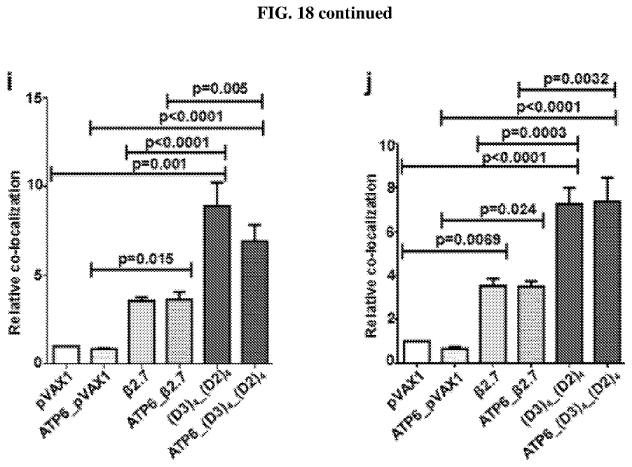

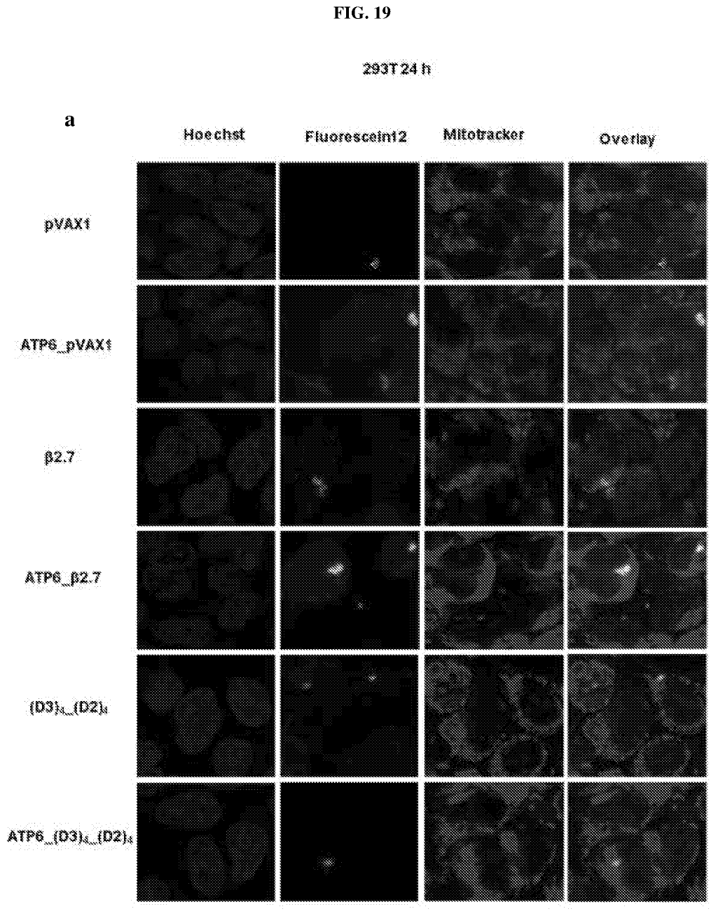

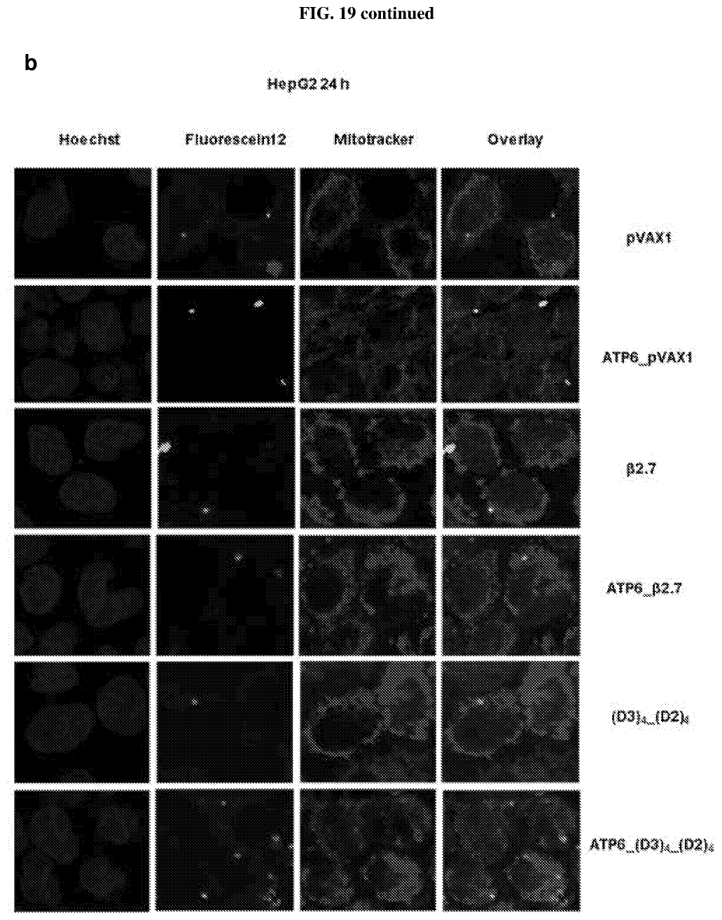

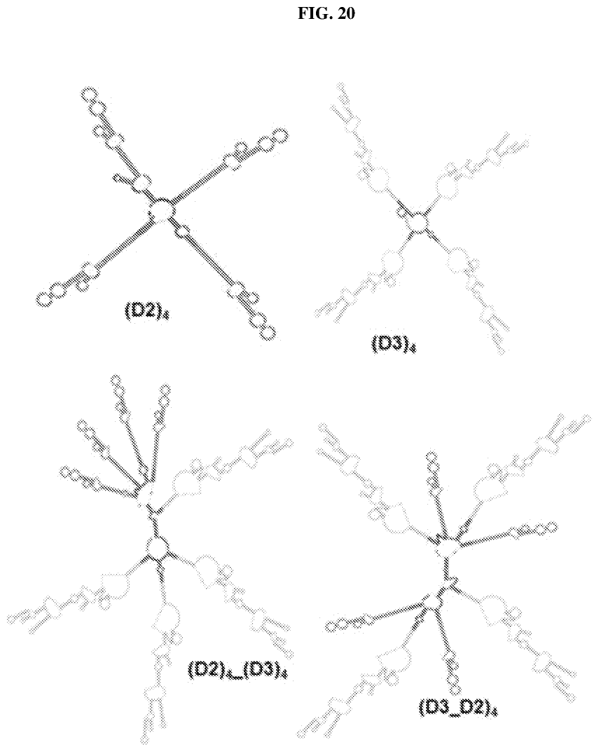

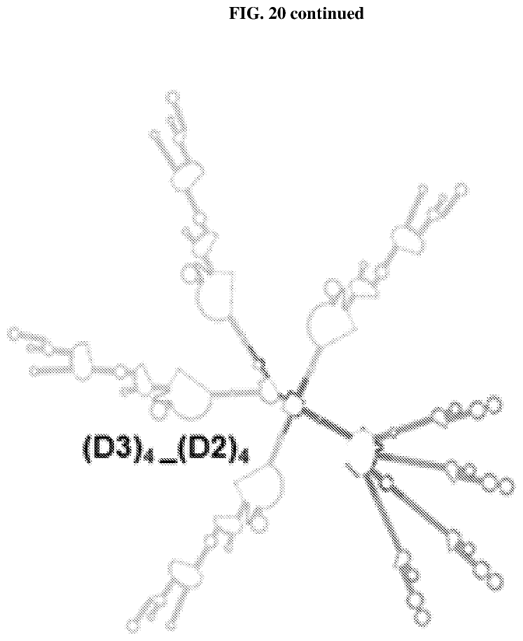

The potential of the most active RNA, (D3)4_(D2)4, to co-deliver the MT-ATP6-directed antisense RNA into the mitochondria was investigated. To this end, the MT-ATP6 antisense RNA was fused to the 5′ end of (D3)4_(D2)4 via a spacer, thereby generating the construct ATP6_(D3)4_(D2)4. The spacer was selected to preserve both the open structure of the antisense RNA, as well as the domain structures within (D3)4_(D2)4 according to predictions with mfold (FIG. 18a). HEK293T cells were transfected with the in vitro transcribed RNAs and target gene knockdown was monitored on the protein level using western blot 24 hours post transfection (FIG. 18b,c). Compared with ATP6_β2.7 RNA, the ATP6_(D3)4_(D2)4 RNA triggered a substantially stronger knockdown of the MT-ATP6 protein leading to a 2.1-fold higher reduction of cellular ATP levels (FIG. 18d). This data indicates mitochondrial targeting is scalable and not restricted by an impaired by increasing length of the targeting vector. In order to have direct proof of mitochondrial RNA targeting, RNAs were labelled with fluorescein-12-uracil during in vitro transcription, and the integrity and labelling efficiency of the RNA was assessed using agarose gel electrophoresis (FIG. 18e). Band intensities were quantified using the software ImageJ v1.48 (FIG. 19) and intensity to uracil count or length ratios were calculated (FIG. 18f). These ratios were comparable for all RNAs, indicating similar labelling efficiencies. Subsequently, HEK293T or HepG2 cells were transfected with the labelled RNA, and mitochondria stained with MitoTracker Orange (FIG. 19). The Manders overlap coefficient (MOC) was determined as a metric of co-localisation of the labelled RNA and mitochondria (FIG. 18g,h). All β2.7-RNA/-domain chimeras showed significant levels of co-localisation compared with the β2.7-negative RNAs. RNAs harbouring the (D3)4_(D2)4 tandem repeat structures exhibited a significantly stronger co-localisation effect compared with β2.7-RNA containing RNAs when comparing the RNA-labelling, intensity-adjusted Manders overlap coefficients in HEK293T (FIG. 18i) or HepG2 cells (FIG. 18j).

Also encompassed in the present disclosure are vectors, recombinant cells, recombinant organisms and nucleic acid sequences, which comprise or express the nucleic acid delivery system as disclosed herein. In one example, a vector comprises the nucleic acid delivery system as disclosed herein. In another example, the vector comprises a naked nucleic acid, or a non-viral vector, or a viral vector, or combinations thereof.

In one example, a recombinant cell comprises the nucleic acid sequence as disclosed herein. In another example, the recombinant cell expresses the nucleic acid sequence in a consecutive manner (that is, consecutively). In yet another example, the recombinant cell expresses the nucleic acid sequence in a non-consecutive manner (that is, non-consecutively). In one example, a recombinant organism comprises the nucleic acid sequence as disclosed herein. In another example, the recombinant organism expresses the nucleic acid sequence in a consecutive manner (that is, consecutively). In yet another example, the recombinant organism expresses the nucleic acid sequence in a non-consecutive manner (that is, non-consecutively).

In one example, a nucleic acid sequence comprises at least one or more sense or antisense RNA sequences of the human cytomegalovirus β2.7 RNA. In another example, the RNA sequences of the human cytomegalovirus β2.7 RNA are, but are not limited to, domain 1(D1; SEQ ID NO: 3 or 7), domain 2 (D2; SEQ ID NO: 4 or 8), domain 3 (D3; SEQ ID NO: 5 or 9) and domain 4 (D4; SEQ ID NO: 6 or 10).

Also disclosed within the scope of the present invention are methods of treating diseases. Mitochondrial disorders are usually caused by heterogeneity resulting from unequal segregation of defective mitochondrial DNA (mDNA). Thus, one therapeutic method is to reduce the abundance of defective messenger RNA (mRNA), thereby allowing the wild type messenger RNA to re-populated the mitochondria. The identified sequences can thus be used in gene therapy to suppress mitochondrial malfunction, or to restore mitochondrial gene functions in neurodegenerative, or other mitochondria-associated diseases, or for anti-aging purposes.

The parental human cytomegalovirus β2.7 RNA can protect the mitochondrial complex I from certain inhibitors and, thus protect the mitochondria from oxidative stress and DNA damage, thereby increasing cell viability. The parental human cytomegalovirus β2.7 RNA was also found to prevent death of dopaminergic neurons in the brain. The death of dopaminergic neurons in the brain is considered to be a hallmark of Parkinson's disease. It has been shown that, for example, a short 100 nucleotide long subdomain (domain 2 of the β2.7 RNA) successfully protected the mitochondrial complex Ito a similar extent as the parental β2.7 RNA sequences. Therefore, domain 2, among the other domains disclosed herein, can be implemented in the treatment of Parkinson's disease. The term parental sequence refers to the original β2.7 RNA sequence derived from human cytomegalovirus strain towne (GenBank: FJ616285.1).

Cell penetrating peptides can be used to successfully deliver β2.7 derived sequences into, for example, lung tissue of human and animal models. This delivery serves to treat impaired oxidative phosphorylation (OXPHOS), for example, and in another example, increase reactive oxygen species (ROS) levels associated with chronic obstructive pulmonary disorder (COPD). Additionally, antisense RNA can be used to target hereditary mitochondrial defects in the lungs

β2.7 RNA, or subdomains thereof, can also be used to deliver intact mitochondrial genomes for treatment of disorders with mitochondrial DNA (mDNA) deletions, such as Kearns-Sayre Syndrome (KSS), Pearson Syndrome and progressive opthalmoplegia (PEO), all of which share overlapping phenotypes and which are associated with a common 4977 base pair deletion within the mitochondrial DNA.

Thus, in one example, there is disclosed a method of treating a mitochondrial disorder. In another example, the method of treating a mitochondrial disorder comprises administering to a subject the nucleic acid delivery construct as disclosed herein. In yet another example, the method comprises using the nucleic acid delivery construct to deliver antisense RNA. The mitochondrial disorder can be, but is not limited to, maternally inherited diabetes mellitus, Leber's hereditary optic neuropathy (LHON), neuropathy, ataxia, retinitis pigmentosa, myoclonic epilepsy with ragged red fibers (MERRF), mitochondrial myopathy encephalopathy lactic acidosis and stroke like symptoms (MELAS), Parkinson's disease, chronic obstructive pulmonary disorder (COPD), Kearns-Sayre Syndrome (KSS), Pearson Syndrome and progressive opthalmoplegia (PEO).

Said mitochondrial disorders can be treated in various ways, for example, by targeting one or more mitochondrial genes, which are, but not limited to, MT-TL1 (tRNA leucine), MT-ND1, MT-ND4, MT-ND6, MT-ATP6, MT-TK (tRNA lysine), MT-ND1, MT-NDS, MT-TH (histidine), MT-TL1 (leucine), MT-TV (valine), and combinations thereof.

One example of the treatment of mitochondrial disorders is the antisense-mediated suppression of defective mitochondrial genes/gene functions. This involves, for example, the use of the nucleic acid delivery system as disclosed herein to deliver antisense RNA into the mitochondria. For example, the ability of the human cytomegalovirus β2.7-mediated delivery of antisense RNA to successfully knock down mt-ATP6 mRNA within mitochondria has been demonstrated in the present application. Defects in the mt-ATP6 have been associated with, for example but not limited to, neuropathy, ataxia, and retinitis pigmentosa (NARP). In the example of NARP, the β2.7-mediated antisense RNA delivery system has been used to successfully knock down defective mRNA associated with NARP, thereby allowing the wild type ATP6 mRNA to re-populate the mitochondria. Hence, the nucleic acid system as disclosed herein can be used for treatment of NARP.

Another example of how the claimed invention can be used in the treatment of mitochondrial disorders is the delivery of a mitochondrial gene. Alternatively to suppressing defective mRNAs using antisense RNA, the nucleic acid delivery system as disclosed herein can be used to deliver an intact (mitochondrial) gene into the mitochondrial to increase the ratio of intact mRNA to defective RNA. Provided below is a non-exhaustive list of target genes and their associated diseases.

TABLE 1
A list of target genes and associated diseases.

| Disease | Affected gene |
|---|---|
| Maternally inherited diabetes | MT-TL1 (tRNA leucine) |
| mellitus | |
| Leber's hereditary optic | MT-ND1, MT-ND4, MT-ND6 |
| neuropathy (LHON) | |
| Neuropathy, Ataxia and Retinitis | MT-ATP6 |
| pigmentosa (NARP) | |
| Myoclonic epilepsy with ragged | MT-TK (tRNA lysine) |
| red fibres (MERRF) | |
| Mitochondrial myopathy | MT-ND1, MT-ND5, MT-TH |
| encephalopathy lactic acidosis | (histidine), MT-TL1 (leucine), |
| and stroke like symptoms | MT-TV (valine). |
| (MELAS) | |

MT-TL1: Mitochondrial encoded tRNA leucine; MT- TV: Mitochondrial encoded tRNA valine; MT-TK: Mitochondrial encoded tRNA lysine; MT-TH: Mitochondrial encoded tRNA histidine; MT-ND1: Mitochondrial encoded NADH dehydrogenase 1; MT-ND4: Mitochondrial encoded NADH dehydrogenase 1; MT-ND5: Mitochondrial encoded NADH dehydrogenase 5; MT-ND6: Mitochondrial encoded NADH dehydrogenase 6; MT-ATP6: Mitochondrial encoded ATP synthase 6.

Yet another example of the treatment of mitochondrial disorders is a combination of both suppressing defective mitochondrial gene function and the delivery of intact genes into the mitochondria to increase the ratio between intact and defective genes.

As disclosed herein, a therapeutic application of the invention can be either the delivery of antisense sequences to suppress defective gene expression, or, alternatively, to deliver intact genes to complement the correct gene function, or a combination of both.

Thus in one example, there is disclosed a method of enhancing mitochondrial gene function, or suppressing defective mitochondrial gene function, or both (provided that in this case, the mitochondrial genes are different from each other). In the case of both enhancing mitochondrial gene function and suppressing defective mitochondrial gene function, the enhancing and suppressing of gene function can take place simultaneously or sequentially. In one example, the enhancing and suppressing of gene function takes place simultaneously. In another example, the mitochondrial gene functions are different from each other. In yet another example, the method comprises administering to a subject the nucleic acid delivery sequence as disclosed herein.

For example, domain 3 (D3X4) is shown to exhibit enhanced mitochondrial localisation potential. Combination of tandem repeats are constructed as, for example, D3X4_D2X4 or D2X4_D3X4, whereby D3X4_D2X4 is shown to exhibit the highest mitochondrial targeting potential. It is further shown that domains 1 and 4 exhibit similar structures on the antisense transcript, and that the antisense domains AS1 and AS4 exhibit substantial mitochondrial localization potential.

Delivery of CMV β2.7 RNA-derived sequences with coding RNA into mitochondria leads to recombinant mitochondrial gene expression. For example, the dual tetramer of domains D3 and D2 (denoted as D3x4_D2x4) protects mitochondrial complex I with higher efficiency than, for example the wild type β2.7 RNA.

Using computational methods, four thermodynamically conserved structural sub-domains within the β2.7 RNA were identified. All four domains showed substantial mitochondrial localization, and it was shown that the complete mitochondrial localization activity of the full-length β2.7 RNA could also be achieved by, for example, use of a single sub-domain termed domain 3. Furthermore, two of the four domains (for example, domains 1 and 4) exhibited highly similar structures on the antisense transcript and the antisense domains AS1 and AS4 exhibited substantial mitochondrial localization potential. A tetramer of, for example, sense domain 3 was found to have a twice higher mitochondrial localization activity and, in another example, a tetramer of domains 3 followed by a tetramer of domain 2 exhibited a three-fold higher activity compared with, for example the β 2.7 RNA or domain β2.7 RNA-derived sequences were used to deliver recombinant nucleic acids into mitochondria in order to trigger mitochondria-specific phenotypes: Firstly, in one example, a coding RNA was furnished with mitochondria-specific start and stop codons, leading to mitochondria-specific recombinant gene expression; secondly, antisense RNAs targeting mitochondria-specific genes were used to trigger functional knockdown of mitochondria-specific gene expression. This technology therefore finds use in mitochondrial gene therapy or, for example, for mitochondrial delivery of non-nucleic acid compounds.

As an example of the use of the claimed invention, an exemplary method involves delivery of CMV β2.7 RNA-derived sequences with coding RNA into mitochondria, which in turn leads to recombinant mitochondrial gene expression. Delivery of CMV β 2.7 RNA-derived sequences, for example, with antisense RNA into mitochondria triggers functional knockdown of mitochondrial gene expression. One example of such a delivery construct is a tetrameric repeat of the β 2.7 RNA subdomain 3, which has been shown to exhibit enhanced mitochondrial localization potential. Exemplary arrangement of, for example two tetrameric repeats of β2.7 RNA subdomains 3 and 2 (D3x4_D2x4), which exhibit high mitochondrial targeting potential. Exemplary dual tetrameric of domains D3 and D2 (D3x4_D2x4) are shown to protect mitochondrial complex I with higher efficiency than the wildtype β2.7 RNA. Exemplary application of the outlined method is in genetic therapy, for example, to suppress mitochondrial malfunction or, in another example, to restore mitochondrial gene functions. Examples of application also include use in neurodegenerative or other mitochondria-associated diseases or for anti-aging.

Another example of the use of the claimed invention includes the use of the claimed nucleic acid delivery system together with CRISPR/Cas technology, that is using the claimed nucleic acid delivery system for delivery of the mRNA coding for the Cas9 endonuclease together with a single guide (sg)RNA, or for delivery of the respective genes coding for these components. The Cas9 enzyme together with the sgRNA can then form a ribonucleoprotein complex that can specifically cleave and functionally inactive defect mitochondrial genes or genomes.

A further application is to provide plasmid-based mitochondrial targeting vectors. A sequence of interest for the transcription of a coding or a non-coding RNA can be inserted either upstream or downstream to the mitochondrial targeting sequences. The chimeric RNAs can then be transcribed from the DNA templates either in vitro and then delivered into the target cells, or endogenously after transfection of target cells with the DNA vector. Variations of these vectors for the production of viral delivery particles, for example, but not limited to, lentiviral, adenoviral, adeno-associated virus, are envisioned as well.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Experimental Section

Identification of Thermodynamically Conserved Structural Subdomains with the CMV β2.7 RNA

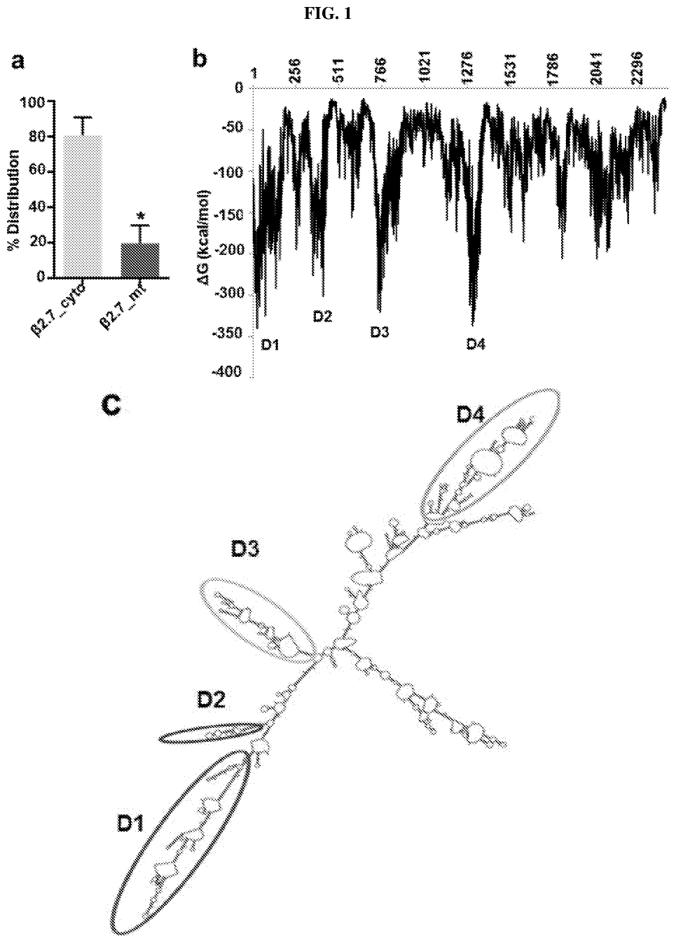

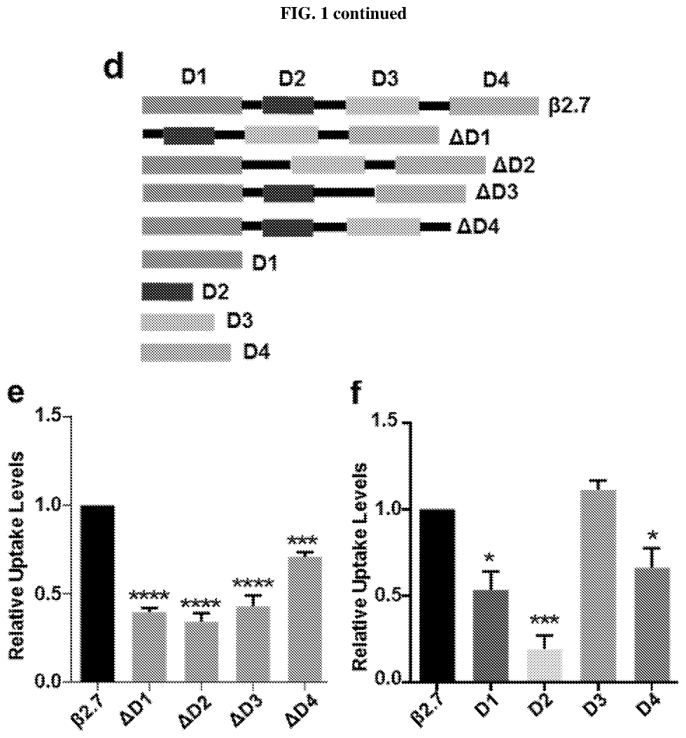

Using the software foldsplit, four thermodynamically conserved structural subdomains (named D1 to D4, respectively) within the non-coding β2.7 RNA of CMV were identified (FIG. 1b). Thermodynamic conservation frequently correlates with RNA function. Each of these four domains can be assigned to a well-defined structural subdomain within the RNA secondary structure predicted for the complete β2.7 RNA (FIG. 1c).

The CMV β2.7 RNA and Distinct Functional Subdomains thereof Localize to the Mitochondria of Human Cells

Mitochondrial localization, for example, (i) of the full-length β2.7 RNA, (ii) of each of the four single domains D1 to D4, and (iii) of single domain deletion constructs (FIG. 1d-f) were investigated. While all of the deletion mutants and three of four single domains showed reduced mitochondrial targeting, domain 3 exhibited the complete mitochondrial targeting potential of the full-length β2.7 RNA.

### Distinct CMV β2.7 RNA Antisense Domains Localize to the Mitochondria of Human Cells

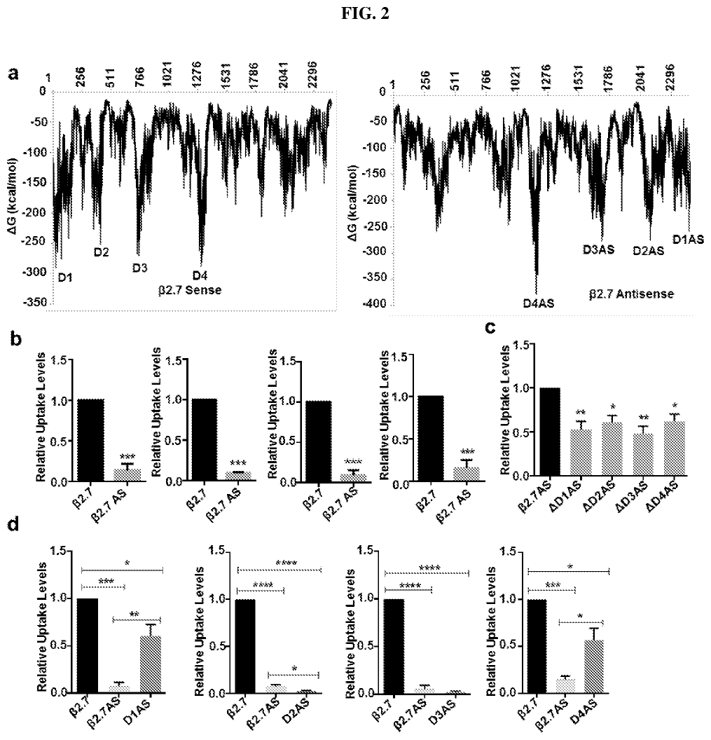

The antisense sequences of the constructs disclosed herein were considered for use as negative controls and therefore were also investigated in terms of their structures and thermodynamic conservation. Unexpectedly, the structure of the antisense β2.7 RNA was highly symmetric compared with the structure of the sense β2.7 RNA. A highly similar thermodynamic conservation was observed and it was possible to identify four conserved structural subdomains (D1_AS to D4_AS) in the corresponding position within the β2.7 RNA antisense sequence (FIG. 2a). A detailed structural analysis revealed in particular antisense domains D1_AS and D4_AS were structurally almost identical compared with the corresponding sense domains D1 and D4. The mitochondrial localization (i) of the full-length antisense β2.7 RNA, (ii) of each of the four single antisense domains D1_AS to D4_AS, and (iii) of antisense single domain deletion constructs (FIG. 2b-d) were investigated. The full-length antisense β2.7 RNA showed significantly weaker mitochondrial uptake compared with the sense RNA (FIG. 2b) and the deletion of each of the four antisense domains D1_AS to D4_AS further reduced mitochondrial targeting (FIG. 2c). When testing the individual antisense domains, it was shown that antisense domains D1_AS and D4_AS exhibited an about 5-fold higher activity compared with the full-length antisense RNA reaching about 70% of the mitochondrial targeting activity of the sense full-length β2.7 RNA (FIG. 2d).

The CMV β2.7 RNA Co-Delivers Recombinant Coding RNA into the Mitochondria Leading to Mitochondrial Expression of a Recombinant Protein

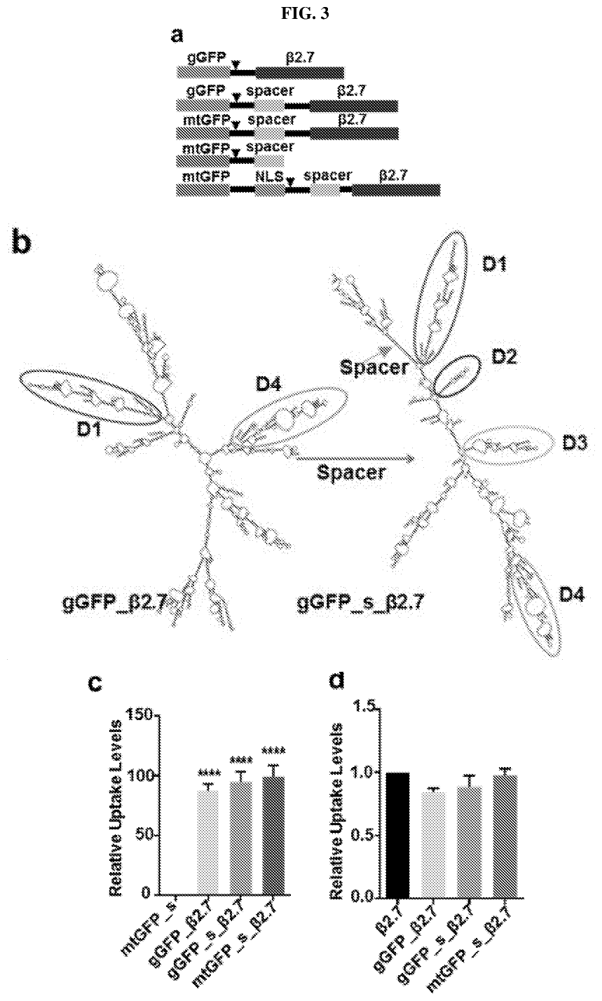

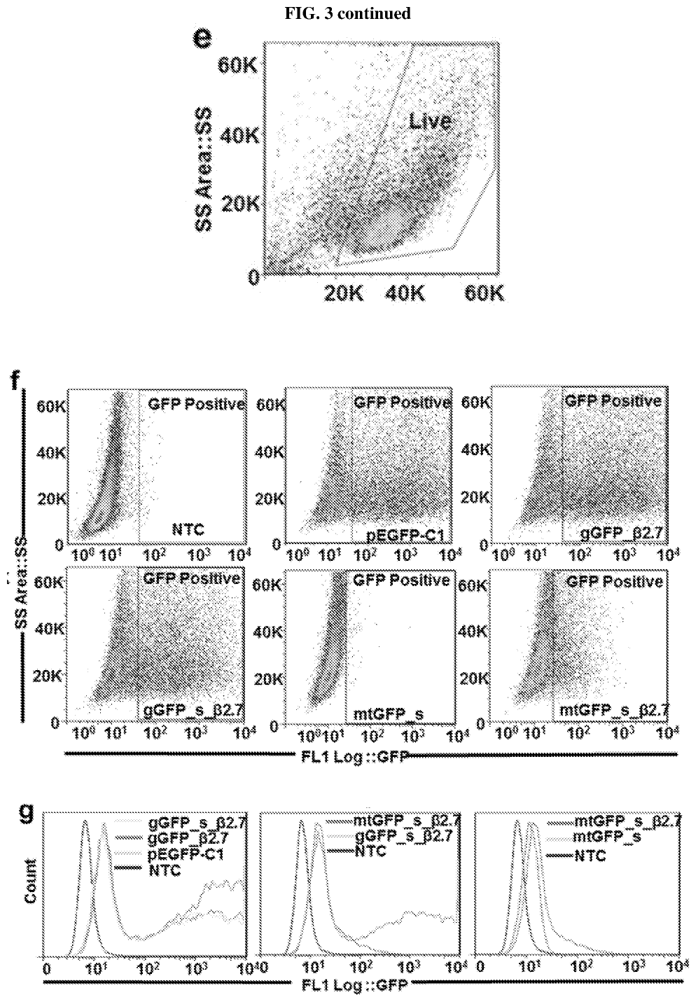

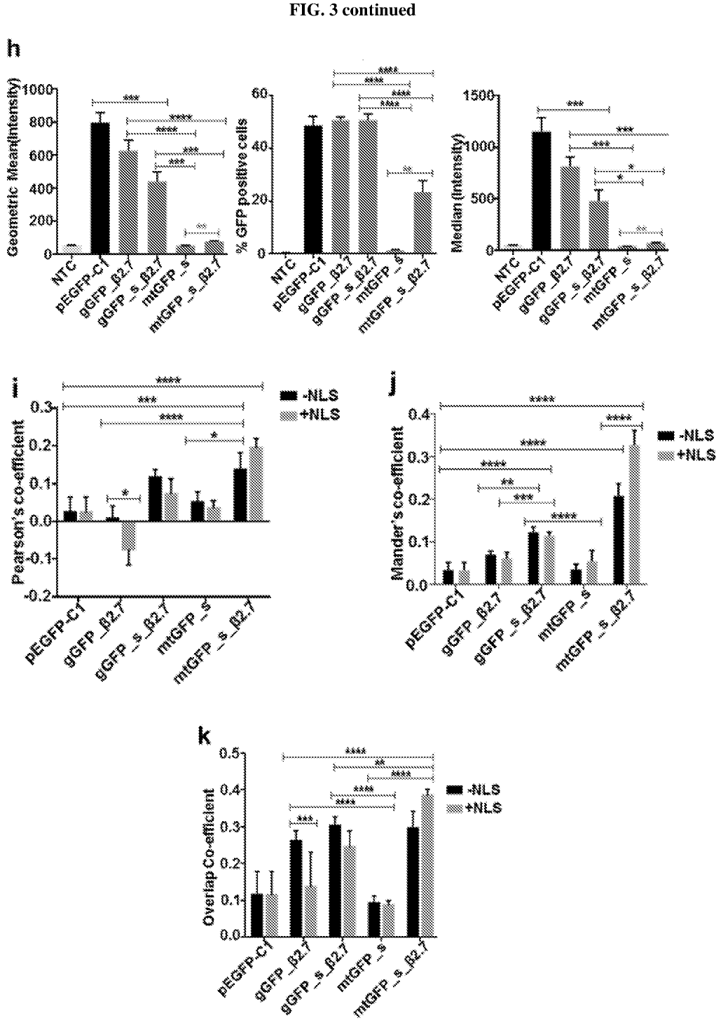

A β2.7 RNA full-length RNA was fused to the 3′ end of EGFP mRNA via a spacer sequence, which ensured that the active structure of the β2.7 RNA was not changing upon fusing it to the EGFP sequence (FIG. 3a,b). Two different version of the EGFP mRNA were considered: 1. The conventional mRNA equipped with regular start and stop codon for cytoplasmic expression; and 2. A modified version equipped with mitochondrial start and stop codon which can only be translated in the mitochondria but not in the cytoplasm of cells. In addition, a version of the mitochondrial EGFP sequence was generated in which the EGFP protein was fused to a nuclear localization peptide so that any EGFP protein that reached the cytoplasm would be targeted to the nucleus in order to suppress any overlapping EGFP signals originating from the cytoplasm and the mitochondria. All sequences and controls were then tested for mitochondrial targeting using real-time RT-PCR (FIG. 3c) or EGFP expression using flow cytometry (FACS) and confocal microscopy (FIG. 3d-j). Mitochondrial targeting of the β2.7 RNA was not hampered by fusing it to the EGFP mRNA (FIG. 3c). FACS analyses indicated that the mitochondrial egfp sequence (mtGFP_s) alone did not trigger any EGFP expression. Significant EGFP expression, which must originate from the mitochondria, was able to be detected after fusing it to the β2.7 RNA (mtGFP_s_β2.7) (FIG. 3g). Confocal imaging was not sensitive enough to visualize co-localization of EGFP and a mitochondria-specific stain (MitoTracker Red). However, three different co-localization coefficients indicated significant co-localization of the EGFP and MitoTracker Red in case of the mtGFP_s_β2.7 but not for the mtGFP_s control (FIG. 3h-j).

The CMV β2.7 RNA Co-Delivers Antisense RNA into the Mitochondria Leading to Suppression of Mitochondrial Expression of a Recombinant Protein

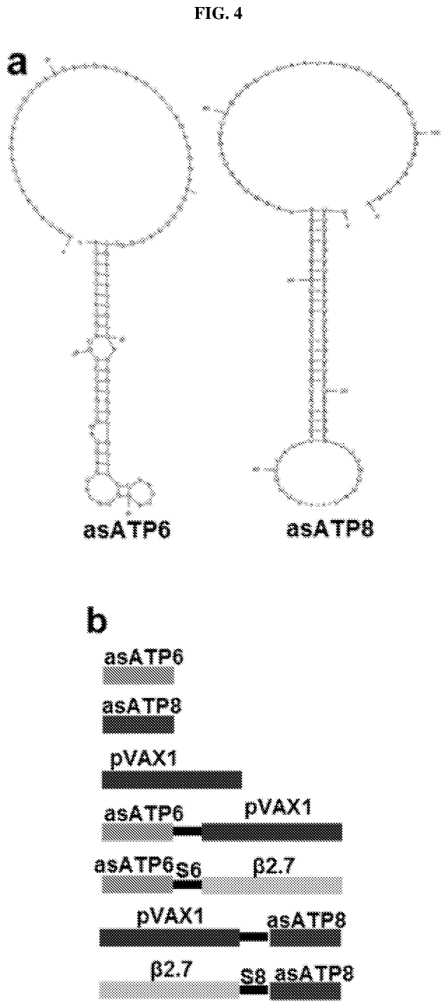

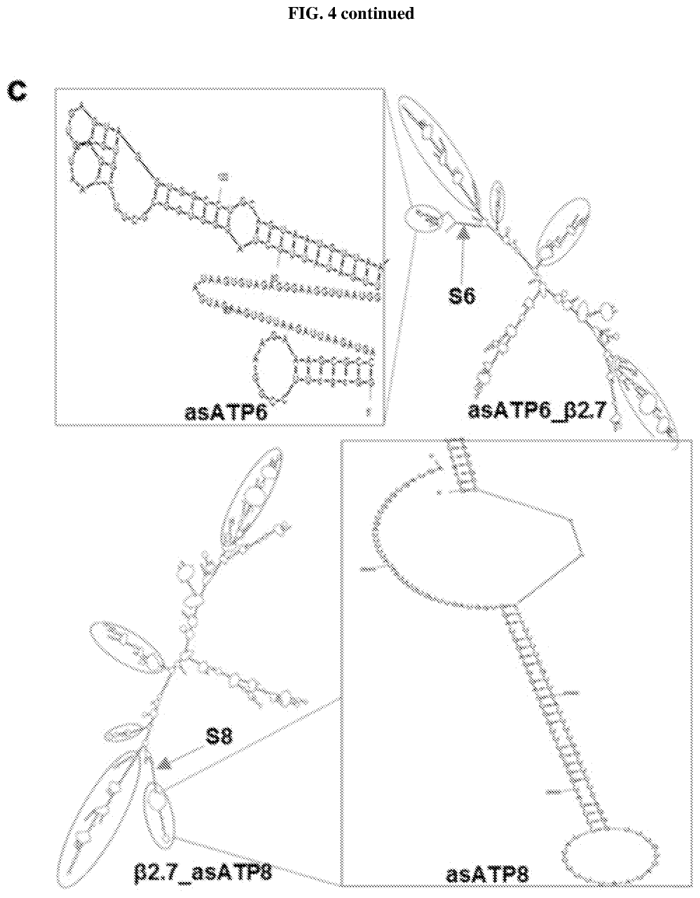

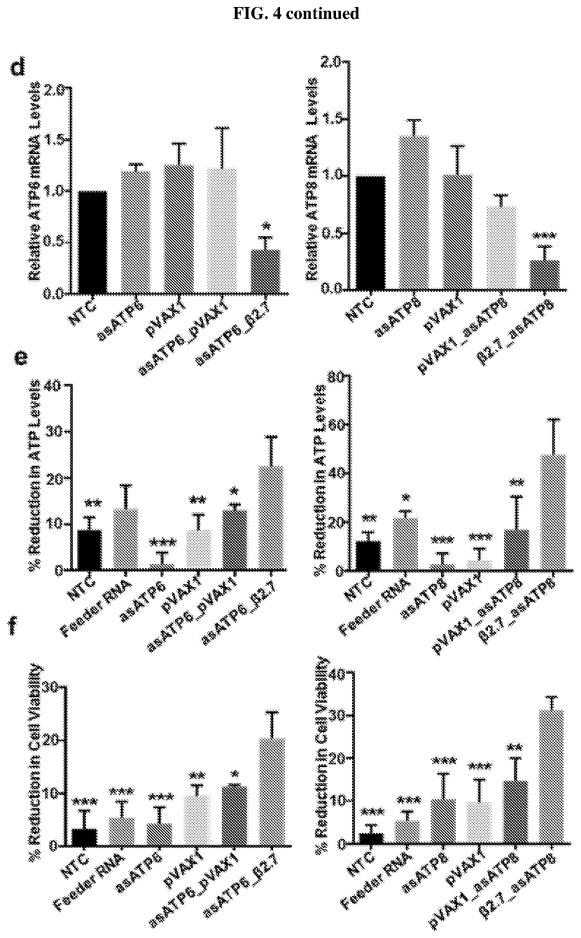

Next, the β2.7 RNA was fused to computationally selected, unstructured antisense RNAs targeting the mitochondrial gene, for example, MT-ATP6 and MT-ATP8 which are both involved in mitochondrial ATP synthesis (FIG. 4). The antisense RNAs were fused to the β2.7 RNA via spacers to ensure the active structures of both sequences (antisense and β2.7 RNA) were maintained during the fusion process. The mitochondrial RNA targeting was then measured using rtRT-PCR (FIG. 4e), knockdown of mitochondrial ATP synthesis (FIG. 4f), and reduction of cell viability as a consequence of reduced ATP levels (FIG. 4g). Fusion of the β2.7 RNA targeted the antisense RNAs to the mitochondria, triggered knockdown of mitochondrial MT-ATP6 or MT-ATP8 mRNA levels, and significantly reduced cell viability.

### A Tetrameric Repeat of the β2.7 RNA Subdomain 3 Exhibits Enhanced Mitochondrial Localization Potential

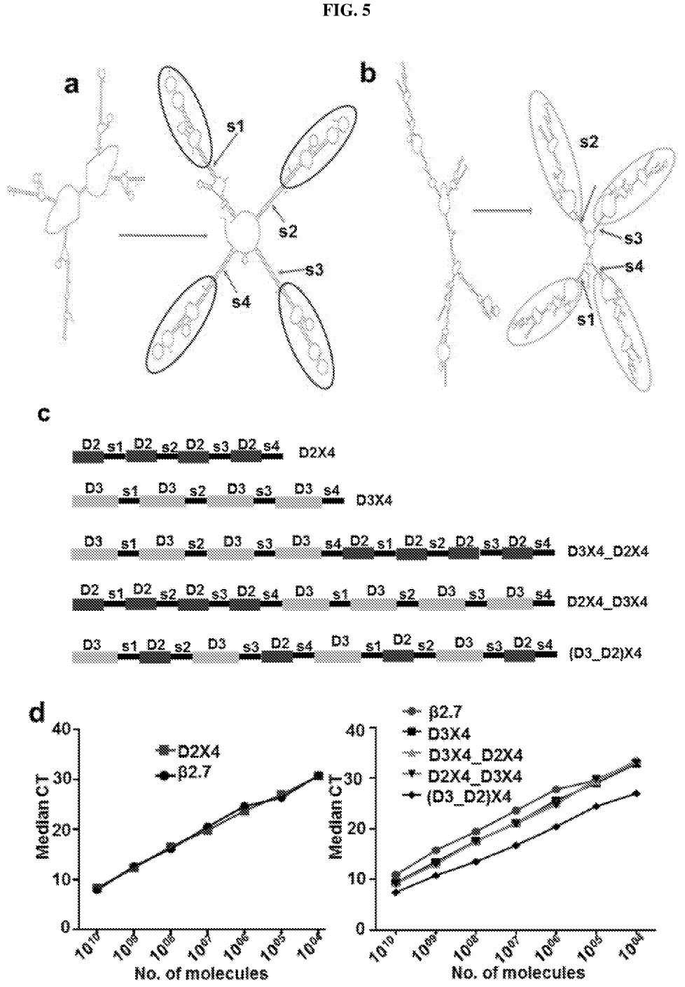

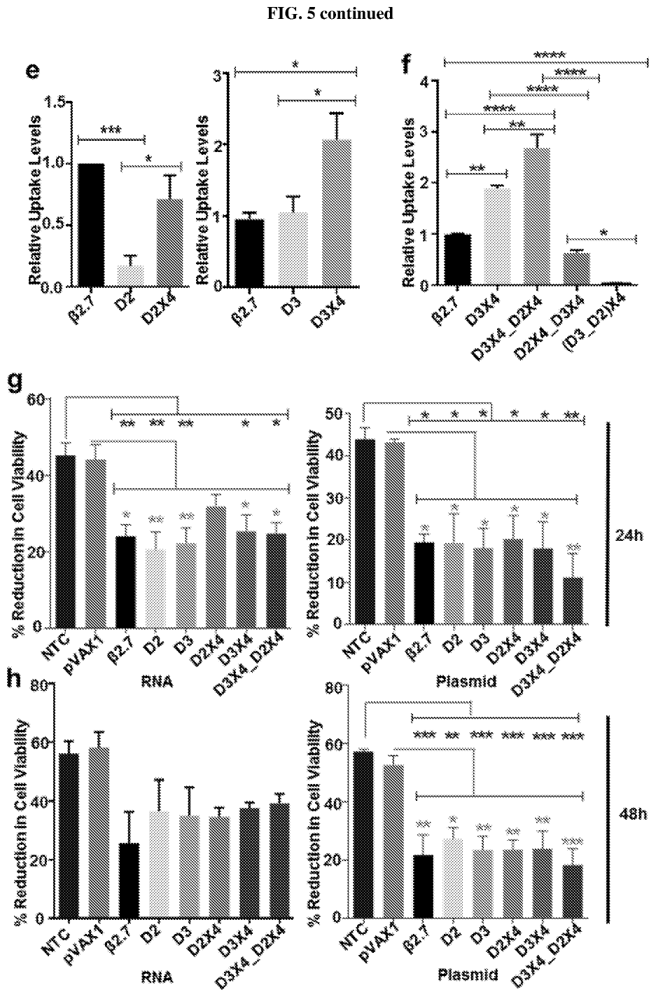

It was aimed to improve the mitochondrial targeting potential of the β2.7 RNA, for example by using multiple repeats of the most active β2.7 RNA subdomains, in one example D3 and D2. Tetrameric repeats of domains D3 or D2 were constructed and investigated for mitochondrial targeting using rtRT-PCR (FIG. 5a-f). While the D2 tetramer mitochondrial targeting was comparable to that of the full-length β2.7 RNA, the D3 tetramer showed a two-times higher mitochondrial targeting activity.

The Arrangement of Two Tetrameric Repeats of β2.7 RNA subdomains 3 and 2 (D3x4_D2x4) Exhibits Highest Mitochondrial Targeting Potential

Next constructs comprising (i) four repeats of D3 followed by four repeats of D2 (D3x4_D2x4), or (ii) four repeats of D2 followed by four repeats of D3 (D2x4_D3x4), or (iii) alternating domains D3 and D2 [(D3_D2)x4] were tested. While D2x4_D3x4 and (D3_D2)x4 exhibited reduced mitochondrial targeting activities comparable with the full-length β2.7 RNA, construct D3x4_D2x4 was found to be about 3-fold more active (FIG. 5g).

The Dual Tetrameric of Domains D3 and D2 (D3x4_D2x4) Protects Mitochondrial Ccomplex I with Higher Efficiency than the Wwildtype β2.7 RNA

The 5′ terminal part of the β 2.7 RNA was reported earlier to protect the mitochondrial complex I from inhibitors such as rotenone. It was investigated as to whether domains D3 or D2 alone, or their multimeric arrangements, were able to protect complex I against rotenone when delivering either, for example, the in vitro synthesized RNAs or alternatively plasmids expressing the RNAs endogenously (FIG. 5h,i). While the D2, D3, D3x4, and D3x4_D2x4 RNAs were as protective as the full-length β 2.7 RNA when delivering RNA directly, D3x4_D2x4 was found to be more active when delivering RNA expressing plasmid DNA.

Strategies of Delivering Recombinant Nucleic Acids into Mitochondria using Mitochondrial Targeting Sequences

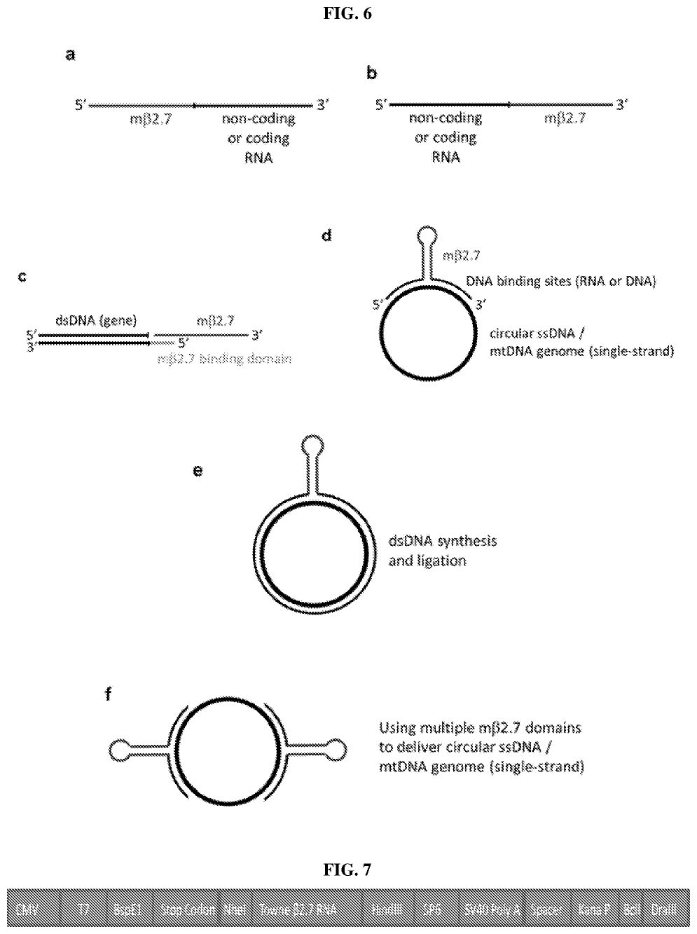

Recombinant RNA or DNA can be either covalently linked to mitochondrial targeting sequences or alternatively be non-covalently linked via complementary base pairing. The recombinant nucleic acid can be either single-stranded, linear double-stranded, circular single-stranded, or circular double-stranded. One or multiple mitochondrial targeting sequences can be used to form one mitochondrial targeting complex. (FIG. 6)

### Materials and Methods

### Design of the Parental Construct

The purpose of designing the construct was to clone it into the pVAX1 (Invitrogen) and pEGFP-C1 (Addgene) vectors to study domain characteristics and transgene delivery respectively. The restriction sites were selected using the online algorithm NEBcutter v2.0. The construct was synthesized by Geneart (Life Technologies). The construct was subsequently cloned into the pVAX1 vector. The CMV promoter sequence was obtained from the pVAX1 plasmid vector. The CMV promoter was to facilitate expression of the RNA in animal cells. The T7 promoter was to facilitate in vitro transcription of the parental constructer whereas the SP6 promoter was inverted to facilitate in vitro transcription of the antisense RNA sequence. HindIII was used to linearize plasmid for T7 transcription and BspEI was used to linearize the plasmid for SP6 transcription. NheI, XbaI, and BcII sites were used for cloning, The SV40 poly A signal was added to facilitate Polyadenylation and nuclear export of mRNA. The spacer represents the vector sequence between the kanamycin promoter and the polyA signal and KanaP is the part of the promoter which is initially removed from the vector during cloning using BcII. DraIII was included to facilitate cloning into the pEGFP-C1 vector (see FIG. 7).

### Synthesis of the Single Domain Deletion Constructs (SDDC)

To study the effect of individual domains on β2.7 RNA function, constructs were generated having individual domains deleted. The strategy used to delete the domains was overlap extension PCR as indicated in FIG. 8.

### Overlap Eextension (OE) PCR Strategy for Synthesis of SDDC

Overlap extension polymerase chain reaction (OE PCR) was carried out in two steps. In the first step, the region upstream of the deletion domain (DD) was PCR amplified using a common forward primer which carries the NheI site and the OE reverse primer, which introduces the priming site for the region downstream of the DD. Subsequently, the domain downstream of the DD was PCR amplified using an OE forward primer, which introduces the priming site for the region upstream of the DD. and a common reverse primer, which carries the BamH1 site. Both PCR products were then gel-purified and mixed together in equimolar amounts and then re-amplified using PCR, in which the OE introduced priming sites acted as primers for their respective counterparts, thereby giving the full length construct with the desired deletion.

### Synthesis of the Single Domain Constructs (SDC)

The Single domains were PCR amplified from the parental β2.7_pVAX1. The PCR conditions for each of the single domains were as follows: Single Domains 1(SD1_pVAX1): Using β2.7_pVAX1 as template PCR was carried out using D1F and D1R following which the PCR product was cloned using the BspEI and HindIII sites. The PCR conditions reaction mixture included 10 ng of template, 5 μl of 10× Taq buffer, 4 mM of MgCl2,0.2 mM of each dNTP, 300 nM of each primer and 0.25 μl of Taq Polymerase (ThermoScientific).The final volume was made up to 50 μl with Ultrapure Nuclease free water (Invitrogen). The reaction conditions used were 94° C. for 5 minutes, 25 cycles of 94° C. for 30 seconds, 56° C. for 30 seconds, 72° C. for 30 seconds, 72° C. for 5 minutes. Single Domain 2 (SD2_pVAX1). Single Domain 3(SD3_pVAX1), and Single Domain 4 (SD4_pVAX1) constructs were generated the same way but using the specific PCR primer D2F and D2R, D3F and D3R or D4F and D4R instead (see FIG. 9).

### Strategy for Single Domain Amplification

The forward and the reverse primers were designed to introduce the BspEI and HindIII sites respectively. The PCR products were subsequently digested with BspEI and HindIII purified and cloned into the pVAX1 vector using the BspEI and HindIII sites.

### Synthesis of the GFP Fusion Constructs

Design of the GFP Fusion constructs: All the GFP fusion constructs were designed using the pEGFP-C1 (Addgene) plasmid vector. The β2.7 RNA sequence was cloned downstream of the eGFP sequence in the plasmid using restriction sites BspEI and DraIII. Next the eGFP sequence was PCR amplified from the plasmid to introduce the restriction site at either end along with the spacer sequence. To enable mitochondria-specific gene expression, the start and stop codons of the eGFP message were modified with mitochondria specific start and stop codons. Since the analysis of GFP expression would be carried out using plasmid vectors primarily the structure of the CMV transcript was analysed for structural preservation using mfold. The PCR conditions are described below. 50 μl PCR reaction was set up having 10 ng of pEGFP-C1 template, 5 mM of 10Xtaq buffer, 4 mM Mgcl2, 300 nm of each primer, 0.2 mM of each dNTP and 0.25 μl of Taq Polymerase (ThermoScientific). The cycler was set at 94° C. for 5 minutes, 25 cycles of 94° C. for 30 s, 55° C. for 30 s, 72° C. for 1 minute, 72° C. for 10 minutes. The parental β2.7 sequence with the SV40 poly A was cut out from the β2.7_pVAX1 vector, gel purified and ligated downstream of the eGFP sequence using the BspEI and DraIII sites as indicated in FIG. 10.

The cloning process described above creates the Genomic GFP+β2.7. The 2nd control namely genomic GFP+Spacer+β2.7 was created by amplifying the eGFP sequence from the original pEGFP-C1 vector by PCR using PCR primers with the reverse primer introducing the spacer sequence and the NheI site following which it was cloned back into the Genomic GFP+β2.7 vector. To enable mitochondria specific expression, the start and stop codons of the eGFP sequence were modified using PCR primers with the forward primer introducing the mt start codon and the reverse primer introducing the spacer sequence and the mitochondria specific stop codon following which it was cloned back into the pEGFP-C1 vector carrying the β2.7 using NheI site to generate mtGFP+Spacer+β2.7. In both these cases the GFP sequences were cloned into the vector using a single NheI restriction site and hence to prevent vector backbone re-ligation, it was dephosphorylated simultaneously with Alkaline phosphatase during digestion with NheI. As a negative control the mtGFP+spacer sequence with a portion of the CMV promoter was PCR amplified from the mtGFP+Spacer+β2.7 plasmid using primers which carried NdeI and BamHI sites respectively. Simultaneously the pEGFP-C1 plasmid vector was digested between the NdeI site and BamHI site to remove the original GFP sequence and a part of the CMV promoter. The PCR product was then digested with NdeI and BamHI, purified and ligated back into the gel purified vector backbone which re-constitutes the CMV promoter and replaces the eGFP sequence with the mtGFP+spacer sequence.

### Adding a SV40 Nuclear Localization Signal to the GFP Fusion Constructs

To facilitate imaging by confocal microscopy, a SV40 nuclear localization signal (NLS) was PCR amplified from the pEBFP_NUC (Addgene) and cloned downstream of the GFP sequence in all GFP expressing constructs, including the mitochondrial constructs. The NLS carries a mitochondria specific stop codon in frame and, as a result, only cytoplasmic GFP is targeted to the nucleus, whereas intra-mitochondrial GFP remains localized in the mitochondria. This is because translation in the mitochondria generates an incomplete NLS. The pEBFP-NUC plasmid carries 3 tandem repeats of the NLS sequence and hence the PCR primers were designed to flank the tandem repeats. PCR amplification and design strategy is explained in figure. Post-cloning, the constructs were screened using a single XhoI digestion. The PCR conditions were as follows. Using the pEBFP_NUC as a template PCR was carried out using the NLS_cloning Fw and NLS_cloning Rv following which the samples were PCR purified and cloned downstream of the GFP sequence between the Bsp1407I and the BspEI sites. The PCR conditions used were as follows: 10 ng of pEBFP-NUC template, 5 mM of Taq buffer, 4 mM Mgcl2, 0.2 mM of each dNTP, 300 nM of NLS_cloning Fw and NLS_cloning Rv respectively, 0.25 μl of Taq Polymerase (ThermoScientific) and the volume was made up to 50 μl with ultrapure Nuclease Free Water (Invitrogen). The cycling conditions used were 94° C. for 5 minutes, 25 cycles of 94° C. for 30 seconds, 52° C. for 30 seconds, 72° C. for 5 minutes.

The NLS sequence was PCR amplified from the pEBFP-NUC plasmid by PCR using forward and reverse primers which introduced BsrGI and BspEI sites, respectively (see FIG. 11). Additionally the forward primer also destroyed an extra BspEI site upstream of the NLS by a single T-G substitution. The PCR product was then subsequently digested with BsRGI and BspEI respectively and ligated downstream of the GFP sequence in all the constructs. The PCR product carries an XhoI site, which was used to screen the clones by digestion.

### Synthesis of the Tandem Repeat Constructs

To study the interaction of the domains tandem repeats of two functional domains, in this example namely domains 2 and 3, were created. Four different arrangements were created as follows below.

Constructs having Four Copies of Domain 3(D3X4) and Domain 2(D2X4), Respectively

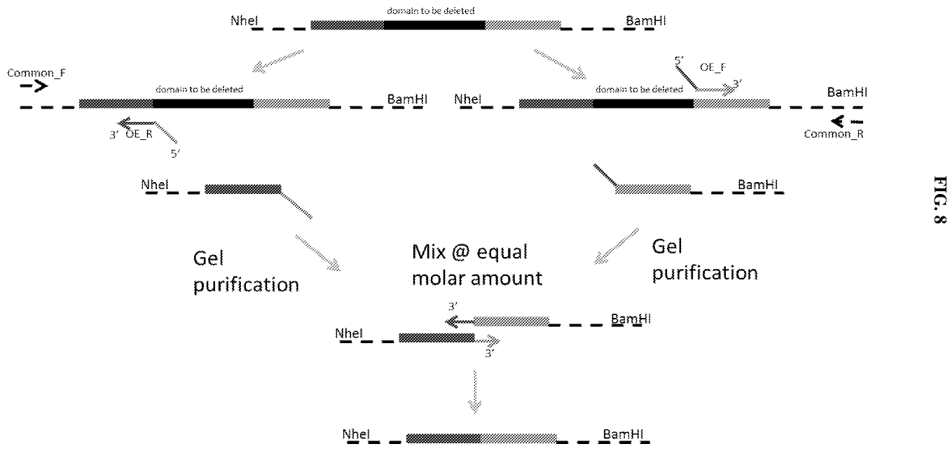

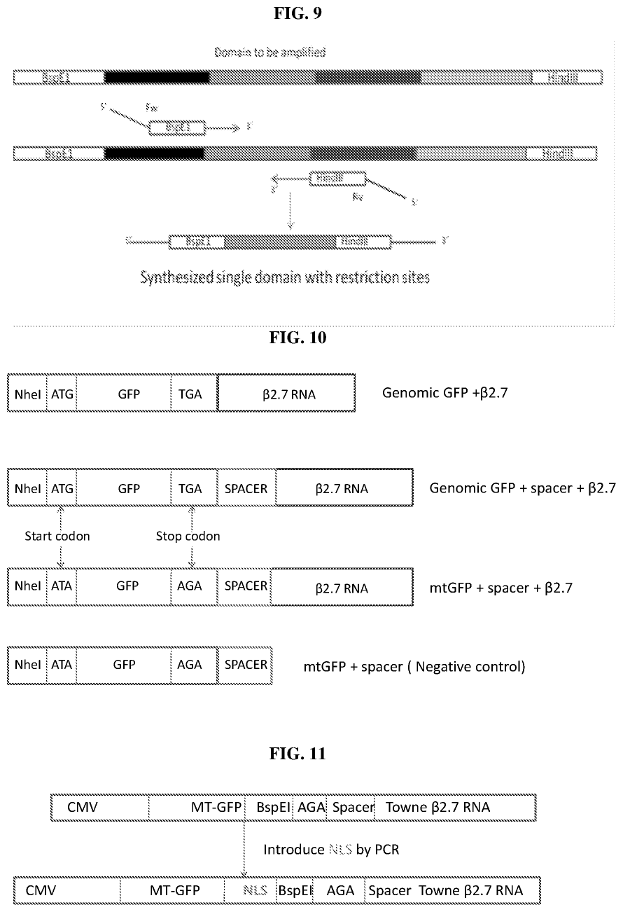

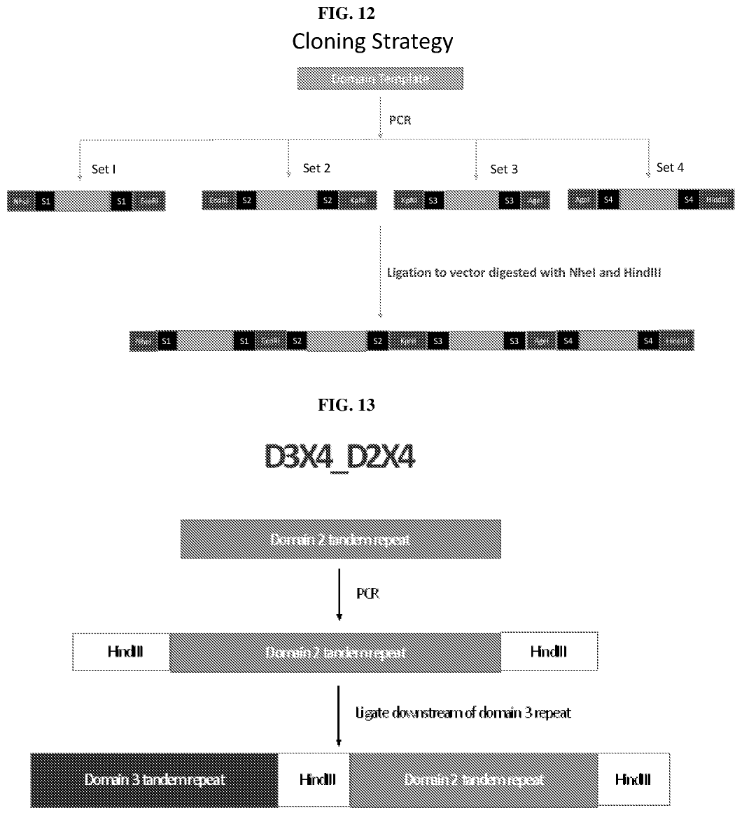

The strategy for generating the constructs were created as shown in FIG. 12. The restriction sites were chosen using NEB cutterV2.0. The structures of the T7 and CMV transcripts of these tandem repeats were stabilized using spacer sequences and were validated using RNAfold and mfold. Each repeat and its associated spacer were synthesized using PCR primers (4 sets) which inserted the spacer and the respective restriction site and then cloned into the pVAX1 vector. Since the primers for each set within the same tandem repeat had the same binding region, the annealing temperature remained the same for all PCR sets for a particular tandem repeat. The cycling conditions used in PCR are provided below

### Domain 2 Tandem Repeats

### Construction of Tandem Repeat Using a Multiple Ligation Process.

The individual copies were first synthesized is 4 sets using PCR primers (see FIG. 12). Since all primers for a particular tandem repeat share the same binding region hence the Tm is the same for all sets for a particular tandem repeat. Each set has its own set of spacers as indicated by S1-S4 in the figure. The restriction sites were chosen such that the site at the 3′end of one set matches the one at the 5′ end of the next set within the tandem repeat. After PCR the products were PCR purified and digested with their respective restriction sites. At the same time the pVAX1 vector was digested with NheI and HindIII and the vector backbone was gel purified. The digested PCR products were then mixed with equimolar amount of the digested vector backbone and Ligated using T4 DNA ligase.

### Construction Strategy for D3X4_D2X4

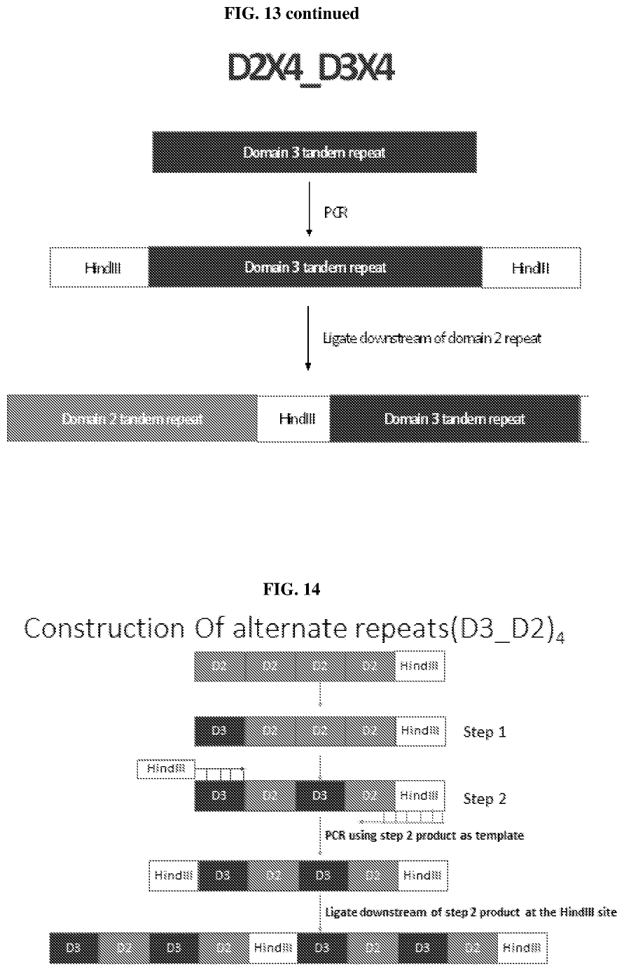

Similarly, the domain 2 tandem repeat (D2X4) was PCR amplified from the pVAX1 vector carrying D2X4 (see FIG. 13). To prevent amplification of shorter fragments, primers were so designed that majority of the binding region of the primer lay on the vector backbone itself. Each of the primers introduced HindIII sites at either end. The PCR product was digested and re-ligated within the HindIII site of the pVAX1 vector carrying the D3X4 sequence. To prevent re-ligation of the vector, the vector was dephosphorylated with Alkaline phosphatase.

### Construction Strategy for D2X4_D3X4

Similarly, the domain 3 tandem repeat (D3X4) was PCR amplified from the pVAX1 vector carrying D3X4.The primer design strategy employed was the same as used for D3X4_D2X4. The PCR product was digested and re-ligated within the HindIII site of the de-phosphorylated pVAX1 vector carrying the D2X4 sequence.

Constructs having Alternate Repeats of Domain 3 and Domain 2 each (D3_D2)4

The alternative repeat construct was generated in four steps. Both D3X4 and D2x4 share the same set of restriction sites. Prior to the cloning the CMV and the T7 transcripts were validated using mfold and RNAfold. A detailed cloning scheme is described in FIG. 14.

In the first step a single copy of domain 3 was cloned using the NheI and Ecorl site. In the 2nd step another copy of domain 3 was cloned between the KpnI and the AgeI sites. The product thus obtained is now used as PCR template using Domain 3 tandem Fw and Domain 2 tandem Rv both of which carry HindIII sites. The PCR product of the correct size is gel-purified and ligated back into the de-phosphorylated pVAX1 plasmid carrying the step 2 product. The clones were then screened by digestion and verified by sequencing (AITBiotech).

### Design of Antisense RNA for Mitochondrial Targeting

### Choice of Target and Selection of Candidates

The candidates for mitochondrial targeting used were Mt-ATP6 and Mt-ATP8, which are essentially subunits of the Complex V (ATPase). The antisense RNA targeting the two genes were designed using HUSAR foldanalyze at window sizes of 100, 200 and 300 and a shift of 1 nucleotide following which candidates with maximum number of unpaired bases at either the 5′ or 3′end were selected. These sequences were subsequently fused to the β2.7 RNA, either at the 5′ end or the 3′ end based on location of the open ends, and structural preservation was analysed using mfold.

### Purification of the Target Sequences

Target sequences were obtained from isolated mitochondrial RNA using a procedure described in the figure below. The reaction conditions for reverse transcription were as follows: 500 ng of mtRNA was mixed with 1 μl of 10 mM dNTP mix, 1 μl of 2 uM gene specific reverse primer made up to a final volume of 14 μl with RNase free water. The mixture was heated at 65c for 5 mins followed by snap chill on ice for 2 mins. This reaction mixture was then mixed with 4 μl of 5× first strand buffer, 1 μl of 100 mM DTT, 20U of RNaseOUT (Invitrogen), 0.5 μl of SuperScriptlV, the final volume of the reaction being 20 μl and incubated at 55° C. for 1 hour, followed by heat inactivation at 70° C. for 15 minutes.

### Isolation of Target Sequences for Antisense Generation

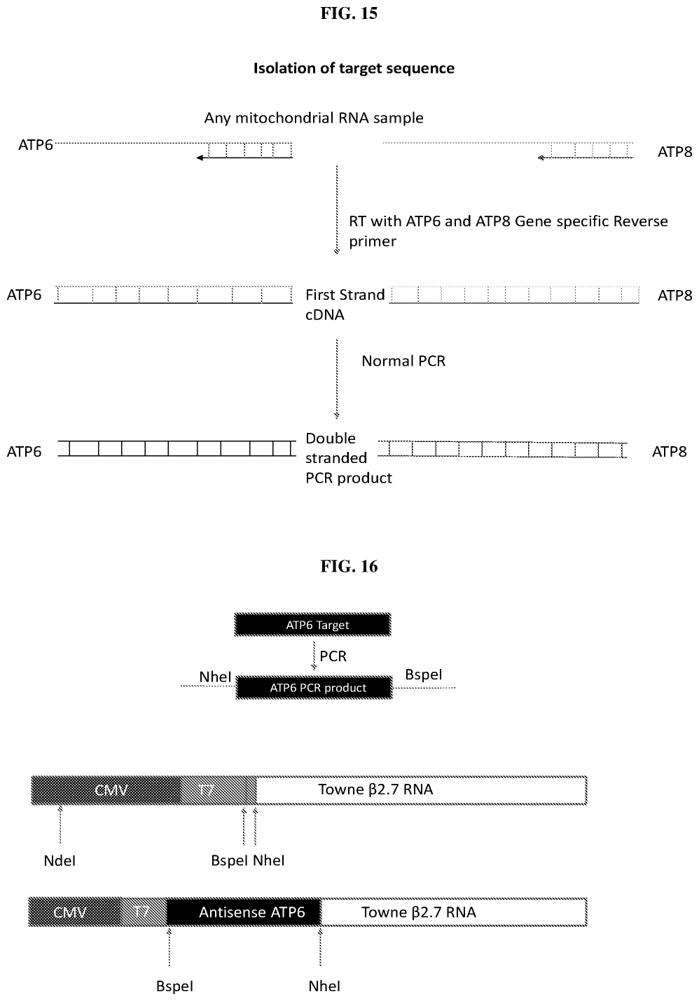

Mitochondrial RNA was reverse transcribed with gene specific reverse primer sequences (ATP6Rv and ATP8Rv) to obtain first strand ATP6 and ATP8 cDNA pools. Subsequently these pools were used as templates for PCR to obtain double stranded DNA sequences representing the target elements (see FIG. 15).

### Cloning of Target Sequences to Generate the Antisense

### Cloning of the ATP6 Antisense Sequence

Since the ATP6 antisense sequence had the maximum number of unpaired bases at the 5′ end, it had to be fused to the 5′ end of β2.7 sequence. To obtain the antisense sequence, the target sequence obtained from mitochondrial RNA pool was reversed and cloned into the β2.7_pVAX1 vector, downstream of the T7 promoter and upstream of the β2.7 sequence. The purified ATP6 target was PCR amplified using ATP6_Cloning_Fw and ATP6_Cloning_Rv.The cloning strategy is described in FIG. 16.

### Synthesis and Cloning of the ATP6 Antisense Sequence

The ATP6 target sequence was re-amplified by PCR to introduce restriction sites in an opposite orientation to that in β2.7_pVAX1 sequence. The resulting product was digested with NheI and BspeI and then ligated into the β2.7_pVAX1 vector backbone. The products were screened by digestion with NdeI and NheI. Subsequently a spacer sequence was introduced downstream of the this construct by nested PCR and then verified by sequencing (AITBiotech).

### Cloning of the ATP8 Antisense Sequence

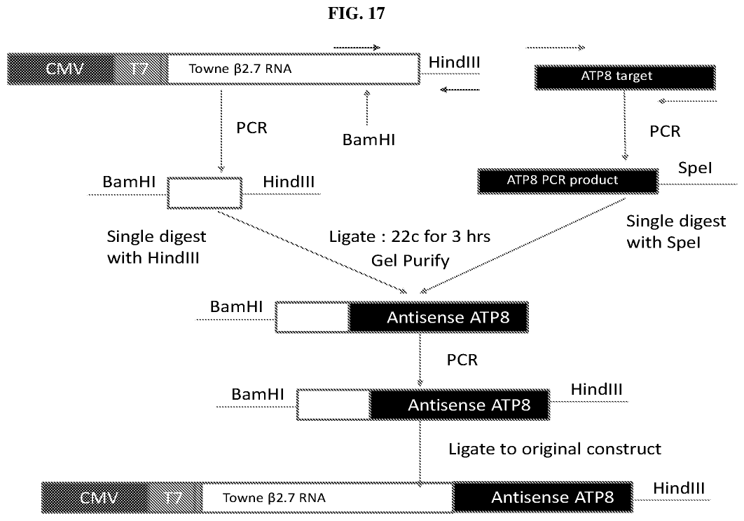

The ATP8 target sequence was cloned downstream of the β2.7 sequence, since the unpaired bases were primarily at the 3′ end. The ATP8 target was amplified by nested PCR to introduce the desired restriction sites and the stabilizing spacer sequence, and cloned downstream of the β2.7 sequence (see FIG. 17).

### Cloning Strategy for Generating β2.7_ATP8 Fusion Construct

The Purified ATP8 target sequence was PCR amplified with the reverse primer introducing a SpeI site. Simultaneously, a fragment was amplified from β2.7 by PCR so that the resulting product carried a BamHI and HindIII site at the 5′ end and at the 3′ end, respectively. The β2.7 PCR product and the ATP8 PCR product were single digested with HindII and SpeI respectively, mixed in equimolar amounts and ligated at 22° C. for 3 hours using T4 DNA ligase (ThermoScientific). SpeI site can be ligated to HindIII site by a 2 base fill-in, which effectively destroys both restriction sites. Post-ligation, the product of the correct size was purified from the gel, the product this having the β2.7 fragment fused to the antisense ATP8 sequence. This ligation product was the PCR-amplified to introduce the BamHI and HindIII sites, respectively, and cloned back into the β2.7_pVAX1 vector backbone to generate the intact sequence. The clones were verified by BamHI and HindIII double digest, and then verified by sequencing (AITBiotech).

### Synthesis of RNA by in Vitro Transcription

In vitro transcription was carried out using T7 (ThermoScientific) and SP6 RNA polymerase (ThermoScientific). SP6 Polymerase was used for synthesizing antisense RNAs and the β2.7_GFP RNAs. The rest of the RNAs were synthesized using T7 RNA polymerase. The last 6 bases of the T7 and the SP6 promoter were selected, respectively, as previously described, and ultimately become a part of the T7 and the SP6 transcripts, respectively.

### Preparation of Templates for in Vitro Transcription

All plasmid vectors to be used for in vitro transcription were extracted overnight with Phenol:Chloroform:Isoamyl alcohol (25:24:1) and precipitated the next day using ethanol. The templates for in vitro transcription were prepared by PCR or by plasmid linearization. All restriction enzymes used for plasmid linearization generated 5′ overhangs to prevent formation of runaway transcripts. In case of PCR synthesized templates, the forward primer introduced the sequence of the T7/SP6 promoter for in vitro transcription. Both PCR templates and linearized plasmids were purified by PCR purification kit (Qiagen).

### In Vitro Transcription (IVT)

IVT was carried out using T7/SP6 RNA polymerase (ThermoScientific). lug of linearized plasmid/purified PCR template was incubated with 1× Transcription buffer, 10 mM NTP mix, 20U of RNaseOUT (Invitrogen) and 30U of SP6/T7 RNA polymerase in a final volume of 50 μl at 37c for 2 hours. Post 2 hour incubation, 3U of DNase I was added to the reaction mixture and incubated for at least 30 minutes at 37° C. RNA was then purified by Phenol chloroform extraction.

### Transfection

All transfections were carried out using Lipofectamine 2000 (Invitrogen) in Opti-MEM (GIBCO).

### Transfection of HepG2 Cells for Analysis of RNA Uptake

HepG2 cells were grown in T75 flasks in DMEM with antibiotics and transfected at 90% confluency. Transfection was carried out in Opti-MEM (GIBCO). lug equivalent of β2.7 RNA and its derivatives was transfected as per manufacturer's protocol. Media was changed 6 hours after transfection. 24 hours after transfection Mitochondrial RNA was isolated for analysis by Real Time PCR.

### Transfection of Hek293T Cells for Analysis of Mitochondrial RNA Knockdown

10⁵ cells of 293T Hek293T were grown in 24-well plates in DMEM with antibiotics and transfected the next day. Transfection was carried out in Opti-MEM (GIBCO). 800 ng equivalent of antisense_β2.7 fusion RNA was transfected as per manufacturer's protocol. Media was changed 6 hours after transfection. 24 hours after transfection, the total RNA was isolated and analysed by real time PCR.

### Transfection of HepG2 Cells for FACS Analysis and Confocal Microscopy

For FACS analysis 30,000 HepG2 cells were grown in 24-well plates and transfected at 30% confluency. Transfection was carried out in Opti-MEM (GIBCO). 800 ng of the control pEGFP-c1 and β2.7_GFP fusion plasmids (+NLS) was transfected as per manufacturer's protocol. Media was changed 6 hours after transfection. 24 hours after transfection, the cells were trypsinised and analysed for GFP expression using flow cytometry. For confocal microscopy analysis 20,000 HepG2 cells were seeded in 1.5 uM Chamber Slides (iBIO). Area of a chamber in the slides has the same as that of wells in 48-well plates and thus, transfection was carried out accordingly. 400 ng of the control pEGFP_C1 and β2.7_GFP fusion plasmids (+NLS) were transfected as per manufacturer's protocol. 24 hours after transfection, the cells were stained with respective dyes and analysed using confocal microscopy.

### Transfection of HEK293T for Cell Viability Analysis

50000 HEK293T cells were seeded in 24-well plates. 800 ng equivalent of the β2.7 RNA was added to each well and total RNA/well was adjusted to 800 ng using RNA previously isolated from untreated HEK293T cells. Media was changed after 6 hours. 10⁵ Hek293T cells were seeded in 24-well plates and transfected with 1.5 μg of β2.7 RNA_antisense fusion RNA. Since previously isolated RNA may contain target sequences hence the total RNA/well was adjusted to 1.5 μg using feeder RNA (yeast tRNA) instead of isolated RNA. Media was subsequently changed 6 hours after transfection.

### Transfection of Hek293T for CellTiter-Glo Assay

25000 Hek293T cells were seeded in 96-well plates and transfected with 300 ng equivalent of the β2.7 RNA_antisense RNA. Amount of RNA per well was adjusted using feeder RNA. Media was changed 6 hours after transfection.

### Isolation of RNA and Real Time PCR

### Isolation of Mitochondrial RNA

Mitochondria were isolated from HepG2 cells using the Mitochondria Isolation Kit (Biochain) as per manufacturer's guidelines. The isolated mitochondria were re-suspended in 1× Mitochondria Isolation Buffer. To remove contaminating cytoplasmic RNA, the mitochondrial suspension was treated with RNaseA (ThermoScientific) as previously described. Post-incubation, RNase A was inactivated by addition of 2× volumes of Trizol Reagent, following which the RNA was extracted using Trizol Reagent (Invitrogen), as per manufacturer's guidelines.

### Isolation of Total RNA

Hek293T cells seeded in 24-well plates were washed with 1× PBS. Subsequently, 200 μl of Trizol Reagent was added. The mixture in the 24-well was resuspended until the solution loses viscosity. The components were then transferred to an Eppendorf tube and RNA was isolated as per manufacturer's guidelines.

DNase Treatment and cDNA Synthesis

500 ng of isolated RNA (mitochondrial/total) was incubated with 1u DNase I (ThermoScientific) and 20U of RnaseOUT at 37° C. for 30 minutes. Post-incubation, ethylenediaaminetetraacetric acid (EDTA) was added to a final concentration of 3.75 mM and incubated at 75c for 12 minutes to inactivate DNase I. This reaction mixture was then reverse transcribed with 1× RT buffer, 5.5 mM MgCl2, 20U of RNaseOUT, 500 uM of each dNTP, 200 ng of Random Primers(Invitrogen), and 25U of multiscribe reverse transcriptase (ABI) at a final volume of 20 μl at 37° C. for 2 hours, followed by heat inactivation at 85° C. for 15 minutes.

### Real Time PCR

1 μl of cDNA was mixed with 5 μl of 2× SYBR CFX master mix and 400 nm of each (forward and reverse) primer. Real Time PCR was carried out in ABI 7900HT Real Time PCR machine using the following Thermal cycling conditions: 50° C. for 2 minutes, 95° C. for 10 minutes, 40 cycles of 95° C. for 15 seconds and 60° C. for 60 seconds. Each sample was run in duplicates.

### Relative Quantification

12 s rRNA was used as an internal control for normalization. Relative RNA levels were determined using the ΔΔCT method. Levels of cytoplasmic contaminant β-Actin were determined by comparison with an untreated mitochondrial sample.

### Absolute Quantification

In vitro transcribed RNA (to be transfected RNA) was serially diluted from 10¹⁰ to 10⁰¹ molecules, reverse transcribed and subjected to real-time PCR as described above. RNA standard curves were prepared by plotting Median CT values against the number of molecules per reaction. The copy number of each RNA (No. of molecules per micrograms (μg) of isolated RNA) was determined by comparison with the respective standard curve using the SDS2.4 software.

### Analysis of GFP Expression by Flow Cytometry

### Sample Preparation

HepG2 cells seeded in 24-wells were trypsinized, following which the trypsin was inactivated by the addition of complete DMEM. Cells in DMEM were resuspended 8 to 10 times to free cell clumps, and subsequently pelleted in a centrifuge at 6000 g for 5 minutes. Cells were then washed with 1× PBS and re-suspended in complete DMEM for flow cytometry analysis.

### Flow Cytometry Analysis

Flow cytometry analysis was carried out using a Beckmann Coulter CyAnADP flow cytometer. Cells were illuminated with a 488 nm laser, and gated using forward (FSC-A) and side scatter (SSC-A), along with doublet exclusion using FSC pulse width analysis. GFP expression was measured using a 510 nm to 540 nm bandpass filter. Up to 20,000 cells were measured on days 1, 3 and 5 post-transfection. Data was analysed using FlowJO 7.6.1.

### Confocal Microscopy

Cells were transfected in chamber slides and analysed on 3^{rd} day after transfection. Cells were stained with Hoechst 33342 (molecular probes) and/or Mitotracker Orange CMH2TMRos (Molecular Probes), as per manufacturer's guidelines. Cells were then counterstained with HCS CellMask Deep Red stain (Molecular Probes). Images were captured with Olympus FluoView FV1000 (Olympus, Japan) laser scanning confocal microscope, using a 60×/1.00 water objective, with 405 nm solid state laser diode (Hoechst), 488 nm argon ion laser (GFP), 543 nm HeNe Green laser (Mitoctracker) and 633 nm HeNe Red (cell mask). Images were subsequently analysed using ImageJ V1.48.

### Rotenone Induced Cell Death

200 mM rotenone stock solutions were prepared in anhydrous DMSO (Sigma). Rotenone stock solution was diluted to a final concentration of 200 uM in compete DMEM and filtered using a 0.22 uM filter. 24 hours after transfection, rotenone_DMEM was administered to Hek293T cells and cell death was determined 24, 48 and 72 hours after transfection using an alamar blue assay. After cell death assessment, cells were washed with PBS and fresh drug was administered for analysis on the subsequent time point.

### Alamar Blue Cell Viability Assay

Hek293T cells seeded in 24-well plates were subjected to alamar blue (Invitrogen) cell viability assay, as per manufacturer's guidelines. Fluorescence was measured at emission/excitation (530/590) using Biotek Synergy H1 Reader, with the sensitivity set to 60. The percentage (%) reduction in cell viability was determined by comparison with a cell-only control.

### Measurement of ATP Levels

Hek293T cells were transfected in 96-well plates and ATP levels were determined 24 hours after transfection using a CellTiter-Glo® Luminescent Cell Viability Assay (Promega). Luminescence was measured using a TECAN infinite M200PRO plate reader. Absolute ATP levels were determined from ATP standard curve, prepared as per manufacturer's guidelines. Relative changes in ATP levels were determined by comparison with a cell-only control.

### Statistical Analysis

Statistical analysis was carried out using GraphPad Prism Software 6.0. All numerical values presented as mean+SD (means plus standard deviation) of three independent experiments. Statistical significance was determined using Student's t-test and ANOVA.

### Generation of Construct ATP6_(D3)4_(D2)4

A stabilizing spacer sequence was inserted in (D2)4 downstream of the final D2 repeat to yield the (D2)4_S construct. The (D2)4_S was PCR amplified with primers introducing HindIII sites at either end. Subsequently the PCR product was HindIII digested and cloned within the HindIII site to yield the ATP6_(D3)4_(D2)4 construct.

### In Vitro Ttranscription (IVT) of Fluorescein-12 Uracil-Labelled RNA

Fluorescent RNA was synthesized by T7 RNA polymerase (Thermo Scientiflc™) via in vitro transcription using fluorescein-12-UTP (Enzo). 1 μg of linearized plasmid/purified PCR template was incubated with 5 μl of 5× transcription buffer, 5 μL of 10 mM NTP mix (10 mM GTP, 10 mM CTP, 10 mM ATP, 7.5 mM UTP, 2.5 mM fluorescein-12 UTP) 10 U of RNaseOUT™ (Invitrogen) and 20 U of T7 RNA polymerase in a final volume of 25 μL at 37° C. for 3 h. 3 U of DNase I was the added to the reaction mixture and incubated for at least 30 min at 37° C. to remove DNA template. RNA was then purified using the PCR purification kit (Qiagen). Quality of RNA was analyzed on ethidium bromide-free 1.5% agarose gel. Gels were illuminated on UV trans-illuminator and captured using a Samsung galaxy S7 smartphone. Intensity of the bands was determined using ImageJ v1.48.

### Confocal Microscopy

Cells were transfected in chamber slides and analyzed 24 h after transfection. Cells were stained with Hoechst 33342 (Molecular Probes), MitoTracker Orange CMH2TMRos (Molecular Probes) as per manufacturer's guidelines. Images were captured with Olympus FluoView FV1000 (Olympus, Japan) laser scanning confocal microscope using a 60×/1.00 water objective , with 405 nm solid state laser diode (Hoechst) ,488 nm argon ion laser(GFP), 543 nm HeNe Green laser (Mitotracker) Images were subsequently analyzed using ImageJ V1.48. The extent of co-localization of GFP (green) within the mitochondrial (Red) fraction was determined using the plugin JACOP and the mander's overlap coefficient (MOC) was reported.

### Tables

TABLE 2
Overview of sequences presented in the attached sequence listing. “AS” stands
for antisense; “endo” stands for endogenously transcribed RNA. These sequences
were tested as plasmids. “_core” stands for core sequences, which are the minimum
polynucleotide sequences (without spacers) that are operable.

| | | | | | | |
|---|---|---|---|---|---|---|
| SEQ ID | | | | Restriction | | |
| NO: | Name | Core | Promoter | sites | Spacer | Poly A |

Tested as in vitro transcribed RNA (no polyA)

| | | | | | | |
|---|---|---|---|---|---|---|
| 1 | Full length β2.7 | Yes | Yes | Yes | No | No |
| | RNA (sense) | | | | | |
| 2 | Full length β2.7 | Yes | Yes | Yes | No | No |
| | RNA_AS (antisense) | | | | | |
| 3 | Sense Domain 1: | Yes | Yes | Yes | No | No |
| | D1 | | | | | |
| 4 | D2 | Yes | Yes | Yes | No | No |
| 5 | D3 | Yes | Yes | Yes | No | No |
| 6 | D4 | Yes | Yes | Yes | No | No |
| 7 | Antisense Domain 1: | Yes | Yes | Yes | No | No |
| | D1AS | | | | | |
| 8 | D2AS | Yes | Yes | Yes | No | No |
| 9 | D3AS | Yes | Yes | Yes | No | No |
| 10 | D4AS | Yes | Yes | Yes | No | No |
| 11 | Sense Domain 1 Deletion | Yes | Yes | Yes | No | No |
| | Mutant: ΔD1 | | | | | |
| 12 | ΔD2 | Yes | Yes | Yes | No | No |
| 13 | ΔD3 | Yes | Yes | Yes | No | No |
| 14 | ΔD4 | Yes | Yes | Yes | No | No |
| 15 | Multimer Sequence | Yes | Yes | Yes | Yes | No |
| | D2 × 4 | | | | | |
| 16 | Multimer Sequence | Yes | Yes | Yes | Yes | No |
| | D3 × 4 | | | | | |
| 17 | Multimer Sequence | Yes | Yes | Yes | Yes | No |
| | D3 × 4_D2 × 4 | | | | | |
| 18 | Multimer Sequence | Yes | Yes | Yes | Yes | No |
| | D2 × 4_D3 × 4 | | | | | |
| 19 | Multimer Sequence | Yes | Yes | Yes | Yes | No |
| | (D3_D2) × 4 | | | | | |
| 20 | gGFP_β2.7 | Yes | Yes | Yes | Yes | No |
| 21 | gGFP_s_rβ2.7 | Yes | Yes | Yes | Yes | No |
| 22 | mtGFP_s_β2.7 | Yes | Yes | Yes | Yes | No |
| 23 | mtGFP_s | Yes | Yes | Yes | Yes | No |
| 24 | asATP6_s6_β2.7 | Yes | Yes | Yes | Yes | No |
| 25 | β2.7_s8_asATP8 | Yes | Yes | Yes | Yes | No |
| 26 | asATP6_D3₄_D2₄ | Yes | Yes | Yes | Yes | No |

Spacers (only)

| | | | | | | |
|---|---|---|---|---|---|---|
| 27 | S1a | No | No | No | Yes | No |
| 28 | S1b | No | No | No | Yes | No |
| 29 | S2a | No | No | No | Yes | No |
| 30 | S2b | No | No | No | Yes | No |
| 31 | S3a | No | No | No | Yes | No |
| 32 | S3b | No | No | No | Yes | No |
| 33 | S4a | No | No | No | Yes | No |
| 34 | S4b | No | No | No | Yes | No |
| 35 | S6a | No | No | No | Yes | No |
| 36 | S6b | No | No | No | Yes | No |
| 37 | S8a | No | No | No | Yes | No |
| 38 | S8b | No | No | No | Yes | No |
| 39 | Spacer FIG. 3a | No | No | No | Yes | No |

Tested as in vitro transcribed RNA (no polyA)

| | | | | | | |
|---|---|---|---|---|---|---|
| 40 | Antisense Domain 1 | Yes | Yes | Yes | No | No |
| | Deletion Mutant: ΔD1AS | | | | | |
| 41 | ΔD2AS | Yes | Yes | Yes | No | No |
| 42 | ΔD3AS | Yes | Yes | Yes | No | No |
| 43 | ΔD4AS | Yes | Yes | Yes | No | No |

Tested as plasmid DNA (endogenous transcript, all features)

| | | | | | | |
|---|---|---|---|---|---|---|
| 44 | gGFP_β2.7_endo | Yes | Yes | Yes | Yes | Yes |
| 45 | gGFP_s_β2.7_endo | Yes | Yes | Yes | Yes | Yes |
| 46 | mtGFP_s_β2.7_endo | Yes | Yes | Yes | Yes | Yes |
| 47 | gGFP_NLS_s_β2.7_endo | Yes | Yes | Yes | Yes | Yes |
| 48 | mtGFP_s_endo | Yes | Yes | Yes | Yes | Yes |
| 49 | Full length β2.7 | Yes | Yes | Yes | Yes | Yes |
| | RNA_endo (sense) | | | | | |
| 50 | β2.7 RNA domain | Yes | Yes | Yes | Yes | Yes |
| | 2_endo (sense) | | | | | |
| 51 | β2.7 RNA domain | Yes | Yes | Yes | Yes | Yes |
| | 3_endo (sense) | | | | | |
| 52 | Multimer sequence | Yes | Yes | Yes | Yes | Yes |
| | D2 × 4_endo | | | | | |
| 53 | Multimer sequence | Yes | Yes | Yes | Yes | Yes |
| | D3 × 4_endo | | | | | |
| 54 | Multimer sequence | Yes | Yes | Yes | Yes | Yes |
| | D3 × 4_D2 × 4_endo | | | | | |
| 55 | β2.7 core | Yes | No | No | No | No |
| 56 | D1_core | Yes | No | No | No | No |
| 57 | D2_core | Yes | No | No | No | No |
| 58 | D3_core | Yes | No | No | No | No |
| 59 | D4_core | Yes | No | No | No | No |
| 60 | ΔD1 _core | Yes | No | No | No | No |
| 61 | ΔD2_core | Yes | No | No | No | No |
| 62 | ΔD3_core | Yes | No | No | No | No |
| 63 | ΔD4_core | Yes | No | No | No | No |
| 64 | β2.7 RNA_AS_core | Yes | No | No | No | No |
| 65 | Domain 1 antisense: | Yes | No | No | No | No |
| | D1AS_core | | | | | |
| 66 | D2AS_core | Yes | No | No | No | No |
| 67 | D3AS_core | Yes | No | No | No | No |
| 68 | D4AS_core | Yes | No | No | No | No |
| 69 | ΔD1AS_core | Yes | No | No | No | No |
| 70 | ΔD2AS_core | Yes | No | No | No | No |
| 71 | ΔD3AS_core | Yes | No | No | No | No |
| 72 | ΔD4AS_core | Yes | No | No | No | No |
| 73 | Multimer sequence | Yes | No | No | No | No |
| | D2 × 4_core | | | | | |
| 74 | Multimer sequence | Yes | No | No | No | No |
| | D3 × 4_core | | | | | |
| 75 | Multimer sequence | Yes | No | No | No | No |
| | D3 × 4_D2 × 4_core | | | | | |
| 76 | Multimer sequence | Yes | No | No | No | No |
| | D2 × 4_D3 × 4_core | | | | | |
| 77 | Multimer sequence | Yes | No | No | No | No |
| | (D3_D2) × 4_core | | | | | |

Fusions of the sense β2.7 RNA to different payloads but excluding
promoter, spacer, restriction site, and polyA sequences

| | | | | | | |
|---|---|---|---|---|---|---|
| 78 | gGFP_β2.7 (core) | Yes | No | No | No | No |
| 79 | gGFP_s_β2.7 (core) | Yes | No | No | No | No |
| 80 | mtGFP_s_β2.7 (core) | Yes | No | No | No | No |
| 81 | mtGFP_s (core) | Yes | No | No | No | No |
| 82 | asATP6_s6_β2.7 (core) | Yes | No | No | No | No |
| 83 | β2.7 _s8_asATP8 (core) | Yes | No | No | No | No |
| 84 | ATP6_(D3)₄_(D2)₄ | Yes | No | No | No | No |
| | (core) | | | | | |

TABLE 3
Sequences. The following sequences 1 to 26 and 44 to 47 were tested as in vitro
transcribed RNA using as DNA template either linearized DNA (RNA ending with
cleavage site) or PCT products (RNA ending with the end of the PCR template).
As promoters, either the T7 RNA (RNA starts with GGCGCU) or the SP6 (RNA starts
with GGAGUC) promoter was used. After the 6 transcribed promoter nucleotides,
there is either a restriction site or not.

| SEQ | | |
|---|---|---|
| ID | Name | |
| NO: | (type) | Sequence |
| 1 | Full length | GGCGCUUCCGGAAGAGCUAGCUCCCCAGAUCGCUGCUGCCCCGGC |
| | B2.7 RNA | GUUCUCCAGAAGCCCCGGCGGGCGAAUCGGCCGGCUGGUCGGUCG |
| | (sense) | GCGCUCGGACGGAUGGGGAGAACGGCGGUGACUUAGCCGCCCGUG |
| | | GCCGGGAGAAGAUGGAGGAGCCGAGAUGACAACGGCAGUCGUGGA |
| | | AGGGUCGCCAAGCCCCGGUCCUUCUCUUCUGUCUGGUCGAAUCUC |
| | | GUUUUCUUUUUUCAACCGCUCUUUUUAUCACCUUUUUAUGUGAGU |
| | | UUCUCUUCCGCGUCUCCCGGCCGUACCAUCCACCCAUGCAGCAUGC |
| | | ACGCGUGUAUGUAUGCAUCGUCUCUCCUCCGUCCCGACUACCAUC |
| | | AGCAGCACCACUACCGCCACCCCCAGCGCCACCACCGCUGCCGUCG |
| | | CCACCGCGUUAUCCGUUCCUCGUAGGCUGGUCCUGGGGAACGGGU |
| | | CGGCGGCCGGUCGGCUUCUGUUUUAUUAUUUUUUUUUAUUUUUUA |
| | | UCUUCUCCUUUCCUUAAUCUCGGAUUAUCAUUUCCCUCUCCUACC |
| | | UACCACGAAUCGCAGAUGAUAAACAAGAGGGUAAAAAGAAAAAA |
| | | GCUACAGACAUUUGGGUACCUCAGCUUUCCGAUAACUCGAAGAAU |
| | | UCAAAGUCGACGAUUCCCAACAAGAGAAAACAGAACAAAAACAAG |
| | | GUCAUUUUUAUUUAUCCUCAUCGUCAACAACAACUACCGACAACA |
| | | ACGAAACACCACCAAGAAUGUCAAUCCGCAAGGGUGUUCCUGCCC |
| | | CCUCGACGCGCCUGUCGCGAUCCUCAUGGCGAGGACCGCGAUCUC |
| | | CGUAUAGGUAGAUGAAAUUAUCCCGUGUCCGGUCCUGAUUCCCCG |
| | | CAUGCCCUGCACAUCCUGACGCGUCGGUCAGCAGCCAAACAAUCA |
| | | UAGGAAAUGAACCAGAAGAACAAAAAGAUCAUCUCUCUCGGUGUA |
| | | UAGCAACACCAACAACAACCGCAUCGCAACAUCUUCAUCCGCAAG |
| | | ACGGAAAGAAAACAACAAUAAUGAGAAUGAAAUCACCACAACCAA |
| | | GCCAGAUUUCACGUCCAUGAGUUUUUAUUAUAUUAUUAUCAAAAC |
| | | GAAAAACAGAAAAACUGUCAUAGAUAAAUAUAAAAAAAAAUAGA |
| | | AACCACAAACGACUACUAGUACUCCAAUCUUAGAUGUAUAUGCUC |
| | | CUAGAUAAGAUUUAGUAUUACCAUAAUCAUCGAAGAAUGAAAGA |
| | | CGACGAUGAUUCCUUACCGCUCCUGCCACCCGGUCUGUAUGUAGA |
| | | GAGAGAAGAGAGAAAACGGUGAAUCCAAGAUCCCCGGGUCGGCGU |
| | | CGGCAUGCCGCUGAUCGCAGUGGCCCCACCUCGGCAUGCCGGCGCC |
| | | GGGCGAGGAAUUGCUCAUGAAAAAAGUAUCUUUCUGUAAAAAAA |
| | | GAAAACAAUACAUGAUUAACCGAAAAGAAACCAACAAAAAGAACC |
| | | CGAGAUCAGUCGAUUUCGAUCACUACGAUAAACACAUGGAAGAUU |
| | | UCUUGAAAAAAGAAAAGAGAAAGAGACCACCUUCCCGGCGGCGGA |
| | | CACGCUCCUCUCCGUCGCCGUUCUGCACCAUGAUUCGAUCAAUAA |
| | | CAACAUCAUCAUCGGAGACCAUCUUUUAAUCAAUCAGCGUUGCAG |
| | | UAGUCGACUCCCUGGACACGAAGGAGUCAUCCAUUUUUAUCCUCG |
| | | CACUUCUUCGCUCUCAAAGCCGCCUUUAAAGUUGAAAUGAAAGGA |
| | | UGGAAACAUGGAAUACAGUUUUAAUUGCACGUAUCACCAUUUUAC |
| | | UACAAAAAGAAAAAAAAACAACUUACACAUAGUAUUACCUUAGGU |
| | | UUACGGAUAAGUAGAGUGUAGGCGUUUUUGAAACAGUUCAGCCA |
| | | AUGCAAUCUUGUCUCGGCAUAAUCACUCUUUCUGCAUAUAAUAGU |
| | | AGUAGUAGAUUUAUUCACAUCAACACAGCGAAAAACUCCAGCAUC |
| | | AAAGUACACCUAGAGACAGCCCUUAAAAUAUAGUUUGCAGCUUUU |
| | | AGAUGUACUUACACCAAAGAAGAUUACCGUCCUUACGAGAAAACA |
| | | GAUACUCGGAUAUAGGAAUCAAGACAGCUCUGCACUGAAAACACA |
| | | CUCUCCUGUCACGACACCGCGCCACACCAGAGGCGUACGCGUGAC |
| | | UUCAUCGCAACGAUCCAUCGUGAUGUCCCUCGCAGAACCUAAAAA |
| | | GACCAAAAAAAAAUCUUGGACCACAGUUGUCGAUUCUUGAAGACA |
| | | AUAUUCUCGUGAGAACUUUGAGAUUCGCACUUGAAACCUCUUAGG |
| | | AUCCACAAAAACAACAACCUCUGUAUGGAAAAUGCGCUAUUUUAU |
| | | CUCAGCUUUUCUCCCAAACCUCGGUUUCUUCCUAUUCUUAAGUUU |
| | | UCCCUAGUAUAUUUGCCUCCUUAUAAGAAAAGAAGCACAAGCUCG |
| | | GUCGCACGGAUUAUUCCUUCUGCUAAUCUAUUAUUUUGUUCCUUU |
| | | UUUUUUUGUUUGCCUUCACCCCCUUCACUCCCUGUAGCAACACAG |
| | | AGUAGUAGACACAAUAAAUGAGAAGUUUGCAUGCAAGCU |
| 2 | Full length | GGAGUCAAGCUUGCAUGCAAACUUCUCAUUUAUUGUGUCUACUAC |
| | B2.7 RNA | UCUGUGUUGCUACAGGGAGUGAAGGGGGUGAAGGCAAACAAAAA |
| | (antisense) | AAAAAGGAACAAAAUAAUAGAUUAGCAGAAGGAAUAAUCCGUGC |
| | | GACCGAGCUUGUGCUUCUUUUCUUAUAAGGAGGCAAAUAUACUAG |
| | | GGAAAACUUAAGAAUAGGAAGAAACCGAGGUUUGGGAGAAAAGC |
| | | UGAGAUAAAAUAGCGCAUUUUCCAUACAGAGGUUGUUGUUUUUG |
| | | UGGAUCCUAAGAGGUUUCAAGUGCGAAUCUCAAAGUUCUCACGAG |
| | | AAUAUUGUCUUCAAGAAUCGACAACUGUGGUCCAAGAUUUUUUUU |
| | | UGGUCUUUUUAGGUUCUGCGAGGGACAUCACGAUGGAUCGUUGCG |
| | | AUGAAGUCACGCGUACGCCUCUGGUGUGGCGCGGUGUCGUGACAG |
| | | GAGAGUGUGUUUUCAGUGCAGAGCUGUCUUGAUUCCUAUAUCCGA |
| | | GUAUCUGUUUUCUCGUAAGGACGGUAAUCUUCUUUGGUGUAAGU |
| | | ACAUCUAAAAGCUGCAAACUAUAUUUUAAGGGCUGUCUCUAGGUG |
| | | UACUUUGAUGCUGGAGUUUUUCGCUGUGUUGAUGUGAAUAAAUC |
| | | UACUACUACUAUUAUAUGCAGAAAGAGUGAUUAUGCCGAGACAAG |
| | | AUUGCAUUGGCUGAACUGUUUCAAAAACGCCUACACUCUACUUAU |
| | | CCGUAAACCUAAGGUAAUACUAUGUGUAAGUUGUUUUUUUUUCU |
| | | UUUUGUAGUAAAAUGGUGAUACGUGCAAUUAAAACUGUAUUCCA |
| | | UGUUUCCAUCCUUUCAUUUCAACUUUAAAGGCGGCUUUGAGAGCG |
| | | AAGAAGUGCGAGGAUAAAAAUGGAUGACUCCUUCGUGUCCAGGGA |
| | | GUCGACUACUGCAACGCUGAUUGAUUAAAAGAUGGUCUCCGAUGA |
| | | UGAUGUUGUUAUUGAUCGAAUCAUGGUGCAGAACGGCGACGGAG |
| | | AGGAGCGUGUCCGCCGCCGGGAAGGUGGUCUCUUUCUCUUUUCUU |
| | | UUUUCAAGAAAUCUUCCAUGUGUUUAUCGUAGUGAUCGAAAUCGA |
| | | CUGAUCUCGGGUUCUUUUUGUUGGUUUCUUUUCGGUUAAUCAUGU |
| | | AUUGUUUUCUUUUUUUACAGAAAGAUACUUUUUUCAUGAGCAAU |
| | | UCCUCGCCCGGCGCCGGCAUGCCGAGGUGGGGCCACUGCGAUCAG |
| | | CGGCAUGCCGACGCCGACCCGGGGAUCUUGGAUUCACCGUUUUCU |
| | | CUCUUCUCUCUCUACAUACAGACCGGGUGGCAGGAGCGGUAAGGA |
| | | AUCAUCGUCGUCUUUCAUUCUUCGAUGAUUAUGGUAAUACUAAAU |
| | | CUUAUCUAGGAGCAUAUACAUCUAAGAUUGGAGUACUAGUAGUCG |
| | | UUUGUGGUUUCUAUUUUUUUUUAUAUUUAUCUAUGACAGUUUUU |
| | | CUGUUUUUCGUUUUGAUAAUAAUAUAAUAAAAACUCAUGGACGU |
| | | GAAAUCUGGCUUGGUUGUGGUGAUUUCAUUCUCAUUAUUGUUGU |
| | | UUUCUUUCCGUCUUGCGGAUGAAGAUGUUGCGAUGCGGUUGUUGU |
| | | UGGUGUUGCUAUACACCGAGAGAGAUGAUCUUUUUGUUCUUCUGG |
| | | UUCAUUUCCUAUGAUUGUUUGGCUGCUGACCGACGCGUCAGGAUG |
| | | UGCAGGGCAUGCGGGGAAUCAGGACCGGACACGGGAUAAUUUCAU |
| | | CUACCUAUACGGAGAUCGCGGUCCUCGCCAUGAGGAUCGCGACAG |
| | | GCGCGUCGAGGGGGCAGGAACACCCUUGCGGAUUGACAUUCUUGG |
| | | UGGUGUUUCGUUGUUGUCGGUAGUUGUUGUUGACGAUGAGGAUA |
| | | AAUAAAAAUGACCUUGUUUUUGUUCUGUUUUCUCUUGUUGGGAA |
| | | UCGUCGACUUUGAAUUCUUCGAGUUAUCGGAAAGCUGAGGUACCC |
| | | AAAUGUCUGUAGCUUUUUUCUUUUUACCCUCUUGUUUAUCAUCUG |
| | | CGAUUCGUGGUAGGUAGGAGAGGGAAAUGAUAAUCCGAGAUUAA |
| | | GGAAAGGAGAAGAUAAAAAAUAAAAAAAAAUAAUAAAACAGAAG |
| | | CCGACCGGCCGCCGACCCGUUCCCCAGGACCAGCCUACGAGGAACG |
| | | GAUAACGCGGUGGCGACGGCAGCGGUGGUGGCGCUGGGGGUGGCG |
| | | GUAGUGGUGCUGCUGAUGGUAGUCGGGACGGAGGAGAGACGAUG |
| | | CAUACAUACACGCGUGCAUGCUGCAUGGGUGGAUGGUACGGCCGG |
| | | GAGACGCGGAAGAGAAACUCACAUAAAAAGGUGAUAAAAAGAGC |
| | | GGUUGAAAAAAGAAAACGAGAUUCGACCAGACAGAAGAGAAGGA |
| | | CCGGGGCUUGGCGACCCUUCCACGACUGCCGUUGUCAUCUCGGCU |
| | | CCUCCAUCUUCUCCCGGCCACGGGCGGCUAAGUCACCGCCGUUCUC |
| | | CCCAUCCGUCCGAGCGCCGACCGACCAGCCGGCCGAUUCGCCCGCC |
| | | GGGGCUUCUGGAGAACGCCGGGGCAGCAGCGAUCUGGGGAUGUGC |
| | | UAGCUCCGG |
| 3 | Sense | GGCGCUUCCGGACAGAAGCCCCGGCGGGCGAAUCGGCCGGCUGGU |
| | domain 1: | CGGUCGGCGCUCGGACGGAUGGGGAGAACGGCGGUGACUUAGCCG |
| | D1 | CCCGUGGCCGGGAGAAGAUGGAGGAGCCGAGAUGACAACGGCAGU |
| | | CGUGGAAGGGUCGCCAAGCCCCGGUCCUUCUCUUCUGUCUGGUCG |
| | | AAUCUCGUUUUCUUUUUUCAACCGCUCUUUUUAUCACCUUUUUAU |
| | | GUGAGUUUCUCUUCCGCGUCUCCCGGCCGUACCAUCCACCCAUGC |
| | | AGCAUGCACGCGUGUAUGUAUGCAUCGUCUCUCCUCCGUCCCGAC |
| | | UACCAUCAGCAGCACCACUACCGCCACCCCCAGCGCCACCACCGCU |
| | | GCCGUCGCCACCGCGUUAUCCGUUCCUCGUAGGCUGGUCCUGGGG |
| | | AACGGGUCGGCGGCCGGUCGGCUUCUGAAGCU |
| 4 | Sense | GGCGCUUCCGGAUUUUAUUUUUUAUCUUCUCCUUUCCUUAAUCUC |
| | domain 2: | GGAUUAUCAUUUCCCUCUCCUACCUACCACGAAUCGCAGAUGAUA |
| | D2 | AACAAGAGGGUAAAAAGAAAAAAAGCU |
| 5 | Sense | GGCGCUUCCGGAGGUCAUUUUUAUUUAUCCUCAUCGUCAACAACA |
| | domain 3: | ACUACCGACAACAACGAAACACCACCAAGAAUGUCAAUCCGCAAG |
| | D3 | GGUGUUCCUGCCCCCUCGACGCGCCUGUCGCGAUCCUCAUGGCGA |
| | | GGACCGCGAUCUCCGUAUAGGUAGAUGAAAUUAUCCCGUGUCCGG |
| | | UCCUGAUUCCCCGCAUGCCCUGCACAUCCUGACGCGUCGGUCAGC |
| | | AGCCAAACAAUCAUAGGAAAUGAACAAGCU |
| 6 | Sense | GGCGCUUCCGGACGGCGUCGGCAUGCCGCUGAUCGCAGUGGCCCC |
| | domain 4: | ACCUCGGCAUGCCGGCGCCGGGCGAGGAAUUGCUCAUGAAAAAAG |
| | D4 | UAUCUUUCUGUAAAAAAAGAAAACAAUACAUGAUUAACCGAAAA |
| | | GAAACCAACAAAAAGAACCCGAGAUCAGUCGAUUUCGAUCACUAC |
| | | GAUAAACACAUGGAAGAUUUCUUGAAAAAAGAAAAGAGAAAGAG |
| | | ACCACCUUCCCGGCGGCGGACACGCUCCUCUCCGUCGCCGUUCUGC |
| | | ACCAUGAUUCGAUCAAUAACAACAUCAUCAUCGGAGACCAUCUUU |
| | | UAAUCAAUCAGCGUUGCAGUAGUCGACUCCCUGGACACGAAGGAG |
| | | UCAUCCAUUUUUAUCCUCGCAAGCU |
| 7 | Antisense | GGAGUCAAGCUUCAGAAGCCGACCGGCCGCCGACCCGUUCCCCAG |
| | domain 1: | GACCAGCCUACGAGGAACGGAUAACGCGGUGGCGACGGCAGCGGU |
| | D1AS | GGUGGCGCUGGGGGUGGCGGUAGUGGUGCUGCUGAUGGUAGUCG |
| | | GGACGGAGGAGAGACGAUGCAUACAUACACGCGUGCAUGCUGCAU |
| | | GGGUGGAUGGUACGGCCGGGAGACGCGGAAGAGAAACUCACAUAA |
| | | AAAGGUGAUAAAAAGAGCGGUUGAAAAAAGAAAACGAGAUUCGA |
| | | CCAGACAGAAGAGAAGGACCGGGGCUUGGCGACCCUUCCACGACU |
| | | GCCGUUGUCAUCUCGGCUCCUCCAUCUUCUCCCGGCCACGGGCGGC |
| | | UAAGUCACCGCCGUUCUCCCCAUCCGUCCGAGCGCCGACCGACCAG |
| | | CCGGCCGAUUCGCCCGCCGGGGCUUCUGUCCGG |
| 8 | Antisense | GGAGUCAAGCUUUUUUUCUUUUUACCCUCUUGUUUAUCAUCUGCG |
| | domain 2: | AUUCGUGGUAGGUAGGAGAGGGAAAUGAUAAUCCGAGAUUAAGG |
| | D2AS | AAAGGAGAAGAUAAAAAAUAAAAUCCGG |
| 9 | Antisense | GGAGUCAAGCUUGUUCAUUUCCUAUGAUUGUUUGGCUGCUGACCG |
| | domain 3: | ACGCGUCAGGAUGUGCAGGGCAUGCGGGGAAUCAGGACCGGACAC |
| | D3AS | GGGAUAAUUUCAUCUACCUAUACGGAGAUCGCGGUCCUCGCCAUG |
| | | AGGAUCGCGACAGGCGCGUCGAGGGGGCAGGAACACCCUUGCGGA |
| | | UUGACAUUCUUGGUGGUGUUUCGUUGUUGUCGGUAGUUGUUGUU |
| | | GACGAUGAGGAUAAAUAAAAAUGACCUCCGG |
| 10 | Antisense | GGAGUCAAGCUUGCGAGGAUAAAAAUGGAUGACUCCUUCGUGUCC |
| | domain 4: | AGGGAGUCGACUACUGCAACGCUGAUUGAUUAAAAGAUGGUCUCC |
| | D4AS | GAUGAUGAUGUUGUUAUUGAUCGAAUCAUGGUGCAGAACGGCGA |
| | | CGGAGAGGAGCGUGUCCGCCGCCGGGAAGGUGGUCUCUUUCUCUU |
| | | UUCUUUUUUCAAGAAAUCUUCCAUGUGUUUAUCGUAGUGAUCGAA |
| | | AUCGACUGAUCUCGGGUUCUUUUUGUUGGUUUCUUUUCGGUUAAU |
| | | CAUGUAUUGUUUUCUUUUUUUACAGAAAGAUACUUUUUUCAUGA |
| | | GCAAUUCCUCGCCCGGCGCCGGCAUGCCGAGGUGGGGCCACUGCG |
| | | AUCAGCGGCAUGCCGACGCCGUCCGG |
| 11 | Sense | GGCGCUUCCGGAAGAGCUAGCUCCCCAGAUCGCUGCUGCCCCGGC |
| | domain 1 | GUUCUCUUUUAUUAUUUUUUUUUAUUUUUUAUCUUCUCCUUUCCU |
| | deletion | UAAUCUCGGAUUAUCAUUUCCCUCUCCUACCUACCACGAAUCGCA |
| | mutant: | GAUGAUAAACAAGAGGGUAAAAAGAAAAAAGCUACAGACAUUUG |
| | ΔD1 | GGUACCUCAGCUUUCCGAUAACUCGAAGAAUUCAAAGUCGACGAU |
| | | UCCCAACAAGAGAAAACAGAACAAAAACAAGGUCAUUUUUAUUUA |
| | | UCCUCAUCGUCAACAACAACUACCGACAACAACGAAACACCACCA |
| | | AGAAUGUCAAUCCGCAAGGGUGUUCCUGCCCCCUCGACGCGCCUG |
| | | UCGCGAUCCUCAUGGCGAGGACCGCGAUCUCCGUAUAGGUAGAUG |
| | | AAAUUAUCCCGUGUCCGGUCCUGAUUCCCCGCAUGCCCUGCACAU |
| | | CCUGACGCGUCGGUCAGCAGCCAAACAAUCAUAGGAAAUGAACCA |
| | | GAAGAACAAAAAGAUCAUCUCUCUCGGUGUAUAGCAACACCAACA |
| | | ACAACCGCAUCGCAACAUCUUCAUCCGCAAGACGGAAAGAAAACA |
| | | ACAAUAAUGAGAAUGAAAUCACCACAACCAAGCCAGAUUUCACGU |
| | | CCAUGAGUUUUUAUUAUAUUAUUAUCAAAACGAAAAACAGAAAA |
| | | ACUGUCAUAGAUAAAUAUAAAAAAAAAUAGAAACCACAAACGACU |
| | | ACUAGUACUCCAAUCUUAGAUGUAUAUGCUCCUAGAUAAGAUUUA |
| | | GUAUUACCAUAAUCAUCGAAGAAUGAAAGACGACGAUGAUUCCUU |
| | | ACCGCUCCUGCCACCCGGUCUGUAUGUAGAGAGAGAAGAGAGAAA |
| | | ACGGUGAAUCCAAGAUCCCCGGGUCGGCGUCGGCAUGCCGCUGAU |
| | | CGCAGUGGCCCCACCUCGGCAUGCCGGCGCCGGGCGAGGAAUUGC |
| | | UCAUGAAAAAAGUAUCUUUCUGUAAAAAAAGAAAACAAUACAUG |
| | | AUUAACCGAAAAGAAACCAACAAAAAGAACCCGAGAUCAGUCGAU |
| | | UUCGAUCACUACGAUAAACACAUGGAAGAUUUCUUGAAAAAAGAA |
| | | AAGAGAAAGAGACCACCUUCCCGGCGGCGGACACGCUCCUCUCCG |
| | | UCGCCGUUCUGCACCAUGAUUCGAUCAAUAACAACAUCAUCAUCG |
| | | GAGACCAUCUUUUAAUCAAUCAGCGUUGCAGUAGUCGACUCCCUG |
| | | GACACGAAGGAGUCAUCCAUUUUUAUCCUCGCACUUCUUCGCUCU |
| | | CAAAGCCGCCUUUAAAGUUGAAAUGAAAGGAUGGAAACAUGGAA |
| | | UACAGUUUUAAUUGCACGUAUCACCAUUUUACUACAAAAAGAAAA |
| | | AAAAACAACUUACACAUAGUAUUACCUUAGGUUUACGGAUAAGUA |
| | | GAGUGUAGGCGUUUUUGAAACAGUUCAGCCAAUGCAAUCUUGUCU |
| | | CGGCAUAAUCACUCUUUCUGCAUAUAAUAGUAGUAGUAGAUUUAU |
| | | UCACAUCAACACAGCGAAAAACUCCAGCAUCAAAGUACACCUAGA |
| | | GACAGCCCUUAAAAUAUAGUUUGCAGCUUUUAGAUGUACUUACAC |
| | | CAAAGAAGAUUACCGUCCUUACGAGAAAACAGAUACUCGGAUAUA |
| | | GGAAUCAAGACAGCUCUGCACUGAAAACACACUCUCCUGUCACGA |
| | | CACCGCGCCACACCAGAGGCGUACGCGUGACUUCAUCGCAACGAU |
| | | CCAUCGUGAUGUCCCUCGCAGAACCUAAAAAGACCAAAAAAAAAU |
| | | CUUGGACCACAGUUGUCGAUUCUUGAAGACAAUAUUCUCGUGAGA |
| | | ACUUUGAGAUUCGCACUUGAAACCUCUUAGGAUCCACAAAAACAA |
| | | CAACCUCUGUAUGGAAAAUGCGCUAUUUUAUCUCAGCUUUUCUCC |
| | | CAAACCUCGGUUUCUUCCUAUUCUUAAGUUUUCCCUAGUAUAUUU |
| | | GCCUCCUUAUAAGAAAAGAAGCACAAGCUCGGUCGCACGGAUUAU |
| | | UCCUUCUGCUAAUCUAUUAUUUUGUUCCUUUUUUUUUUGUUUGCC |
| | | UUCACCCCCUUCACUCCCUGUAGCAACACAGAGUAGUAGACACAA |
| | | UAAAUGAGAAGUUUGCAUGCAAGCU |
| 12 | Sense | GGCGCUUCCGGAAGAGCUAGCUCCCCAGAUCGCUGCUGCCCCGGC |
| | domain 2 | GUUCUCCAGAAGCCCCGGCGGGCGAAUCGGCCGGCUGGUCGGUCG |
| | deletion | GCGCUCGGACGGAUGGGGAGAACGGCGGUGACUUAGCCGCCCGUG |
| | mutant: | GCCGGGAGAAGAUGGAGGAGCCGAGAUGACAACGGCAGUCGUGGA |
| | ΔD2 | AGGGUCGCCAAGCCCCGGUCCUUCUCUUCUGUCUGGUCGAAUCUC |
| | | GUUUUCUUUUUUCAACCGCUCUUUUUAUCACCUUUUUAUGUGAGU |
| | | UUCUCUUCCGCGUCUCCCGGCCGUACCAUCCACCCAUGCAGCAUGC |
| | | ACGCGUGUAUGUAUGCAUCGUCUCUCCUCCGUCCCGACUACCAUC |
| | | AGCAGCACCACUACCGCCACCCCCAGCGCCACCACCGCUGCCGUCG |
| | | CCACCGCGUUAUCCGUUCCUCGUAGGCUGGUCCUGGGGAACGGGU |
| | | CGGCGGCCGGUCGGCUUCUGUUUUAUUAUUUUUAGCUACAGACAU |
| | | UUGGGUACCUCAGCUUUCCGAUAACUCGAAGAAUUCAAAGUCGAC |
| | | GAUUCCCAACAAGAGAAAACAGAACAAAAACAAGGUCAUUUUUAU |
| | | UUAUCCUCAUCGUCAACAACAACUACCGACAACAACGAAACACCA |
| | | CCAAGAAUGUCAAUCCGCAAGGGUGUUCCUGCCCCCUCGACGCGC |
| | | CUGUCGCGAUCCUCAUGGCGAGGACCGCGAUCUCCGUAUAGGUAG |
| | | AUGAAAUUAUCCCGUGUCCGGUCCUGAUUCCCCGCAUGCCCUGCA |
| | | CAUCCUGACGCGUCGGUCAGCAGCCAAACAAUCAUAGGAAAUGAA |
| | | CCAGAAGAACAAAAAGAUCAUCUCUCUCGGUGUAUAGCAACACCA |
| | | ACAACAACCGCAUCGCAACAUCUUCAUCCGCAAGACGGAAAGAAA |
| | | ACAACAAUAAUGAGAAUGAAAUCACCACAACCAAGCCAGAUUUCA |
| | | CGUCCAUGAGUUUUUAUUAUAUUAUUAUCAAAACGAAAAACAGA |
| | | AAAACUGUCAUAGAUAAAUAUAAAAAAAAAUAGAAACCACAAAC |
| | | GACUACUAGUACUCCAAUCUUAGAUGUAUAUGCUCCUAGAUAAGA |
| | | UUUAGUAUUACCAUAAUCAUCGAAGAAUGAAAGACGACGAUGAU |
| | | UCCUUACCGCUCCUGCCACCCGGUCUGUAUGUAGAGAGAGAAGAG |
| | | AGAAAACGGUGAAUCCAAGAUCCCCGGGUCGGCGUCGGCAUGCCG |
| | | CUGAUCGCAGUGGCCCCACCUCGGCAUGCCGGCGCCGGGCGAGGA |
| | | AUUGCUCAUGAAAAAAGUAUCUUUCUGUAAAAAAAGAAAACAAU |
| | | ACAUGAUUAACCGAAAAGAAACCAACAAAAAGAACCCGAGAUCAG |
| | | UCGAUUUCGAUCACUACGAUAAACACAUGGAAGAUUUCUUGAAAA |
| | | AAGAAAAGAGAAAGAGACCACCUUCCCGGCGGCGGACACGCUCCU |
| | | CUCCGUCGCCGUUCUGCACCAUGAUUCGAUCAAUAACAACAUCAU |
| | | CAUCGGAGACCAUCUUUUAAUCAAUCAGCGUUGCAGUAGUCGACU |
| | | CCCUGGACACGAAGGAGUCAUCCAUUUUUAUCCUCGCACUUCUUC |
| | | GCUCUCAAAGCCGCCUUUAAAGUUGAAAUGAAAGGAUGGAAACAU |
| | | GGAAUACAGUUUUAAUUGCACGUAUCACCAUUUUACUACAAAAAG |
| | | AAAAAAAAACAACUUACACAUAGUAUUACCUUAGGUUUACGGAUA |
| | | AGUAGAGUGUAGGCGUUUUUGAAACAGUUCAGCCAAUGCAAUCUU |
| | | GUCUCGGCAUAAUCACUCUUUCUGCAUAUAAUAGUAGUAGUAGAU |
| | | UUAUUCACAUCAACACAGCGAAAAACUCCAGCAUCAAAGUACACC |
| | | UAGAGACAGCCCUUAAAAUAUAGUUUGCAGCUUUUAGAUGUACUU |
| | | ACACCAAAGAAGAUUACCGUCCUUACGAGAAAACAGAUACUCGGA |
| | | UAUAGGAAUCAAGACAGCUCUGCACUGAAAACACACUCUCCUGUC |
| | | ACGACACCGCGCCACACCAGAGGCGUACGCGUGACUUCAUCGCAA |
| | | CGAUCCAUCGUGAUGUCCCUCGCAGAACCUAAAAAGACCAAAAAA |
| | | AAAUCUUGGACCACAGUUGUCGAUUCUUGAAGACAAUAUUCUCGU |
| | | GAGAACUUUGAGAUUCGCACUUGAAACCUCUUAGGAUCCACAAAA |
| | | ACAACAACCUCUGUAUGGAAAAUGCGCUAUUUUAUCUCAGCUUUU |
| | | CUCCCAAACCUCGGUUUCUUCCUAUUCUUAAGUUUUCCCUAGUAU |
| | | AUUUGCCUCCUUAUAAGAAAAGAAGCACAAGCUCGGUCGCACGGA |
| | | UUAUUCCUUCUGCUAAUCUAUUAUUUUGUUCCUUUUUUUUUUGUU |
| | | UGCCUUCACCCCCUUCACUCCCUGUAGCAACACAGAGUAGUAGAC |
| | | ACAAUAAAUGAGAAGUUUGCAUGCAAGCU |
| 13 | Sense | GGCGCUUCCGGAAGAGCUAGCUCCCCAGAUCGCUGCUGCCCCGGC |
| | domain 3 | GUUCUCCAGAAGCCCCGGCGGGCGAAUCGGCCGGCUGGUCGGUCG |
| | deletion | GCGCUCGGACGGAUGGGGAGAACGGCGGUGACUUAGCCGCCCGUG |
| | mutant: | GCCGGGAGAAGAUGGAGGAGCCGAGAUGACAACGGCAGUCGUGGA |
| | ΔD3 | AGGGUCGCCAAGCCCCGGUCCUUCUCUUCUGUCUGGUCGAAUCUC |
| | | GUUUUCUUUUUUCAACCGCUCUUUUUAUCACCUUUUUAUGUGAGU |
| | | UUCUCUUCCGCGUCUCCCGGCCGUACCAUCCACCCAUGCAGCAUGC |
| | | ACGCGUGUAUGUAUGCAUCGUCUCUCCUCCGUCCCGACUACCAUC |
| | | AGCAGCACCACUACCGCCACCCCCAGCGCCACCACCGCUGCCGUCG |
| | | CCACCGCGUUAUCCGUUCCUCGUAGGCUGGUCCUGGGGAACGGGU |
| | | CGGCGGCCGGUCGGCUUCUGUUUUAUUAUUUUUUUUUAUUUUUUA |
| | | UCUUCUCCUUUCCUUAAUCUCGGAUUAUCAUUUCCCUCUCCUACC |
| | | UACCACGAAUCGCAGAUGAUAAACAAGAGGGUAAAAAGAAAAAA |
| | | GCUACAGACAUUUGGGUACCUCAGCUUUCCGAUAACUCGAAGAAU |
| | | UCAAAGUCGACGAUUCCCAACAAGAGAAAACAGAACAAAAACAAA |
| | | GAAGAACAAAAAGAUCAUCUCUCUCGGUGUAUAGCAACACCAACA |
| | | ACAACCGCAUCGCAACAUCUUCAUCCGCAAGACGGAAAGAAAACA |
| | | ACAAUAAUGAGAAUGAAAUCACCACAACCAAGCCAGAUUUCACGU |
| | | CCAUGAGUUUUUAUUAUAUUAUUAUCAAAACGAAAAACAGAAAA |
| | | ACUGUCAUAGAUAAAUAUAAAAAAAAAUAGAAACCACAAACGACU |
| | | ACUAGUACUCCAAUCUUAGAUGUAUAUGCUCCUAGAUAAGAUUUA |
| | | GUAUUACCAUAAUCAUCGAAGAAUGAAAGACGACGAUGAUUCCUU |
| | | ACCGCUCCUGCCACCCGGUCUGUAUGUAGAGAGAGAAGAGAGAAA |
| | | ACGGUGAAUCCAAGAUCCCCGGGUCGGCGUCGGCAUGCCGCUGAU |
| | | CGCAGUGGCCCCACCUCGGCAUGCCGGCGCCGGGCGAGGAAUUGC |
| | | UCAUGAAAAAAGUAUCUUUCUGUAAAAAAAGAAAACAAUACAUG |
| | | AUUAACCGAAAAGAAACCAACAAAAAGAACCCGAGAUCAGUCGAU |
| | | UUCGAUCACUACGAUAAACACAUGGAAGAUUUCUUGAAAAAAGAA |
| | | AAGAGAAAGAGACCACCUUCCCGGCGGCGGACACGCUCCUCUCCG |
| | | UCGCCGUUCUGCACCAUGAUUCGAUCAAUAACAACAUCAUCAUCG |
| | | GAGACCAUCUUUUAAUCAAUCAGCGUUGCAGUAGUCGACUCCCUG |
| | | GACACGAAGGAGUCAUCCAUUUUUAUCCUCGCACUUCUUCGCUCU |
| | | CAAAGCCGCCUUUAAAGUUGAAAUGAAAGGAUGGAAACAUGGAA |
| | | UACAGUUUUAAUUGCACGUAUCACCAUUUUACUACAAAAAGAAAA |
| | | AAAAACAACUUACACAUAGUAUUACCUUAGGUUUACGGAUAAGUA |
| | | GAGUGUAGGCGUUUUUGAAACAGUUCAGCCAAUGCAAUCUUGUCU |
| | | CGGCAUAAUCACUCUUUCUGCAUAUAAUAGUAGUAGUAGAUUUAU |
| | | UCACAUCAACACAGCGAAAAACUCCAGCAUCAAAGUACACCUAGA |
| | | GACAGCCCUUAAAAUAUAGUUUGCAGCUUUUAGAUGUACUUACAC |
| | | CAAAGAAGAUUACCGUCCUUACGAGAAAACAGAUACUCGGAUAUA |
| | | GGAAUCAAGACAGCUCUGCACUGAAAACACACUCUCCUGUCACGA |
| | | CACCGCGCCACACCAGAGGCGUACGCGUGACUUCAUCGCAACGAU |
| | | CCAUCGUGAUGUCCCUCGCAGAACCUAAAAAGACCAAAAAAAAAU |
| | | CUUGGACCACAGUUGUCGAUUCUUGAAGACAAUAUUCUCGUGAGA |
| | | ACUUUGAGAUUCGCACUUGAAACCUCUUAGGAUCCACAAAAACAA |
| | | CAACCUCUGUAUGGAAAAUGCGCUAUUUUAUCUCAGCUUUUCUCC |
| | | CAAACCUCGGUUUCUUCCUAUUCUUAAGUUUUCCCUAGUAUAUUU |
| | | GCCUCCUUAUAAGAAAAGAAGCACAAGCUCGGUCGCACGGAUUAU |
| | | UCCUUCUGCUAAUCUAUUAUUUUGUUCCUUUUUUUUUUGUUUGCC |
| | | UUCACCCCCUUCACUCCCUGUAGCAACACAGAGUAGUAGACACAA |
| | | UAAAUGAGAAGUUUGCAUGCAAGCU |
| 14 | Sense | GGCGCUUCCGGAAGAGCUAGCUCCCCAGAUCGCUGCUGCCCCGGC |
| | domain 4 | GUUCUCCAGAAGCCCCGGCGGGCGAAUCGGCCGGCUGGUCGGUCG |
| | deletion | GCGCUCGGACGGAUGGGGAGAACGGCGGUGACUUAGCCGCCCGUG |
| | mutant: | GCCGGGAGAAGAUGGAGGAGCCGAGAUGACAACGGCAGUCGUGGA |
| | ΔD4 | AGGGUCGCCAAGCCCCGGUCCUUCUCUUCUGUCUGGUCGAAUCUC |
| | | GUUUUCUUUUUUCAACCGCUCUUUUUAUCACCUUUUUAUGUGAGU |
| | | UUCUCUUCCGCGUCUCCCGGCCGUACCAUCCACCCAUGCAGCAUGC |
| | | ACGCGUGUAUGUAUGCAUCGUCUCUCCUCCGUCCCGACUACCAUC |
| | | AGCAGCACCACUACCGCCACCCCCAGCGCCACCACCGCUGCCGUCG |
| | | CCACCGCGUUAUCCGUUCCUCGUAGGCUGGUCCUGGGGAACGGGU |
| | | CGGCGGCCGGUCGGCUUCUGUUUUAUUAUUUUUUUUUAUUUUUUA |
| | | UCUUCUCCUUUCCUUAAUCUCGGAUUAUCAUUUCCCUCUCCUACC |
| | | UACCACGAAUCGCAGAUGAUAAACAAGAGGGUAAAAAGAAAAAA |
| | | GCUACAGACAUUUGGGUACCUCAGCUUUCCGAUAACUCGAAGAAU |
| | | UCAAAGUCGACGAUUCCCAACAAGAGAAAACAGAACAAAAACAAG |
| | | GUCAUUUUUAUUUAUCCUCAUCGUCAACAACAACUACCGACAACA |
| | | ACGAAACACCACCAAGAAUGUCAAUCCGCAAGGGUGUUCCUGCCC |
| | | CCUCGACGCGCCUGUCGCGAUCCUCAUGGCGAGGACCGCGAUCUC |
| | | CGUAUAGGUAGAUGAAAUUAUCCCGUGUCCGGUCCUGAUUCCCCG |
| | | CAUGCCCUGCACAUCCUGACGCGUCGGUCAGCAGCCAAACAAUCA |
| | | UAGGAAAUGAACCAGAAGAACAAAAAGAUCAUCUCUCUCGGUGUA |
| | | UAGCAACACCAACAACAACCGCAUCGCAACAUCUUCAUCCGCAAG |
| | | ACGGAAAGAAAACAACAAUAAUGAGAAUGAAAUCACCACAACCAA |
| | | GCCAGAUUUCACGUCCAUGAGUUUUUAUUAUAUUAUUAUCAAAAC |
| | | GAAAAACAGAAAAACUGUCAUAGAUAAAUAUAAAAAAAAAUAGA |
| | | AACCACAAACGACUACUAGUACUCCAAUCUUAGAUGUAUAUGCUC |
| | | CUAGAUAAGAUUUAGUAUUACCAUAAUCAUCGAAGAAUGAAAGA |
| | | CGACGAUGAUUCCUUACCGCUCCUGCCACCCGGUCUGUAUGUAGA |
| | | GAGAGAAGAGAGAAAACGGUGAAUCCAAGAUCCCCGGGUACUUCU |
| | | UCGCUCUCAAAGCCGCCUUUAAAGUUGAAAUGAAAGGAUGGAAAC |
| | | AUGGAAUACAGUUUUAAUUGCACGUAUCACCAUUUUACUACAAAA |
| | | AGAAAAAAAAACAACUUACACAUAGUAUUACCUUAGGUUUACGGA |
| | | UAAGUAGAGUGUAGGCGUUUUUGAAACAGUUCAGCCAAUGCAAUC |
| | | UUGUCUCGGCAUAAUCACUCUUUCUGCAUAUAAUAGUAGUAGUAG |
| | | AUUUAUUCACAUCAACACAGCGAAAAACUCCAGCAUCAAAGUACA |
| | | CCUAGAGACAGCCCUUAAAAUAUAGUUUGCAGCUUUUAGAUGUAC |
| | | UUACACCAAAGAAGAUUACCGUCCUUACGAGAAAACAGAUACUCG |
| | | GAUAUAGGAAUCAAGACAGCUCUGCACUGAAAACACACUCUCCUG |
| | | UCACGACACCGCGCCACACCAGAGGCGUACGCGUGACUUCAUCGC |
| | | AACGAUCCAUCGUGAUGUCCCUCGCAGAACCUAAAAAGACCAAAA |
| | | AAAAAUCUUGGACCACAGUUGUCGAUUCUUGAAGACAAUAUUCUC |
| | | GUGAGAACUUUGAGAUUCGCACUUGAAACCUCUUAGGAUCCACAA |
| | | AAACAACAACCUCUGUAUGGAAAAUGCGCUAUUUUAUCUCAGCUU |
| | | UUCUCCCAAACCUCGGUUUCUUCCUAUUCUUAAGUUUUCCCUAGU |
| | | AUAUUUGCCUCCUUAUAAGAAAAGAAGCACAAGCUCGGUCGCACG |
| | | GAUUAUUCCUUCUGCUAAUCUAUUAUUUUGUUCCUUUUUUUUUUG |
| | | UUUGCCUUCACCCCCUUCACUCCCUGUAGCAACACAGAGUAGUAG |
| | | ACACAAUAAAUGAGAAGUUUGCAUGCAAGCU |
| 15 | Multimer | GGCGCUUCCGGAAGAGCUAGCUUUUAUUUUUUAUCUUCUCCUUUC |
| | sequence | CUUAAUCUCGGAUUAUCAUUUCCCUCUCCUACCUACCACGAAUCG |
| | D2x4 | CAGAUGAUAAACAAGAGGGUAAAAAGAAAAAAGCGCGAAUUCCG |
| | | AUCUUUUAUUUUUUAUCUUCUCCUUUCCUUAAUCUCGGAUUAUCA |
| | | UUUCCCUCUCCUACCUACCACGAAUCGCAGAUGAUAAACAAGAGG |
| | | GUAAAAAGAAAAAGAUCGUAUCCGGUACCUGUGGUUUUAUUUUU |
| | | UAUCUUCUCCUUUCCUUAAUCUCGGAUUAUCAUUUCCCUCUCCUA |
| | | CCUACCACGAAUCGCAGAUGAUAAACAAGAGGGUAAAAAGAAAAA |
| | | CCACACCUCCACCGGUGGGCCGUUUUAUUUUUUAUCUUCUCCUUU |
| | | CCUUAAUCUCGGAUUAUCAUUUCCCUCUCCUACCUACCACGAAUC |
| | | GCAGAUGAUAAACAAGAGGGUAAAAAGAAAAACGGCCCAAGCU |
| 16 | Multimer | GGCGCUUCCGGAAGAGCUAGCCCGGCGGUCAUUUUUAUUUAUCCU |
| | sequence | CAUCGUCAACAACAACUACCGACAACAACGAAACACCACCAAGAA |
| | D3x4 | UGUCAAUCCGCAAGGGUGUUCCUGCCCCCUCGACGCGCCUGUCGC |
| | | GAUCCUCAUGGCGAGGACCGCGAUCUCCGUAUAGGUAGAUGAAAU |
| | | UAUCCCGUGUCCGGUCCUGAUUCCCCGCAUGCCCUGCACAUCCUG |
| | | ACGCGUCGGUCAGCAGCCAAACAAUCAUAGGAAAUGAACCGCCGG |
| | | GAAGCGCCGAAUUCAUAUCGAUCGGUCAUUUUUAUUUAUCCUCAU |
| | | CGUCAACAACAACUACCGACAACAACGAAACACCACCAAGAAUGU |
| | | CAAUCCGCAAGGGUGUUCCUGCCCCCUCGACGCGCCUGUCGCGAU |
| | | CCUCAUGGCGAGGACCGCGAUCUCCGUAUAGGUAGAUGAAAUUAU |
| | | CCCGUGUCCGGUCCUGAUUCCCCGCAUGCCCUGCACAUCCUGACGC |
| | | GUCGGUCAGCAGCCAAACAAUCAUAGGAAAUGAACCGAUCGAAUA |
| | | AAGGUACCUGUGGGGUCAUUUUUAUUUAUCCUCAUCGUCAACAAC |
| | | AACUACCGACAACAACGAAACACCACCAAGAAUGUCAAUCCGCAA |
| | | GGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAG |
| | | CAGCCAAACAAUCAUAGGAAAUGAACCCCACAUUUUACCGGUAAU |
| | | ACCGGGGGGUCAUUUUUAUUUAUCCUCAUCGUCAACAACAACUAC |
| | | CGACAACAACGAAACACCACCAAGAAUGUCAAUCCGCAAGGGUGU |
| | | UCCUGCCCCCUCGACGCGCCUGUCGCGAUCCUCAUGGCGAGGACCG |
| | | CGAUCUCCGUAUAGGUAGAUGAAAUUAUCCCGUGUCCGGUCCUGA |
| | | UUCCCCGCAUGCCCUGCACAUCCUGACGCGUCGGUCAGCAGCCAA |
| | | ACAAUCAUAGGAAAUGAACCCCCCGGAAGCU |
| 17 | Multimer | GGCGCUUCCGGAAGAGCUAGCCCGGCGGUCAUUUUUAUUUAUCCU |
| | sequence | CAUCGUCAACAACAACUACCGACAACAACGAAACACCACCAAGAA |
| | D3x4_D2x4 | UGUCAAUCCGCAAGGGUGUUCCUGCCCCCUCGACGCGCCUGUCGC |
| | | GAUCCUCAUGGCGAGGACCGCGAUCUCCGUAUAGGUAGAUGAAAU |
| | | UAUCCCGUGUCCGGUCCUGAUUCCCCGCAUGCCCUGCACAUCCUG |
| | | ACGCGUCGGUCAGCAGCCAAACAAUCAUAGGAAAUGAACCGCCGG |
| | | GAAGCGCCGAAUUCAUAUCGAUCGGUCAUUUUUAUUUAUCCUCAU |
| | | CGUCAACAACAACUACCGACAACAACGAAACACCACCAAGAAUGU |
| | | CAAUCCGCAAGGGUGUUCCUGCCCCCUCGACGCGCCUGUCGCGAU |
| | | CCUCAUGGCGAGGACCGCGAUCUCCGUAUAGGUAGAUGAAAUUAU |
| | | CCCGUGUCCGGUCCUGAUUCCCCGCAUGCCCUGCACAUCCUGACGC |
| | | GUCGGUCAGCAGCCAAACAAUCAUAGGAAAUGAACCGAUCGAAUA |
| | | AAGGUACCUGUGGGGUCAUUUUUAUUUAUCCUCAUCGUCAACAAC |
| | | AACUACCGACAACAACGAAACACCACCAAGAAUGUCAAUCCGCAA |
| | | GGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAG |
| | | CAGCCAAACAAUCAUAGGAAAUGAACCCCACAUUUUACCGGUAAU |
| | | ACCGGGGGGUCAUUUUUAUUUAUCCUCAUCGUCAACAACAACUAC |
| | | CGACAACAACGAAACACCACCAAGAAUGUCAAUCCGCAAGGGUGU |
| | | UCCUGCCCCCUCGACGCGCCUGUCGCGAUCCUCAUGGCGAGGACCG |
| | | CGAUCUCCGUAUAGGUAGAUGAAAUUAUCCCGUGUCCGGUCCUGA |
| | | UUCCCCGCAUGCCCUGCACAUCCUGACGCGUCGGUCAGCAGCCAA |
| | | ACAAUCAUAGGAAAUGAACCCCCCGGAAGCUUUCCGGAAGAGCUA |
| | | GCUUUUAUUUUUUAUCUUCUCCUUUCCUUAAUCUCGGAUUAUCAU |
| | | UUCCCUCUCCUACCUACCACGAAUCGCAGAUGAUAAACAAGAGGG |
| | | UAAAAAGAAAAAAGCGCGAAUUCCGAUCUUUUAUUUUUUAUCUUC |
| | | UCCUUUCCUUAAUCUCGGAUUAUCAUUUCCCUCUCCUACCUACCA |
| | | CGAAUCGCAGAUGAUAAACAAGAGGGUAAAAAGAAAAAGAUCGU |
| | | AUCCGGUACCUGUGGUUUUAUUUUUUAUCUUCUCCUUUCCUUAAU |
| | | CUCGGAUUAUCAUUUCCCUCUCCUACCUACCACGAAUCGCAGAUG |
| | | AUAAACAAGAGGGUAAAAAGAAAAACCACACCUCCACCGGUGGGC |
| | | CGUUUUAUUUUUUAUCUUCUCCUUUCCUUAAUCUCGGAUUAUCAU |
| | | UUCCCUCUCCUACCUACCACGAAUCGCAGAUGAUAAACAAGAGGG |
| | | UAAAAAGAAAAACGGCCCAAGCUUGACUCCUAUAGUGUCACCUAA |
| | | AUGUCUAG |
| 18 | Multimer | GGCGCUUCCGGAAGAGCUAGCUUUUAUUUUUUAUCUUCUCCUUUC |
| | sequence | CUUAAUCUCGGAUUAUCAUUUCCCUCUCCUACCUACCACGAAUCG |
| | D2x4_D3x4 | CAGAUGAUAAACAAGAGGGUAAAAAGAAAAAAGCGCGAAUUCCG |
| | | AUCUUUUAUUUUUUAUCUUCUCCUUUCCUUAAUCUCGGAUUAUCA |
| | | UUUCCCUCUCCUACCUACCACGAAUCGCAGAUGAUAAACAAGAGG |
| | | GUAAAAAGAAAAAGAUCGUAUCCGGUACCUGUGGUUUUAUUUUU |
| | | UAUCUUCUCCUUUCCUUAAUCUCGGAUUAUCAUUUCCCUCUCCUA |
| | | CCUACCACGAAUCGCAGAUGAUAAACAAGAGGGUAAAAAGAAAAA |
| | | CCACACCUCCACCGGUGGGCCGUUUUAUUUUUUAUCUUCUCCUUU |
| | | CCUUAAUCUCGGAUUAUCAUUUCCCUCUCCUACCUACCACGAAUC |
| | | GCAGAUGAUAAACAAGAGGGUAAAAAGAAAAACGGCCCAAGCUUC |
| | | CGGCGGUCAUUUUUAUUUAUCCUCAUCGUCAACAACAACUACCGA |
| | | CAACAACGAAACACCACCAAGAAUGUCAAUCCGCAAGGGUGUUCC |
| | | UGCCCCCUCGACGCGCCUGUCGCGAUCCUCAUGGCGAGGACCGCG |
| | | AUCUCCGUAUAGGUAGAUGAAAUUAUCCCGUGUCCGGUCCUGAUU |
| | | CCCCGCAUGCCCUGCACAUCCUGACGCGUCGGUCAGCAGCCAAACA |
| | | AUCAUAGGAAAUGAACCGCCGGGAAGCGCCGAAUUCAUAUCGAUC |
| | | GGUCAUUUUUAUUUAUCCUCAUCGUCAACAACAACUACCGACAAC |
| | | AACGAAACACCACCAAGAAUGUCAAUCCGCAAGGGUGUUCCUGCC |
| | | CCCUCGACGCGCCUGUCGCGAUCCUCAUGGCGAGGACCGCGAUCU |
| | | CCGUAUAGGUAGAUGAAAUUAUCCCGUGUCCGGUCCUGAUUCCCC |
| | | GCAUGCCCUGCACAUCCUGACGCGUCGGUCAGCAGCCAAACAAUC |
| | | AUAGGAAAUGAACCGAUCGAAUAAAGGUACCUGUGGGGUCAUUU |
| | | UUAUUUAUCCUCAUCGUCAACAACAACUACCGACAACAACGAAAC |
| | | ACCACCAAGAAUGUCAAUCCGCAAGGGUGUUCCUGCCCCCUCGAC |
| | | GCGCCUGUCGCGAUCCUCAUGGCGAGGACCGCGAUCUCCGUAUAG |
| | | GUAGAUGAAAUUAUCCCGUGUCCGGUCCUGAUUCCCCGCAUGCCC |
| | | UGCACAUCCUGACGCGUCGGUCAGCAGCCAAACAAUCAUAGGAAA |
| | | UGAACCCCACAUUUUACCGGUAAUACCGGGGGGUCAUUUUUAUUU |
| | | AUCCUCAUCGUCAACAACAACUACCGACAACAACGAAACACCACC |
| | | AAGAAUGUCAAUCCGCAAGGGUGUUCCUGCCCCCUCGACGCGCCU |
| | | GUCGCGAUCCUCAUGGCGAGGACCGCGAUCUCCGUAUAGGUAGAU |
| | | GAAAUUAUCCCGUGUCCGGUCCUGAUUCCCCGCAUGCCCUGCACA |
| | | UCCUGACGCGUCGGUCAGCAGCCAAACAAUCAUAGGAAAUGAACC |
| | | CCCCGGAAGCUUGACUCCUAUAGUGUCACCUAAAUGUCUAG |
| 19 | Multimer | GGCGCUUCCGGAAGAGCUAGCCCGGCGGUCAUUUUUAUUUAUCCU |
| | sequence | CAUCGUCAACAACAACUACCGACAACAACGAAACACCACCAAGAA |
| | (D3_D2)x4 | UGUCAAUCCGCAAGGGUGUUCCUGCCCCCUCGACGCGCCUGUCGC |
| | | GAUCCUCAUGGCGAGGACCGCGAUCUCCGUAUAGGUAGAUGAAAU |
| | | UAUCCCGUGUCCGGUCCUGAUUCCCCGCAUGCCCUGCACAUCCUG |
| | | ACGCGUCGGUCAGCAGCCAAACAAUCAUAGGAAAUGAACCGCCGG |
| | | GAAGCGCCGAAUUCCGAUCUUUUAUUUUUUAUCUUCUCCUUUCCU |
| | | UAAUCUCGGAUUAUCAUUUCCCUCUCCUACCUACCACGAAUCGCA |
| | | GAUGAUAAACAAGAGGGUAAAAAGAAAAAGAUCGUAUCCGGUAC |
| | | CUGUGGGGUCAUUUUUAUUUAUCCUCAUCGUCAACAACAACUACC |
| | | GACAACAACGAAACACCACCAAGAAUGUCAAUCCGCAAGGGUGUU |
| | | CCUGCCCCCUCGACGCGCCUGUCGCGAUCCUCAUGGCGAGGACCGC |
| | | GAUCUCCGUAUAGGUAGAUGAAAUUAUCCCGUGUCCGGUCCUGAU |
| | | UCCCCGCAUGCCCUGCACAUCCUGACGCGUCGGUCAGCAGCCAAAC |
| | | AAUCAUAGGAAAUGAACCCCACAUUUUACCGGUGGGCCGUUUUAU |
| | | UUUUUAUCUUCUCCUUUCCUUAAUCUCGGAUUAUCAUUUCCCUCU |
| | | CCUACCUACCACGAAUCGCAGAUGAUAAACAAGAGGGUAAAAAGA |
| | | AAAACGGCCCAAGCUUUCCGGAAGAGCUAGCCCGGCGGUCAUUUU |
| | | UAUUUAUCCUCAUCGUCAACAACAACUACCGACAACAACGAAACA |
| | | CCACCAAGAAUGUCAAUCCGCAAGGGUGUUCCUGCCCCCUCGACG |
| | | CGCCUGUCGCGAUCCUCAUGGCGAGGACCGCGAUCUCCGUAUAGG |
| | | UAGAUGAAAUUAUCCCGUGUCCGGUCCUGAUUCCCCGCAUGCCCU |
| | | GCACAUCCUGACGCGUCGGUCAGCAGCCAAACAAUCAUAGGAAAU |
| | | GAACCGCCGGGAAGCGCCGAAUUCCGAUCUUUUAUUUUUUAUCUU |
| | | CUCCUUUCCUUAAUCUCGGAUUAUCAUUUCCCUCUCCUACCUACC |
| | | ACGAAUCGCAGAUGAUAAACAAGAGGGUAAAAAGAAAAAGAUCG |
| | | UAUCCGGUACCUGUGGGGUCAUUUUUAUUUAUCCUCAUCGUCAAC |
| | | AACAACUACCGACAACAACGAAACACCACCAAGAAUGUCAAUCCG |
| | | CAAGGGUGUUCCUGCCCCCUCGACGCGCCUGUCGCGAUCCUCAUG |
| | | GCGAGGACCGCGAUCUCCGUAUAGGUAGAUGAAAUUAUCCCGUGU |
| | | CCGGUCCUGAUUCCCCGCAUGCCCUGCACAUCCUGACGCGUCGGUC |
| | | AGCAGCCAAACAAUCAUAGGAAAUGAACCCCACAUUUUACCGGUG |
| | | GGCCGUUUUAUUUUUUAUCUUCUCCUUUCCUUAAUCUCGGAUUAU |
| | | CAUUUCCCUCUCCUACCUACCACGAAUCGCAGAUGAUAAACAAGA |
| | | GGGUAAAAAGAAAAACGGCCCAAGCUUGACUCCUAUAGUGUCACC |
| | | UAAAUGUCUAG |
| 20 | gGFP_β2.7 | GGAGUCUCAGAUCCGCUAGCGCUACCGGUCGCCACCAUGGUGAGC |
| | | AAGGGCGAGGAGCUGUUCACCGGGGUGGUGCCCAUCCUGGUCGAG |
| | | CUGGACGGCGACGUAAACGGCCACAAGUUCAGCGUGUCCGGCGAG |
| | | GGCGAGGGCGAUGCCACCUACGGCAAGCUGACCCUGAAGUUCAUC |
| | | UGCACCACCGGCAAGCUGCCCGUGCCCUGGCCCACCCUCGUGACCA |
| | | CCCUGACCUACGGCGUGCAGUGCUUCAGCCGCUACCCCGACCACAU |
| | | GAAGCAGCACGACUUCUUCAAGUCCGCCAUGCCCGAAGGCUACGU |
| | | CCAGGAGCGCACCAUCUUCUUCAAGGACGACGGCAACUACAAGAC |
| | | CCGCGCCGAGGUGAAGUUCGAGGGCGACACCCUGGUGAACCGCAU |
| | | CGAGCUGAAGGGCAUCGACUUCAAGGAGGACGGCAACAUCCUGGG |
| | | GCACAAGCUGGAGUACAACUACAACAGCCACAACGUCUAUAUCAU |
| | | GGCCGACAAGCAGAAGAACGGCAUCAAGGUGAACUUCAAGAUCCG |
| | | CCACAACAUCGAGGACGGCAGCGUGCAGCUCGCCGACCACUACCA |
| | | GCAGAACACCCCCAUCGGCGACGGCCCCGUGCUGCUGCCCGACAAC |
| | | CACUACCUGAGCACCCAGUCCGCCCUGAGCAAAGACCCCAACGAG |
| | | AAGCGCGAUCACAUGGUCCUGCUGGAGUUCGUGACCGCCGCCGGG |
| | | AUCACUCUCGGCAUGGACGAGCUGUACAAGUCCGGAUGAGCUAGC |
| | | UCCCCAGAUCGCUGCUGCCCCGGCGUUCUCCAGAAGCCCCGGCGGG |
| | | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCU |
| | | UCUCUUCUGUCUGGUCGAAUCUCGUUUUCUUUUUUCAACCGCUCU |
| | | UUUUAUCACCUUUUUAUGUGAGUUUCUCUUCCGCGUCUCCCGGCC |
| | | GUACCAUCCACCCAUGCAGCAUGCACGCGUGUAUGUAUGCAUCGU |
| | | CUCUCCUCCGUCCCGACUACCAUCAGCAGCACCACUACCGCCACCC |
| | | CCAGCGCCACCACCGCUGCCGUCGCCACCGCGUUAUCCGUUCCUCG |
| | | UAGGCUGGUCCUGGGGAACGGGUCGGCGGCCGGUCGGCUUCUGUU |
| | | UUAUUAUUUUUUUUUAUUUUUUAUCUUCUCCUUUCCUUAAUCUCG |
| | | GAUUAUCAUUUCCCUCUCCUACCUACCACGAAUCGCAGAUGAUAA |
| | | ACAAGAGGGUAAAAAGAAAAAAGCUACAGACAUUUGGGUACCUCA |
| | | GCUUUCCGAUAACUCGAAGAAUUCAAAGUCGACGAUUCCCAACAA |
| | | GAGAAAACAGAACAAAAACAAGGUCAUUUUUAUUUAUCCUCAUCG |
| | | UCAACAACAACUACCGACAACAACGAAACACCACCAAGAAUGUCA |
| | | AUCCGCAAGGGUGUUCCUGCCCCCUCGACGCGCCUGUCGCGAUCC |
| | | UCAUGGCGAGGACCGCGAUCUCCGUAUAGGUAGAUGAAAUUAUCC |
| | | CGUGUCCGGUCCUGAUUCCCCGCAUGCCCUGCACAUCCUGACGCG |
| | | UCGGUCAGCAGCCAAACAAUCAUAGGAAAUGAACCAGAAGAACAA |
| | | AAAGAUCAUCUCUCUCGGUGUAUAGCAACACCAACAACAACCGCA |
| | | UCGCAACAUCUUCAUCCGCAAGACGGAAAGAAAACAACAAUAAUG |
| | | AGAAUGAAAUCACCACAACCAAGCCAGAUUUCACGUCCAUGAGUU |
| | | UUUAUUAUAUUAUUAUCAAAACGAAAAACAGAAAAACUGUCAUA |
| | | GAUAAAUAUAAAAAAAAAUAGAAACCACAAACGACUACUAGUACU |
| | | CCAAUCUUAGAUGUAUAUGCUCCUAGAUAAGAUUUAGUAUUACCA |
| | | UAAUCAUCGAAGAAUGAAAGACGACGAUGAUUCCUUACCGCUCCU |
| | | GCCACCCGGUCUGUAUGUAGAGAGAGAAGAGAGAAAACGGUGAAU |
| | | CCAAGAUCCCCGGGUCGGCGUCGGCAUGCCGCUGAUCGCAGUGGC |
| | | CCCACCUCGGCAUGCCGGCGCCGGGCGAGGAAUUGCUCAUGAAAA |
| | | AAGUAUCUUUCUGUAAAAAAAGAAAACAAUACAUGAUUAACCGA |
| | | AAAGAAACCAACAAAAAGAACCCGAGAUCAGUCGAUUUCGAUCAC |
| | | UACGAUAAACACAUGGAAGAUUUCUUGAAAAAAGAAAAGAGAAA |
| | | GAGACCACCUUCCCGGCGGCGGACACGCUCCUCUCCGUCGCCGUUC |
| | | UGCACCAUGAUUCGAUCAAUAACAACAUCAUCAUCGGAGACCAUC |
| | | UUUUAAUCAAUCAGCGUUGCAGUAGUCGACUCCCUGGACACGAAG |
| | | GAGUCAUCCAUUUUUAUCCUCGCACUUCUUCGCUCUCAAAGCCGC |
| | | CUUUAAAGUUGAAAUGAAAGGAUGGAAACAUGGAAUACAGUUUU |
| | | AAUUGCACGUAUCACCAUUUUACUACAAAAAGAAAAAAAAACAAC |
| | | UUACACAUAGUAUUACCUUAGGUUUACGGAUAAGUAGAGUGUAG |
| | | GCGUUUUUGAAACAGUUCAGCCAAUGCAAUCUUGUCUCGGCAUAA |
| | | UCACUCUUUCUGCAUAUAAUAGUAGUAGUAGAUUUAUUCACAUCA |
| | | ACACAGCGAAAAACUCCAGCAUCAAAGUACACCUAGAGACAGCCC |
| | | UUAAAAUAUAGUUUGCAGCUUUUAGAUGUACUUACACCAAAGAA |
| | | GAUUACCGUCCUUACGAGAAAACAGAUACUCGGAUAUAGGAAUCA |
| | | AGACAGCUCUGCACUGAAAACACACUCUCCUGUCACGACACCGCG |
| | | CCACACCAGAGGCGUACGCGUGACUUCAUCGCAACGAUCCAUCGU |
| | | GAUGUCCCUCGCAGAACCUAAAAAGACCAAAAAAAAAUCUUGGAC |
| | | CACAGUUGUCGAUUCUUGAAGACAAUAUUCUCGUGAGAACUUUGA |
| | | GAUUCGCACUUGAAACCUCUUAGGAUCCACAAAAACAACAACCUC |
| | | UGUAUGGAAAAUGCGCUAUUUUAUCUCAGCUUUUCUCCCAAACCU |
| | | CGGUUUCUUCCUAUUCUUAAGUUUUCCCUAGUAUAUUUGCCUCCU |
| | | UAUAAGAAAAGAAGCACAAGCUCGGUCGCACGGAUUAUUCCUUCU |
| | | GCUAAUCUAUUAUUUUGUUCCUUUUUUUUUUGUUUGCCUUCACCC |
| | | CCUUCACUCCCUGUAGCAACACAGAGUAGUAGACACAAUAAAUGA |
| | | GAAGUAAGCU |
| 21 | gGFP_s_β2.7 | GGAGUCUCAGAUCCGCUAGCGCUACCGGUCGCCACCAUGGUGAGC |
| | | AAGGGCGAGGAGCUGUUCACCGGGGUGGUGCCCAUCCUGGUCGAG |
| | | CUGGACGGCGACGUAAACGGCCACAAGUUCAGCGUGUCCGGCGAG |
| | | GGCGAGGGCGAUGCCACCUACGGCAAGCUGACCCUGAAGUUCAUC |
| | | UGCACCACCGGCAAGCUGCCCGUGCCCUGGCCCACCCUCGUGACCA |
| | | CCCUGACCUACGGCGUGCAGUGCUUCAGCCGCUACCCCGACCACAU |
| | | GAAGCAGCACGACUUCUUCAAGUCCGCCAUGCCCGAAGGCUACGU |
| | | CCAGGAGCGCACCAUCUUCUUCAAGGACGACGGCAACUACAAGAC |
| | | CCGCGCCGAGGUGAAGUUCGAGGGCGACACCCUGGUGAACCGCAU |
| | | CGAGCUGAAGGGCAUCGACUUCAAGGAGGACGGCAACAUCCUGGG |
| | | GCACAAGCUGGAGUACAACUACAACAGCCACAACGUCUAUAUCAU |
| | | GGCCGACAAGCAGAAGAACGGCAUCAAGGUGAACUUCAAGAUCCG |
| | | CCACAACAUCGAGGACGGCAGCGUGCAGCUCGCCGACCACUACCA |
| | | GCAGAACACCCCCAUCGGCGACGGCCCCGUGCUGCUGCCCGACAAC |
| | | CACUACCUGAGCACCCAGUCCGCCCUGAGCAAAGACCCCAACGAG |
| | | AAGCGCGAUCACAUGGUCCUGCUGGAGUUCGUGACCGCCGCCGGG |
| | | AUCACUCUCGGCAUGGACGAGCUGUACAAGUCCGGAUGACACCAC |
| | | CCCGGAGAACAGCUCCUCGCUAGCUCCCCAGAUCGCUGCUGCCCCG |
| | | GCGUUCUCCAGAAGCCCCGGCGGGCGAAUCGGCCGGCUGGUCGGU |
| | | CGGCGCUCGGACGGAUGGGGAGAACGGCGGUGACUUAGCCGCCCG |
| | | UGGCCGGGAGAAGAUGGAGGAGCCGAGAUGACAACGGCAGUCGUG |
| | | GAAGGGUCGCCAAGCCCCGGUCCUUCUCUUCUGUCUGGUCGAAUC |
| | | UCGUUUUCUUUUUUCAACCGCUCUUUUUAUCACCUUUUUAUGUGA |
| | | GUUUCUCUUCCGCGUCUCCCGGCCGUACCAUCCACCCAUGCAGCAU |
| | | GCACGCGUGUAUGUAUGCAUCGUCUCUCCUCCGUCCCGACUACCA |
| | | UCAGCAGCACCACUACCGCCACCCCCAGCGCCACCACCGCUGCCGU |
| | | CGCCACCGCGUUAUCCGUUCCUCGUAGGCUGGUCCUGGGGAACGG |
| | | GUCGGCGGCCGGUCGGCUUCUGUUUUAUUAUUUUUUUUUAUUUUU |
| | | UAUCUUCUCCUUUCCUUAAUCUCGGAUUAUCAUUUCCCUCUCCUA |
| | | CCUACCACGAAUCGCAGAUGAUAAACAAGAGGGUAAAAAGAAAAA |
| | | AGCUACAGACAUUUGGGUACCUCAGCUUUCCGAUAACUCGAAGAA |
| | | UUCAAAGUCGACGAUUCCCAACAAGAGAAAACAGAACAAAAACAA |
| | | GGUCAUUUUUAUUUAUCCUCAUCGUCAACAACAACUACCGACAAC |
| | | AACGAAACACCACCAAGAAUGUCAAUCCGCAAGGGUGUUCCUGCC |
| | | CCCUCGACGCGCCUGUCGCGAUCCUCAUGGCGAGGACCGCGAUCU |
| | | CCGUAUAGGUAGAUGAAAUUAUCCCGUGUCCGGUCCUGAUUCCCC |
| | | GCAUGCCCUGCACAUCCUGACGCGUCGGUCAGCAGCCAAACAAUC |
| | | AUAGGAAAUGAACCAGAAGAACAAAAAGAUCAUCUCUCUCGGUGU |
| | | AUAGCAACACCAACAACAACCGCAUCGCAACAUCUUCAUCCGCAA |
| | | GACGGAAAGAAAACAACAAUAAUGAGAAUGAAAUCACCACAACCA |
| | | AGCCAGAUUUCACGUCCAUGAGUUUUUAUUAUAUUAUUAUCAAAA |
| | | CGAAAAACAGAAAAACUGUCAUAGAUAAAUAUAAAAAAAAAUAG |
| | | AAACCACAAACGACUACUAGUACUCCAAUCUUAGAUGUAUAUGCU |
| | | CCUAGAUAAGAUUUAGUAUUACCAUAAUCAUCGAAGAAUGAAAG |
| | | ACGACGAUGAUUCCUUACCGCUCCUGCCACCCGGUCUGUAUGUAG |
| | | AGAGAGAAGAGAGAAAACGGUGAAUCCAAGAUCCCCGGGUCGGCG |
| | | UCGGCAUGCCGCUGAUCGCAGUGGCCCCACCUCGGCAUGCCGGCG |
| | | CCGGGCGAGGAAUUGCUCAUGAAAAAAGUAUCUUUCUGUAAAAAA |
| | | AGAAAACAAUACAUGAUUAACCGAAAAGAAACCAACAAAAAGAAC |
| | | CCGAGAUCAGUCGAUUUCGAUCACUACGAUAAACACAUGGAAGAU |
| | | UUCUUGAAAAAAGAAAAGAGAAAGAGACCACCUUCCCGGCGGCGG |
| | | ACACGCUCCUCUCCGUCGCCGUUCUGCACCAUGAUUCGAUCAAUA |
| | | ACAACAUCAUCAUCGGAGACCAUCUUUUAAUCAAUCAGCGUUGCA |
| | | GUAGUCGACUCCCUGGACACGAAGGAGUCAUCCAUUUUUAUCCUC |
| | | GCACUUCUUCGCUCUCAAAGCCGCCUUUAAAGUUGAAAUGAAAGG |
| | | AUGGAAACAUGGAAUACAGUUUUAAUUGCACGUAUCACCAUUUUA |
| | | CUACAAAAAGAAAAAAAAACAACUUACACAUAGUAUUACCUUAGG |
| | | UUUACGGAUAAGUAGAGUGUAGGCGUUUUUGAAACAGUUCAGCC |
| | | AAUGCAAUCUUGUCUCGGCAUAAUCACUCUUUCUGCAUAUAAUAG |
| | | UAGUAGUAGAUUUAUUCACAUCAACACAGCGAAAAACUCCAGCAU |
| | | CAAAGUACACCUAGAGACAGCCCUUAAAAUAUAGUUUGCAGCUUU |
| | | UAGAUGUACUUACACCAAAGAAGAUUACCGUCCUUACGAGAAAAC |
| | | AGAUACUCGGAUAUAGGAAUCAAGACAGCUCUGCACUGAAAACAC |
| | | ACUCUCCUGUCACGACACCGCGCCACACCAGAGGCGUACGCGUGA |
| | | CUUCAUCGCAACGAUCCAUCGUGAUGUCCCUCGCAGAACCUAAAA |
| | | AGACCAAAAAAAAAUCUUGGACCACAGUUGUCGAUUCUUGAAGAC |
| | | AAUAUUCUCGUGAGAACUUUGAGAUUCGCACUUGAAACCUCUUAG |
| | | GAUCCACAAAAACAACAACCUCUGUAUGGAAAAUGCGCUAUUUUA |
| | | UCUCAGCUUUUCUCCCAAACCUCGGUUUCUUCCUAUUCUUAAGUU |
| | | UUCCCUAGUAUAUUUGCCUCCUUAUAAGAAAAGAAGCACAAGCUC |
| | | GGUCGCACGGAUUAUUCCUUCUGCUAAUCUAUUAUUUUGUUCCUU |
| | | UUUUUUUUGUUUGCCUUCACCCCCUUCACUCCCUGUAGCAACACA |
| | | GAGUAGUAGACACAAUAAAUGAGAAGUAAGCU |
| 22 | mtGFP_s_β2.7 | GGAGUCUCAGAUCCGCUAGCGCUACCGGUCGCCACCAUAGUGAGC |
| | | AAGGGCGAGGAGCUGUUCACCGGGGUGGUGCCCAUCCUGGUCGAG |
| | | CUGGACGGCGACGUAAACGGCCACAAGUUCAGCGUGUCCGGCGAG |
| | | GGCGAGGGCGAUGCCACCUACGGCAAGCUGACCCUGAAGUUCAUC |
| | | UGCACCACCGGCAAGCUGCCCGUGCCCUGGCCCACCCUCGUGACCA |
| | | CCCUGACCUACGGCGUGCAGUGCUUCAGCCGCUACCCCGACCACAU |
| | | GAAGCAGCACGACUUCUUCAAGUCCGCCAUGCCCGAAGGCUACGU |
| | | CCAGGAGCGCACCAUCUUCUUCAAGGACGACGGCAACUACAAGAC |
| | | CCGCGCCGAGGUGAAGUUCGAGGGCGACACCCUGGUGAACCGCAU |
| | | CGAGCUGAAGGGCAUCGACUUCAAGGAGGACGGCAACAUCCUGGG |
| | | GCACAAGCUGGAGUACAACUACAACAGCCACAACGUCUAUAUCAU |
| | | GGCCGACAAGCAGAAGAACGGCAUCAAGGUGAACUUCAAGAUCCG |
| | | CCACAACAUCGAGGACGGCAGCGUGCAGCUCGCCGACCACUACCA |
| | | GCAGAACACCCCCAUCGGCGACGGCCCCGUGCUGCUGCCCGACAAC |
| | | CACUACCUGAGCACCCAGUCCGCCCUGAGCAAAGACCCCAACGAG |
| | | AAGCGCGAUCACAUGGUCCUGCUGGAGUUCGUGACCGCCGCCGGG |
| | | AUCACUCUCGGCAUGGACGAGCUGUACAAGUCCGGAAAGACACCA |
| | | CCCCGGAGAACAGCUCCUCGCUAGCUCCCCAGAUCGCUGCUGCCCC |
| | | GGCGUUCUCCAGAAGCCCCGGCGGGCGAAUCGGCCGGCUGGUCGG |
| | | UCGGCGCUCGGACGGAUGGGGAGAACGGCGGUGACUUAGCCGCCC |
| | | GUGGCCGGGAGAAGAUGGAGGAGCCGAGAUGACAACGGCAGUCGU |
| | | GGAAGGGUCGCCAAGCCCCGGUCCUUCUCUUCUGUCUGGUCGAAU |
| | | CUCGUUUUCUUUUUUCAACCGCUCUUUUUAUCACCUUUUUAUGUG |
| | | AGUUUCUCUUCCGCGUCUCCCGGCCGUACCAUCCACCCAUGCAGCA |
| | | UGCACGCGUGUAUGUAUGCAUCGUCUCUCCUCCGUCCCGACUACC |
| | | AUCAGCAGCACCACUACCGCCACCCCCAGCGCCACCACCGCUGCCG |
| | | UCGCCACCGCGUUAUCCGUUCCUCGUAGGCUGGUCCUGGGGAACG |
| | | GGUCGGCGGCCGGUCGGCUUCUGUUUUAUUAUUUUUUUUUAUUUU |
| | | UUAUCUUCUCCUUUCCUUAAUCUCGGAUUAUCAUUUCCCUCUCCU |
| | | ACCUACCACGAAUCGCAGAUGAUAAACAAGAGGGUAAAAAGAAAA |
| | | AAGCUACAGACAUUUGGGUACCUCAGCUUUCCGAUAACUCGAAGA |
| | | AUUCAAAGUCGACGAUUCCCAACAAGAGAAAACAGAACAAAAACA |
| | | AGGUCAUUUUUAUUUAUCCUCAUCGUCAACAACAACUACCGACAA |
| | | CAACGAAACACCACCAAGAAUGUCAAUCCGCAAGGGUGUUCCUGC |
| | | CCCCUCGACGCGCCUGUCGCGAUCCUCAUGGCGAGGACCGCGAUC |
| | | UCCGUAUAGGUAGAUGAAAUUAUCCCGUGUCCGGUCCUGAUUCCC |
| | | CGCAUGCCCUGCACAUCCUGACGCGUCGGUCAGCAGCCAAACAAU |
| | | CAUAGGAAAUGAACCAGAAGAACAAAAAGAUCAUCUCUCUCGGUG |
| | | UAUAGCAACACCAACAACAACCGCAUCGCAACAUCUUCAUCCGCA |
| | | AGACGGAAAGAAAACAACAAUAAUGAGAAUGAAAUCACCACAACC |
| | | AAGCCAGAUUUCACGUCCAUGAGUUUUUAUUAUAUUAUUAUCAAA |
| | | ACGAAAAACAGAAAAACUGUCAUAGAUAAAUAUAAAAAAAAAUA |
| | | GAAACCACAAACGACUACUAGUACUCCAAUCUUAGAUGUAUAUGC |
| | | UCCUAGAUAAGAUUUAGUAUUACCAUAAUCAUCGAAGAAUGAAA |
| | | GACGACGAUGAUUCCUUACCGCUCCUGCCACCCGGUCUGUAUGUA |
| | | GAGAGAGAAGAGAGAAAACGGUGAAUCCAAGAUCCCCGGGUCGGC |
| | | GUCGGCAUGCCGCUGAUCGCAGUGGCCCCACCUCGGCAUGCCGGC |
| | | GCCGGGCGAGGAAUUGCUCAUGAAAAAAGUAUCUUUCUGUAAAAA |
| | | AAGAAAACAAUACAUGAUUAACCGAAAAGAAACCAACAAAAAGAA |
| | | CCCGAGAUCAGUCGAUUUCGAUCACUACGAUAAACACAUGGAAGA |
| | | UUUCUUGAAAAAAGAAAAGAGAAAGAGACCACCUUCCCGGCGGCG |
| | | GACACGCUCCUCUCCGUCGCCGUUCUGCACCAUGAUUCGAUCAAU |
| | | AACAACAUCAUCAUCGGAGACCAUCUUUUAAUCAAUCAGCGUUGC |
| | | AGUAGUCGACUCCCUGGACACGAAGGAGUCAUCCAUUUUUAUCCU |
| | | CGCACUUCUUCGCUCUCAAAGCCGCCUUUAAAGUUGAAAUGAAAG |
| | | GAUGGAAACAUGGAAUACAGUUUUAAUUGCACGUAUCACCAUUUU |
| | | ACUACAAAAAGAAAAAAAAACAACUUACACAUAGUAUUACCUUAG |
| | | GUUUACGGAUAAGUAGAGUGUAGGCGUUUUUGAAACAGUUCAGC |
| | | CAAUGCAAUCUUGUCUCGGCAUAAUCACUCUUUCUGCAUAUAAUA |
| | | GUAGUAGUAGAUUUAUUCACAUCAACACAGCGAAAAACUCCAGCA |
| | | UCAAAGUACACCUAGAGACAGCCCUUAAAAUAUAGUUUGCAGCUU |
| | | UUAGAUGUACUUACACCAAAGAAGAUUACCGUCCUUACGAGAAAA |
| | | CAGAUACUCGGAUAUAGGAAUCAAGACAGCUCUGCACUGAAAACA |
| | | CACUCUCCUGUCACGACACCGCGCCACACCAGAGGCGUACGCGUG |
| | | ACUUCAUCGCAACGAUCCAUCGUGAUGUCCCUCGCAGAACCUAAA |
| | | AAGACCAAAAAAAAAUCUUGGACCACAGUUGUCGAUUCUUGAAGA |
| | | CAAUAUUCUCGUGAGAACUUUGAGAUUCGCACUUGAAACCUCUUA |
| | | GGAUCCACAAAAACAACAACCUCUGUAUGGAAAAUGCGCUAUUUU |
| | | AUCUCAGCUUUUCUCCCAAACCUCGGUUUCUUCCUAUUCUUAAGU |
| | | UUUCCCUAGUAUAUUUGCCUCCUUAUAAGAAAAGAAGCACAAGCU |
| | | CGGUCGCACGGAUUAUUCCUUCUGCUAAUCUAUUAUUUUGUUCCU |
| | | UUUUUUUUUGUUUGCCUUCACCCCCUUCACUCCCUGUAGCAACAC |
| | | AGAGUAGUAGACACAAUAAAUGAGAAGUAAGCU |
| 23 | mtGFP_s | GGAGUCUCAGAUCCGCUAGCGCUACCGGUCGCCACCAUAGUGAGC |
| | | AAGGGCGAGGAGCUGUUCACCGGGGUGGUGCCCAUCCUGGUCGAG |
| | | CUGGACGGCGACGUAAACGGCCACAAGUUCAGCGUGUCCGGCGAG |
| | | GGCGAGGGCGAUGCCACCUACGGCAAGCUGACCCUGAAGUUCAUC |
| | | UGCACCACCGGCAAGCUGCCCGUGCCCUGGCCCACCCUCGUGACCA |
| | | CCCUGACCUACGGCGUGCAGUGCUUCAGCCGCUACCCCGACCACAU |
| | | GAAGCAGCACGACUUCUUCAAGUCCGCCAUGCCCGAAGGCUACGU |
| | | CCAGGAGCGCACCAUCUUCUUCAAGGACGACGGCAACUACAAGAC |
| | | CCGCGCCGAGGUGAAGUUCGAGGGCGACACCCUGGUGAACCGCAU |
| | | CGAGCUGAAGGGCAUCGACUUCAAGGAGGACGGCAACAUCCUGGG |
| | | GCACAAGCUGGAGUACAACUACAACAGCCACAACGUCUAUAUCAU |
| | | GGCCGACAAGCAGAAGAACGGCAUCAAGGUGAACUUCAAGAUCCG |
| | | CCACAACAUCGAGGACGGCAGCGUGCAGCUCGCCGACCACUACCA |
| | | GCAGAACACCCCCAUCGGCGACGGCCCCGUGCUGCUGCCCGACAAC |
| | | CACUACCUGAGCACCCAGUCCGCCCUGAGCAAAGACCCCAACGAG |
| | | AAGCGCGAUCACAUGGUCCUGCUGGAGUUCGUGACCGCCGCCGGG |
| | | AUCACUCUCGGCAUGGACGAGCUGUACAAGUCCGGAAGACACCAC |
| | | CCCGGAGAACAGCUCCUC |
| 24 | asATP6_s6_ | GGAGUCUCCGGAAUAAGCGCCAGUAGAAUUAGAAUUGUGAAGAU |
| | β2.7 | GAUAAGUGUAGAGGGAAGGUUAAUGGUUGAUAUUGCUAGGGUGG |
| | | CGCUUCCAAUUAGGUGCGUGAGUAGGUGGCCUGCAGUAAUGUUAA |
| | | AGCAAGGGUUAUUCCGAAUUGGGCUAGCUCCCCAGAUCGCUGCUG |
| | | CCCCGGCGUUCUCCAGAAGCCCCGGCGGGCGAAUCGGCCGGCUGG |
| | | UCGGUCGGCGCUCGGACGGAUGGGGAGAACGGCGGUGACUUAGCC |
| | | GCCCGUGGCCGGGAGAAGAUGGAGGAGCCGAGAUGACAACGGCAG |
| | | UCGUGGAAGGGUCGCCAAGCCCCGGUCCUUCUCUUCUGUCUGGUC |
| | | GAAUCUCGUUUUCUUUUUUCAACCGCUCUUUUUAUCACCUUUUUA |
| | | UGUGAGUUUCUCUUCCGCGUCUCCCGGCCGUACCAUCCACCCAUG |
| | | CAGCAUGCACGCGUGUAUGUAUGCAUCGUCUCUCCUCCGUCCCGA |
| | | CUACCAUCAGCAGCACCACUACCGCCACCCCCAGCGCCACCACCGC |
| | | UGCCGUCGCCACCGCGUUAUCCGUUCCUCGUAGGCUGGUCCUGGG |
| | | GAACGGGUCGGCGGCCGGUCGGCUUCUGUUUUAUUAUUUUUUUUU |
| | | AUUUUUUAUCUUCUCCUUUCCUUAAUCUCGGAUUAUCAUUUCCCU |
| | | CUCCUACCUACCACGAAUCGCAGAUGAUAAACAAGAGGGUAAAAA |
| | | GAAAAAAGCUACAGACAUUUGGGUACCUCAGCUUUCCGAUAACUC |
| | | GAAGAAUUCAAAGUCGACGAUUCCCAACAAGAGAAAACAGAACAA |
| | | AAACAAGGUCAUUUUUAUUUAUCCUCAUCGUCAACAACAACUACC |
| | | GACAACAACGAAACACCACCAAGAAUGUCAAUCCGCAAGGGUGUU |
| | | CCUGCCCCCUCGACGCGCCUGUCGCGAUCCUCAUGGCGAGGACCGC |
| | | GAUCUCCGUAUAGGUAGAUGAAAUUAUCCCGUGUCCGGUCCUGAU |
| | | UCCCCGCAUGCCCUGCACAUCCUGACGCGUCGGUCAGCAGCCAAAC |
| | | AAUCAUAGGAAAUGAACCAGAAGAACAAAAAGAUCAUCUCUCUCG |
| | | GUGUAUAGCAACACCAACAACAACCGCAUCGCAACAUCUUCAUCC |
| | | GCAAGACGGAAAGAAAACAACAAUAAUGAGAAUGAAAUCACCACA |
| | | ACCAAGCCAGAUUUCACGUCCAUGAGUUUUUAUUAUAUUAUUAUC |
| | | AAAACGAAAAACAGAAAAACUGUCAUAGAUAAAUAUAAAAAAAA |
| | | AUAGAAACCACAAACGACUACUAGUACUCCAAUCUUAGAUGUAUA |
| | | UGCUCCUAGAUAAGAUUUAGUAUUACCAUAAUCAUCGAAGAAUGA |
| | | AAGACGACGAUGAUUCCUUACCGCUCCUGCCACCCGGUCUGUAUG |
| | | UAGAGAGAGAAGAGAGAAAACGGUGAAUCCAAGAUCCCCGGGUCG |
| | | GCGUCGGCAUGCCGCUGAUCGCAGUGGCCCCACCUCGGCAUGCCG |
| | | GCGCCGGGCGAGGAAUUGCUCAUGAAAAAAGUAUCUUUCUGUAAA |
| | | AAAAGAAAACAAUACAUGAUUAACCGAAAAGAAACCAACAAAAAG |
| | | AACCCGAGAUCAGUCGAUUUCGAUCACUACGAUAAACACAUGGAA |
| | | GAUUUCUUGAAAAAAGAAAAGAGAAAGAGACCACCUUCCCGGCGG |
| | | CGGACACGCUCCUCUCCGUCGCCGUUCUGCACCAUGAUUCGAUCA |
| | | AUAACAACAUCAUCAUCGGAGACCAUCUUUUAAUCAAUCAGCGUU |
| | | GCAGUAGUCGACUCCCUGGACACGAAGGAGUCAUCCAUUUUUAUC |
| | | CUCGCACUUCUUCGCUCUCAAAGCCGCCUUUAAAGUUGAAAUGAA |
| | | AGGAUGGAAACAUGGAAUACAGUUUUAAUUGCACGUAUCACCAUU |
| | | UUACUACAAAAAGAAAAAAAAACAACUUACACAUAGUAUUACCUU |
| | | AGGUUUACGGAUAAGUAGAGUGUAGGCGUUUUUGAAACAGUUCA |
| | | GCCAAUGCAAUCUUGUCUCGGCAUAAUCACUCUUUCUGCAUAUAA |
| | | UAGUAGUAGUAGAUUUAUUCACAUCAACACAGCGAAAAACUCCAG |
| | | CAUCAAAGUACACCUAGAGACAGCCCUUAAAAUAUAGUUUGCAGC |
| | | UUUUAGAUGUACUUACACCAAAGAAGAUUACCGUCCUUACGAGAA |
| | | AACAGAUACUCGGAUAUAGGAAUCAAGACAGCUCUGCACUGAAAA |
| | | CACACUCUCCUGUCACGACACCGCGCCACACCAGAGGCGUACGCGU |
| | | GACUUCAUCGCAACGAUCCAUCGUGAUGUCCCUCGCAGAACCUAA |
| | | AAAGACCAAAAAAAAAUCUUGGACCACAGUUGUCGAUUCUUGAAG |
| | | ACAAUAUUCUCGUGAGAACUUUGAGAUUCGCACUUGAAACCUCUU |
| | | AGGAUCCACAAAAACAACAACCUCUGUAUGGAAAAUGCGCUAUUU |
| | | UAUCUCAGCUUUUCUCCCAAACCUCGGUUUCUUCCUAUUCUUAAG |
| | | UUUUCCCUAGUAUAUUUGCCUCCUUAUAAGAAAAGAAGCACAAGC |
| | | UCGGUCGCACGGAUUAUUCCUUCUGCUAAUCUAUUAUUUUGUUCC |
| | | UUUUUUUUUUGUUUGCCUUCACCCCCUUCACUCCCUGUAGCAACA |
| | | CAGAGUAGUAGACACAAUAAAUGAGAAGUCCAAUUCGGAAUAACC |
| | | CAAGCU |
| 25 | β2.7_s8_as | GGCGCUUCCGGAAUAGGUUGGAUUGGGGGCUAGCUCCCCAGAUCG |
| | ATP8 | CUGCUGCCCCGGCGUUCUCCAGAAGCCCCGGCGGGCGAAUCGGCC |
| | | GGCUGGUCGGUCGGCGCUCGGACGGAUGGGGAGAACGGCGGUGAC |
| | | UUAGCCGCCCGUGGCCGGGAGAAGAUGGAGGAGCCGAGAUGACAA |
| | | CGGCAGUCGUGGAAGGGUCGCCAAGCCCCGGUCCUUCUCUUCUGU |
| | | CUGGUCGAAUCUCGUUUUCUUUUUUCAACCGCUCUUUUUAUCACC |
| | | UUUUUAUGUGAGUUUCUCUUCCGCGUCUCCCGGCCGUACCAUCCA |
| | | CCCAUGCAGCAUGCACGCGUGUAUGUAUGCAUCGUCUCUCCUCCG |
| | | UCCCGACUACCAUCAGCAGCACCACUACCGCCACCCCCAGCGCCAC |
| | | CACCGCUGCCGUCGCCACCGCGUUAUCCGUUCCUCGUAGGCUGGU |
| | | CCUGGGGAACGGGUCGGCGGCCGGUCGGCUUCUGUUUUAUUAUUU |
| | | UUUUUUAUUUUUUAUCUUCUCCUUUCCUUAAUCUCGGAUUAUCAU |
| | | UUCCCUCUCCUACCUACCACGAAUCGCAGAUGAUAAACAAGAGGG |
| | | UAAAAAGAAAAAAGCUACAGACAUUUGGGUACCUCAGCUUUCCGA |
| | | UAACUCGAAGAAUUCAAAGUCGACGAUUCCCAACAAGAGAAAACA |
| | | GAACAAAAACAAGGUCAUUUUUAUUUAUCCUCAUCGUCAACAACA |
| | | ACUACCGACAACAACGAAACACCACCAAGAAUGUCAAUCCGCAAG |
| | | GGUGUUCCUGCCCCCUCGACGCGCCUGUCGCGAUCCUCAUGGCGA |
| | | GGACCGCGAUCUCCGUAUAGGUAGAUGAAAUUAUCCCGUGUCCGG |
| | | UCCUGAUUCCCCGCAUGCCCUGCACAUCCUGACGCGUCGGUCAGC |
| | | AGCCAAACAAUCAUAGGAAAUGAACCAGAAGAACAAAAAGAUCAU |
| | | CUCUCUCGGUGUAUAGCAACACCAACAACAACCGCAUCGCAACAU |
| | | CUUCAUCCGCAAGACGGAAAGAAAACAACAAUAAUGAGAAUGAAA |
| | | UCACCACAACCAAGCCAGAUUUCACGUCCAUGAGUUUUUAUUAUA |
| | | UUAUUAUCAAAACGAAAAACAGAAAAACUGUCAUAGAUAAAUAU |
| | | AAAAAAAAAUAGAAACCACAAACGACUACUAGUACUCCAAUCUUA |
| | | GAUGUAUAUGCUCCUAGAUAAGAUUUAGUAUUACCAUAAUCAUCG |
| | | AAGAAUGAAAGACGACGAUGAUUCCUUACCGCUCCUGCCACCCGG |
| | | UCUGUAUGUAGAGAGAGAAGAGAGAAAACGGUGAAUCCAAGAUC |
| | | CCCGGGUCGGCGUCGGCAUGCCGCUGAUCGCAGUGGCCCCACCUC |
| | | GGCAUGCCGGCGCCGGGCGAGGAAUUGCUCAUGAAAAAAGUAUCU |
| | | UUCUGUAAAAAAAGAAAACAAUACAUGAUUAACCGAAAAGAAACC |
| | | AACAAAAAGAACCCGAGAUCAGUCGAUUUCGAUCACUACGAUAAA |
| | | CACAUGGAAGAUUUCUUGAAAAAAGAAAAGAGAAAGAGACCACCU |
| | | UCCCGGCGGCGGACACGCUCCUCUCCGUCGCCGUUCUGCACCAUGA |
| | | UUCGAUCAAUAACAACAUCAUCAUCGGAGACCAUCUUUUAAUCAA |
| | | UCAGCGUUGCAGUAGUCGACUCCCUGGACACGAAGGAGUCAUCCA |
| | | UUUUUAUCCUCGCACUUCUUCGCUCUCAAAGCCGCCUUUAAAGUU |
| | | GAAAUGAAAGGAUGGAAACAUGGAAUACAGUUUUAAUUGCACGU |
| | | AUCACCAUUUUACUACAAAAAGAAAAAAAAACAACUUACACAUAG |
| | | UAUUACCUUAGGUUUACGGAUAAGUAGAGUGUAGGCGUUUUUGA |
| | | AACAGUUCAGCCAAUGCAAUCUUGUCUCGGCAUAAUCACUCUUUC |
| | | UGCAUAUAAUAGUAGUAGUAGAUUUAUUCACAUCAACACAGCGAA |
| | | AAACUCCAGCAUCAAAGUACACCUAGAGACAGCCCUUAAAAUAUA |
| | | GUUUGCAGCUUUUAGAUGUACUUACACCAAAGAAGAUUACCGUCC |
| | | UUACGAGAAAACAGAUACUCGGAUAUAGGAAUCAAGACAGCUCUG |
| | | CACUGAAAACACACUCUCCUGUCACGACACCGCGCCACACCAGAG |
| | | GCGUACGCGUGACUUCAUCGCAACGAUCCAUCGUGAUGUCCCUCG |
| | | CAGAACCUAAAAAGACCAAAAAAAAAUCUUGGACCACAGUUGUCG |
| | | AUUCUUGAAGACAAUAUUCUCGUGAGAACUUUGAGAUUCGCACUU |
| | | GAAACCUCUUAGGAUCCACAAAAACAACAACCUCUGUAUGGAAAA |
| | | UGCGCUAUUUUAUCUCAGCUUUUCUCCCAAACCUCGGUUUCUUCC |
| | | UAUUCUUAAGUUUUCCCUAGUAUAUUUGCCUCCUUAUAAGAAAAG |
| | | AAGCACAAGCUCGGUCGCACGGAUUAUUCCUUCUGCUAAUCUAUU |
| | | AUUUUGUUCCUUUUUUUUUUGUUUGCCUUCACCCCCUUCACUCCC |
| | | UGUAGCAACACAGAGUAGUAGACACAAUAAAUGAGAAGUAAGCU |
| | | UCCCCAAACCAACCUCUACCGGAAGCGCCUUUUUUUCGUUCAUUU |
| | | UGGUUCUCAGGGUUUGUUAUAAUUUUUUAUUUUUAUGGGCUUUG |
| | | GUGAGGGAGGUAGGUGGUAGUUUGUGUUUAAUAUUUUUAGUUGG |
| | | GUGAUGA |
| 26 | ATP6_D3₄_ | GGAGUCUCCGGAAUAAGCGCCAGUAGAAUUAGAAUUGUGAAGAU |
| | D2₄ | GAUAAGUGUAGAGGGAAGGUUAAUGGUUGAUAUUGCUAGGGUGG |
| | | CGCUUCCAAUUAGGUGCGUGAGUAGGUGGCCUGCAGUAAUGUUAA |
| | | AGCAAGGGUUAUUCCGAAUUGGGCUAGCCCGGCGGUCAUUUUUAU |
| | | UUAUCCUCAUCGUCAACAACAACUACCGACAACAACGAAACACCA |
| | | CCAAGAAUGUCAAUCCGCAAGGGUGUUCCUGCCCCCUCGACGCGC |
| | | CUGUCGCGAUCCUCAUGGCGAGGACCGCGAUCUCCGUAUAGGUAG |
| | | AUGAAAUUAUCCCGUGUCCGGUCCUGAUUCCCCGCAUGCCCUGCA |
| | | CAUCCUGACGCGUCGGUCAGCAGCCAAACAAUCAUAGGAAAUGAA |
| | | CCGCCGGGAAGCGCCGAAUUCAUAUCGAUCGGUCAUUUUUAUUUA |
| | | UCCUCAUCGUCAACAACAACUACCGACAACAACGAAACACCACCA |
| | | AGAAUGUCAAUCCGCAAGGGUGUUCCUGCCCCCUCGACGCGCCUG |
| | | UCGCGAUCCUCAUGGCGAGGACCGCGAUCUCCGUAUAGGUAGAUG |
| | | AAAUUAUCCCGUGUCCGGUCCUGAUUCCCCGCAUGCCCUGCACAU |
| | | CCUGACGCGUCGGUCAGCAGCCAAACAAUCAUAGGAAAUGAACCG |
| | | AUCGAAUAAAGGUACCUGUGGGGUCAUUUUUAUUUAUCCUCAUC |
| | | GUCAACAACAACUACCGACAACAACGAAACACCACCAAGAAUGUC |
| | | AAUCCGCAAGGGUGUUCCUGCCCCCUCGACGCGCCUGUCGCGAUC |
| | | CUCAUGGCGAGGACCGCGAUCUCCGUAUAGGUAGAUGAAAUUAUC |
| | | CCGUGUCCGGUCCUGAUUCCCCGCAUGCCCUGCACAUCCUGACGCG |
| | | UCGGUCAGCAGCCAAACAAUCAUAGGAAAUGAACCCCACAUUUUA |
| | | CCGGUAAUACCGGGGGGUCAUUUUUAUUUAUCCUCAUCGUCAACA |
| | | ACAACUACCGACAACAACGAAACACCACCAAGAAUGUCAAUCCGC |
| | | AAGGGUGUUCCUGCCCCCUCGACGCGCCUGUCGCGAUCCUCAUGG |
| | | CGAGGACCGCGAUCUCCGUAUAGGUAGAUGAAAUUAUCCCGUGUC |
| | | CGGUCCUGAUUCCCCGCAUGCCCUGCACAUCCUGACGCGUCGGUC |
| | | AGCAGCCAAACAAUCAUAGGAAAUGAACCCCCCGGAAGCUUUCCG |
| | | GAAGAGCUAGCUUUUAUUUUUUAUCUUCUCCUUUCCUUAAUCUC |
| | | GGAUUAUCAUUUCCCUCUCCUACCUACCACGAAUCGCAGAUGAUA |
| | | AACAAGAGGGUAAAAAGAAAAAAGCGCGAAUUCCGAUCUUUUAU |
| | | UUUUUAUCUUCUCCUUUCCUUAAUCUCGGAUUAUCAUUUCCCUCU |
| | | CCUACCUACCACGAAUCGCAGAUGAUAAACAAGAGGGUAAAAAGA |
| | | AAAAGAUCGUAUCCGGUACCUGUGGUUUUAUUUUUUAUCUUCUC |
| | | CUUUCCUUAAUCUCGGAUUAUCAUUUCCCUCUCCUACCUACCACG |
| | | AAUCGCAGAUGAUAAACAAGAGGGUAAAAAGAAAAACCACACCUC |
| | | CACCGGUGGGCCGUUUUAUUUUUUAUCUUCUCCUUUCCUUAAUCU |
| | | CGGAUUAUCAUUUCCCUCUCCUACCUACCACGAAUCGCAGAUGAU |
| | | AAACAAGAGGGUAAAAAGAAAAACGGCCCCCAUGAAUAAGGCCCA |
| | | AGCUUGACUCCUAUAGUGUCACCUAAAUGUCUAGAUACUAAGGGA |
| | | GUCUUGC |
| 27 | S1a | CCGGC |
| 28 | S1b | GCCGG |
| 29 | S2a | CGAUC |
| 30 | S2b | GAUCG |
| 31 | S3a | UGUGG |
| 32 | S3b | CCACA |
| 33 | S4a | CCGGGG |
| 34 | S4b | CCCCGG |
| 35 | S6a | AAGCAAGGGUUAUUCCGAAUUGG |
| 36 | S6b | CCAAUUCGGAAUAACCC |
| 37 | S8a | GGUUGGAUUGGGG |
| 38 | S8b | CCCCAAACCAACCUCUACCGGAAGCGCCUUUU |
| 39 | Spacer | CACCACCCCGGAGAACAGCTCCTC |
| | FIG. 3a | The same spacer was found to stabilize all the GFP fusion constructs |
| 40 | Antisense | GGAGUCAAGCUUGCAUGCAAACUUCUCAUUUAUUGUGUCUACUAC |
| | domain 1 | UCUGUGUUGCUACAGGGAGUGAAGGGGGUGAAGGCAAACAAAAA |
| | deletion | AAAAAGGAACAAAAUAAUAGAUUAGCAGAAGGAAUAAUCCGUGC |
| | mutant: | GACCGAGCUUGUGCUUCUUUUCUUAUAAGGAGGCAAAUAUACUAG |
| | ΔD1AS | GGAAAACUUAAGAAUAGGAAGAAACCGAGGUUUGGGAGAAAAGC |
| | | UGAGAUAAAAUAGCGCAUUUUCCAUACAGAGGUUGUUGUUUUUG |
| | | UGGAUCCUAAGAGGUUUCAAGUGCGAAUCUCAAAGUUCUCACGAG |
| | | AAUAUUGUCUUCAAGAAUCGACAACUGUGGUCCAAGAUUUUUUUU |
| | | UGGUCUUUUUAGGUUCUGCGAGGGACAUCACGAUGGAUCGUUGCG |
| | | AUGAAGUCACGCGUACGCCUCUGGUGUGGCGCGGUGUCGUGACAG |
| | | GAGAGUGUGUUUUCAGUGCAGAGCUGUCUUGAUUCCUAUAUCCGA |
| | | GUAUCUGUUUUCUCGUAAGGACGGUAAUCUUCUUUGGUGUAAGU |
| | | ACAUCUAAAAGCUGCAAACUAUAUUUUAAGGGCUGUCUCUAGGUG |
| | | UACUUUGAUGCUGGAGUUUUUCGCUGUGUUGAUGUGAAUAAAUC |
| | | UACUACUACUAUUAUAUGCAGAAAGAGUGAUUAUGCCGAGACAAG |
| | | AUUGCAUUGGCUGAACUGUUUCAAAAACGCCUACACUCUACUUAU |
| | | CCGUAAACCUAAGGUAAUACUAUGUGUAAGUUGUUUUUUUUUCU |
| | | UUUUGUAGUAAAAUGGUGAUACGUGCAAUUAAAACUGUAUUCCA |
| | | UGUUUCCAUCCUUUCAUUUCAACUUUAAAGGCGGCUUUGAGAGCG |
| | | AAGAAGUGCGAGGAUAAAAAUGGAUGACUCCUUCGUGUCCAGGGA |
| | | GUCGACUACUGCAACGCUGAUUGAUUAAAAGAUGGUCUCCGAUGA |
| | | UGAUGUUGUUAUUGAUCGAAUCAUGGUGCAGAACGGCGACGGAG |
| | | AGGAGCGUGUCCGCCGCCGGGAAGGUGGUCUCUUUCUCUUUUCUU |
| | | UUUUCAAGAAAUCUUCCAUGUGUUUAUCGUAGUGAUCGAAAUCGA |
| | | CUGAUCUCGGGUUCUUUUUGUUGGUUUCUUUUCGGUUAAUCAUGU |
| | | AUUGUUUUCUUUUUUUACAGAAAGAUACUUUUUUCAUGAGCAAU |
| | | UCCUCGCCCGGCGCCGGCAUGCCGAGGUGGGGCCACUGCGAUCAG |
| | | CGGCAUGCCGACGCCGACCCGGGGAUCUUGGAUUCACCGUUUUCU |
| | | CUCUUCUCUCUCUACAUACAGACCGGGUGGCAGGAGCGGUAAGGA |
| | | AUCAUCGUCGUCUUUCAUUCUUCGAUGAUUAUGGUAAUACUAAAU |
| | | CUUAUCUAGGAGCAUAUACAUCUAAGAUUGGAGUACUAGUAGUCG |
| | | UUUGUGGUUUCUAUUUUUUUUUAUAUUUAUCUAUGACAGUUUUU |
| | | CUGUUUUUCGUUUUGAUAAUAAUAUAAUAAAAACUCAUGGACGU |
| | | GAAAUCUGGCUUGGUUGUGGUGAUUUCAUUCUCAUUAUUGUUGU |
| | | UUUCUUUCCGUCUUGCGGAUGAAGAUGUUGCGAUGCGGUUGUUGU |
| | | UGGUGUUGCUAUACACCGAGAGAGAUGAUCUUUUUGUUCUUCUGG |
| | | UUCAUUUCCUAUGAUUGUUUGGCUGCUGACCGACGCGUCAGGAUG |
| | | UGCAGGGCAUGCGGGGAAUCAGGACCGGACACGGGAUAAUUUCAU |
| | | CUACCUAUACGGAGAUCGCGGUCCUCGCCAUGAGGAUCGCGACAG |
| | | GCGCGUCGAGGGGGCAGGAACACCCUUGCGGAUUGACAUUCUUGG |
| | | UGGUGUUUCGUUGUUGUCGGUAGUUGUUGUUGACGAUGAGGAUA |
| | | AAUAAAAAUGACCUUGUUUUUGUUCUGUUUUCUCUUGUUGGGAA |
| | | UCGUCGACUUUGAAUUCUUCGAGUUAUCGGAAAGCUGAGGUACCC |
| | | AAAUGUCUGUAGCUUUUUUCUUUUUACCCUCUUGUUUAUCAUCUG |
| | | CGAUUCGUGGUAGGUAGGAGAGGGAAAUGAUAAUCCGAGAUUAA |
| | | GGAAAGGAGAAGAUAAAAAAUAAAAAAAAAUAAUAAAAGAGAAC |
| | | GCCGGGGCAGCAGCGAUCUGGGGAUGUGCUAGCUCCGG |
| 41 | Antisense | GGAGUCAAGCUUGCAUGCAAACUUCUCAUUUAUUGUGUCUACUAC |
| | domain 2 | UCUGUGUUGCUACAGGGAGUGAAGGGGGUGAAGGCAAACAAAAA |
| | deletion | AAAAAGGAACAAAAUAAUAGAUUAGCAGAAGGAAUAAUCCGUGC |
| | mutant: | GACCGAGCUUGUGCUUCUUUUCUUAUAAGGAGGCAAAUAUACUAG |
| | ΔD2AS | GGAAAACUUAAGAAUAGGAAGAAACCGAGGUUUGGGAGAAAAGC |
| | | UGAGAUAAAAUAGCGCAUUUUCCAUACAGAGGUUGUUGUUUUUG |
| | | UGGAUCCUAAGAGGUUUCAAGUGCGAAUCUCAAAGUUCUCACGAG |
| | | AAUAUUGUCUUCAAGAAUCGACAACUGUGGUCCAAGAUUUUUUUU |
| | | UGGUCUUUUUAGGUUCUGCGAGGGACAUCACGAUGGAUCGUUGCG |
| | | AUGAAGUCACGCGUACGCCUCUGGUGUGGCGCGGUGUCGUGACAG |
| | | GAGAGUGUGUUUUCAGUGCAGAGCUGUCUUGAUUCCUAUAUCCGA |
| | | GUAUCUGUUUUCUCGUAAGGACGGUAAUCUUCUUUGGUGUAAGU |
| | | ACAUCUAAAAGCUGCAAACUAUAUUUUAAGGGCUGUCUCUAGGUG |
| | | UACUUUGAUGCUGGAGUUUUUCGCUGUGUUGAUGUGAAUAAAUC |
| | | UACUACUACUAUUAUAUGCAGAAAGAGUGAUUAUGCCGAGACAAG |
| | | AUUGCAUUGGCUGAACUGUUUCAAAAACGCCUACACUCUACUUAU |
| | | CCGUAAACCUAAGGUAAUACUAUGUGUAAGUUGUUUUUUUUUCU |
| | | UUUUGUAGUAAAAUGGUGAUACGUGCAAUUAAAACUGUAUUC CA |
| | | UGUUUCCAUCCUUUCAUUUCAACUUUAAAGGCGGCUUUGAGAGCG |
| | | AAGAAGUGCGAGGAUAAAAAUGGAUGACUCCUUCGUGUCCAGGGA |
| | | GUCGACUACUGCAACGCUGAUUGAUUAAAAGAUGGUCUCCGAUGA |
| | | UGAUGUUGUUAUUGAUCGAAUCAUGGUGCAGAACGGCGACGGAG |
| | | AGGAGCGUGUCCGCCGCCGGGAAGGUGGUCUCUUUCUCUUUUCUU |
| | | UUUUCAAGAAAUCUUCCAUGUGUUUAUCGUAGUGAUCGAAAUCGA |
| | | CUGAUCUCGGGUUCUUUUUGUUGGUUUCUUUUCGGUUAAUCAUGU |
| | | AUUGUUUUCUUUUUUUACAGAAAGAUACUUUUUUCAUGAGCAAU |
| | | UCCUCGCCCGGCGCCGGCAUGCCGAGGUGGGGCCACUGCGAUCAG |
| | | CGGCAUGCCGACGCCGACCCGGGGAUCUUGGAUUCACCGUUUUCU |
| | | CUCUUCUCUCUCUACAUACAGACCGGGUGGCAGGAGCGGUAAGGA |
| | | AUCAUCGUCGUCUUUCAUUCUUCGAUGAUUAUGGUAAUACUAAAU |
| | | CUUAUCUAGGAGCAUAUACAUCUAAGAUUGGAGUACUAGUAGUCG |
| | | UUUGUGGUUUCUAUUUUUUUUUAUAUUUAUCUAUGACAGUUUUU |
| | | CUGUUUUUCGUUUUGAUAAUAAUAUAAUAAAAACUCAUGGACGU |
| | | GAAAUCUGGCUUGGUUGUGGUGAUUUCAUUCUCAUUAUUGUUGU |
| | | UUUCUUUCCGUCUUGCGGAUGAAGAUGUUGCGAUGCGGUUGUUGU |
| | | UGGUGUUGCUAUACACCGAGAGAGAUGAUCUUUUUGUUCUUCUGG |
| | | UUCAUUUCCUAUGAUUGUUUGGCUGCUGACCGACGCGUCAGGAUG |
| | | UGCAGGGCAUGCGGGGAAUCAGGACCGGACACGGGAUAAUUUCAU |
| | | CUACCUAUACGGAGAUCGCGGUCCUCGCCAUGAGGAUCGCGACAG |
| | | GCGCGUCGAGGGGGCAGGAACACCCUUGCGGAUUGACAUUCUUGG |
| | | UGGUGUUUCGUUGUUGUCGGUAGUUGUUGUUGACGAUGAGGAUA |
| | | AAUAAAAAUGACCUUGUUUUUGUUCUGUUUUCUCUUGUUGGGAA |
| | | UCGUCGACUUUGAAUUCUUCGAGUUAUCGGAAAGCUGAGGUACCC |
| | | AAAUGUCUGUAGCUAAAAAUAAUAAAACAGAAGCCGACCGGCCGC |
| | | CGACCCGUUCCCCAGGACCAGCCUACGAGGAACGGAUAACGCGGU |
| | | GGCGACGGCAGCGGUGGUGGCGCUGGGGGUGGCGGUAGUGGUGCU |
| | | GCUGAUGGUAGUCGGGACGGAGGAGAGACGAUGCAUACAUACACG |
| | | CGUGCAUGCUGCAUGGGUGGAUGGUACGGCCGGGAGACGCGGAAG |
| | | AGAAACUCACAUAAAAAGGUGAUAAAAAGAGCGGUUGAAAAAAG |
| | | AAAACGAGAUUCGACCAGACAGAAGAGAAGGACCGGGGCUUGGCG |
| | | ACCCUUCCACGACUGCCGUUGUCAUCUCGGCUCCUCCAUCUUCUCC |
| | | CGGCCACGGGCGGCUAAGUCACCGCCGUUCUCCCCAUCCGUCCGAG |
| | | CGCCGACCGACCAGCCGGCCGAUUCGCCCGCCGGGGCUUCUGGAG |
| | | AACGCCGGGGCAGCAGCGAUCUGGGGAUGUGCUAGCUCCGG |
| 42 | Antisense | GGAGUCAAGCUUGCAUGCAAACUUCUCAUUUAUUGUGUCUACUAC |
| | domain 3 | UCUGUGUUGCUACAGGGAGUGAAGGGGGUGAAGGCAAACAAAAA |
| | deletion | AAAAAGGAACAAAAUAAUAGAUUAGCAGAAGGAAUAAUCCGUGC |
| | mutant: | GACCGAGCUUGUGCUUCUUUUCUUAUAAGGAGGCAAAUAUACUAG |
| | ΔD3AS | GGAAAACUUAAGAAUAGGAAGAAACCGAGGUUUGGGAGAAAAGC |
| | | UGAGAUAAAAUAGCGCAUUUUCCAUACAGAGGUUGUUGUUUUUG |
| | | UGGAUCCUAAGAGGUUUCAAGUGCGAAUCUCAAAGUUCUCACGAG |
| | | AAUAUUGUCUUCAAGAAUCGACAACUGUGGUCCAAGAUUUUUUUU |
| | | UGGUCUUUUUAGGUUCUGCGAGGGACAUCACGAUGGAUCGUUGCG |
| | | AUGAAGUCACGCGUACGCCUCUGGUGUGGCGCGGUGUCGUGACAG |
| | | GAGAGUGUGUUUUCAGUGCAGAGCUGUCUUGAUUCCUAUAUCCGA |
| | | GUAUCUGUUUUCUCGUAAGGACGGUAAUCUUCUUUGGUGUAAGU |
| | | ACAUCUAAAAGCUGCAAACUAUAUUUUAAGGGCUGUCUCUAGGUG |
| | | UACUUUGAUGCUGGAGUUUUUCGCUGUGUUGAUGUGAAUAAAUC |
| | | UACUACUACUAUUAUAUGCAGAAAGAGUGAUUAUGCCGAGACAAG |
| | | AUUGCAUUGGCUGAACUGUUUCAAAAACGCCUACACUCUACUUAU |
| | | CCGUAAACCUAAGGUAAUACUAUGUGUAAGUUGUUUUUUUUUCU |
| | | UUUUGUAGUAAAAUGGUGAUACGUGCAAUUAAAACUGUAUUCCA |
| | | UGUUUCCAUCCUUUCAUUUCAACUUUAAAGGCGGCUUUGAGAGCG |
| | | AAGAAGUGCGAGGAUAAAAAUGGAUGACUCCUUCGUGUCCAGGGA |
| | | GUCGACUACUGCAACGCUGAUUGAUUAAAAGAUGGUCUCCGAUGA |
| | | UGAUGUUGUUAUUGAUCGAAUCAUGGUGCAGAACGGCGACGGAG |
| | | AGGAGCGUGUCCGCCGCCGGGAAGGUGGUCUCUUUCUCUUUUCUU |
| | | UUUUCAAGAAAUCUUCCAUGUGUUUAUCGUAGUGAUCGAAAUCGA |
| | | CUGAUCUCGGGUUCUUUUUGUUGGUUUCUUUUCGGUUAAUCAUGU |
| | | AUUGUUUUCUUUUUUUACAGAAAGAUACUUUUUUCAUGAGCAAU |
| | | UCCUCGCCCGGCGCCGGCAUGCCGAGGUGGGGCCACUGCGAUCAG |
| | | CGGCAUGCCGACGCCGACCCGGGGAUCUUGGAUUCACCGUUUUCU |
| | | CUCUUCUCUCUCUACAUACAGACCGGGUGGCAGGAGCGGUAAGGA |
| | | AUCAUCGUCGUCUUUCAUUCUUCGAUGAUUAUGGUAAUACUAAAU |
| | | CUUAUCUAGGAGCAUAUACAUCUAAGAUUGGAGUACUAGUAGUCG |
| | | UUUGUGGUUUCUAUUUUUUUUUAUAUUUAUCUAUGACAGUUUUU |
| | | CUGUUUUUCGUUUUGAUAAUAAUAUAAUAAAAACUCAUGGACGU |
| | | GAAAUCUGGCUUGGUUGUGGUGAUUUCAUUCUCAUUAUUGUUGU |
| | | UUUCUUUCCGUCUUGCGGAUGAAGAUGUUGCGAUGCGGUUGUUGU |
| | | UGGUGUUGCUAUACACCGAGAGAGAUGAUCUUUUUGUUCUUCUUU |
| | | GUUUUUGUUCUGUUUUCUCUUGUUGGGAAUCGUCGACUUUGAAU |
| | | UCUUCGAGUUAUCGGAAAGCUGAGGUACCCAAAUGUCUGUAGCUU |
| | | UUUUCUUUUUACCCUCUUGUUUAUCAUCUGCGAUUCGUGGUAGGU |
| | | AGGAGAGGGAAAUGAUAAUCCGAGAUUAAGGAAAGGAGAAGAUA |
| | | AAAAAUAAAAAAAAAUAAUAAAACAGAAGCCGACCGGCCGCCGAC |
| | | CCGUUCCCCAGGACCAGCCUACGAGGAACGGAUAACGCGGUGGCG |
| | | ACGGCAGCGGUGGUGGCGCUGGGGGUGGCGGUAGUGGUGCUGCUG |
| | | AUGGUAGUCGGGACGGAGGAGAGACGAUGCAUACAUACACGCGUG |
| | | CAUGCUGCAUGGGUGGAUGGUACGGCCGGGAGACGCGGAAGAGAA |
| | | ACUCACAUAAAAAGGUGAUAAAAAGAGCGGUUGAAAAAAGAAAA |
| | | CGAGAUUCGACCAGACAGAAGAGAAGGACCGGGGCUUGGCGACCC |
| | | UUCCACGACUGCCGUUGUCAUCUCGGCUCCUCCAUCUUCUCCCGGC |
| | | CACGGGCGGCUAAGUCACCGCCGUUCUCCCCAUCCGUCCGAGCGCC |
| | | GACCGACCAGCCGGCCGAUUCGCCCGCCGGGGCUUCUGGAGAACG |
| | | CCGGGGCAGCAGCGAUCUGGGGAUGUGCUAGCUCCGG |
| 43 | Antisense | GGAGUCAAGCUUGCAUGCAAACUUCUCAUUUAUUGUGUCUACUAC |
| | domain 4 | UCUGUGUUGCUACAGGGAGUGAAGGGGGUGAAGGCAAACAAAAA |
| | deletion | AAAAAGGAACAAAAUAAUAGAUUAGCAGAAGGAAUAAUCCGUGC |
| | mutant: | GACCGAGCUUGUGCUUCUUUUCUUAUAAGGAGGCAAAUAUACUAG |
| | ΔD4AS | GGAAAACUUAAGAAUAGGAAGAAACCGAGGUUUGGGAGAAAAGC |
| | | UGAGAUAAAAUAGCGCAUUUUCCAUACAGAGGUUGUUGUUUUUG |
| | | UGGAUCCUAAGAGGUUUCAAGUGCGAAUCUCAAAGUUCUCACGAG |
| | | AAUAUUGUCUUCAAGAAUCGACAACUGUGGUCCAAGAUUUUUUUU |
| | | UGGUCUUUUUAGGUUCUGCGAGGGACAUCACGAUGGAUCGUUGCG |
| | | AUGAAGUCACGCGUACGCCUCUGGUGUGGCGCGGUGUCGUGACAG |
| | | GAGAGUGUGUUUUCAGUGCAGAGCUGUCUUGAUUCCUAUAUCCGA |
| | | GUAUCUGUUUUCUCGUAAGGACGGUAAUCUUCUUUGGUGUAAGU |
| | | ACAUCUAAAAGCUGCAAACUAUAUUUUAAGGGCUGUCUCUAGGUG |
| | | UACUUUGAUGCUGGAGUUUUUCGCUGUGUUGAUGUGAAUAAAUC |
| | | UACUACUACUAUUAUAUGCAGAAAGAGUGAUUAUGCCGAGACAAG |
| | | AUUGCAUUGGCUGAACUGUUUCAAAAACGCCUACACUCUACUUAU |
| | | CCGUAAACCUAAGGUAAUACUAUGUGUAAGUUGUUUUUUUUUCU |
| | | UUUUGUAGUAAAAUGGUGAUACGUGCAAUUAAAACUGUAUUCCA |
| | | UGUUUCCAUCCUUUCAUUUCAACUUUAAAGGCGGCUUUGAGAGCG |
| | | AAGAAGUACCCGGGGAUCUUGGAUUCACCGUUUUCUCUCUUCUCU |
| | | CUCUACAUACAGACCGGGUGGCAGGAGCGGUAAGGAAUCAUCGUC |
| | | GUCUUUCAUUCUUCGAUGAUUAUGGUAAUACUAAAUCUUAUCUAG |
| | | GAGCAUAUACAUCUAAGAUUGGAGUACUAGUAGUCGUUUGUGGU |
| | | UUCUAUUUUUUUUUAUAUUUAUCUAUGACAGUUUUUCUGUUUUU |
| | | CGUUUUGAUAAUAAUAUAAUAAAAACUCAUGGACGUGAAAUCUG |
| | | GCUUGGUUGUGGUGAUUUCAUUCUCAUUAUUGUUGUUUUCUUUCC |
| | | GUCUUGCGGAUGAAGAUGUUGCGAUGCGGUUGUUGUUGGUGUUG |
| | | CUAUACACCGAGAGAGAUGAUCUUUUUGUUCUUCUGGUUCAUUUC |
| | | CUAUGAUUGUUUGGCUGCUGACCGACGCGUCAGGAUGUGCAGGGC |
| | | AUGCGGGGAAUCAGGACCGGACACGGGAUAAUUUCAUCUACCUAU |
| | | ACGGAGAUCGCGGUCCUCGCCAUGAGGAUCGCGACAGGCGCGUCG |
| | | AGGGGGCAGGAACACCCUUGCGGAUUGACAUUCUUGGUGGUGUUU |
| | | CGUUGUUGUCGGUAGUUGUUGUUGACGAUGAGGAUAAAUAAAAA |
| | | UGACCUUGUUUUUGUUCUGUUUUCUCUUGUUGGGAAUCGUCGACU |
| | | UUGAAUUCUUCGAGUUAUCGGAAAGCUGAGGUACCCAAAUGUCUG |
| | | UAGCUUUUUUCUUUUUACCCUCUUGUUUAUCAUCUGCGAUUCGUG |
| | | GUAGGUAGGAGAGGGAAAUGAUAAUCCGAGAUUAAGGAAAGGAG |
| | | AAGAUAAAAAAUAAAAAAAAAUAAUAAAACAGAAGCCGACCGGCC |
| | | GCCGACCCGUUCCCCAGGACCAGCCUACGAGGAACGGAUAACGCG |
| | | GUGGCGACGGCAGCGGUGGUGGCGCUGGGGGUGGCGGUAGUGGUG |
| | | CUGCUGAUGGUAGUCGGGACGGAGGAGAGACGAUGCAUACAUACA |
| | | CGCGUGCAUGCUGCAUGGGUGGAUGGUACGGCCGGGAGACGCGGA |
| | | AGAGAAACUCACAUAAAAAGGUGAUAAAAAGAGCGGUUGAAAAA |
| | | AGAAAACGAGAUUCGACCAGACAGAAGAGAAGGACCGGGGCUUGG |
| | | CGACCCUUCCACGACUGCCGUUGUCAUCUCGGCUCCUCCAUCUUCU |
| | | CCCGGCCACGGGCGGCUAAGUCACCGCCGUUCUCCCCAUCCGUCCG |
| | | AGCGCCGACCGACCAGCCGGCCGAUUCGCCCGCCGGGGCUUCUGG |
| | | AGAACGCCGGGGCAGCAGCGAUCUGGGGAUGUGCUAGCUCCGG |

The following sequences were transfected as plasmid DNA using a CMV promoter and the SV40 polyA site. Hence the endogenously transcribed RNAs all start with UCAGAUCC which are the transcribed nucleotides of the CMV promoter then followed by a cleavage site, and they all end with UUUUUUUCACUGC(A)ₙ. The bold Cs indicate the two polyA cleavage sites which are then being followed by a polyA stretch (A)ₙ. so the correct end of the sequence might be either ..UUUUUUUCACUGC(A)ₙ or . . . U(A)ₙ.

| SEQ | | |
|---|---|---|
| ID | | |
| NO: | Name | Sequence |
| 44 | gGFP_β2.7_ | UCAGAUCGCUAGCGCUACCGGUCGCCACCAUGGUGAGCAAGGGCGA |
| | endo | GGAGCUGUUCACCGGGGUGGUGCCCAUCCUGGUCGAGCUGGACGGC |
| | | GACGUAAACGGCCACAAGUUCAGCGUGUCCGGCGAGGGCGAGGGC |
| | | GAUGCCACCUACGGCAAGCUGACCCUGAAGUUCAUCUGCACCACCG |
| | | GCAAGCUGCCCGUGCCCUGGCCCACCCUCGUGACCACCCUGACCUA |
| | | CGGCGUGCAGUGCUUCAGCCGCUACCCCGACCACAUGAAGCAGCAC |
| | | GACUUCUUCAAGUCCGCCAUGCCCGAAGGCUACGUCCAGGAGCGCA |
| | | CCAUCUUCUUCAAGGACGACGGCAACUACAAGACCCGCGCCGAGGU |
| | | GAAGUUCGAGGGCGACACCCUGGUGAACCGCAUCGAGCUGAAGGG |
| | | CAUCGACUUCAAGGAGGACGGCAACAUCCUGGGGCACAAGCUGGA |
| | | GUACAACUACAACAGCCACAACGUCUAUAUCAUGGCCGACAAGCAG |
| | | AAGAACGGCAUCAAGGUGAACUUCAAGAUCCGCCACAACAUCGAG |
| | | GACGGCAGCGUGCAGCUCGCCGACCACUACCAGCAGAACACCCCCA |
| | | UCGGCGACGGCCCCGUGCUGCUGCCCGACAACCACUACCUGAGCAC |
| | | CCAGUCCGCCCUGAGCAAAGACCCCAACGAGAAGCGCGAUCACAUG |
| | | GUCCUGCUGGAGUUCGUGACCGCCGCCGGGAUCACUCUCGGCAUGG |
| | | ACGAGCUGUACAAGUCCGGAAGAGCUAGCUCCCCAGAUCGCUGCUG |
| | | CCCCGGCGUUCUCCAGAAGCCCCGGCGGGCGAAUCGGCCGGCUGGU |
| | | CGGUCGGCGCUCGGACGGAUGGGGAGAACGGCGGUGACUUAGCCG |
| | | CCCGUGGCCGGGAGAAGAUGGAGGAGCCGAGAUGACAACGGCAGU |
| | | CGUGGAAGGGUCGCCAAGCCCCGGUCCUUCUCUUCUGUCUGGUCGA |
| | | AUCUCGUUUUCUUUUUUCAACCGCUCUUUUUAUCACCUUUUUAUG |
| | | UGAGUUUCUCUUCCGCGUCUCCCGGCCGUACCAUCCACCCAUGCAG |
| | | CAUGCACGCGUGUAUGUAUGCAUCGUCUCUCCUCCGUCCCGACUAC |
| | | CAUCAGCAGCACCACUACCGCCACCCCCAGCGCCACCACCGCUGCCG |
| | | UCGCCACCGCGUUAUCCGUUCCUCGUAGGCUGGUCCUGGGGAACGG |
| | | GUCGGCGGCCGGUCGGCUUCUGUUUUAUUAUUUUUUUUUAUUUUU |
| | | UAUCUUCUCCUUUCCUUAAUCUCGGAUUAUCAUUUCCCUCUCCUAC |
| | | CUACCACGAAUCGCAGAUGAUAAACAAGAGGGUAAAAAGAAAAAA |
| | | GCUACAGACAUUUGGGUACCUCAGCUUUCCGAUAACUCGAAGAAU |
| | | UCAAAGUCGACGAUUCCCAACAAGAGAAAACAGAACAAAAACAAG |
| | | GUCAUUUUUAUUUAUCCUCAUCGUCAACAACAACUACCGACAACAA |
| | | CGAAACACCACCAAGAAUGUCAAUCCGCAAGGGUGUUCCUGCCCCC |
| | | UCGACGCGCCUGUCGCGAUCCUCAUGGCGAGGACCGCGAUCUCCGU |
| | | AUAGGUAGAUGAAAUUAUCCCGUGUCCGGUCCUGAUUCCCCGCAU |
| | | GCCCUGCACAUCCUGACGCGUCGGUCAGCAGCCAAACAAUCAUAGG |
| | | AAAUGAACCAGAAGAACAAAAAGAUCAUCUCUCUCGGUGUAUAGC |
| | | AACACCAACAACAACCGCAUCGCAACAUCUUCAUCCGCAAGACGGA |
| | | AAGAAAACAACAAUAAUGAGAAUGAAAUCACCACAACCAAGCCAG |
| | | AUUUCACGUCCAUGAGUUUUUAUUAUAUUAUUAUCAAAACGAAAA |
| | | ACAGAAAAACUGUCAUAGAUAAAUAUAAAAAAAAAUAGAAACCAC |
| | | AAACGACUACUAGUACUCCAAUCUUAGAUGUAUAUGCUCCUAGAU |
| | | AAGAUUUAGUAUUACCAUAAUCAUCGAAGAAUGAAAGACGACGAU |
| | | GAUUCCUUACCGCUCCUGCCACCCGGUCUGUAUGUAGAGAGAGAAG |
| | | AGAGAAAACGGUGAAUCCAAGAUCCCCGGGUCGGCGUCGGCAUGCC |
| | | GCUGAUCGCAGUGGCCCCACCUCGGCAUGCCGGCGCCGGGCGAGGA |
| | | AUUGCUCAUGAAAAAAGUAUCUUUCUGUAAAAAAAGAAAACAAUA |
| | | CAUGAUUAACCGAAAAGAAACCAACAAAAAGAACCCGAGAUCAGU |
| | | CGAUUUCGAUCACUACGAUAAACACAUGGAAGAUUUCUUGAAAAA |
| | | AGAAAAGAGAAAGAGACCACCUUCCCGGCGGCGGACACGCUCCUCU |
| | | CCGUCGCCGUUCUGCACCAUGAUUCGAUCAAUAACAACAUCAUCAU |
| | | CGGAGACCAUCUUUUAAUCAAUCAGCGUUGCAGUAGUCGACUCCCU |
| | | GGACACGAAGGAGUCAUCCAUUUUUAUCCUCGCACUUCUUCGCUCU |
| | | CAAAGCCGCCUUUAAAGUUGAAAUGAAAGGAUGGAAACAUGGAAU |
| | | ACAGUUUUAAUUGCACGUAUCACCAUUUUACUACAAAAAGAAAAA |
| | | AAAACAACUUACACAUAGUAUUACCUUAGGUUUACGGAUAAGUAG |
| | | AGUGUAGGCGUUUUUGAAACAGUUCAGCCAAUGCAAUCUUGUCUC |
| | | GGCAUAAUCACUCUUUCUGCAUAUAAUAGUAGUAGUAGAUUUAUU |
| | | CACAUCAACACAGCGAAAAACUCCAGCAUCAAAGUACACCUAGAGA |
| | | CAGCCCUUAAAAUAUAGUUUGCAGCUUUUAGAUGUACUUACACCA |
| | | AAGAAGAUUACCGUCCUUACGAGAAAACAGAUACUCGGAUAUAGG |
| | | AAUCAAGACAGCUCUGCACUGAAAACACACUCUCCUGUCACGACAC |
| | | CGCGCCACACCAGAGGCGUACGCGUGACUUCAUCGCAACGAUCCAU |
| | | CGUGAUGUCCCUCGCAGAACCUAAAAAGACCAAAAAAAAAUCUUG |
| | | GACCACAGUUGUCGAUUCUUGAAGACAAUAUUCUCGUGAGAACUU |
| | | UGAGAUUCGCACUUGAAACCUCUUAGGAUCCACAAAAACAACAACC |
| | | UCUGUAUGGAAAAUGCGCUAUUUUAUCUCAGCUUUUCUCCCAAAC |
| | | CUCGGUUUCUUCCUAUUCUUAAGUUUUCCCUAGUAUAUUUGCCUCC |
| | | UUAUAAGAAAAGAAGCACAAGCUCGGUCGCACGGAUUAUUCCUUC |
| | | UGCUAAUCUAUUAUUUUGUUCCUUUUUUUUUUGUUUGCCUUCACC |
| | | CCCUUCACUCCCUGUAGCAACACAGAGUAGUAGACACAAUAAAUGA |
| | | GAAGUAAGCUUGACUCCUAUAGUGUCACCUAAAUGUCUAGAAACU |
| | | UGUUUAUUGCAGCUUAUAAUGGUUACAAAUAAAGCAAUAGCAUCA |
| | | CAAAUUUCACAAAUAAAGCAUUUUUUUCACUGC(A)ₙ |
| 45 | gGFP_s_β2.7_ | UCAGAUCGCUAGCGCUACCGGUCGCCACCAUGGUGAGCAAGGGCGA |
| | endo | GGAGCUGUUCACCGGGGUGGUGCCCAUCCUGGUCGAGCUGGACGGC |
| | | GACGUAAACGGCCACAAGUUCAGCGUGUCCGGCGAGGGCGAGGGC |
| | | GAUGCCACCUACGGCAAGCUGACCCUGAAGUUCAUCUGCACCACCG |
| | | GCAAGCUGCCCGUGCCCUGGCCCACCCUCGUGACCACCCUGACCUA |
| | | CGGCGUGCAGUGCUUCAGCCGCUACCCCGACCACAUGAAGCAGCAC |
| | | GACUUCUUCAAGUCCGCCAUGCCCGAAGGCUACGUCCAGGAGCGCA |
| | | CCAUCUUCUUCAAGGACGACGGCAACUACAAGACCCGCGCCGAGGU |
| | | GAAGUUCGAGGGCGACACCCUGGUGAACCGCAUCGAGCUGAAGGG |
| | | CAUCGACUUCAAGGAGGACGGCAACAUCCUGGGGCACAAGCUGGA |
| | | GUACAACUACAACAGCCACAACGUCUAUAUCAUGGCCGACAAGCAG |
| | | AAGAACGGCAUCAAGGUGAACUUCAAGAUCCGCCACAACAUCGAG |
| | | GACGGCAGCGUGCAGCUCGCCGACCACUACCAGCAGAACACCCCCA |
| | | UCGGCGACGGCCCCGUGCUGCUGCCCGACAACCACUACCUGAGCAC |
| | | CCAGUCCGCCCUGAGCAAAGACCCCAACGAGAAGCGCGAUCACAUG |
| | | GUCCUGCUGGAGUUCGUGACCGCCGCCGGGAUCACUCUCGGCAUGG |
| | | ACGAGCUGUACAAGUCCGGAUGACACCACCCCGGAGAACAGCUCCU |
| | | CGCUAGCUCCCCAGAUCGCUGCUGCCCCGGCGUUCUCCAGAAGCCC |
| | | CGGCGGGCGAAUCGGCCGGCUGGUCGGUCGGCGCUCGGACGGAUGG |
| | | GGAGAACGGCGGUGACUUAGCCGCCCGUGGCCGGGAGAAGAUGGA |
| | | GGAGCCGAGAUGACAACGGCAGUCGUGGAAGGGUCGCCAAGCCCCG |
| | | GUCCUUCUCUUCUGUCUGGUCGAAUCUCGUUUUCUUUUUUCAACCG |
| | | CUCUUUUUAUCACCUUUUUAUGUGAGUUUCUCUUCCGCGUCUCCCG |
| | | GCCGUACCAUCCACCCAUGCAGCAUGCACGCGUGUAUGUAUGCAUC |
| | | GUCUCUCCUCCGUCCCGACUACCAUCAGCAGCACCACUACCGCCAC |
| | | CCCCAGCGCCACCACCGCUGCCGUCGCCACCGCGUUAUCCGUUCCU |
| | | CGUAGGCUGGUCCUGGGGAACGGGUCGGCGGCCGGUCGGCUUCUG |
| | | UUUUAUUAUUUUUUUUUAUUUUUUAUCUUCUCCUUUCCUUAAUCU |
| | | CGGAUUAUCAUUUCCCUCUCCUACCUACCACGAAUCGCAGAUGAUA |
| | | AACAAGAGGGUAAAAAGAAAAAAGCUACAGACAUUUGGGUACCUC |
| | | AGCUUUCCGAUAACUCGAAGAAUUCAAAGUCGACGAUUCCCAACA |
| | | AGAGAAAACAGAACAAAAACAAGGUCAUUUUUAUUUAUCCUCAUC |
| | | GUCAACAACAACUACCGACAACAACGAAACACCACCAAGAAUGUCA |
| | | AUCCGCAAGGGUGUUCCUGCCCCCUCGACGCGCCUGUCGCGAUCCU |
| | | CAUGGCGAGGACCGCGAUCUCCGUAUAGGUAGAUGAAAUUAUCCC |
| | | GUGUCCGGUCCUGAUUCCCCGCAUGCCCUGCACAUCCUGACGCGUC |
| | | GGUCAGCAGCCAAACAAUCAUAGGAAAUGAACCAGAAGAACAAAA |
| | | AGAUCAUCUCUCUCGGUGUAUAGCAACACCAACAACAACCGCAUCG |
| | | CAACAUCUUCAUCCGCAAGACGGAAAGAAAACAACAAUAAUGAGA |
| | | AUGAAAUCACCACAACCAAGCCAGAUUUCACGUCCAUGAGUUUUU |
| | | AUUAUAUUAUUAUCAAAACGAAAAACAGAAAAACUGUCAUAGAUA |
| | | AAUAUAAAAAAAAAUAGAAACCACAAACGACUACUAGUACUCCAA |
| | | UCUUAGAUGUAUAUGCUCCUAGAUAAGAUUUAGUAUUACCAUAAU |
| | | CAUCGAAGAAUGAAAGACGACGAUGAUUCCUUACCGCUCCUGCCAC |
| | | CCGGUCUGUAUGUAGAGAGAGAAGAGAGAAAACGGUGAAUCCAAG |
| | | AUCCCCGGGUCGGCGUCGGCAUGCCGCUGAUCGCAGUGGCCCCACC |
| | | UCGGCAUGCCGGCGCCGGGCGAGGAAUUGCUCAUGAAAAAAGUAU |
| | | CUUUCUGUAAAAAAAGAAAACAAUACAUGAUUAACCGAAAAGAAA |
| | | CCAACAAAAAGAACCCGAGAUCAGUCGAUUUCGAUCACUACGAUA |
| | | AACACAUGGAAGAUUUCUUGAAAAAAGAAAAGAGAAAGAGACCAC |
| | | CUUCCCGGCGGCGGACACGCUCCUCUCCGUCGCCGUUCUGCACCAU |
| | | GAUUCGAUCAAUAACAACAUCAUCAUCGGAGACCAUCUUUUAAUC |
| | | AAUCAGCGUUGCAGUAGUCGACUCCCUGGACACGAAGGAGUCAUCC |
| | | AUUUUUAUCCUCGCACUUCUUCGCUCUCAAAGCCGCCUUUAAAGUU |
| | | GAAAUGAAAGGAUGGAAACAUGGAAUACAGUUUUAAUUGCACGUA |
| | | UCACCAUUUUACUACAAAAAGAAAAAAAAACAACUUACACAUAGU |
| | | AUUACCUUAGGUUUACGGAUAAGUAGAGUGUAGGCGUUUUUGAAA |
| | | CAGUUCAGCCAAUGCAAUCUUGUCUCGGCAUAAUCACUCUUUCUGC |
| | | AUAUAAUAGUAGUAGUAGAUUUAUUCACAUCAACACAGCGAAAAA |
| | | CUCCAGCAUCAAAGUACACCUAGAGACAGCCCUUAAAAUAUAGUU |
| | | UGCAGCUUUUAGAUGUACUUACACCAAAGAAGAUUACCGUCCUUA |
| | | CGAGAAAACAGAUACUCGGAUAUAGGAAUCAAGACAGCUCUGCAC |
| | | UGAAAACACACUCUCCUGUCACGACACCGCGCCACACCAGAGGCGU |
| | | ACGCGUGACUUCAUCGCAACGAUCCAUCGUGAUGUCCCUCGCAGAA |
| | | CCUAAAAAGACCAAAAAAAAAUCUUGGACCACAGUUGUCGAUUCU |
| | | UGAAGACAAUAUUCUCGUGAGAACUUUGAGAUUCGCACUUGAAAC |
| | | CUCUUAGGAUCCACAAAAACAACAACCUCUGUAUGGAAAAUGCGC |
| | | UAUUUUAUCUCAGCUUUUCUCCCAAACCUCGGUUUCUUCCUAUUCU |
| | | UAAGUUUUCCCUAGUAUAUUUGCCUCCUUAUAAGAAAAGAAGCAC |
| | | AAGCUCGGUCGCACGGAUUAUUCCUUCUGCUAAUCUAUUAUUUUG |
| | | UUCCUUUUUUUUUUGUUUGCCUUCACCCCCUUCACUCCCUGUAGCA |
| | | ACACAGAGUAGUAGACACAAUAAAUGAGAAGUAAGCUUGACUCCU |
| | | AUAGUGUCACCUAAAUGUCUAGAAACUUGUUUAUUGCAGCUUAUA |
| | | AUGGUUACAAAUAAAGCAAUAGCAUCACAAAUUUCACAAAUAAAG |
| | | CAUUUUUUUCACUGC(A)ₙ |
| 46 | mtGFP_ | UCAGAUCGCUAGCGCUACCGGUCGCCACCAUAGUGAGCAAGGGCGA |
| | s_β2.7_ | GGAGCUGUUCACCGGGGUGGUGCCCAUCCUGGUCGAGCUGGACGGC |
| | endo | GACGUAAACGGCCACAAGUUCAGCGUGUCCGGCGAGGGCGAGGGC |
| | | GAUGCCACCUACGGCAAGCUGACCCUGAAGUUCAUCUGCACCACCG |
| | | GCAAGCUGCCCGUGCCCUGGCCCACCCUCGUGACCACCCUGACCUA |
| | | CGGCGUGCAGUGCUUCAGCCGCUACCCCGACCACAUGAAGCAGCAC |
| | | GACUUCUUCAAGUCCGCCAUGCCCGAAGGCUACGUCCAGGAGCGCA |
| | | CCAUCUUCUUCAAGGACGACGGCAACUACAAGACCCGCGCCGAGGU |
| | | GAAGUUCGAGGGCGACACCCUGGUGAACCGCAUCGAGCUGAAGGG |
| | | CAUCGACUUCAAGGAGGACGGCAACAUCCUGGGGCACAAGCUGGA |
| | | GUACAACUACAACAGCCACAACGUCUAUAUCAUGGCCGACAAGCAG |
| | | AAGAACGGCAUCAAGGUGAACUUCAAGAUCCGCCACAACAUCGAG |
| | | GACGGCAGCGUGCAGCUCGCCGACCACUACCAGCAGAACACCCCCA |
| | | UCGGCGACGGCCCCGUGCUGCUGCCCGACAACCACUACCUGAGCAC |
| | | CCAGUCCGCCCUGAGCAAAGACCCCAACGAGAAGCGCGAUCACAUG |
| | | GUCCUGCUGGAGUUCGUGACCGCCGCCGGGAUCACUCUCGGCAUGG |
| | | ACGAGCUGUACAAGUCCGGAAGACACCACCCCGGAGAACAGCUCCU |
| | | CGCUAGCUCCCCAGAUCGCUGCUGCCCCGGCGUUCUCCAGAAGCCC |
| | | CGGCGGGCGAAUCGGCCGGCUGGUCGGUCGGCGCUCGGACGGAUGG |
| | | GGAGAACGGCGGUGACUUAGCCGCCCGUGGCCGGGAGAAGAUGGA |
| | | GGAGCCGAGAUGACAACGGCAGUCGUGGAAGGGUCGCCAAGCCCCG |
| | | GUCCUUCUCUUCUGUCUGGUCGAAUCUCGUUUUCUUUUUUCAACCG |
| | | CUCUUUUUAUCACCUUUUUAUGUGAGUUUCUCUUCCGCGUCUCCCG |
| | | GCCGUACCAUCCACCCAUGCAGCAUGCACGCGUGUAUGUAUGCAUC |
| | | GUCUCUCCUCCGUCCCGACUACCAUCAGCAGCACCACUACCGCCAC |
| | | CCCCAGCGCCACCACCGCUGCCGUCGCCACCGCGUUAUCCGUUCCU |
| | | CGUAGGCUGGUCCUGGGGAACGGGUCGGCGGCCGGUCGGCUUCUG |
| | | UUUUAUUAUUUUUUUUUAUUUUUUAUCUUCUCCUUUCCUUAAUCU |
| | | CGGAUUAUCAUUUCCCUCUCCUACCUACCACGAAUCGCAGAUGAUA |
| | | AACAAGAGGGUAAAAAGAAAAAAGCUACAGACAUUUGGGUACCUC |
| | | AGCUUUCCGAUAACUCGAAGAAUUCAAAGUCGACGAUUCCCAACA |
| | | AGAGAAAACAGAACAAAAACAAGGUCAUUUUUAUUUAUCCUCAUC |
| | | GUCAACAACAACUACCGACAACAACGAAACACCACCAAGAAUGUCA |
| | | AUCCGCAAGGGUGUUCCUGCCCCCUCGACGCGCCUGUCGCGAUCCU |
| | | CAUGGCGAGGACCGCGAUCUCCGUAUAGGUAGAUGAAAUUAUCCC |
| | | GUGUCCGGUCCUGAUUCCCCGCAUGCCCUGCACAUCCUGACGCGUC |
| | | GGUCAGCAGCCAAACAAUCAUAGGAAAUGAACCAGAAGAACAAAA |
| | | AGAUCAUCUCUCUCGGUGUAUAGCAACACCAACAACAACCGCAUCG |
| | | CAACAUCUUCAUCCGCAAGACGGAAAGAAAACAACAAUAAUGAGA |
| | | AUGAAAUCACCACAACCAAGCCAGAUUUCACGUCCAUGAGUUUUU |
| | | AUUAUAUUAUUAUCAAAACGAAAAACAGAAAAACUGUCAUAGAUA |
| | | AAUAUAAAAAAAAAUAGAAACCACAAACGACUACUAGUACUCCAA |
| | | UCUUAGAUGUAUAUGCUCCUAGAUAAGAUUUAGUAUUACCAUAAU |
| | | CAUCGAAGAAUGAAAGACGACGAUGAUUCCUUACCGCUCCUGCCAC |
| | | CCGGUCUGUAUGUAGAGAGAGAAGAGAGAAAACGGUGAAUCCAAG |
| | | AUCCCCGGGUCGGCGUCGGCAUGCCGCUGAUCGCAGUGGCCCCACC |
| | | UCGGCAUGCCGGCGCCGGGCGAGGAAUUGCUCAUGAAAAAAGUAU |
| | | CUUUCUGUAAAAAAAGAAAACAAUACAUGAUUAACCGAAAAGAAA |
| | | CCAACAAAAAGAACCCGAGAUCAGUCGAUUUCGAUCACUACGAUA |
| | | AACACAUGGAAGAUUUCUUGAAAAAAGAAAAGAGAAAGAGACCAC |
| | | CUUCCCGGCGGCGGACACGCUCCUCUCCGUCGCCGUUCUGCACCAU |
| | | GAUUCGAUCAAUAACAACAUCAUCAUCGGAGACCAUCUUUUAAUC |
| | | AAUCAGCGUUGCAGUAGUCGACUCCCUGGACACGAAGGAGUCAUCC |
| | | AUUUUUAUCCUCGCACUUCUUCGCUCUCAAAGCCGCCUUUAAAGUU |
| | | GAAAUGAAAGGAUGGAAACAUGGAAUACAGUUUUAAUUGCACGUA |
| | | UCACCAUUUUACUACAAAAAGAAAAAAAAACAACUUACACAUAGU |
| | | AUUACCUUAGGUUUACGGAUAAGUAGAGUGUAGGCGUUUUUGAAA |
| | | CAGUUCAGCCAAUGCAAUCUUGUCUCGGCAUAAUCACUCUUUCUGC |
| | | AUAUAAUAGUAGUAGUAGAUUUAUUCACAUCAACACAGCGAAAAA |
| | | CUCCAGCAUCAAAGUACACCUAGAGACAGCCCUUAAAAUAUAGUU |
| | | UGCAGCUUUUAGAUGUACUUACACCAAAGAAGAUUACCGUCCUUA |
| | | CGAGAAAACAGAUACUCGGAUAUAGGAAUCAAGACAGCUCUGCAC |
| | | UGAAAACACACUCUCCUGUCACGACACCGCGCCACACCAGAGGCGU |
| | | ACGCGUGACUUCAUCGCAACGAUCCAUCGUGAUGUCCCUCGCAGAA |
| | | CCUAAAAAGACCAAAAAAAAAUCUUGGACCACAGUUGUCGAUUCU |
| | | UGAAGACAAUAUUCUCGUGAGAACUUUGAGAUUCGCACUUGAAAC |
| | | CUCUUAGGAUCCACAAAAACAACAACCUCUGUAUGGAAAAUGCGC |
| | | UAUUUUAUCUCAGCUUUUCUCCCAAACCUCGGUUUCUUCCUAUUCU |
| | | UAAGUUUUCCCUAGUAUAUUUGCCUCCUUAUAAGAAAAGAAGCAC |
| | | AAGCUCGGUCGCACGGAUUAUUCCUUCUGCUAAUCUAUUAUUUUG |
| | | UUCCUUUUUUUUUUGUUUGCCUUCACCCCCUUCACUCCCUGUAGCA |
| | | ACACAGAGUAGUAGACACAAUAAAUGAGAAGUAAGCUUGACUCCU |
| | | AUAGUGUCACCUAAAUGUCUAGAAACUUGUUUAUUGCAGCUUAUA |
| | | AUGGUUACAAAUAAAGCAAUAGCAUCACAAAUUUCACAAAUAAAG |
| | | CAUUUUUUUCACUGC(A)ₙ |
| 47 | mtGFP_ | UCAGAUCGCUAGCGCUACCGGUCGCCACCAUAGUGAGCAAGGGCGA |
| | NLS_s_ | GGAGCUGUUCACCGGGGUGGUGCCCAUCCUGGUCGAGCUGGACGGC |
| | β2.7_endo | GACGUAAACGGCCACAAGUUCAGCGUGUCCGGCGAGGGCGAGGGC |
| | | GAUGCCACCUACGGCAAGCUGACCCUGAAGUUCAUCUGCACCACCG |
| | | GCAAGCUGCCCGUGCCCUGGCCCACCCUCGUGACCACCCUGACCUA |
| | | CGGCGUGCAGUGCUUCAGCCGCUACCCCGACCACAUGAAGCAGCAC |
| | | GACUUCUUCAAGUCCGCCAUGCCCGAAGGCUACGUCCAGGAGCGCA |
| | | CCAUCUUCUUCAAGGACGACGGCAACUACAAGACCCGCGCCGAGGU |
| | | GAAGUUCGAGGGCGACACCCUGGUGAACCGCAUCGAGCUGAAGGG |
| | | CAUCGACUUCAAGGAGGACGGCAACAUCCUGGGGCACAAGCUGGA |
| | | GUACAACUACAACAGCCACAACGUCUAUAUCAUGGCCGACAAGCAG |
| | | AAGAACGGCAUCAAGGUGAACUUCAAGAUCCGCCACAACAUCGAG |
| | | GACGGCAGCGUGCAGCUCGCCGACCACUACCAGCAGAACACCCCCA |
| | | UCGGCGACGGCCCCGUGCUGCUGCCCGACAACCACUACCUGAGCAC |
| | | CCAGUCCGCCCUGAGCAAAGACCCCAACGAGAAGCGCGAUCACAUG |
| | | GUCCUGCUGGAGUUCGUGACCGCCGCCGGGAUCACUCUCGGCAUGG |
| | | ACGAGCUGUACAAGGCCGGACUCAGAUCUCGAGCUGAUCCAAAAA |
| | | AGAAGAGAAAGGUAGAUCCAAAAAAGAAGAGAAAGGUAGAUCCAA |
| | | AAAAGAAGAGAAAGGUAAGGAUCCACCGGAUCUAGAUAACUGUCC |
| | | GGAAGACACCACCCCGGAGAACAGCUCCUCGCUAGCUCCCCAGAUC |
| | | GCUGCUGCCCCGGCGUUCUCCAGAAGCCCCGGCGGGCGAAUCGGCC |
| | | GGCUGGUCGGUCGGCGCUCGGACGGAUGGGGAGAACGGCGGUGAC |
| | | UUAGCCGCCCGUGGCCGGGAGAAGAUGGAGGAGCCGAGAUGACAA |
| | | CGGCAGUCGUGGAAGGGUCGCCAAGCCCCGGUCCUUCUCUUCUGUC |
| | | UGGUCGAAUCUCGUUUUCUUUUUUCAACCGCUCUUUUUAUCACCU |
| | | UUUUAUGUGAGUUUCUCUUCCGCGUCUCCCGGCCGUACCAUCCACC |
| | | CAUGCAGCAUGCACGCGUGUAUGUAUGCAUCGUCUCUCCUCCGUCC |
| | | CGACUACCAUCAGCAGCACCACUACCGCCACCCCCAGCGCCACCACC |
| | | GCUGCCGUCGCCACCGCGUUAUCCGUUCCUCGUAGGCUGGUCCUGG |
| | | GGAACGGGUCGGCGGCCGGUCGGCUUCUGUUUUAUUAUUUUUUUU |
| | | UAUUUUUUAUCUUCUCCUUUCCUUAAUCUCGGAUUAUCAUUUCCC |
| | | UCUCCUACCUACCACGAAUCGCAGAUGAUAAACAAGAGGGUAAAA |
| | | AGAAAAAAGCUACAGACAUUUGGGUACCUCAGCUUUCCGAUAACU |
| | | CGAAGAAUUCAAAGUCGACGAUUCCCAACAAGAGAAAACAGAACA |
| | | AAAACAAGGUCAUUUUUAUUUAUCCUCAUCGUCAACAACAACUAC |
| | | CGACAACAACGAAACACCACCAAGAAUGUCAAUCCGCAAGGGUGUU |
| | | CCUGCCCCCUCGACGCGCCUGUCGCGAUCCUCAUGGCGAGGACCGC |
| | | GAUCUCCGUAUAGGUAGAUGAAAUUAUCCCGUGUCCGGUCCUGAU |
| | | UCCCCGCAUGCCCUGCACAUCCUGACGCGUCGGUCAGCAGCCAAAC |
| | | AAUCAUAGGAAAUGAACCAGAAGAACAAAAAGAUCAUCUCUCUCG |
| | | GUGUAUAGCAACACCAACAACAACCGCAUCGCAACAUCUUCAUCCG |
| | | CAAGACGGAAAGAAAACAACAAUAAUGAGAAUGAAAUCACCACAA |
| | | CCAAGCCAGAUUUCACGUCCAUGAGUUUUUAUUAUAUUAUUAUCA |
| | | AAACGAAAAACAGAAAAACUGUCAUAGAUAAAUAUAAAAAAAAAU |
| | | AGAAACCACAAACGACUACUAGUACUCCAAUCUUAGAUGUAUAUG |
| | | CUCCUAGAUAAGAUUUAGUAUUACCAUAAUCAUCGAAGAAUGAAA |
| | | GACGACGAUGAUUCCUUACCGCUCCUGCCACCCGGUCUGUAUGUAG |
| | | AGAGAGAAGAGAGAAAACGGUGAAUCCAAGAUCCCCGGGUCGGCG |
| | | UCGGCAUGCCGCUGAUCGCAGUGGCCCCACCUCGGCAUGCCGGCGC |
| | | CGGGCGAGGAAUUGCUCAUGAAAAAAGUAUCUUUCUGUAAAAAAA |
| | | GAAAACAAUACAUGAUUAACCGAAAAGAAACCAACAAAAAGAACC |
| | | CGAGAUCAGUCGAUUUCGAUCACUACGAUAAACACAUGGAAGAUU |
| | | UCUUGAAAAAAGAAAAGAGAAAGAGACCACCUUCCCGGCGGCGGA |
| | | CACGCUCCUCUCCGUCGCCGUUCUGCACCAUGAUUCGAUCAAUAAC |
| | | AACAUCAUCAUCGGAGACCAUCUUUUAAUCAAUCAGCGUUGCAGU |
| | | AGUCGACUCCCUGGACACGAAGGAGUCAUCCAUUUUUAUCCUCGCA |
| | | CUUCUUCGCUCUCAAAGCCGCCUUUAAAGUUGAAAUGAAAGGAUG |
| | | GAAACAUGGAAUACAGUUUUAAUUGCACGUAUCACCAUUUUACUA |
| | | CAAAAAGAAAAAAAAACAACUUACACAUAGUAUUACCUUAGGUUU |
| | | ACGGAUAAGUAGAGUGUAGGCGUUUUUGAAACAGUUCAGCCAAUG |
| | | CAAUCUUGUCUCGGCAUAAUCACUCUUUCUGCAUAUAAUAGUAGU |
| | | AGUAGAUUUAUUCACAUCAACACAGCGAAAAACUCCAGCAUCAAA |
| | | GUACACCUAGAGACAGCCCUUAAAAUAUAGUUUGCAGCUUUUAGA |
| | | UGUACUUACACCAAAGAAGAUUACCGUCCUUACGAGAAAACAGAU |
| | | ACUCGGAUAUAGGAAUCAAGACAGCUCUGCACUGAAAACACACUC |
| | | UCCUGUCACGACACCGCGCCACACCAGAGGCGUACGCGUGACUUCA |
| | | UCGCAACGAUCCAUCGUGAUGUCCCUCGCAGAACCUAAAAAGACCA |
| | | AAAAAAAAUCUUGGACCACAGUUGUCGAUUCUUGAAGACAAUAUU |
| | | CUCGUGAGAACUUUGAGAUUCGCACUUGAAACCUCUUAGGAUCCA |
| | | CAAAAACAACAACCUCUGUAUGGAAAAUGCGCUAUUUUAUCUCAG |
| | | CUUUUCUCCCAAACCUCGGUUUCUUCCUAUUCUUAAGUUUUCCCUA |
| | | GUAUAUUUGCCUCCUUAUAAGAAAAGAAGCACAAGCUCGGUCGCA |
| | | CGGAUUAUUCCUUCUGCUAAUCUAUUAUUUUGUUCCUUUUUUUUU |
| | | UGUUUGCCUUCACCCCCUUCACUCCCUGUAGCAACACAGAGUAGUA |
| | | GACACAAUAAAUGAGAAGUAAGCUUGACUCCUAUAGUGUCACCUA |
| | | AAUGUCUAGAAACUUGUUUAUUGCAGCUUAUAAUGGUUACAAAUA |
| | | AAGCAAUAGCAUCACAAAUUUCACAAAUAAAGCAUUUUUUUCACU |
| | | GC(A)ₙ |
| 48 | mtGFP_s_ | UCAGAUCGCUAGCGCUACCGGUCGCCACCAUAGUGAGCAAGGGCGA |
| | endo | GGAGCUGUUCACCGGGGUGGUGCCCAUCCUGGUCGAGCUGGACGGC |
| | | GACGUAAACGGCCACAAGUUCAGCGUGUCCGGCGAGGGCGAGGGC |
| | | GAUGCCACCUACGGCAAGCUGACCCUGAAGUUCAUCUGCACCACCG |
| | | GCAAGCUGCCCGUGCCCUGGCCCACCCUCGUGACCACCCUGACCUA |
| | | CGGCGUGCAGUGCUUCAGCCGCUACCCCGACCACAUGAAGCAGCAC |
| | | GACUUCUUCAAGUCCGCCAUGCCCGAAGGCUACGUCCAGGAGCGCA |
| | | CCAUCUUCUUCAAGGACGACGGCAACUACAAGACCCGCGCCGAGGU |
| | | GAAGUUCGAGGGCGACACCCUGGUGAACCGCAUCGAGCUGAAGGG |
| | | CAUCGACUUCAAGGAGGACGGCAACAUCCUGGGGCACAAGCUGGA |
| | | GUACAACUACAACAGCCACAACGUCUAUAUCAUGGCCGACAAGCAG |
| | | AAGAACGGCAUCAAGGUGAACUUCAAGAUCCGCCACAACAUCGAG |
| | | GACGGCAGCGUGCAGCUCGCCGACCACUACCAGCAGAACACCCCCA |
| | | UCGGCGACGGCCCCGUGCUGCUGCCCGACAACCACUACCUGAGCAC |
| | | CCAGUCCGCCCUGAGCAAAGACCCCAACGAGAAGCGCGAUCACAUG |
| | | GUCCUGCUGGAGUUCGUGACCGCCGCCGGGAUCACUCUCGGCAUGG |
| | | ACGAGCUGUACAAGUCCGGAAGACACCACCCCGGAGAACAGCUCCU |
| | | CGGAUCCACCGGAUCUAGAUAACUGAUCAUAAUCAGCCAUACCACA |
| | | UUUGUAGAGGUUUUACUUGCUUUAAAAAACCUCCCACACCUCCCCC |
| | | UGAACCUGAAACAUAAAAUGAAUGCAAUUGUUGUUGUUAACUUGU |
| | | UUAUUGCAGCUUAUAAUGGUUACAAAUAAAGCAAUAGCAUCACAA |
| | | AUUUCACAAAUAAAGCAUUUUUUUCACUGC(A)ₙ |
| 49 | Full length | UCAGAUCUAAUACGACUCACUAUAGGCGCUUCCGGAAGAGCUAGC |
| | β2.7 | UCCCCAGAUCGCUGCUGCCCCGGCGUUCUCCAGAAGCCCCGGCGGG |
| | RNA_endo | CGAAUCGGCCGGCUGGUCGGUCGGCGCUCGGACGGAUGGGGAGAA |
| | (sense) | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAAAGCUACAGACAUUUGGGUACCUCAGCUUUC |
| | | CGAUAACUCGAAGAAUUCAAAGUCGACGAUUCCCAACAAGAGAAA |
| | | ACAGAACAAAAACAAGGUCAUUUUUAUUUAUCCUCAUCGUCAACA |
| | | ACAACUACCGACAACAACGAAACACCACCAAGAAUGUCAAUCCGCA |
| | | AGGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAGC |
| | | AGCCAAACAAUCAUAGGAAAUGAACCAGAAGAACAAAAAGAUCAU |
| | | CUCUCUCGGUGUAUAGCAACACCAACAACAACCGCAUCGCAACAUC |
| | | UUCAUCCGCAAGACGGAAAGAAAACAACAAUAAUGAGAAUGAAAU |
| | | CACCACAACCAAGCCAGAUUUCACGUCCAUGAGUUUUUAUUAUAU |
| | | UAUUAUCAAAACGAAAAACAGAAAAACUGUCAUAGAUAAAUAUAA |
| | | AAAAAAAUAGAAACCACAAACGACUACUAGUACUCCAAUCUUAGA |
| | | UGUAUAUGCUCCUAGAUAAGAUUUAGUAUUACCAUAAUCAUCGAA |
| | | GAAUGAAAGACGACGAUGAUUCCUUACCGCUCCUGCCACCCGGUCU |
| | | GUAUGUAGAGAGAGAAGAGAGAAAACGGUGAAUCCAAGAUCCCCG |
| | | GGUCGGCGUCGGCAUGCCGCUGAUC9GCAGUGGCCCCACCUCGGCA |
| | | UGCCGGCGCCGGGCGAGGAAUUGCUCAUGAAAAAAGUAUCUUUCU |
| | | GUAAAAAAAGAAAACAAUACAUGAUUAACCGAAAAGAAACCAACA |
| | | AAAAGAACCCGAGAUCAGUCGAUUUCGAUCACUACGAUAAACACA |
| | | UGGAAGAUUUCUUGAAAAAAGAAAAGAGAAAGAGACCACCUUCCC |
| | | GGCGGCGGACACGCUCCUCUCCGUCGCCGUUCUGCACCAUGAUUCG |
| | | AUCAAUAACAACAUCAUCAUCGGAGACCAUCUUUUAAUCAAUCAG |
| | | CGUUGCAGUAGUCGACUCCCUGGACACGAAGGAGUCAUCCAUUUU |
| | | UAUCCUCGCACUUCUUCGCUCUCAAAGCCGCCUUUAAAGUUGAAAU |
| | | GAAAGGAUGGAAACAUGGAAUACAGUUUUAAUUGCACGUAUCACC |
| | | AUUUUACUACAAAAAGAAAAAAAAACAACUUACACAUAGUAUUAC |
| | | CUUAGGUUUACGGAUAAGUAGAGUGUAGGCGUUUUUGAAACAGUU |
| | | CAGCCAAUGCAAUCUUGUCUCGGCAUAAUCACUCUUUCUGCAUAUA |
| | | AUAGUAGUAGUAGAUUUAUUCACAUCAACACAGCGAAAAACUCCA |
| | | GCAUCAAAGUACACCUAGAGACAGCCCUUAAAAUAUAGUUUGCAG |
| | | CUUUUAGAUGUACUUACACCAAAGAAGAUUACCGUCCUUACGAGA |
| | | AAACAGAUACUCGGAUAUAGGAAUCAAGACAGCUCUGCACUGAAA |
| | | ACACACUCUCCUGUCACGACACCGCGCCACACCAGAGGCGUACGCG |
| | | UGACUUCAUCGCAACGAUCCAUCGUGAUGUCCCUCGCAGAACCUAA |
| | | AAAGACCAAAAAAAAAUCUUGGACCACAGUUGUCGAUUCUUGAAG |
| | | ACAAUAUUCUCGUGAGAACUUUGAGAUUCGCACUUGAAACCUCUU |
| | | AGGAUCCACAAAAACAACAACCUCUGUAUGGAAAAUGCGCUAUUU |
| | | UAUCUCAGCUUUUCUCCCAAACCUCGGUUUCUUCCUAUUCUUAAGU |
| | | UUUCCCUAGUAUAUUUGCCUCCUUAUAAGAAAAGAAGCACAAGCU |
| | | CGGUCGCACGGAUUAUUCCUUCUGCUAAUCUAUUAUUUUGUUCCU |
| | | UUUUUUUUUGUUUGCCUUCACCCCCUUCACUCCCUGUAGCAACACA |
| | | GAGUAGUAGACACAAUAAAUGAGAAGUUUGCAUGCAGCUUGACUC |
| | | CUAUAGUGUCACCUAAAUGUCUAGAAACUUGUUUAUUGCAGCUUA |
| | | UAAUGGUUACAAAUAAAGCAAUAGCAUCACAAAUUUCACAAAUAA |
| | | AGCAUUUUUUUCACUGC(A)ₙ |
| 50 | β2.7 RNA | UCAGAUCUAAUACGACUCACUAUAGGCGCUUCCGGAAGAGCUAGC |
| | domain | UUUUAUUUUUUAUCUUCUCCUUUCCUUAAUCUCGGAUUAUCAUUU |
| | 2_endo | CCCUCUCCUACCUACCACGAAUCGCAGAUGAUAAACAAGAGGGUAA |
| | (sense) | AAAGAAAAAAAGCUUGACUCCUAUAGUGUCACCUAAAUGUCUAGA |
| | | AACUUGUUUAUUGCAGCUUAUAAUGGUUACAAAUAAAGCAAUAGC |
| | | AUCACAAAUUUCACAAAUAAAGCAUUUUUUUCACUGC(A)ₙ |
| 51 | β2.7 RNA | UCAGAUCUAAUACGACUCACUAUAGGCGCUUCCGGAAGAGCUAGC |
| | domain | GGUCAUUUUUAUUUAUCCUCAUCGUCAACAACAACUACCGACAACA |
| | 3_endo | ACGAAACACCACCAAGAAUGUCAAUCCGCAAGGGUGUUCCUGCCCC |
| | (sense) | CUCGACGCGCCUGUCGCGAUCCUCAUGGCGAGGACCGCGAUCUCCG |
| | | UAUAGGUAGAUGAAAUUAUCCCGUGUCCGGUCCUGAUUCCCCGCA |
| | | UGCCCUGCACAUCCUGACGCGUCGGUCAGCAGCCAAACAAUCAUAG |
| | | GAAAUGAACCAAGCUUGACUCCUAUAGUGUCACCUAAAUGUCUAG |
| | | AAACUUGUUUAUUGCAGCUUAUAAUGGUUACAAAUAAAGCAAUAG |
| | | CAUCACAAAUUUCACAAAUAAAGCAUUUUUUUCACUGC(A)ₙ |
| 52 | Multimer | UCAGAUCUAAUACGACUCACUAUAGGCGCUUCCGGAAGAGCUAGC |
| | sequence | UUUUAUUUUUUAUCUUCUCCUUUCCUUAAUCUCGGAUUAUCAUUU |
| | D2x4_endo | CCCUCUCCUACCUACCACGAAUCGCAGAUGAUAAACAAGAGGGUAA |
| | | AAAGAAAAAAGCGCGAAUUCCGAUCUUUUAUUUUUUAUCUUCUCC |
| | | UUUCCUUAAUCUCGGAUUAUCAUUUCCCUCUCCUACCUACCACGAA |
| | | UCGCAGAUGAUAAACAAGAGGGUAAAAAGAAAAAGAUCGUAUCCG |
| | | GUACCUGUGGUUUUAUUUUUUAUCUUCUCCUUUCCUUAAUCUCGG |
| | | AUUAUCAUUUCCCUCUCCUACCUACCACGAAUCGCAGAUGAUAAAC |
| | | AAGAGGGUAAAAAGAAAAACCACACCUCCACCGGUGGGCCGUUUU |
| | | AUUUUUUAUCUUCUCCUUUCCUUAAUCUCGGAUUAUCAUUUCCCUC |
| | | UCCUACCUACCACGAAUCGCAGAUGAUAAACAAGAGGGUAAAAAG |
| | | AAAAACGGCCCAAGCUUGACUCCUAUAGUGUCACCUAAAUGUCUA |
| | | GAAACUUGUUUAUUGCAGCUUAUAAUGGUUACAAAUAAAGCAAUA |
| | | GCAUCACAAAUUUCACAAAUAAAGCAUUUUUUUCACUGC(A)ₙ |
| 53 | Multimer | UCAGAUCUAAUACGACUCACUAUAGGCGCUUCCGGAAGAGCUAGC |
| | sequence | CCGGCGGUCAUUUUUAUUUAUCCUCAUCGUCAACAACAACUACCGA |
| | D3x4_endo | CAACAACGAAACACCACCAAGAAUGUCAAUCCGCAAGGGUGUUCCU |
| | | GCCCCCUCGACGCGCCUGUCGCGAUCCUCAUGGCGAGGACCGCGAU |
| | | CUCCGUAUAGGUAGAUGAAAUUAUCCCGUGUCCGGUCCUGAUUCCC |
| | | CGCAUGCCCUGCACAUCCUGACGCGUCGGUCAGCAGCCAAACAAUC |
| | | AUAGGAAAUGAACCGCCGGGAAGCGCCGAAUUCAUAUCGAUCGGU |
| | | CAUUUUUAUUUAUCCUCAUCGUCAACAACAACUACCGACAACAACG |
| | | AAACACCACCAAGAAUGUCAAUCCGCAAGGGUGUUCCUGCCCCCUC |
| | | GACGCGCCUGUCGCGAUCCUCAUGGCGAGGACCGCGAUCUCCGUAU |
| | | AGGUAGAUGAAAUUAUCCCGUGUCCGGUCCUGAUUCCCCGCAUGCC |
| | | CUGCACAUCCUGACGCGUCGGUCAGCAGCCAAACAAUCAUAGGAAA |
| | | UGAACCGAUCGAAUAAAGGUACCUGUGGGGUCAUUUUUAUUUAUC |
| | | CUCAUCGUCAACAACAACUACCGACAACAACGAAACACCACCAAGA |
| | | AUGUCAAUCCGCAAGGGUGUUCCUGCCCCCUCGACGCGCCUGUCGC |
| | | GAUCCUCAUGGCGAGGACCGCGAUCUCCGUAUAGGUAGAUGAAAU |
| | | UAUCCCGUGUCCGGUCCUGAUUCCCCGCAUGCCCUGCACAUCCUGA |
| | | CGCGUCGGUCAGCAGCCAAACAAUCAUAGGAAAUGAACCCCACAUU |
| | | UUACCGGUAAUACCGGGGGGUCAUUUUUAUUUAUCCUCAUCGUCA |
| | | ACAACAACUACCGACAACAACGAAACACCACCAAGAAUGUCAAUCC |
| | | GCAAGGGUGUUCCUGCCCCCUCGACGCGCCUGUCGCGAUCCUCAUG |
| | | GCGAGGACCGCGAUCUCCGUAUAGGUAGAUGAAAUUAUCCCGUGU |
| | | CCGGUCCUGAUUCCCCGCAUGCCCUGCACAUCCUGACGCGUCGGUC |
| | | AGCAGCCAAACAAUCAUAGGAAAUGAACCCCCCGGAAGCUUGACUC |
| | | CUAUAGUGUCACCUAAAUGUCUAGAAACUUGUUUAUUGCAGCUUA |
| | | UAAUGGUUACAAAUAAAGCAAUAGCAUCACAAAUUUCACAAAUAA |
| | | AGCAUUUUUUUCACUGC(A)ₙ |
| 54 | Multimer | UCAGAUCUAAUACGACUCACUAUAGGCGCUUCCGGAAGAGCUAGC |
| | sequence | CCGGCGGUCAUUUUUAUUUAUCCUCAUCGUCAACAACAACUACCGA |
| | D3x4_D2x4_ | CAACAACGAAACACCACCAAGAAUGUCAAUCCGCAAGGGUGUUCCU |
| | endo | GCCCCCUCGACGCGCCUGUCGCGAUCCUCAUGGCGAGGACCGCGAU |
| | | CUCCGUAUAGGUAGAUGAAAUUAUCCCGUGUCCGGUCCUGAUUCCC |
| | | CGCAUGCCCUGCACAUCCUGACGCGUCGGUCAGCAGCCAAACAAUC |
| | | AUAGGAAAUGAACCGCCGGGAAGCGCCGAAUUCAUAUCGAUCGGU |
| | | CAUUUUUAUUUAUCCUCAUCGUCAACAACAACUACCGACAACAACG |
| | | AAACACCACCAAGAAUGUCAAUCCGCAAGGGUGUUCCUGCCCCCUC |
| | | GACGCGCCUGUCGCGAUCCUCAUGGCGAGGACCGCGAUCUCCGUAU |
| | | AGGUAGAUGAAAUUAUCCCGUGUCCGGUCCUGAUUCCCCGCAUGCC |
| | | CUGCACAUCCUGACGCGUCGGUCAGCAGCCAAACAAUCAUAGGAAA |
| | | UGAACCGAUCGAAUAAAGGUACCUGUGGGGUCAUUUUUAUUUAUC |
| | | CUCAUCGUCAACAACAACUACCGACAACAACGAAACACCACCAAGA |
| | | AUGUCAAUCCGCAAGGGUGUUCCUGCCCCCUCGACGCGCCUGUCGC |
| | | GAUCCUCAUGGCGAGGACCGCGAUCUCCGUAUAGGUAGAUGAAAU |
| | | UAUCCCGUGUCCGGUCCUGAUUCCCCGCAUGCCCUGCACAUCCUGA |
| | | CGCGUCGGUCAGCAGCCAAACAAUCAUAGGAAAUGAACCCCACAUU |
| | | UUACCGGUAAUACCGGGGGGUCAUUUUUAUUUAUCCUCAUCGUCA |
| | | ACAACAACUACCGACAACAACGAAACACCACCAAGAAUGUCAAUCC |
| | | GCAAGGGUGUUCCUGCCCCCUCGACGCGCCUGUCGCGAUCCUCAUG |
| | | GCGAGGACCGCGAUCUCCGUAUAGGUAGAUGAAAUUAUCCCGUGU |
| | | CCGGUCCUGAUUCCCCGCAUGCCCUGCACAUCCUGACGCGUCGGUC |
| | | AGCAGCCAAACAAUCAUAGGAAAUGAACCCCCCGGAAGCUUUCCGG |
| | | AAGAGCUAGCUUUUAUUUUUUAUCUUCUCCUUUCCUUAAUCUCGG |
| | | AUUAUCAUUUCCCUCUCCUACCUACCACGAAUCGCAGAUGAUAAAC |
| | | AAGAGGGUAAAAAGAAAAAAGCGCGAAUUCCGAUCUUUUAUUUUU |
| | | UAUCUUCUCCUUUCCUUAAUCUCGGAUUAUCAUUUCCCUCUCCUAC |
| | | CUACCACGAAUCGCAGAUGAUAAACAAGAGGGUAAAAAGAAAAAG |
| | | AUCGUAUCCGGUACCUGUGGUUUUAUUUUUUAUCUUCUCCUUUCC |
| | | UUAAUCUCGGAUUAUCAUUUCCCUCUCCUACCUACCACGAAUCGCA |
| | | GAUGAUAAACAAGAGGGUAAAAAGAAAAACCACACCUCCACCGGU |
| | | GGGCCGUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAACGGCCCAAGCUUGACUCCUAUAGUGUCACC |
| | | UAAAUGUCUAGAAACUUGUUUAUUGCAGCUUAUAAUGGUUACAAA |
| | | UAAAGCAAUAGCAUCACAAAUUUCACAAAUAAAGCAUUUUUUUCA |
| | | CUGC(A)ₙ |
| 55 | β2.7 core | UCCCCAGAUCGCUGCUGCCCCGGCGUUCUCCAGAAGCCCCGGCGGG |
| | | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAAAGCUACAGACAUUUGGGUACCUCAGCUUUC |
| | | CGAUAACUCGAAGAAUUCAAAGUCGACGAUUCCCAACAAGAGAAA |
| | | ACAGAACAAAAACAAGGUCAUUUUUAUUUAUCCUCAUCGUCAACA |
| | | ACAACUACCGACAACAACGAAACACCACCAAGAAUGUCAAUCCGCA |
| | | AGGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAGC |
| | | AGCCAAACAAUCAUAGGAAAUGAACCAGAAGAACAAAAAGAUCAU |
| | | CUCUCUCGGUGUAUAGCAACACCAACAACAACCGCAUCGCAACAUC |
| | | UUCAUCCGCAAGACGGAAAGAAAACAACAAUAAUGAGAAUGAAAU |
| | | CACCACAACCAAGCCAGAUUUCACGUCCAUGAGUUUUUAUUAUAU |
| | | UAUUAUCAAAACGAAAAACAGAAAAACUGUCAUAGAUAAAUAUAA |
| | | AAAAAAAUAGAAACCACAAACGACUACUAGUACUCCAAUCUUAGA |
| | | UGUAUAUGCUCCUAGAUAAGAUUUAGUAUUACCAUAAUCAUCGAA |
| | | GAAUGAAAGACGACGAUGAUUCCUUACCGCUCCUGCCACCCGGUCU |
| | | GUAUGUAGAGAGAGAAGAGAGAAAACGGUGAAUCCAAGAUCCCCG |
| | | GGUCGGCGUCGGCAUGCCGCUGAUC9GCAGUGGCCCCACCUCGGCA |
| | | UGCCGGCGCCGGGCGAGGAAUUGCUCAUGAAAAAAGUAUCUUUCU |
| | | GUAAAAAAAGAAAACAAUACAUGAUUAACCGAAAAGAAACCAACA |
| | | AAAAGAACCCGAGAUCAGUCGAUUUCGAUCACUACGAUAAACACA |
| | | UGGAAGAUUUCUUGAAAAAAGAAAAGAGAAAGAGACCACCUUCCC |
| | | GGCGGCGGACACGCUCCUCUCCGUCGCCGUUCUGCACCAUGAUUCG |
| | | AUCAAUAACAACAUCAUCAUCGGAGACCAUCUUUUAAUCAAUCAG |
| | | CGUUGCAGUAGUCGACUCCCUGGACACGAAGGAGUCAUCCAUUUU |
| | | UAUCCUCGCACUUCUUCGCUCUCAAAGCCGCCUUUAAAGUUGAAAU |
| | | GAAAGGAUGGAAACAUGGAAUACAGUUUUAAUUGCACGUAUCACC |
| | | AUUUUACUACAAAAAGAAAAAAAAACAACUUACACAUAGUAUUAC |
| | | CUUAGGUUUACGGAUAAGUAGAGUGUAGGCGUUUUUGAAACAGUU |
| | | CAGCCAAUGCAAUCUUGUCUCGGCAUAAUCACUCUUUCUGCAUAUA |
| | | AUAGUAGUAGUAGAUUUAUUCACAUCAACACAGCGAAAAACUCCA |
| | | GCAUCAAAGUACACCUAGAGACAGCCCUUAAAAUAUAGUUUGCAG |
| | | CUUUUAGAUGUACUUACACCAAAGAAGAUUACCGUCCUUACGAGA |
| | | AAACAGAUACUCGGAUAUAGGAAUCAAGACAGCUCUGCACUGAAA |
| | | ACACACUCUCCUGUCACGACACCGCGCCACACCAGAGGCGUACGCG |
| | | UGACUUCAUCGCAACGAUCCAUCGUGAUGUCCCUCGCAGAACCUAA |
| | | AAAGACCAAAAAAAAAUCUUGGACCACAGUUGUCGAUUCUUGAAG |
| | | ACAAUAUUCUCGUGAGAACUUUGAGAUUCGCACUUGAAACCUCUU |
| | | AGGAUCCACAAAAACAACAACCUCUGUAUGGAAAAUGCGCUAUUU |
| | | UAUCUCAGCUUUUCUCCCAAACCUCGGUUUCUUCCUAUUCUUAAGU |
| | | UUUCCCUAGUAUAUUUGCCUCCUUAUAAGAAAAGAAGCACAAGCU |
| | | CGGUCGCACGGAUUAUUCCUUCUGCUAAUCUAUUAUUUUGUUCCU |
| | | UUUUUUUUUGUUUGCCUUCACCCCCUUCACUCCCUGUAGCAACACA |
| | | GAGUAGUAGACACAAUAAAUGAGAAGUUUGCAUGC |
| 56 | D1_core | CAGAAGCCCCGGCGGGCGAAUCGGCCGGCUGGUCGGUCGGCGCUCG |
| | | GACGGAUGGGGAGAACGGCGGUGACUUAGCCGCCCGUGGCCGGGA |
| | | GAAGAUGGAGGAGCCGAGAUGACAACGGCAGUCGUGGAAGGGUCG |
| | | CCAAGCCCCGGUCCUUCUCUUCUGUCUGGUCGAAUCUCGUUUUCUU |
| | | UUUUCAACCGCUCUUUUUAUCACCUUUUUAUGUGAGUUUCUCUUC |
| | | CGCGUCUCCCGGCCGUACCAUCCACCCAUGCAGCAUGCACGCGUGU |
| | | AUGUAUGCAUCGUCUCUCCUCCGUCCCGACUACCAUCAGCAGCACC |
| | | ACUACCGCCACCCCCAGCGCCACCACCGCUGCCGUCGCCACCGCGUU |
| | | AUCCGUUCCUCGUAGGCUGGUCCUGGGGAACGGGUCGGCGGCCGGU |
| | | CGGCUUCUG |
| 57 | D2_core | UUUUAUUUUUUAUCUUCUCCUUUCCUUAAUCUCGGAUUAUCAUUU |
| | | CCCUCUCCUACCUACCACGAAUCGCAGAUGAUAAACAAGAGGGUAA |
| | | AAAGAAAAA |
| 58 | D3_core | GGUCAUUUUUAUUUAUCCUCAUCGUCAACAACAACUACCGACAACA |
| | | ACGAAACACCACCAAGAAUGUCAAUCCGCAAGGGUGUUCCUGCCCC |
| | | CUCGACGCGCCUGUCGCGAUCCUCAUGGCGAGGACCGCGAUCUCCG |
| | | UAUAGGUAGAUGAAAUUAUCCCGUGUCCGGUCCUGAUUCCCCGCA |
| | | UGCCCUGCACAUCCUGACGCGUCGGUCAGCAGCCAAACAAUCAUAG |
| | | GAAAUGAAC |
| 59 | D4_core | CGGCGUCGGCAUGCCGCUGAUCGCAGUGGCCCCACCUCGGCAUGCC |
| | | GGCGCCGGGCGAGGAAUUGCUCAUGAAAAAAGUAUCUUUCUGUAA |
| | | AAAAAGAAAACAAUACAUGAUUAACCGAAAAGAAACCAACAAAAA |
| | | GAACCCGAGAUCAGUCGAUUUCGAUCACUACGAUAAACACAUGGA |
| | | AGAUUUCUUGAAAAAAGAAAAGAGAAAGAGACCACCUUCCCGGCG |
| | | GCGGACACGCUCCUCUCCGUCGCCGUUCUGCACCAUGAUUCGAUCA |
| | | AUAACAACAUCAUCAUCGGAGACCAUCUUUUAAUCAAUCAGCGUU |
| | | GCAGUAGUCGACUCCCUGGACACGAAGGAGUCAUCCAUUUUUAUCC |
| | | UCGC |
| 60 | ΔD1_core | UCCCCAGAUCGCUGCUGCCCCGGCGUUCUCUUUUAUUAUUUUUUUU |
| | | UAUUUUUUAUCUUCUCCUUUCCUUAAUCUCGGAUUAUCAUUUCCC |
| | | UCUCCUACCUACCACGAAUCGCAGAUGAUAAACAAGAGGGUAAAA |
| | | AGAAAAAAGCUACAGACAUUUGGGUACCUCAGCUUUCCGAUAACU |
| | | CGAAGAAUUCAAAGUCGACGAUUCCCAACAAGAGAAAACAGAACA |
| | | AAAACAAGGUCAUUUUUAUUUAUCCUCAUCGUCAACAACAACUAC |
| | | CGACAACAACGAAACACCACCAAGAAUGUCAAUCCGCAAGGGUGUU |
| | | CCUGCCCCCUCGACGCGCCUGUCGCGAUCCUCAUGGCGAGGACCGC |
| | | GAUCUCCGUAUAGGUAGAUGAAAUUAUCCCGUGUCCGGUCCUGAU |
| | | UCCCCGCAUGCCCUGCACAUCCUGACGCGUCGGUCAGCAGCCAAAC |
| | | AAUCAUAGGAAAUGAACCAGAAGAACAAAAAGAUCAUCUCUCUCG |
| | | GUGUAUAGCAACACCAACAACAACCGCAUCGCAACAUCUUCAUCCG |
| | | CAAGACGGAAAGAAAACAACAAUAAUGAGAAUGAAAUCACCACAA |
| | | CCAAGCCAGAUUUCACGUCCAUGAGUUUUUAUUAUAUUAUUAUCA |
| | | AAACGAAAAACAGAAAAACUGUCAUAGAUAAAUAUAAAAAAAAAU |
| | | AGAAACCACAAACGACUACUAGUACUCCAAUCUUAGAUGUAUAUG |
| | | CUCCUAGAUAAGAUUUAGUAUUACCAUAAUCAUCGAAGAAUGAAA |
| | | GACGACGAUGAUUCCUUACCGCUCCUGCCACCCGGUCUGUAUGUAG |
| | | AGAGAGAAGAGAGAAAACGGUGAAUCCAAGAUCCCCGGGUCGGCG |
| | | UCGGCAUGCCGCUGAUCGCAGUGGCCCCACCUCGGCAUGCCGGCGC |
| | | CGGGCGAGGAAUUGCUCAUGAAAAAAGUAUCUUUCUGUAAAAAAA |
| | | GAAAACAAUACAUGAUUAACCGAAAAGAAACCAACAAAAAGAACC |
| | | CGAGAUCAGUCGAUUUCGAUCACUACGAUAAACACAUGGAAGAUU |
| | | UCUUGAAAAAAGAAAAGAGAAAGAGACCACCUUCCCGGCGGCGGA |
| | | CACGCUCCUCUCCGUCGCCGUUCUGCACCAUGAUUCGAUCAAUAAC |
| | | AACAUCAUCAUCGGAGACCAUCUUUUAAUCAAUCAGCGUUGCAGU |
| | | AGUCGACUCCCUGGACACGAAGGAGUCAUCCAUUUUUAUCCUCGCA |
| | | CUUCUUCGCUCUCAAAGCCGCCUUUAAAGUUGAAAUGAAAGGAUG |
| | | GAAACAUGGAAUACAGUUUUAAUUGCACGUAUCACCAUUUUACUA |
| | | CAAAAAGAAAAAAAAACAACUUACACAUAGUAUUACCUUAGGUUU |
| | | ACGGAUAAGUAGAGUGUAGGCGUUUUUGAAACAGUUCAGCCAAUG |
| | | CAAUCUUGUCUCGGCAUAAUCACUCUUUCUGCAUAUAAUAGUAGU |
| | | AGUAGAUUUAUUCACAUCAACACAGCGAAAAACUCCAGCAUCAAA |
| | | GUACACCUAGAGACAGCCCUUAAAAUAUAGUUUGCAGCUUUUAGA |
| | | UGUACUUACACCAAAGAAGAUUACCGUCCUUACGAGAAAACAGAU |
| | | ACUCGGAUAUAGGAAUCAAGACAGCUCUGCACUGAAAACACACUC |
| | | UCCUGUCACGACACCGCGCCACACCAGAGGCGUACGCGUGACUUCA |
| | | UCGCAACGAUCCAUCGUGAUGUCCCUCGCAGAACCUAAAAAGACCA |
| | | AAAAAAAAUCUUGGACCACAGUUGUCGAUUCUUGAAGACAAUAUU |
| | | CUCGUGAGAACUUUGAGAUUCGCACUUGAAACCUCUUAGGAUCCA |
| | | CAAAAACAACAACCUCUGUAUGGAAAAUGCGCUAUUUUAUCUCAG |
| | | CUUUUCUCCCAAACCUCGGUUUCUUCCUAUUCUUAAGUUUUCCCUA |
| | | GUAUAUUUGCCUCCUUAUAAGAAAAGAAGCACAAGCUCGGUCGCA |
| | | CGGAUUAUUCCUUCUGCUAAUCUAUUAUUUUGUUCCUUUUUUUUU |
| | | UGUUUGCCUUCACCCCCUUCACUCCCUGUAGCAACACAGAGUAGUA |
| | | GACACAAUAAAUGAGAAGUUUGCAUGC |
| 61 | ΔD2_core | UCCCCAGAUCGCUGCUGCCCCGGCGUUCUCCAGAAGCCCCGGCGGG |
| | | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUAGCUACAGACAUUUGGGUACCUCAGCUUUCCGAUAACUC |
| | | GAAGAAUUCAAAGUCGACGAUUCCCAACAAGAGAAAACAGAACAA |
| | | AAACAAGGUCAUUUUUAUUUAUCCUCAUCGUCAACAACAACUACC |
| | | GACAACAACGAAACACCACCAAGAAUGUCAAUCCGCAAGGGUGUUC |
| | | CUGCCCCCUCGACGCGCCUGUCGCGAUCCUCAUGGCGAGGACCGCG |
| | | AUCUCCGUAUAGGUAGAUGAAAUUAUCCCGUGUCCGGUCCUGAUU |
| | | CCCCGCAUGCCCUGCACAUCCUGACGCGUCGGUCAGCAGCCAAACA |
| | | AUCAUAGGAAAUGAACCAGAAGAACAAAAAGAUCAUCUCUCUCGG |
| | | UGUAUAGCAACACCAACAACAACCGCAUCGCAACAUCUUCAUCCGC |
| | | AAGACGGAAAGAAAACAACAAUAAUGAGAAUGAAAUCACCACAAC |
| | | CAAGCCAGAUUUCACGUCCAUGAGUUUUUAUUAUAUUAUUAUCAA |
| | | AACGAAAAACAGAAAAACUGUCAUAGAUAAAUAUAAAAAAAAAUA |
| | | GAAACCACAAACGACUACUAGUACUCCAAUCUUAGAUGUAUAUGC |
| | | UCCUAGAUAAGAUUUAGUAUUACCAUAAUCAUCGAAGAAUGAAAG |
| | | ACGACGAUGAUUCCUUACCGCUCCUGCCACCCGGUCUGUAUGUAGA |
| | | GAGAGAAGAGAGAAAACGGUGAAUCCAAGAUCCCCGGGUCGGCGU |
| | | CGGCAUGCCGCUGAUCGCAGUGGCCCCACCUCGGCAUGCCGGCGCC |
| | | GGGCGAGGAAUUGCUCAUGAAAAAAGUAUCUUUCUGUAAAAAAAG |
| | | AAAACAAUACAUGAUUAACCGAAAAGAAACCAACAAAAAGAACCC |
| | | GAGAUCAGUCGAUUUCGAUCACUACGAUAAACACAUGGAAGAUUU |
| | | CUUGAAAAAAGAAAAGAGAAAGAGACCACCUUCCCGGCGGCGGAC |
| | | ACGCUCCUCUCCGUCGCCGUUCUGCACCAUGAUUCGAUCAAUAACA |
| | | ACAUCAUCAUCGGAGACCAUCUUUUAAUCAAUCAGCGUUGCAGUA |
| | | GUCGACUCCCUGGACACGAAGGAGUCAUCCAUUUUUAUCCUCGCAC |
| | | UUCUUCGCUCUCAAAGCCGCCUUUAAAGUUGAAAUGAAAGGAUGG |
| | | AAACAUGGAAUACAGUUUUAAUUGCACGUAUCACCAUUUUACUAC |
| | | AAAAAGAAAAAAAAACAACUUACACAUAGUAUUACCUUAGGUUUA |
| | | CGGAUAAGUAGAGUGUAGGCGUUUUUGAAACAGUUCAGCCAAUGC |
| | | AAUCUUGUCUCGGCAUAAUCACUCUUUCUGCAUAUAAUAGUAGUA |
| | | GUAGAUUUAUUCACAUCAACACAGCGAAAAACUCCAGCAUCAAAG |
| | | UACACCUAGAGACAGCCCUUAAAAUAUAGUUUGCAGCUUUUAGAU |
| | | GUACUUACACCAAAGAAGAUUACCGUCCUUACGAGAAAACAGAUA |
| | | CUCGGAUAUAGGAAUCAAGACAGCUCUGCACUGAAAACACACUCUC |
| | | CUGUCACGACACCGCGCCACACCAGAGGCGUACGCGUGACUUCAUC |
| | | GCAACGAUCCAUCGUGAUGUCCCUCGCAGAACCUAAAAAGACCAAA |
| | | AAAAAAUCUUGGACCACAGUUGUCGAUUCUUGAAGACAAUAUUCU |
| | | CGUGAGAACUUUGAGAUUCGCACUUGAAACCUCUUAGGAUCCACA |
| | | AAAACAACAACCUCUGUAUGGAAAAUGCGCUAUUUUAUCUCAGCU |
| | | UUUCUCCCAAACCUCGGUUUCUUCCUAUUCUUAAGUUUUCCCUAGU |
| | | AUAUUUGCCUCCUUAUAAGAAAAGAAGCACAAGCUCGGUCGCACG |
| | | GAUUAUUCCUUCUGCUAAUCUAUUAUUUUGUUCCUUUUUUUUUUG |
| | | UUUGCCUUCACCCCCUUCACUCCCUGUAGCAACACAGAGUAGUAGA |
| | | CACAAUAAAUGAGAAGUUUGCAUGC |
| 62 | ΔD3_core | UCCCCAGAUCGCUGCUGCCCCGGCGUUCUCCAGAAGCCCCGGCGGG |
| | | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAAAGCUACAGACAUUUGGGUACCUCAGCUUUC |
| | | CGAUAACUCGAAGAAUUCAAAGUCGACGAUUCCCAACAAGAGAAA |
| | | ACAGAACAAAAACAAAGAAGAACAAAAAGAUCAUCUCUCUCGGUG |
| | | UAUAGCAACACCAACAACAACCGCAUCGCAACAUCUUCAUCCGCAA |
| | | GACGGAAAGAAAACAACAAUAAUGAGAAUGAAAUCACCACAACCA |
| | | AGCCAGAUUUCACGUCCAUGAGUUUUUAUUAUAUUAUUAUCAAAA |
| | | CGAAAAACAGAAAAACUGUCAUAGAUAAAUAUAAAAAAAAAUAGA |
| | | AACCACAAACGACUACUAGUACUCCAAUCUUAGAUGUAUAUGCUCC |
| | | UAGAUAAGAUUUAGUAUUACCAUAAUCAUCGAAGAAUGAAAGACG |
| | | ACGAUGAUUCCUUACCGCUCCUGCCACCCGGUCUGUAUGUAGAGAG |
| | | AGAAGAGAGAAAACGGUGAAUCCAAGAUCCCCGGGUCGGCGUCGG |
| | | CAUGCCGCUGAUCGCAGUGGCCCCACCUCGGCAUGCCGGCGCCGGG |
| | | CGAGGAAUUGCUCAUGAAAAAAGUAUCUUUCUGUAAAAAAAGAAA |
| | | ACAAUACAUGAUUAACCGAAAAGAAACCAACAAAAAGAACCCGAG |
| | | AUCAGUCGAUUUCGAUCACUACGAUAAACACAUGGAAGAUUUCUU |
| | | GAAAAAAGAAAAGAGAAAGAGACCACCUUCCCGGCGGCGGACACG |
| | | CUCCUCUCCGUCGCCGUUCUGCACCAUGAUUCGAUCAAUAACAACA |
| | | UCAUCAUCGGAGACCAUCUUUUAAUCAAUCAGCGUUGCAGUAGUC |
| | | GACUCCCUGGACACGAAGGAGUCAUCCAUUUUUAUCCUCGCACUUC |
| | | UUCGCUCUCAAAGCCGCCUUUAAAGUUGAAAUGAAAGGAUGGAAA |
| | | CAUGGAAUACAGUUUUAAUUGCACGUAUCACCAUUUUACUACAAA |
| | | AAGAAAAAAAAACAACUUACACAUAGUAUUACCUUAGGUUUACGG |
| | | AUAAGUAGAGUGUAGGCGUUUUUGAAACAGUUCAGCCAAUGCAAU |
| | | CUUGUCUCGGCAUAAUCACUCUUUCUGCAUAUAAUAGUAGUAGUA |
| | | GAUUUAUUCACAUCAACACAGCGAAAAACUCCAGCAUCAAAGUAC |
| | | ACCUAGAGACAGCCCUUAAAAUAUAGUUUGCAGCUUUUAGAUGUA |
| | | CUUACACCAAAGAAGAUUACCGUCCUUACGAGAAAACAGAUACUC |
| | | GGAUAUAGGAAUCAAGACAGCUCUGCACUGAAAACACACUCUCCU |
| | | GUCACGACACCGCGCCACACCAGAGGCGUACGCGUGACUUCAUCGC |
| | | AACGAUCCAUCGUGAUGUCCCUCGCAGAACCUAAAAAGACCAAAAA |
| | | AAAAUCUUGGACCACAGUUGUCGAUUCUUGAAGACAAUAUUCUCG |
| | | UGAGAACUUUGAGAUUCGCACUUGAAACCUCUUAGGAUCCACAAA |
| | | AACAACAACCUCUGUAUGGAAAAUGCGCUAUUUUAUCUCAGCUUU |
| | | UCUCCCAAACCUCGGUUUCUUCCUAUUCUUAAGUUUUCCCUAGUAU |
| | | AUUUGCCUCCUUAUAAGAAAAGAAGCACAAGCUCGGUCGCACGGA |
| | | UUAUUCCUUCUGCUAAUCUAUUAUUUUGUUCCUUUUUUUUUUGUU |
| | | UGCCUUCACCCCCUUCACUCCCUGUAGCAACACAGAGUAGUAGACA |
| | | CAAUAAAUGAGAAGUUUGCAUGC |
| 63 | ΔD4_core | UCCCCAGAUCGCUGCUGCCCCGGCGUUCUCCAGAAGCCCCGGCGGG |
| | | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAAAGCUACAGACAUUUGGGUACCUCAGCUUUC |
| | | CGAUAACUCGAAGAAUUCAAAGUCGACGAUUCCCAACAAGAGAAA |
| | | ACAGAACAAAAACAAGGUCAUUUUUAUUUAUCCUCAUCGUCAACA |
| | | ACAACUACCGACAACAACGAAACACCACCAAGAAUGUCAAUCCGCA |
| | | AGGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAGC |
| | | AGCCAAACAAUCAUAGGAAAUGAACCAGAAGAACAAAAAGAUCAU |
| | | CUCUCUCGGUGUAUAGCAACACCAACAACAACCGCAUCGCAACAUC |
| | | UUCAUCCGCAAGACGGAAAGAAAACAACAAUAAUGAGAAUGAAAU |
| | | CACCACAACCAAGCCAGAUUUCACGUCCAUGAGUUUUUAUUAUAU |
| | | UAUUAUCAAAACGAAAAACAGAAAAACUGUCAUAGAUAAAUAUAA |
| | | AAAAAAAUAGAAACCACAAACGACUACUAGUACUCCAAUCUUAGA |
| | | UGUAUAUGCUCCUAGAUAAGAUUUAGUAUUACCAUAAUCAUCGAA |
| | | GAAUGAAAGACGACGAUGAUUCCUUACCGCUCCUGCCACCCGGUCU |
| | | GUAUGUAGAGAGAGAAGAGAGAAAACGGUGAAUCCAAGAUCCCCG |
| | | GGUACUUCUUCGCUCUCAAAGCCGCCUUUAAAGUUGAAAUGAAAG |
| | | GAUGGAAACAUGGAAUACAGUUUUAAUUGCACGUAUCACCAUUUU |
| | | ACUACAAAAAGAAAAAAAAACAACUUACACAUAGUAUUACCUUAG |
| | | GUUUACGGAUAAGUAGAGUGUAGGCGUUUUUGAAACAGUUCAGCC |
| | | AAUGCAAUCUUGUCUCGGCAUAAUCACUCUUUCUGCAUAUAAUAG |
| | | UAGUAGUAGAUUUAUUCACAUCAACACAGCGAAAAACUCCAGCAU |
| | | CAAAGUACACCUAGAGACAGCCCUUAAAAUAUAGUUUGCAGCUUU |
| | | UAGAUGUACUUACACCAAAGAAGAUUACCGUCCUUACGAGAAAAC |
| | | AGAUACUCGGAUAUAGGAAUCAAGACAGCUCUGCACUGAAAACAC |
| | | ACUCUCCUGUCACGACACCGCGCCACACCAGAGGCGUACGCGUGAC |
| | | UUCAUCGCAACGAUCCAUCGUGAUGUCCCUCGCAGAACCUAAAAAG |
| | | ACCAAAAAAAAAUCUUGGACCACAGUUGUCGAUUCUUGAAGACAA |
| | | UAUUCUCGUGAGAACUUUGAGAUUCGCACUUGAAACCUCUUAGGA |
| | | UCCACAAAAACAACAACCUCUGUAUGGAAAAUGCGCUAUUUUAUC |
| | | UCAGCUUUUCUCCCAAACCUCGGUUUCUUCCUAUUCUUAAGUUUUC |
| | | CCUAGUAUAUUUGCCUCCUUAUAAGAAAAGAAGCACAAGCUCGGU |
| | | CGCACGGAUUAUUCCUUCUGCUAAUCUAUUAUUUUGUUCCUUUUU |
| | | UUUUUGUUUGCCUUCACCCCCUUCACUCCCUGUAGCAACACAGAGU |
| | | AGUAGACACAAUAAAUGAGAAGUUUGCAUGC |
| 64 | β2.7 | GCAUGCAAACUUCUCAUUUAUUGUGUCUACUACUCUGUGUUGCUA |
| | RNA_AS_ | CAGGGAGUGAAGGGGGUGAAGGCAAACAAAAAAAAAAGGAACAAA |
| | core) | AUAAUAGAUUAGCAGAAGGAAUAAUCCGUGCGACCGAGCUUGUGC |
| | | UUCUUUUCUUAUAAGGAGGCAAAUAUACUAGGGAAAACUUAAGAA |
| | | UAGGAAGAAACCGAGGUUUGGGAGAAAAGCUGAGAUAAAAUAGCG |
| | | CAUUUUCCAUACAGAGGUUGUUGUUUUUGUGGAUCCUAAGAGGUU |
| | | UCAAGUGCGAAUCUCAAAGUUCUCACGAGAAUAUUGUCUUCAAGA |
| | | AUCGACAACUGUGGUCCAAGAUUUUUUUUUGGUCUUUUUAGGUUC |
| | | UGCGAGGGACAUCACGAUGGAUCGUUGCGAUGAAGUCACGCGUAC |
| | | GCCUCUGGUGUGGCGCGGUGUCGUGACAGGAGAGUGUGUUUUCAG |
| | | UGCAGAGCUGUCUUGAUUCCUAUAUCCGAGUAUCUGUUUUCUCGU |
| | | AAGGACGGUAAUCUUCUUUGGUGUAAGUACAUCUAAAAGCUGCAA |
| | | ACUAUAUUUUAAGGGCUGUCUCUAGGUGUACUUUGAUGCUGGAGU |
| | | UUUUCGCUGUGUUGAUGUGAAUAAAUCUACUACUACUAUUAUAUG |
| | | CAGAAAGAGUGAUUAUGCCGAGACAAGAUUGCAUUGGCUGAACUG |
| | | UUUCAAAAACGCCUACACUCUACUUAUCCGUAAACCUAAGGUAAU |
| | | ACUAUGUGUAAGUUGUUUUUUUUUCUUUUUGUAGUAAAAUGGUGA |
| | | UACGUGCAAUUAAAACUGUAUUCCAUGUUUCCAUCCUUUCAUUUC |
| | | AACUUUAAAGGCGGCUUUGAGAGCGAAGAAGUGCGAGGAUAAAAA |
| | | UGGAUGACUCCUUCGUGUCCAGGGAGUCGACUACUGCAACGCUGA |
| | | UUGAUUAAAAGAUGGUCUCCGAUGAUGAUGUUGUUAUUGAUCGAA |
| | | UCAUGGUGCAGAACGGCGACGGAGAGGAGCGUGUCCGCCGCCGGG |
| | | AAGGUGGUCUCUUUCUCUUUUCUUUUUUCAAGAAAUCUUCCAUGU |
| | | GUUUAUCGUAGUGAUCGAAAUCGACUGAUCUCGGGUUCUUUUUGU |
| | | UGGUUUCUUUUCGGUUAAUCAUGUAUUGUUUUCUUUUUUUACAGA |
| | | AAGAUACUUUUUUCAUGAGCAAUUCCUCGCCCGGCGCCGGCAUGCC |
| | | GAGGUGGGGCCACUGCGAUCAGCGGCAUGCCGACGCCGACCCGGGG |
| | | AUCUUGGAUUCACCGUUUUCUCUCUUCUCUCUCUACAUACAGACCG |
| | | GGUGGCAGGAGCGGUAAGGAAUCAUCGUCGUCUUUCAUUCUUCGA |
| | | UGAUUAUGGUAAUACUAAAUCUUAUCUAGGAGCAUAUACAUCUAA |
| | | GAUUGGAGUACUAGUAGUCGUUUGUGGUUUCUAUUUUUUUUUAUA |
| | | UUUAUCUAUGACAGUUUUUCUGUUUUUCGUUUUGAUAAUAAUAUA |
| | | AUAAAAACUCAUGGACGUGAAAUCUGGCUUGGUUGUGGUGAUUUC |
| | | AUUCUCAUUAUUGUUGUUUUCUUUCCGUCUUGCGGAUGAAGAUGU |
| | | UGCGAUGCGGUUGUUGUUGGUGUUGCUAUACACCGAGAGAGAUGA |
| | | UCUUUUUGUUCUUCUGGUUCAUUUCCUAUGAUUGUUUGGCUGCUG |
| | | ACCGACGCGUCAGGAUGUGCAGGGCAUGCGGGGAAUCAGGACCGG |
| | | ACACGGGAUAAUUUCAUCUACCUAUACGGAGAUCGCGGUCCUCGCC |
| | | AUGAGGAUCGCGACAGGCGCGUCGAGGGGGCAGGAACACCCUUGC |
| | | GGAUUGACAUUCUUGGUGGUGUUUCGUUGUUGUCGGUAGUUGUUG |
| | | UUGACGAUGAGGAUAAAUAAAAAUGACCUUGUUUUUGUUCUGUUU |
| | | UCUCUUGUUGGGAAUCGUCGACUUUGAAUUCUUCGAGUUAUCGGA |
| | | AAGCUGAGGUACCCAAAUGUCUGUAGCUUUUUUCUUUUUACCCUC |
| | | UUGUUUAUCAUCUGCGAUUCGUGGUAGGUAGGAGAGGGAAAUGAU |
| | | AAUCCGAGAUUAAGGAAAGGAGAAGAUAAAAAAUAAAAAAAAAUA |
| | | AUAAAACAGAAGCCGACCGGCCGCCGACCCGUUCCCCAGGACCAGC |
| | | CUACGAGGAACGGAUAACGCGGUGGCGACGGCAGCGGUGGUGGCG |
| | | CUGGGGGUGGCGGUAGUGGUGCUGCUGAUGGUAGUCGGGACGGAG |
| | | GAGAGACGAUGCAUACAUACACGCGUGCAUGCUGCAUGGGUGGAU |
| | | GGUACGGCCGGGAGACGCGGAAGAGAAACUCACAUAAAAAGGUGA |
| | | UAAAAAGAGCGGUUGAAAAAAGAAAACGAGAUUCGACCAGACAGA |
| | | AGAGAAGGACCGGGGCUUGGCGACCCUUCCACGACUGCCGUUGUCA |
| | | UCUCGGCUCCUCCAUCUUCUCCCGGCCACGGGCGGCUAAGUCACCG |
| | | CCGUUCUCCCCAUCCGUCCGAGCGCCGACCGACCAGCCGGCCGAUU |
| | | CGCCCGCCGGGGCUUCUGGAGAACGCCGGGGCAGCAGCGAUCUGGG |
| | | GA |
| 65 | D1AS_core | CAGAAGCCGACCGGCCGCCGACCCGUUCCCCAGGACCAGCCUACGA |
| | | GGAACGGAUAACGCGGUGGCGACGGCAGCGGUGGUGGCGCUGGGG |
| | | GUGGCGGUAGUGGUGCUGCUGAUGGUAGUCGGGACGGAGGAGAGA |
| | | CGAUGCAUACAUACACGCGUGCAUGCUGCAUGGGUGGAUGGUACG |
| | | GCCGGGAGACGCGGAAGAGAAACUCACAUAAAAAGGUGAUAAAAA |
| | | GAGCGGUUGAAAAAAGAAAACGAGAUUCGACCAGACAGAAGAGAA |
| | | GGACCGGGGCUUGGCGACCCUUCCACGACUGCCGUUGUCAUCUCGG |
| | | CUCCUCCAUCUUCUCCCGGCCACGGGCGGCUAAGUCACCGCCGUUC |
| | | UCCCCAUCCGUCCGAGCGCCGACCGACCAGCCGGCCGAUUCGCCCG |
| | | CCGGGGCUUCUG |
| 66 | D2AS_core | UUUUUCUUUUUACCCUCUUGUUUAUCAUCUGCGAUUCGUGGUAGG |
| | | UAGGAGAGGGAAAUGAUAAUCCGAGAUUAAGGAAAGGAGAAGAUA |
| | | AAAAAUAAAA |
| 67 | D3AS_core | GUUCAUUUCCUAUGAUUGUUUGGCUGCUGACCGACGCGUCAGGAU |
| | | GUGCAGGGCAUGCGGGGAAUCAGGACCGGACACGGGAUAAUUUCA |
| | | UCUACCUAUACGGAGAUCGCGGUCCUCGCCAUGAGGAUCGCGACAG |
| | | GCGCGUCGAGGGGGCAGGAACACCCUUGCGGAUUGACAUUCUUGG |
| | | UGGUGUUUCGUUGUUGUCGGUAGUUGUUGUUGACGAUGAGGAUAA |
| | | AUAAAAAUGACC |
| 68 | D4AS_core | GCGAGGAUAAAAAUGGAUGACUCCUUCGUGUCCAGGGAGUCGACU |
| | | ACUGCAACGCUGAUUGAUUAAAAGAUGGUCUCCGAUGAUGAUGUU |
| | | GUUAUUGAUCGAAUCAUGGUGCAGAACGGCGACGGAGAGGAGCGU |
| | | GUCCGCCGCCGGGAAGGUGGUCUCUUUCUCUUUUCUUUUUUCAAG |
| | | AAAUCUUCCAUGUGUUUAUCGUAGUGAUCGAAAUCGACUGAUCUC |
| | | GGGUUCUUUUUGUUGGUUUCUUUUCGGUUAAUCAUGUAUUGUUUU |
| | | CUUUUUUUACAGAAAGAUACUUUUUUCAUGAGCAAUUCCUCGCCC |
| | | GGCGCCGGCAUGCCGAGGUGGGGCCACUGCGAUCAGCGGCAUGCCG |
| | | ACGCCG |
| 69 | ΔD1AS_ | GCAUGCAAACUUCUCAUUUAUUGUGUCUACUACUCUGUGUUGCUA |
| | core | CAGGGAGUGAAGGGGGUGAAGGCAAACAAAAAAAAAAGGAACAAA |
| | | AUAAUAGAUUAGCAGAAGGAAUAAUCCGUGCGACCGAGCUUGUGC |
| | | UUCUUUUCUUAUAAGGAGGCAAAUAUACUAGGGAAAACUUAAGAA |
| | | UAGGAAGAAACCGAGGUUUGGGAGAAAAGCUGAGAUAAAAUAGCG |
| | | CAUUUUCCAUACAGAGGUUGUUGUUUUUGUGGAUCCUAAGAGGUU |
| | | UCAAGUGCGAAUCUCAAAGUUCUCACGAGAAUAUUGUCUUCAAGA |
| | | AUCGACAACUGUGGUCCAAGAUUUUUUUUUGGUCUUUUUAGGUUC |
| | | UGCGAGGGACAUCACGAUGGAUCGUUGCGAUGAAGUCACGCGUAC |
| | | GCCUCUGGUGUGGCGCGGUGUCGUGACAGGAGAGUGUGUUUUCAG |
| | | UGCAGAGCUGUCUUGAUUCCUAUAUCCGAGUAUCUGUUUUCUCGU |
| | | AAGGACGGUAAUCUUCUUUGGUGUAAGUACAUCUAAAAGCUGCAA |
| | | ACUAUAUUUUAAGGGCUGUCUCUAGGUGUACUUUGAUGCUGGAGU |
| | | UUUUCGCUGUGUUGAUGUGAAUAAAUCUACUACUACUAUUAUAUG |
| | | CAGAAAGAGUGAUUAUGCCGAGACAAGAUUGCAUUGGCUGAACUG |
| | | UUUCAAAAACGCCUACACUCUACUUAUCCGUAAACCUAAGGUAAU |
| | | ACUAUGUGUAAGUUGUUUUUUUUUCUUUUUGUAGUAAAAUGGUGA |
| | | UACGUGCAAUUAAAACUGUAUUCCAUGUUUCCAUCCUUUCAUUUC |
| | | AACUUUAAAGGCGGCUUUGAGAGCGAAGAAGUGCGAGGAUAAAAA |
| | | UGGAUGACUCCUUCGUGUCCAGGGAGUCGACUACUGCAACGCUGA |
| | | UUGAUUAAAAGAUGGUCUCCGAUGAUGAUGUUGUUAUUGAUCGAA |
| | | UCAUGGUGCAGAACGGCGACGGAGAGGAGCGUGUCCGCCGCCGGG |
| | | AAGGUGGUCUCUUUCUCUUUUCUUUUUUCAAGAAAUCUUCCAUGU |
| | | GUUUAUCGUAGUGAUCGAAAUCGACUGAUCUCGGGUUCUUUUUGU |
| | | UGGUUUCUUUUCGGUUAAUCAUGUAUUGUUUUCUUUUUUUACAGA |
| | | AAGAUACUUUUUUCAUGAGCAAUUCCUCGCCCGGCGCCGGCAUGCC |
| | | GAGGUGGGGCCACUGCGAUCAGCGGCAUGCCGACGCCGACCCGGGG |
| | | AUCUUGGAUUCACCGUUUUCUCUCUUCUCUCUCUACAUACAGACCG |
| | | GGUGGCAGGAGCGGUAAGGAAUCAUCGUCGUCUUUCAUUCUUCGA |
| | | UGAUUAUGGUAAUACUAAAUCUUAUCUAGGAGCAUAUACAUCUAA |
| | | GAUUGGAGUACUAGUAGUCGUUUGUGGUUUCUAUUUUUUUUUAUA |
| | | UUUAUCUAUGACAGUUUUUCUGUUUUUCGUUUUGAUAAUAAUAUA |
| | | AUAAAAACUCAUGGACGUGAAAUCUGGCUUGGUUGUGGUGAUUUC |
| | | AUUCUCAUUAUUGUUGUUUUCUUUCCGUCUUGCGGAUGAAGAUGU |
| | | UGCGAUGCGGUUGUUGUUGGUGUUGCUAUACACCGAGAGAGAUGA |
| | | UCUUUUUGUUCUUCUGGUUCAUUUCCUAUGAUUGUUUGGCUGCUG |
| | | ACCGACGCGUCAGGAUGUGCAGGGCAUGCGGGGAAUCAGGACCGG |
| | | ACACGGGAUAAUUUCAUCUACCUAUACGGAGAUCGCGGUCCUCGCC |
| | | AUGAGGAUCGCGACAGGCGCGUCGAGGGGGCAGGAACACCCUUGC |
| | | GGAUUGACAUUCUUGGUGGUGUUUCGUUGUUGUCGGUAGUUGUUG |
| | | UUGACGAUGAGGAUAAAUAAAAAUGACCUUGUUUUUGUUCUGUUU |
| | | UCUCUUGUUGGGAAUCGUCGACUUUGAAUUCUUCGAGUUAUCGGA |
| | | AAGCUGAGGUACCCAAAUGUCUGUAGCUUUUUUCUUUUUACCCUC |
| | | UUGUUUAUCAUCUGCGAUUCGUGGUAGGUAGGAGAGGGAAAUGAU |
| | | AAUCCGAGAUUAAGGAAAGGAGAAGAUAAAAAAUAAAAAAAAAUA |
| | | AUAAAAGAGAACGCCGGGGCAGCAGCGAUCUGGGGA |
| 70 | ΔD2AS_ | GCAUGCAAACUUCUCAUUUAUUGUGUCUACUACUCUGUGUUGCUA |
| | core | CAGGGAGUGAAGGGGGUGAAGGCAAACAAAAAAAAAAGGAACAAA |
| | | AUAAUAGAUUAGCAGAAGGAAUAAUCCGUGCGACCGAGCUUGUGC |
| | | UUCUUUUCUUAUAAGGAGGCAAAUAUACUAGGGAAAACUUAAGAA |
| | | UAGGAAGAAACCGAGGUUUGGGAGAAAAGCUGAGAUAAAAUAGCG |
| | | CAUUUUCCAUACAGAGGUUGUUGUUUUUGUGGAUCCUAAGAGGUU |
| | | UCAAGUGCGAAUCUCAAAGUUCUCACGAGAAUAUUGUCUUCAAGA |
| | | AUCGACAACUGUGGUCCAAGAUUUUUUUUUGGUCUUUUUAGGUUC |
| | | UGCGAGGGACAUCACGAUGGAUCGUUGCGAUGAAGUCACGCGUAC |
| | | GCCUCUGGUGUGGCGCGGUGUCGUGACAGGAGAGUGUGUUUUCAG |
| | | UGCAGAGCUGUCUUGAUUCCUAUAUCCGAGUAUCUGUUUUCUCGU |
| | | AAGGACGGUAAUCUUCUUUGGUGUAAGUACAUCUAAAAGCUGCAA |
| | | ACUAUAUUUUAAGGGCUGUCUCUAGGUGUACUUUGAUGCUGGAGU |
| | | UUUUCGCUGUGUUGAUGUGAAUAAAUCUACUACUACUAUUAUAUG |
| | | CAGAAAGAGUGAUUAUGCCGAGACAAGAUUGCAUUGGCUGAACUG |
| | | UUUCAAAAACGCCUACACUCUACUUAUCCGUAAACCUAAGGUAAU |
| | | ACUAUGUGUAAGUUGUUUUUUUUUCUUUUUGUAGUAAAAUGGUGA |
| | | UACGUGCAAUUAAAACUGUAUUCCAUGUUUCCAUCCUUUCAUUUC |
| | | AACUUUAAAGGCGGCUUUGAGAGCGAAGAAGUGCGAGGAUAAAAA |
| | | UGGAUGACUCCUUCGUGUCCAGGGAGUCGACUACUGCAACGCUGA |
| | | UUGAUUAAAAGAUGGUCUCCGAUGAUGAUGUUGUUAUUGAUCGAA |
| | | UCAUGGUGCAGAACGGCGACGGAGAGGAGCGUGUCCGCCGCCGGG |
| | | AAGGUGGUCUCUUUCUCUUUUCUUUUUUCAAGAAAUCUUCCAUGU |
| | | GUUUAUCGUAGUGAUCGAAAUCGACUGAUCUCGGGUUCUUUUUGU |
| | | UGGUUUCUUUUCGGUUAAUCAUGUAUUGUUUUCUUUUUUUACAGA |
| | | AAGAUACUUUUUUCAUGAGCAAUUCCUCGCCCGGCGCCGGCAUGCC |
| | | GAGGUGGGGCCACUGCGAUCAGCGGCAUGCCGACGCCGACCCGGGG |
| | | AUCUUGGAUUCACCGUUUUCUCUCUUCUCUCUCUACAUACAGACCG |
| | | GGUGGCAGGAGCGGUAAGGAAUCAUCGUCGUCUUUCAUUCUUCGA |
| | | UGAUUAUGGUAAUACUAAAUCUUAUCUAGGAGCAUAUACAUCUAA |
| | | GAUUGGAGUACUAGUAGUCGUUUGUGGUUUCUAUUUUUUUUUAUA |
| | | UUUAUCUAUGACAGUUUUUCUGUUUUUCGUUUUGAUAAUAAUAUA |
| | | AUAAAAACUCAUGGACGUGAAAUCUGGCUUGGUUGUGGUGAUUUC |
| | | AUUCUCAUUAUUGUUGUUUUCUUUCCGUCUUGCGGAUGAAGAUGU |
| | | UGCGAUGCGGUUGUUGUUGGUGUUGCUAUACACCGAGAGAGAUGA |
| | | UCUUUUUGUUCUUCUGGUUCAUUUCCUAUGAUUGUUUGGCUGCUG |
| | | ACCGACGCGUCAGGAUGUGCAGGGCAUGCGGGGAAUCAGGACCGG |
| | | ACACGGGAUAAUUUCAUCUACCUAUACGGAGAUCGCGGUCCUCGCC |
| | | AUGAGGAUCGCGACAGGCGCGUCGAGGGGGCAGGAACACCCUUGC |
| | | GGAUUGACAUUCUUGGUGGUGUUUCGUUGUUGUCGGUAGUUGUUG |
| | | UUGACGAUGAGGAUAAAUAAAAAUGACCUUGUUUUUGUUCUGUUU |
| | | UCUCUUGUUGGGAAUCGUCGACUUUGAAUUCUUCGAGUUAUCGGA |
| | | AAGCUGAGGUACCCAAAUGUCUGUAGCUAAAAAUAAUAAAACAGA |
| | | AGCCGACCGGCCGCCGACCCGUUCCCCAGGACCAGCCUACGAGGAA |
| | | CGGAUAACGCGGUGGCGACGGCAGCGGUGGUGGCGCUGGGGGUGG |
| | | CGGUAGUGGUGCUGCUGAUGGUAGUCGGGACGGAGGAGAGACGAU |
| | | GCAUACAUACACGCGUGCAUGCUGCAUGGGUGGAUGGUACGGCCG |
| | | GGAGACGCGGAAGAGAAACUCACAUAAAAAGGUGAUAAAAAGAGC |
| | | GGUUGAAAAAAGAAAACGAGAUUCGACCAGACAGAAGAGAAGGAC |
| | | CGGGGCUUGGCGACCCUUCCACGACUGCCGUUGUCAUCUCGGCUCC |
| | | UCCAUCUUCUCCCGGCCACGGGCGGCUAAGUCACCGCCGUUCUCCC |
| | | CAUCCGUCCGAGCGCCGACCGACCAGCCGGCCGAUUCGCCCGCCGG |
| | | GGCUUCUGGAGAACGCCGGGGCAGCAGCGAUCUGGGGA |
| 71 | ΔD3AS_ | GCAUGCAAACUUCUCAUUUAUUGUGUCUACUACUCUGUGUUGCUA |
| | core | CAGGGAGUGAAGGGGGUGAAGGCAAACAAAAAAAAAAGGAACAAA |
| | | AUAAUAGAUUAGCAGAAGGAAUAAUCCGUGCGACCGAGCUUGUGC |
| | | UUCUUUUCUUAUAAGGAGGCAAAUAUACUAGGGAAAACUUAAGAA |
| | | UAGGAAGAAACCGAGGUUUGGGAGAAAAGCUGAGAUAAAAUAGCG |
| | | CAUUUUCCAUACAGAGGUUGUUGUUUUUGUGGAUCCUAAGAGGUU |
| | | UCAAGUGCGAAUCUCAAAGUUCUCACGAGAAUAUUGUCUUCAAGA |
| | | AUCGACAACUGUGGUCCAAGAUUUUUUUUUGGUCUUUUUAGGUUC |
| | | UGCGAGGGACAUCACGAUGGAUCGUUGCGAUGAAGUCACGCGUAC |
| | | GCCUCUGGUGUGGCGCGGUGUCGUGACAGGAGAGUGUGUUUUCAG |
| | | UGCAGAGCUGUCUUGAUUCCUAUAUCCGAGUAUCUGUUUUCUCGU |
| | | AAGGACGGUAAUCUUCUUUGGUGUAAGUACAUCUAAAAGCUGCAA |
| | | ACUAUAUUUUAAGGGCUGUCUCUAGGUGUACUUUGAUGCUGGAGU |
| | | UUUUCGCUGUGUUGAUGUGAAUAAAUCUACUACUACUAUUAUAUG |
| | | CAGAAAGAGUGAUUAUGCCGAGACAAGAUUGCAUUGGCUGAACUG |
| | | UUUCAAAAACGCCUACACUCUACUUAUCCGUAAACCUAAGGUAAU |
| | | ACUAUGUGUAAGUUGUUUUUUUUUCUUUUUGUAGUAAAAUGGUGA |
| | | UACGUGCAAUUAAAACUGUAUUCCAUGUUUCCAUCCUUUCAUUUC |
| | | AACUUUAAAGGCGGCUUUGAGAGCGAAGAAGUGCGAGGAUAAAAA |
| | | UGGAUGACUCCUUCGUGUCCAGGGAGUCGACUACUGCAACGCUGA |
| | | UUGAUUAAAAGAUGGUCUCCGAUGAUGAUGUUGUUAUUGAUCGAA |
| | | UCAUGGUGCAGAACGGCGACGGAGAGGAGCGUGUCCGCCGCCGGG |
| | | AAGGUGGUCUCUUUCUCUUUUCUUUUUUCAAGAAAUCUUCCAUGU |
| | | GUUUAUCGUAGUGAUCGAAAUCGACUGAUCUCGGGUUCUUUUUGU |
| | | UGGUUUCUUUUCGGUUAAUCAUGUAUUGUUUUCUUUUUUUACAGA |
| | | AAGAUACUUUUUUCAUGAGCAAUUCCUCGCCCGGCGCCGGCAUGCC |
| | | GAGGUGGGGCCACUGCGAUCAGCGGCAUGCCGACGCCGACCCGGGG |
| | | AUCUUGGAUUCACCGUUUUCUCUCUUCUCUCUCUACAUACAGACCG |
| | | GGUGGCAGGAGCGGUAAGGAAUCAUCGUCGUCUUUCAUUCUUCGA |
| | | UGAUUAUGGUAAUACUAAAUCUUAUCUAGGAGCAUAUACAUCUAA |
| | | GAUUGGAGUACUAGUAGUCGUUUGUGGUUUCUAUUUUUUUUUAUA |
| | | UUUAUCUAUGACAGUUUUUCUGUUUUUCGUUUUGAUAAUAAUAUA |
| | | AUAAAAACUCAUGGACGUGAAAUCUGGCUUGGUUGUGGUGAUUUC |
| | | AUUCUCAUUAUUGUUGUUUUCUUUCCGUCUUGCGGAUGAAGAUGU |
| | | UGCGAUGCGGUUGUUGUUGGUGUUGCUAUACACCGAGAGAGAUGA |
| | | UCUUUUUGUUCUUCUUUGUUUUUGUUCUGUUUUCUCUUGUUGGGA |
| | | AUCGUCGACUUUGAAUUCUUCGAGUUAUCGGAAAGCUGAGGUACC |
| | | CAAAUGUCUGUAGCUUUUUUCUUUUUACCCUCUUGUUUAUCAUCU |
| | | GCGAUUCGUGGUAGGUAGGAGAGGGAAAUGAUAAUCCGAGAUUAA |
| | | GGAAAGGAGAAGAUAAAAAAUAAAAAAAAAUAAUAAAACAGAAGC |
| | | CGACCGGCCGCCGACCCGUUCCCCAGGACCAGCCUACGAGGAACGG |
| | | AUAACGCGGUGGCGACGGCAGCGGUGGUGGCGCUGGGGGUGGCGG |
| | | UAGUGGUGCUGCUGAUGGUAGUCGGGACGGAGGAGAGACGAUGCA |
| | | UACAUACACGCGUGCAUGCUGCAUGGGUGGAUGGUACGGCCGGGA |
| | | GACGCGGAAGAGAAACUCACAUAAAAAGGUGAUAAAAAGAGCGGU |
| | | UGAAAAAAGAAAACGAGAUUCGACCAGACAGAAGAGAAGGACCGG |
| | | GGCUUGGCGACCCUUCCACGACUGCCGUUGUCAUCUCGGCUCCUCC |
| | | AUCUUCUCCCGGCCACGGGCGGCUAAGUCACCGCCGUUCUCCCCAU |
| | | CCGUCCGAGCGCCGACCGACCAGCCGGCCGAUUCGCCCGCCGGGGC |
| | | UUCUGGAGAACGCCGGGGCAGCAGCGAUCUGGGGA |
| 72 | ΔD4AS_ | GCAUGCAAACUUCUCAUUUAUUGUGUCUACUACUCUGUGUUGCUA |
| | core | CAGGGAGUGAAGGGGGUGAAGGCAAACAAAAAAAAAAGGAACAAA |
| | | AUAAUAGAUUAGCAGAAGGAAUAAUCCGUGCGACCGAGCUUGUGC |
| | | UUCUUUUCUUAUAAGGAGGCAAAUAUACUAGGGAAAACUUAAGAA |
| | | UAGGAAGAAACCGAGGUUUGGGAGAAAAGCUGAGAUAAAAUAGCG |
| | | CAUUUUCCAUACAGAGGUUGUUGUUUUUGUGGAUCCUAAGAGGUU |
| | | UCAAGUGCGAAUCUCAAAGUUCUCACGAGAAUAUUGUCUUCAAGA |
| | | AUCGACAACUGUGGUCCAAGAUUUUUUUUUGGUCUUUUUAGGUUC |
| | | UGCGAGGGACAUCACGAUGGAUCGUUGCGAUGAAGUCACGCGUAC |
| | | GCCUCUGGUGUGGCGCGGUGUCGUGACAGGAGAGUGUGUUUUCAG |
| | | UGCAGAGCUGUCUUGAUUCCUAUAUCCGAGUAUCUGUUUUCUCGU |
| | | AAGGACGGUAAUCUUCUUUGGUGUAAGUACAUCUAAAAGCUGCAA |
| | | ACUAUAUUUUAAGGGCUGUCUCUAGGUGUACUUUGAUGCUGGAGU |
| | | UUUUCGCUGUGUUGAUGUGAAUAAAUCUACUACUACUAUUAUAUG |
| | | CAGAAAGAGUGAUUAUGCCGAGACAAGAUUGCAUUGGCUGAACUG |
| | | UUUCAAAAACGCCUACACUCUACUUAUCCGUAAACCUAAGGUAAU |
| | | ACUAUGUGUAAGUUGUUUUUUUUUCUUUUUGUAGUAAAAUGGUGA |
| | | UACGUGCAAUUAAAACUGUAUUCCAUGUUUCCAUCCUUUCAUUUC |
| | | AACUUUAAAGGCGGCUUUGAGAGCGAAGAAGUACCCGGGGAUCUU |
| | | GGAUUCACCGUUUUCUCUCUUCUCUCUCUACAUACAGACCGGGUGG |
| | | CAGGAGCGGUAAGGAAUCAUCGUCGUCUUUCAUUCUUCGAUGAUU |
| | | AUGGUAAUACUAAAUCUUAUCUAGGAGCAUAUACAUCUAAGAUUG |
| | | GAGUACUAGUAGUCGUUUGUGGUUUCUAUUUUUUUUUAUAUUUAU |
| | | CUAUGACAGUUUUUCUGUUUUUCGUUUUGAUAAUAAUAUAAUAAA |
| | | AACUCAUGGACGUGAAAUCUGGCUUGGUUGUGGUGAUUUCAUUCU |
| | | CAUUAUUGUUGUUUUCUUUCCGUCUUGCGGAUGAAGAUGUUGCGA |
| | | UGCGGUUGUUGUUGGUGUUGCUAUACACCGAGAGAGAUGAUCUUU |
| | | UUGUUCUUCUGGUUCAUUUCCUAUGAUUGUUUGGCUGCUGACCGA |
| | | CGCGUCAGGAUGUGCAGGGCAUGCGGGGAAUCAGGACCGGACACG |
| | | GGAUAAUUUCAUCUACCUAUACGGAGAUCGCGGUCCUCGCCAUGA |
| | | GGAUCGCGACAGGCGCGUCGAGGGGGCAGGAACACCCUUGCGGAU |
| | | UGACAUUCUUGGUGGUGUUUCGUUGUUGUCGGUAGUUGUUGUUGA |
| | | CGAUGAGGAUAAAUAAAAAUGACCUUGUUUUUGUUCUGUUUUCUC |
| | | UUGUUGGGAAUCGUCGACUUUGAAUUCUUCGAGUUAUCGGAAAGC |
| | | UGAGGUACCCAAAUGUCUGUAGCUUUUUUCUUUUUACCCUCUUGU |
| | | UUAUCAUCUGCGAUUCGUGGUAGGUAGGAGAGGGAAAUGAUAAUC |
| | | CGAGAUUAAGGAAAGGAGAAGAUAAAAAAUAAAAAAAAAUAAUAA |
| | | AACAGAAGCCGACCGGCCGCCGACCCGUUCCCCAGGACCAGCCUAC |
| | | GAGGAACGGAUAACGCGGUGGCGACGGCAGCGGUGGUGGCGCUGG |
| | | GGGUGGCGGUAGUGGUGCUGCUGAUGGUAGUCGGGACGGAGGAGA |
| | | GACGAUGCAUACAUACACGCGUGCAUGCUGCAUGGGUGGAUGGUA |
| | | CGGCCGGGAGACGCGGAAGAGAAACUCACAUAAAAAGGUGAUAAA |
| | | AAGAGCGGUUGAAAAAAGAAAACGAGAUUCGACCAGACAGAAGAG |
| | | AAGGACCGGGGCUUGGCGACCCUUCCACGACUGCCGUUGUCAUCUC |
| | | GGCUCCUCCAUCUUCUCCCGGCCACGGGCGGCUAAGUCACCGCCGU |
| | | UCUCCCCAUCCGUCCGAGCGCCGACCGACCAGCCGGCCGAUUCGCC |
| | | CGCCGGGGCUUCUGGAGAACGCCGGGGCAGCAGCGAUCUGGGGA |
| 73 | Multimer | UUUUAUUUUUUAUCUUCUCCUUUCCUUAAUCUCGGAUUAUCAUUU |
| | sequence | CCCUCUCCUACCUACCACGAAUCGCAGAUGAUAAACAAGAGGGUAA |
| | D2x4_core | AAAGAAAAAUUUUAUUUUUUAUCUUCUCCUUUCCUUAAUCUCGGA |
| | | UUAUCAUUUCCCUCUCCUACCUACCACGAAUCGCAGAUGAUAAACA |
| | | AGAGGGUAAAAAGAAAAAUUUUAUUUUUUAUCUUCUCCUUUCCUU |
| | | AAUCUCGGAUUAUCAUUUCCCUCUCCUACCUACCACGAAUCGCAGA |
| | | UGAUAAACAAGAGGGUAAAAAGAAAAAUUUUAUUUUUUAUCUUCU |
| | | CCUUUCCUUAAUCUCGGAUUAUCAUUUCCCUCUCCUACCUACCACG |
| | | AAUCGCAGAUGAUAAACAAGAGGGUAAAAAGAAAAA |
| 74 | Multimer | GGUCAUUUUUAUUUAUCCUCAUCGUCAACAACAACUACCGACAACA |
| | sequence | ACGAAACACCACCAAGAAUGUCAAUCCGCAAGGGUGUUCCUGCCCC |
| | D3x4_core | CUCGACGCGCCUGUCGCGAUCCUCAUGGCGAGGACCGCGAUCUCCG |
| | | UAUAGGUAGAUGAAAUUAUCCCGUGUCCGGUCCUGAUUCCCCGCA |
| | | UGCCCUGCACAUCCUGACGCGUCGGUCAGCAGCCAAACAAUCAUAG |
| | | GAAAUGAACCGGUCAUUUUUAUUUAUCCUCAUCGUCAACAACAAC |
| | | UACCGACAACAACGAAACACCACCAAGAAUGUCAAUCCGCAAGGGU |
| | | GUUCCUGCCCCCUCGACGCGCCUGUCGCGAUCCUCAUGGCGAGGAC |
| | | CGCGAUCUCCGUAUAGGUAGAUGAAAUUAUCCCGUGUCCGGUCCU |
| | | GAUUCCCCGCAUGCCCUGCACAUCCUGACGCGUCGGUCAGCAGCCA |
| | | AACAAUCAUAGGAAAUGAAGGUCAUUUUUAUUUAUCCUCAUCGUC |
| | | AACAACAACUACCGACAACAACGAAACACCACCAAGAAUGUCAAUC |
| | | CGCAAGGGUGUUCCUGCCCCCUCGACGCGCCUGUCGCGAUCCUCAU |
| | | GGCGAGGACCGCGAUCUCCGUAUAGGUAGAUGAAAUUAUCCCGUG |
| | | UCCGGUCCUGAUUCCCCGCAUGCCCUGCACAUCCUGACGCGUCGGU |
| | | CAGCAGCCAAACAAUCAUAGGAAAUGAACCGGUCAUUUUUAUUUA |
| | | UCCUCAUCGUCAACAACAACUACCGACAACAACGAAACACCACCAA |
| | | GAAUGUCAAUCCGCAAGGGUGUUCCUGCCCCCUCGACGCGCCUGUC |
| | | GCGAUCCUCAUGGCGAGGACCGCGAUCUCCGUAUAGGUAGAUGAA |
| | | AUUAUCCCGUGUCCGGUCCUGAUUCCCCGCAUGCCCUGCACAUCCU |
| | | GACGCGUCGGUCAGCAGCCAAACAAUCAUAGGAAAUGAACC |
| 75 | Multimer | GGUCAUUUUUAUUUAUCCUCAUCGUCAACAACAACUACCGACAACA |
| | sequence | ACGAAACACCACCAAGAAUGUCAAUCCGCAAGGGUGUUCCUGCCCC |
| | D3x4_D2x4_ | CUCGACGCGCCUGUCGCGAUCCUCAUGGCGAGGACCGCGAUCUCCG |
| | core | UAUAGGUAGAUGAAAUUAUCCCGUGUCCGGUCCUGAUUCCCCGCA |
| | | UGCCCUGCACAUCCUGACGCGUCGGUCAGCAGCCAAACAAUCAUAG |
| | | GAAAUGAACCGGUCAUUUUUAUUUAUCCUCAUCGUCAACAACAAC |
| | | UACCGACAACAACGAAACACCACCAAGAAUGUCAAUCCGCAAGGGU |
| | | GUUCCUGCCCCCUCGACGCGCCUGUCGCGAUCCUCAUGGCGAGGAC |
| | | CGCGAUCUCCGUAUAGGUAGAUGAAAUUAUCCCGUGUCCGGUCCU |
| | | GAUUCCCCGCAUGCCCUGCACAUCCUGACGCGUCGGUCAGCAGCCA |
| | | AACAAUCAUAGGAAAUGAAGGUCAUUUUUAUUUAUCCUCAUCGUC |
| | | AACAACAACUACCGACAACAACGAAACACCACCAAGAAUGUCAAUC |
| | | CGCAAGGGUGUUCCUGCCCCCUCGACGCGCCUGUCGCGAUCCUCAU |
| | | GGCGAGGACCGCGAUCUCCGUAUAGGUAGAUGAAAUUAUCCCGUG |
| | | UCCGGUCCUGAUUCCCCGCAUGCCCUGCACAUCCUGACGCGUCGGU |
| | | CAGCAGCCAAACAAUCAUAGGAAAUGAACCGGUCAUUUUUAUUUA |
| | | UCCUCAUCGUCAACAACAACUACCGACAACAACGAAACACCACCAA |
| | | GAAUGUCAAUCCGCAAGGGUGUUCCUGCCCCCUCGACGCGCCUGUC |
| | | GCGAUCCUCAUGGCGAGGACCGCGAUCUCCGUAUAGGUAGAUGAA |
| | | AUUAUCCCGUGUCCGGUCCUGAUUCCCCGCAUGCCCUGCACAUCCU |
| | | GACGCGUCGGUCAGCAGCCAAACAAUCAUAGGAAAUGAACCUUUU |
| | | AUUUUUUAUCUUCUCCUUUCCUUAAUCUCGGAUUAUCAUUUCCCUC |
| | | UCCUACCUACCACGAAUCGCAGAUGAUAAACAAGAGGGUAAAAAG |
| | | AAAAAUUUUAUUUUUUAUCUUCUCCUUUCCUUAAUCUCGGAUUAU |
| | | CAUUUCCCUCUCCUACCUACCACGAAUCGCAGAUGAUAAACAAGAG |
| | | GGUAAAAAGAAAAAUUUUAUUUUUUAUCUUCUCCUUUCCUUAAUC |
| | | UCGGAUUAUCAUUUCCCUCUCCUACCUACCACGAAUCGCAGAUGAU |
| | | AAACAAGAGGGUAAAAAGAAAAAUUUUAUUUUUUAUCUUCUCCUU |
| | | UCCUUAAUCUCGGAUUAUCAUUUCCCUCUCCUACCUACCACGAAUC |
| | | GCAGAUGAUAAACAAGAGGGUAAAAAGAAAAACGGCCC |
| 76 | Multimer | UUUUAUUUUUUAUCUUCUCCUUUCCUUAAUCUCGGAUUAUCAUUU |
| | sequence | CCCUCUCCUACCUACCACGAAUCGCAGAUGAUAAACAAGAGGGUAA |
| | D2x4_D3X4_ | AAAGAAAAAUUUUAUUUUUUAUCUUCUCCUUUCCUUAAUCUCGGA |
| | core | UUAUCAUUUCCCUCUCCUACCUACCACGAAUCGCUUUUAUUUUUUA |
| | | UCUUCUCCUUUCCUUAAUCUCGGAUUAUCAUUUCCCUCUCCUACCU |
| | | ACCACGAAUCGCAGAUGAUAAACAAGAGGGUAAAAAGAAAAAUUU |
| | | UAUUUUUUAUCUUCUCCUUUCCUUAAUCUCGGAUUAUCAUUUCCC |
| | | UCUCCUACCUACCACGAAUCGCAGAUGAUAAACAAGAGGGUAAAA |
| | | AGAAAAAGGUCAUUUUUAUUUAUCCUCAUCGUCAACAACAACUAC |
| | | CGACAACAACGAAACACCACCAAGAAUGUCAAUCCGCAAGGGUGUU |
| | | CCUGCCCCCUCGACGCGCCUGUCGCGAUCCUCAUGGCGAGGACCGC |
| | | GAUCUCCGUAUAGGUAGAUGAAAUUAUCCCGUGUCCGGUCCUGAU |
| | | UCCCCGCAUGCCCUGCACAUCCUGACGCGUCGGUCAGCAGCCAAAC |
| | | AAUCAUAGGAAAUGAACCGGUCAUUUUUAUUUAUCCUCAUCGUCA |
| | | ACAACAACUACCGACAACAACGAAACACCACCAAGAAUGUCAAUCC |
| | | GCAAGGGUGUUCCUGCCCCCUCGACGCGCCUGUCGCGAUCCUCAUG |
| | | GCGAGGACCGCGAUCUCCGUAUAGGUAGAUGAAAUUAUCCCGUGU |
| | | CCGGUCCUGAUUCCCCGCAUGCCCUGCACAUCCUGACGCGUCGGUC |
| | | AGCAGCCAAACAAUCAUAGGAAAUGAACCGGUCAUUUUUAUUUAU |
| | | CCUCAUCGUCAACAACAACUACCGACAACAACGAAACACCACCAAG |
| | | AAUGUCAAUCCGCAAGGGUGUUCCUGCCCCCUCGACGCGCCUGUCG |
| | | CGAUCCUCAUGGCGAGGACCGCGAUCUCCGUAUAGGUAGAUGAAA |
| | | UUAUCCCGUGUCCGGUCCUGAUUCCCCGCAUGCCCUGCACAUCCUG |
| | | ACGCGUCGGUCAGCAGCCAAACAAUCAUAGGAAAUGAACCGGUCA |
| | | UUUUUAUUUAUCCUCAUCGUCAACAACAACUACCGACAACAACGAA |
| | | ACACCACCAAGAAUGUCAAUCCGCAAGGGUGUUCCUGCCCCCUCGA |
| | | CGCGCCUGUCGCGAUCCUCAUGGCGAGGACCGCGAUCUCCGUAUAG |
| | | GUAGAUGAAAUUAUCCCGUGUCCGGUCCUGAUUCCCCGCAUGC CCU |
| | | GCACAUCCUGACGCGUCGGUCAGCAGCCAAACAAUCAUAGGAAAUG |
| | | AACC |
| 77 | Multimer | GGUCAUUUUUAUUUAUCCUCAUCGUCAACAACAACUACCGACAACA |
| | sequence | ACGAAACACCACCAAGAAUGUCAAUCCGCAAGGGUGUUCCUGCCCC |
| | (D3_D2)x4_ | CUCGACGCGCCUGUCGCGAUCCUCAUGGCGAGGACCGCGAUCUCCG |
| | core | UAUAGGUAGAUGAAAUUAUCCCGUGUCCGGUCCUGAUUCCCCGCA |
| | | UGCCCUGCACAUCCUGACGCGUCGGUCAGCAGCCAAACAAUCAUAG |
| | | GAAAUGAACCUUUUAUUUUUUAUCUUCUCCUUUCCUUAAUCUCGG |
| | | AUUAUCAUUUCCCUCUCCUACCUACCACGAAUCGCAGAUGAUAAAC |
| | | AAGAGGGUAAAAAGAAAAAGGUCAUUUUUAUUUAUCCUCAUCGUC |
| | | AACAACAACUACCGACAACAACGAAACACCACCAAGAAUGUCAAUC |
| | | CGCAAGGGUGUUCCUGCCCCCUCGACGCGCCUGUCGCGAUCCUCAU |
| | | GGCGAGGACCGCGAUCUCCGUAUAGGUAGAUGAAAUUAUCCCGUG |
| | | UCCGGUCCUGAUUCCCCGCAUGCCCUGCACAUCCUGACGCGUCGGU |
| | | CAGCAGCCAAACAAUCAUAGGAAAUGAACCUUUUAUUUUUUAUCU |
| | | UCUCCUUUCCUUAAUCUCGGAUUAUCAUUUCCCUCUCCUACCUACC |
| | | ACGAAUCGCAGAUGAUAAACAAGAGGGUAAAAAGAAAAAGGUCAU |
| | | UUUUAUUUAUCCUCAUCGUCAACAACAACUACCGACAACAACGAAA |
| | | CACCACCAAGAAUGUCAAUCCGCAAGGGUGUUCCUGCCCCCUCGAC |
| | | GCGCCUGUCGCGAUCCUCAUGGCGAGGACCGCGAUCUCCGUAUAGG |
| | | UAGAUGAAAUUAUCCCGUGUCCGGUCCUGAUUCCCCGCAUGCCCUG |
| | | CACAUCCUGACGCGUCGGUCAGCAGCCAAACAAUCAUAGGAAAUGA |
| | | ACCUUUUAUUUUUUAUCUUCUCCUUUCCUUAAUCUCGGAUUAUCA |
| | | UUUCCCUCUCCUACCUACCACGAAUCGCAGAUGAUAAACAAGAGGG |
| | | UAAAAAGAAAAAGGUCAUUUUUAUUUAUCCUCAUCGUCAACAACA |
| | | ACUACCGACAACAACGAAACACCACCAAGAAUGUCAAUCCGCAAGG |
| | | GUGUUCCUGCCCCCUCGACGCGCCUGUCGCGAUCCUCAUGGCGAGG |
| | | ACCGCGAUCUCCGUAUAGGUAGAUGAAAUUAUCCCGUGUCCGGUCC |
| | | UGAUUCCCCGCAUGCCCUGCACAUCCUGACGCGUCGGUCAGCAGCC |
| | | AAACAAUCAUAGGAAAUGAACCUUUUAUUUUUUAUCUUCUCCUUU |
| | | CCUUAAUCUCGGAUUAUCAUUUCCCUCUCCUACCUACCACGAAUCG |
| | | CAGAUGAUAAACAAGAGGGUAAAAAGAAAAA |
| 78 | gGFP_β2.7 | AUGGUGAGCAAGGGCGAGGAGCUGUUCACCGGGGUGGUGCCCAUC |
| | core | CUGGUCGAGCUGGACGGCGACGUAAACGGCCACAAGUUCAGCGUG |
| | | UCCGGCGAGGGCGAGGGCGAUGCCACCUACGGCAAGCUGACCCUGA |
| | | AGUUCAUCUGCACCACCGGCAAGCUGCCCGUGCCCUGGCCCACCCU |
| | | CGUGACCACCCUGACCUACGGCGUGCAGUGCUUCAGCCGCUACCCC |
| | | GACCACAUGAAGCAGCACGACUUCUUCAAGUCCGCCAUGCCCGAAG |
| | | GCUACGUCCAGGAGCGCACCAUCUUCUUCAAGGACGACGGCAACUA |
| | | CAAGACCCGCGCCGAGGUGAAGUUCGAGGGCGACACCCUGGUGAAC |
| | | CGCAUCGAGCUGAAGGGCAUCGACUUCAAGGAGGACGGCAACAUCC |
| | | UGGGGCACAAGCUGGAGUACAACUACAACAGCCACAACGUCUAUA |
| | | UCAUGGCCGACAAGCAGAAGAACGGCAUCAAGGUGAACUUCAAGA |
| | | UCCGCCACAACAUCGAGGACGGCAGCGUGCAGCUCGCCGACCACUA |
| | | CCAGCAGAACACCCCCAUCGGCGACGGCCCCGUGCUGCUGCCCGAC |
| | | AACCACUACCUGAGCACCCAGUCCGCCCUGAGCAAAGACCCCAACG |
| | | AGAAGCGCGAUCACAUGGUCCUGCUGGAGUUCGUGACCGCCGCCGG |
| | | GAUCACUCUCGGCAUGGACGAGCUGUACAAGUGAUCCCCAGAUCGC |
| | | UGCUGCCCCGGCGUUCUCCAGAAGCCCCGGCGGGCGAAUCGGCCGG |
| | | CUGGUCGGUCGGCGCUCGGACGGAUGGGGAGAACGGCGGUGACUU |
| | | AGCCGCCCGUGGCCGGGAGAAGAUGGAGGAGCCGAGAUGACAACG |
| | | GCAGUCGUGGAAGGGUCGCCAAGCCCCGGUCCUUCUCUUCUGUCUG |
| | | GUCGAAUCUCGUUUUCUUUUUUCAACCGCUCUUUUUAUCACCUUU |
| | | UUAUGUGAGUUUCUCUUCCGCGUCUCCCGGCCGUACCAUCCACCCA |
| | | UGCAGCAUGCACGCGUGUAUGUAUGCAUCGUCUCUCCUCCGUCCCG |
| | | ACUACCAUCAGCAGCACCACUACCGCCACCCCCAGCGCCACCACCGC |
| | | UGCCGUCGCCACCGCGUUAUCCGUUCCUCGUAGGCUGGUCCUGGGG |
| | | AACGGGUCGGCGGCCGGUCGGCUUCUGUUUUAUUAUUUUUUUUUA |
| | | UUUUUUAUCUUCUCCUUUCCUUAAUCUCGGAUUAUCAUUUCCCUCU |
| | | CCUACCUACCACGAAUCGCAGAUGAUAAACAAGAGGGUAAAAAGA |
| | | AAAAAGCUACAGACAUUUGGGUACCUCAGCUUUCCGAUAACUCGA |
| | | AGAAUUCAAAGUCGACGAUUCCCAACAAGAGAAAACAGAACAAAA |
| | | ACAAGGUCAUUUUUAUUUAUCCUCAUCGUCAACAACAACUACCGAC |
| | | AACAACGAAACACCACCAAGAAUGUCAAUCCGCAAGGGUGUUCCUG |
| | | CCCCCUCGACGCGCCUGUCGCGAUCCUCAUGGCGAGGACCGCGAUC |
| | | UCCGUAUAGGUAGAUGAAAUUAUCCCGUGUCCGGUCCUGAUUCCCC |
| | | GCAUGCCCUGCACAUCCUGACGCGUCGGUCAGCAGCCAAACAAUCA |
| | | UAGGAAAUGAACCAGAAGAACAAAAAGAUCAUCUCUCUCGGUGUA |
| | | UAGCAACACCAACAACAACCGCAUCGCAACAUCUUCAUCCGCAAGA |
| | | CGGAAAGAAAACAACAAUAAUGAGAAUGAAAUCACCACAACCAAG |
| | | CCAGAUUUCACGUCCAUGAGUUUUUAUUAUAUUAUUAUCAAAACG |
| | | AAAAACAGAAAAACUGUCAUAGAUAAAUAUAAAAAAAAAUAGAAA |
| | | CCACAAACGACUACUAGUACUCCAAUCUUAGAUGUAUAUGCUCCUA |
| | | GAUAAGAUUUAGUAUUACCAUAAUCAUCGAAGAAUGAAAGACGAC |
| | | GAUGAUUCCUUACCGCUCCUGCCACCCGGUCUGUAUGUAGAGAGAG |
| | | AAGAGAGAAAACGGUGAAUCCAAGAUCCCCGGGUCGGCGUCGGCA |
| | | UGCCGCUGAUCGCAGUGGCCCCACCUCGGCAUGCCGGCGCCGGGCG |
| | | AGGAAUUGCUCAUGAAAAAAGUAUCUUUCUGUAAAAAAAGAAAAC |
| | | AAUACAUGAUUAACCGAAAAGAAACCAACAAAAAGAACCCGAGAU |
| | | CAGUCGAUUUCGAUCACUACGAUAAACACAUGGAAGAUUUCUUGA |
| | | AAAAAGAAAAGAGAAAGAGACCACCUUCCCGGCGGCGGACACGCUC |
| | | CUCUCCGUCGCCGUUCUGCACCAUGAUUCGAUCAAUAACAACAUCA |
| | | UCAUCGGAGACCAUCUUUUAAUCAAUCAGCGUUGCAGUAGUCGAC |
| | | UCCCUGGACACGAAGGAGUCAUCCAUUUUUAUCCUCGCACUUCUUC |
| | | GCUCUCAAAGCCGCCUUUAAAGUUGAAAUGAAAGGAUGGAAACAU |
| | | GGAAUACAGUUUUAAUUGCACGUAUCACCAUUUUACUACAAAAAG |
| | | AAAAAAAAACAACUUACACAUAGUAUUACCUUAGGUUUACGGAUA |
| | | AGUAGAGUGUAGGCGUUUUUGAAACAGUUCAGCCAAUGCAAUCUU |
| | | GUCUCGGCAUAAUCACUCUUUCUGCAUAUAAUAGUAGUAGUAGAU |
| | | UUAUUCACAUCAACACAGCGAAAAACUCCAGCAUCAAAGUACACCU |
| | | AGAGACAGCCCUUAAAAUAUAGUUUGCAGCUUUUAGAUGUACUUA |
| | | CACCAAAGAAGAUUACCGUCCUUACGAGAAAACAGAUACUCGGAU |
| | | AUAGGAAUCAAGACAGCUCUGCACUGAAAACACACUCUCCUGUCAC |
| | | GACACCGCGCCACACCAGAGGCGUACGCGUGACUUCAUCGCAACGA |
| | | UCCAUCGUGAUGUCCCUCGCAGAACCUAAAAAGACCAAAAAAAAA |
| | | UCUUGGACCACAGUUGUCGAUUCUUGAAGACAAUAUUCUCGUGAG |
| | | AACUUUGAGAUUCGCACUUGAAACCUCUUAGGAUCCACAAAAACA |
| | | ACAACCUCUGUAUGGAAAAUGCGCUAUUUUAUCUCAGCUUUUCUC |
| | | CCAAACCUCGGUUUCUUCCUAUUCUUAAGUUUUCCCUAGUAUAUU |
| | | UGCCUCCUUAUAAGAAAAGAAGCACAAGCUCGGUCGCACGGAUUA |
| | | UUCCUUCUGCUAAUCUAUUAUUUUGUUCCUUUUUUUUUUGUUUGC |
| | | CUUCACCCCCUUCACUCCCUGUAGCAACACAGAGUAGUAGACACAA |
| | | UAAAUGAGAAGU |
| 79 | gGFP_s_β2.7_ | AUGGUGAGCAAGGGCGAGGAGCUGUUCACCGGGGUGGUGCCCAUC |
| | core | CUGGUCGAGCUGGACGGCGACGUAAACGGCCACAAGUUCAGCGUG |
| | | UCCGGCGAGGGCGAGGGCGAUGCCACCUACGGCAAGCUGACCCUGA |
| | | AGUUCAUCUGCACCACCGGCAAGCUGCCCGUGCCCUGGCCCACCCU |
| | | CGUGACCACCCUGACCUACGGCGUGCAGUGCUUCAGCCGCUACCCC |
| | | GACCACAUGAAGCAGCACGACUUCUUCAAGUCCGCCAUGCCCGAAG |
| | | GCUACGUCCAGGAGCGCACCAUCUUCUUCAAGGACGACGGCAACUA |
| | | CAAGACCCGCGCCGAGGUGAAGUUCGAGGGCGACACCCUGGUGAAC |
| | | CGCAUCGAGCUGAAGGGCAUCGACUUCAAGGAGGACGGCAACAUCC |
| | | UGGGGCACAAGCUGGAGUACAACUACAACAGCCACAACGUCUAUA |
| | | UCAUGGCCGACAAGCAGAAGAACGGCAUCAAGGUGAACUUCAAGA |
| | | UCCGCCACAACAUCGAGGACGGCAGCGUGCAGCUCGCCGACCACUA |
| | | CCAGCAGAACACCCCCAUCGGCGACGGCCCCGUGCUGCUGCCCGAC |
| | | AACCACUACCUGAGCACCCAGUCCGCCCUGAGCAAAGACCCCAACG |
| | | AGAAGCGCGAUCACAUGGUCCUGCUGGAGUUCGUGACCGCCGCCGG |
| | | GAUCACUCUCGGCAUGGACGAGCUGUACAAGUGAUCCCCAGAUCGC |
| | | UGCUGCCCCGGCGUUCUCCAGAAGCCCCGGCGGGCGAAUCGGCCGG |
| | | CUGGUCGGUCGGCGCUCGGACGGAUGGGGAGAACGGCGGUGACUU |
| | | AGCCGCCCGUGGCCGGGAGAAGAUGGAGGAGCCGAGAUGACAACG |
| | | GCAGUCGUGGAAGGGUCGCCAAGCCCCGGUCCUUCUCUUCUGUCUG |
| | | GUCGAAUCUCGUUUUCUUUUUUCAACCGCUCUUUUUAUCACCUUU |
| | | UUAUGUGAGUUUCUCUUCCGCGUCUCCCGGCCGUACCAUCCACCCA |
| | | UGCAGCAUGCACGCGUGUAUGUAUGCAUCGUCUCUCCUCCGUCCCG |
| | | ACUACCAUCAGCAGCACCACUACCGCCACCCCCAGCGCCACCACCGC |
| | | UGCCGUCGCCACCGCGUUAUCCGUUCCUCGUAGGCUGGUCCUGGGG |
| | | AACGGGUCGGCGGCCGGUCGGCUUCUGUUUUAUUAUUUUUUUUUA |
| | | UUUUUUAUCUUCUCCUUUCCUUAAUCUCGGAUUAUCAUUUCCCUCU |
| | | CCUACCUACCACGAAUCGCAGAUGAUAAACAAGAGGGUAAAAAGA |
| | | AAAAAGCUACAGACAUUUGGGUACCUCAGCUUUCCGAUAACUCGA |
| | | AGAAUUCAAAGUCGACGAUUCCCAACAAGAGAAAACAGAACAAAA |
| | | ACAAGGUCAUUUUUAUUUAUCCUCAUCGUCAACAACAACUACCGAC |
| | | AACAACGAAACACCACCAAGAAUGUCAAUCCGCAAGGGUGUUCCUG |
| | | CCCCCUCGACGCGCCUGUCGCGAUCCUCAUGGCGAGGACCGCGAUC |
| | | UCCGUAUAGGUAGAUGAAAUUAUCCCGUGUCCGGUCCUGAUUCCCC |
| | | GCAUGCCCUGCACAUCCUGACGCGUCGGUCAGCAGCCAAACAAUCA |
| | | UAGGAAAUGAACCAGAAGAACAAAAAGAUCAUCUCUCUCGGUGUA |
| | | UAGCAACACCAACAACAACCGCAUCGCAACAUCUUCAUCCGCAAGA |
| | | CGGAAAGAAAACAACAAUAAUGAGAAUGAAAUCACCACAACCAAG |
| | | CCAGAUUUCACGUCCAUGAGUUUUUAUUAUAUUAUUAUCAAAACG |
| | | AAAAACAGAAAAACUGUCAUAGAUAAAUAUAAAAAAAAAUAGAAA |
| | | CCACAAACGACUACUAGUACUCCAAUCUUAGAUGUAUAUGCUCCUA |
| | | GAUAAGAUUUAGUAUUACCAUAAUCAUCGAAGAAUGAAAGACGAC |
| | | GAUGAUUCCUUACCGCUCCUGCCACCCGGUCUGUAUGUAGAGAGAG |
| | | AAGAGAGAAAACGGUGAAUCCAAGAUCCCCGGGUCGGCGUCGGCA |
| | | UGCCGCUGAUCGCAGUGGCCCCACCUCGGCAUGCCGGCGCCGGGCG |
| | | AGGAAUUGCUCAUGAAAAAAGUAUCUUUCUGUAAAAAAAGAAAAC |
| | | AAUACAUGAUUAACCGAAAAGAAACCAACAAAAAGAACCCGAGAU |
| | | CAGUCGAUUUCGAUCACUACGAUAAACACAUGGAAGAUUUCUUGA |
| | | AAAAAGAAAAGAGAAAGAGACCACCUUCCCGGCGGCGGACACGCUC |
| | | CUCUCCGUCGCCGUUCUGCACCAUGAUUCGAUCAAUAACAACAUCA |
| | | UCAUCGGAGACCAUCUUUUAAUCAAUCAGCGUUGCAGUAGUCGAC |
| | | UCCCUGGACACGAAGGAGUCAUCCAUUUUUAUCCUCGCACUUCUUC |
| | | GCUCUCAAAGCCGCCUUUAAAGUUGAAAUGAAAGGAUGGAAACAU |
| | | GGAAUACAGUUUUAAUUGCACGUAUCACCAUUUUACUACAAAAAG |
| | | AAAAAAAAACAACUUACACAUAGUAUUACCUUAGGUUUACGGAUA |
| | | AGUAGAGUGUAGGCGUUUUUGAAACAGUUCAGCCAAUGCAAUCUU |
| | | GUCUCGGCAUAAUCACUCUUUCUGCAUAUAAUAGUAGUAGUAGAU |
| | | UUAUUCACAUCAACACAGCGAAAAACUCCAGCAUCAAAGUACACCU |
| | | AGAGACAGCCCUUAAAAUAUAGUUUGCAGCUUUUAGAUGUACUUA |
| | | CACCAAAGAAGAUUACCGUCCUUACGAGAAAACAGAUACUCGGAU |
| | | AUAGGAAUCAAGACAGCUCUGCACUGAAAACACACUCUCCUGUCAC |
| | | GACACCGCGCCACACCAGAGGCGUACGCGUGACUUCAUCGCAACGA |
| | | UCCAUCGUGAUGUCCCUCGCAGAACCUAAAAAGACCAAAAAAAAA |
| | | UCUUGGACCACAGUUGUCGAUUCUUGAAGACAAUAUUCUCGUGAG |
| | | AACUUUGAGAUUCGCACUUGAAACCUCUUAGGAUCCACAAAAACA |
| | | ACAACCUCUGUAUGGAAAAUGCGCUAUUUUAUCUCAGCUUUUCUC |
| | | CCAAACCUCGGUUUCUUCCUAUUCUUAAGUUUUCCCUAGUAUAUU |
| | | UGCCUCCUUAUAAGAAAAGAAGCACAAGCUCGGUCGCACGGAUUA |
| | | UUCCUUCUGCUAAUCUAUUAUUUUGUUCCUUUUUUUUUUGUUUGC |
| | | CUUCACCCCCUUCACUCCCUGUAGCAACACAGAGUAGUAGACACAA |
| | | UAAAUGAGAAGU |
| 80 | mtGFP_s_β2.7_ | AUAGUGAGCAAGGGCGAGGAGCUGUUCACCGGGGUGGUGCCCAUC |
| | core | CUGGUCGAGCUGGACGGCGACGUAAACGGCCACAAGUUCAGCGUG |
| | | UCCGGCGAGGGCGAGGGCGAUGCCACCUACGGCAAGCUGACCCUGA |
| | | AGUUCAUCUGCACCACCGGCAAGCUGCCCGUGCCCUGGCCCACCCU |
| | | CGUGACCACCCUGACCUACGGCGUGCAGUGCUUCAGCCGCUACCCC |
| | | GACCACAUGAAGCAGCACGACUUCUUCAAGUCCGCCAUGCCCGAAG |
| | | GCUACGUCCAGGAGCGCACCAUCUUCUUCAAGGACGACGGCAACUA |
| | | CAAGACCCGCGCCGAGGUGAAGUUCGAGGGCGACACCCUGGUGAAC |
| | | CGCAUCGAGCUGAAGGGCAUCGACUUCAAGGAGGACGGCAACAUCC |
| | | UGGGGCACAAGCUGGAGUACAACUACAACAGCCACAACGUCUAUA |
| | | UCAUGGCCGACAAGCAGAAGAACGGCAUCAAGGUGAACUUCAAGA |
| | | UCCGCCACAACAUCGAGGACGGCAGCGUGCAGCUCGCCGACCACUA |
| | | CCAGCAGAACACCCCCAUCGGCGACGGCCCCGUGCUGCUGCCCGAC |
| | | AACCACUACCUGAGCACCCAGUCCGCCCUGAGCAAAGACCCCAACG |
| | | AGAAGCGCGAUCACAUGGUCCUGCUGGAGUUCGUGACCGCCGCCGG |
| | | GAUCACUCUCGGCAUGGACGAGCUGUACAAGAGAUCCCCAGAUCGC |
| | | UGCUGCCCCGGCGUUCUCCAGAAGCCCCGGCGGGCGAAUCGGCCGG |
| | | CUGGUCGGUCGGCGCUCGGACGGAUGGGGAGAACGGCGGUGACUU |
| | | AGCCGCCCGUGGCCGGGAGAAGAUGGAGGAGCCGAGAUGACAACG |
| | | GCAGUCGUGGAAGGGUCGCCAAGCCCCGGUCCUUCUCUUCUGUCUG |
| | | GUCGAAUCUCGUUUUCUUUUUUCAACCGCUCUUUUUAUCACCUUU |
| | | UUAUGUGAGUUUCUCUUCCGCGUCUCCCGGCCGUACCAUCCACCCA |
| | | UGCAGCAUGCACGCGUGUAUGUAUGCAUCGUCUCUCCUCCGUCCCG |
| | | ACUACCAUCAGCAGCACCACUACCGCCACCCCCAGCGCCACCACCGC |
| | | UGCCGUCGCCACCGCGUUAUCCGUUCCUCGUAGGCUGGUCCUGGGG |
| | | AACGGGUCGGCGGCCGGUCGGCUUCUGUUUUAUUAUUUUUUUUUA |
| | | UUUUUUAUCUUCUCCUUUCCUUAAUCUCGGAUUAUCAUUUCCCUCU |
| | | CCUACCUACCACGAAUCGCAGAUGAUAAACAAGAGGGUAAAAAGA |
| | | AAAAAGCUACAGACAUUUGGGUACCUCAGCUUUCCGAUAACUCGA |
| | | AGAAUUCAAAGUCGACGAUUCCCAACAAGAGAAAACAGAACAAAA |
| | | ACAAGGUCAUUUUUAUUUAUCCUCAUCGUCAACAACAACUACCGAC |
| | | AACAACGAAACACCACCAAGAAUGUCAAUCCGCAAGGGUGUUCCUG |
| | | CCCCCUCGACGCGCCUGUCGCGAUCCUCAUGGCGAGGACCGCGAUC |
| | | UCCGUAUAGGUAGAUGAAAUUAUCCCGUGUCCGGUCCUGAUUCCCC |
| | | GCAUGCCCUGCACAUCCUGACGCGUCGGUCAGCAGCCAAACAAUCA |
| | | UAGGAAAUGAACCAGAAGAACAAAAAGAUCAUCUCUCUCGGUGUA |
| | | UAGCAACACCAACAACAACCGCAUCGCAACAUCUUCAUCCGCAAGA |
| | | CGGAAAGAAAACAACAAUAAUGAGAAUGAAAUCACCACAACCAAG |
| | | CCAGAUUUCACGUCCAUGAGUUUUUAUUAUAUUAUUAUCAAAACG |
| | | AAAAACAGAAAAACUGUCAUAGAUAAAUAUAAAAAAAAAUAGAAA |
| | | CCACAAACGACUACUAGUACUCCAAUCUUAGAUGUAUAUGCUCCUA |
| | | GAUAAGAUUUAGUAUUACCAUAAUCAUCGAAGAAUGAAAGACGAC |
| | | GAUGAUUCCUUACCGCUCCUGCCACCCGGUCUGUAUGUAGAGAGAG |
| | | AAGAGAGAAAACGGUGAAUCCAAGAUCCCCGGGUCGGCGUCGGCA |
| | | UGCCGCUGAUCGCAGUGGCCCCACCUCGGCAUGCCGGCGCCGGGCG |
| | | AGGAAUUGCUCAUGAAAAAAGUAUCUUUCUGUAAAAAAAGAAAAC |
| | | AAUACAUGAUUAACCGAAAAGAAACCAACAAAAAGAACCCGAGAU |
| | | CAGUCGAUUUCGAUCACUACGAUAAACACAUGGAAGAUUUCUUGA |
| | | AAAAAGAAAAGAGAAAGAGACCACCUUCCCGGCGGCGGACACGCUC |
| | | CUCUCCGUCGCCGUUCUGCACCAUGAUUCGAUCAAUAACAACAUCA |
| | | UCAUCGGAGACCAUCUUUUAAUCAAUCAGCGUUGCAGUAGUCGAC |
| | | UCCCUGGACACGAAGGAGUCAUCCAUUUUUAUCCUCGCACUUCUUC |
| | | GCUCUCAAAGCCGCCUUUAAAGUUGAAAUGAAAGGAUGGAAACAU |
| | | GGAAUACAGUUUUAAUUGCACGUAUCACCAUUUUACUACAAAAAG |
| | | AAAAAAAAACAACUUACACAUAGUAUUACCUUAGGUUUACGGAUA |
| | | AGUAGAGUGUAGGCGUUUUUGAAACAGUUCAGCCAAUGCAAUCUU |
| | | GUCUCGGCAUAAUCACUCUUUCUGCAUAUAAUAGUAGUAGUAGAU |
| | | UUAUUCACAUCAACACAGCGAAAAACUCCAGCAUCAAAGUACACCU |
| | | AGAGACAGCCCUUAAAAUAUAGUUUGCAGCUUUUAGAUGUACUUA |
| | | CACCAAAGAAGAUUACCGUCCUUACGAGAAAACAGAUACUCGGAU |
| | | AUAGGAAUCAAGACAGCUCUGCACUGAAAACACACUCUCCUGUCAC |
| | | GACACCGCGCCACACCAGAGGCGUACGCGUGACUUCAUCGCAACGA |
| | | UCCAUCGUGAUGUCCCUCGCAGAACCUAAAAAGACCAAAAAAAAA |
| | | UCUUGGACCACAGUUGUCGAUUCUUGAAGACAAUAUUCUCGUGAG |
| | | AACUUUGAGAUUCGCACUUGAAACCUCUUAGGAUCCACAAAAACA |
| | | ACAACCUCUGUAUGGAAAAUGCGCUAUUUUAUCUCAGCUUUUCUC |
| | | CCAAACCUCGGUUUCUUCCUAUUCUUAAGUUUUCCCUAGUAUAUU |
| | | UGCCUCCUUAUAAGAAAAGAAGCACAAGCUCGGUCGCACGGAUUA |
| | | UUCCUUCUGCUAAUCUAUUAUUUUGUUCCUUUUUUUUUUGUUUGC |
| | | CUUCACCCCCUUCACUCCCUGUAGCAACACAGAGUAGUAGACACAA |
| | | UAAAUGAGAAGU |
| 81 | mtGFP_s_ | AUAGUGAGCAAGGGCGAGGAGCUGUUCACCGGGGUGGUGCCCAUC |
| | core | CUGGUCGAGCUGGACGGCGACGUAAACGGCCACAAGUUCAGCGUG |
| | | UCCGGCGAGGGCGAGGGCGAUGCCACCUACGGCAAGCUGACCCUGA |
| | | AGUUCAUCUGCACCACCGGCAAGCUGCCCGUGCCCUGGCCCACCCU |
| | | CGUGACCACCCUGACCUACGGCGUGCAGUGCUUCAGCCGCUACCCC |
| | | GACCACAUGAAGCAGCACGACUUCUUCAAGUCCGCCAUGCCCGAAG |
| | | GCUACGUCCAGGAGCGCACCAUCUUCUUCAAGGACGACGGCAACUA |
| | | CAAGACCCGCGCCGAGGUGAAGUUCGAGGGCGACACCCUGGUGAAC |
| | | CGCAUCGAGCUGAAGGGCAUCGACUUCAAGGAGGACGGCAACAUCC |
| | | UGGGGCACAAGCUGGAGUACAACUACAACAGCCACAACGUCUAUA |
| | | UCAUGGCCGACAAGCAGAAGAACGGCAUCAAGGUGAACUUCAAGA |
| | | UCCGCCACAACAUCGAGGACGGCAGCGUGCAGCUCGCCGACCACUA |
| | | CCAGCAGAACACCCCCAUCGGCGACGGCCCCGUGCUGCUGCCCGAC |
| | | AACCACUACCUGAGCACCCAGUCCGCCCUGAGCAAAGACCCCAACG |
| | | AGAAGCGCGAUCACAUGGUCCUGCUGGAGUUCGUGACCGCCGCCGG |
| | | GAUCACUCUCGGCAUGGACGAGCUGUACAAGAGA |
| 82 | asATP6_s6_ | AGUAGAAUUAGAAUUGUGAAGAUGAUAAGUGUAGAGGGAAGGUUA |
| | β2.7_core | AUGGUUGAUAUUGCUAGGGUGGCGCUUCCAAUUAGGUGCGUGAGU |
| | | AGGUGGCCUGCAGUAAUGUUAUCCCCAGAUCGCUGCUGCCCCGGCG |
| | | UUCUCCAGAAGCCCCGGCGGGCGAAUCGGCCGGCUGGUCGGUCGGC |
| | | GCUCGGACGGAUGGGGAGAACGGCGGUGACUUAGCCGCCCGUGGCC |
| | | GGGAGAAGAUGGAGGAGCCGAGAUGACAACGGCAGUCGUGGAAGG |
| | | GUCGCCAAGCCCCGGUCCUUCUCUUCUGUCUGGUCGAAUCUCGUUU |
| | | UCUUUUUUCAACCGCUCUUUUUAUCACCUUUUUAUGUGAGUUUCU |
| | | CUUCCGCGUCUCCCGGCCGUACCAUCCACCCAUGCAGCAUGCACGC |
| | | GUGUAUGUAUGCAUCGUCUCUCCUCCGUCCCGACUACCAUCAGCAG |
| | | CACCACUACCGCCACCCCCAGCGCCACCACCGCUGCCGUCGCCACCG |
| | | CGUUAUCCGUUCCUCGUAGGCUGGUCCUGGGGAACGGGUCGGCGGC |
| | | CGGUCGGCUUCUGUUUUAUUAUUUUUUUUUAUUUUUUAUCUUCUC |
| | | CUUUCCUUAAUCUCGGAUUAUCAUUUCCCUCUCCUACCUACCACGA |
| | | AUCGCAGAUGAUAAACAAGAGGGUAAAAAGAAAAAAGCUACAGAC |
| | | AUUUGGGUACCUCAGCUUUCCGAUAACUCGAAGAAUUCAAAGUCG |
| | | ACGAUUCCCAACAAGAGAAAACAGAACAAAAACAAGGUCAUUUUU |
| | | AUUUAUCCUCAUCGUCAACAACAACUACCGACAACAACGAAACACC |
| | | ACCAAGAAUGUCAAUCCGCAAGGGUGUUCCUGCCCCCUCGACGCGC |
| | | CUGUCGCGAUCCUCAUGGCGAGGACCGCGAUCUCCGUAUAGGUAGA |
| | | UGAAAUUAUCCCGUGUCCGGUCCUGAUUCCCCGCAUGCCCUGCACA |
| | | UCCUGACGCGUCGGUCAGCAGCCAAACAAUCAUAGGAAAUGAACCA |
| | | GAAGAACAAAAAGAUCAUCUCUCUCGGUGUAUAGCAACACCAACA |
| | | ACAACCGCAUCGCAACAUCUUCAUCCGCAAGACGGAAAGAAAACAA |
| | | CAAUAAUGAGAAUGAAAUCACCACAACCAAGCCAGAUUUCACGUCC |
| | | AUGAGUUUUUAUUAUAUUAUUAUCAAAACGAAAAACAGAAAAACU |
| | | GUCAUAGAUAAAUAUAAAAAAAAAUAGAAACCACAAACGACUACU |
| | | AGUACUCCAAUCUUAGAUGUAUAUGCUCCUAGAUAAGAUUUAGUA |
| | | UUACCAUAAUCAUCGAAGAAUGAAAGACGACGAUGAUUCCUUACC |
| | | GCUCCUGCCACCCGGUCUGUAUGUAGAGAGAGAAGAGAGAAAACG |
| | | GUGAAUCCAAGAUCCCCGGGUCGGCGUCGGCAUGCCGCUGAUCGCA |
| | | GUGGCCCCACCUCGGCAUGCCGGCGCCGGGCGAGGAAUUGCUCAUG |
| | | AAAAAAGUAUCUUUCUGUAAAAAAAGAAAACAAUACAUGAUUAAC |
| | | CGAAAAGAAACCAACAAAAAGAACCCGAGAUCAGUCGAUUUCGAU |
| | | CACUACGAUAAACACAUGGAAGAUUUCUUGAAAAAAGAAAAGAGA |
| | | AAGAGACCACCUUCCCGGCGGCGGACACGCUCCUCUCCGUCGCCGU |
| | | UCUGCACCAUGAUUCGAUCAAUAACAACAUCAUCAUCGGAGACCAU |
| | | CUUUUAAUCAAUCAGCGUUGCAGUAGUCGACUCCCUGGACACGAA |
| | | GGAGUCAUCCAUUUUUAUCCUCGCACUUCUUCGCUCUCAAAGCCGC |
| | | CUUUAAAGUUGAAAUGAAAGGAUGGAAACAUGGAAUACAGUUUUA |
| | | AUUGCACGUAUCACCAUUUUACUACAAAAAGAAAAAAAAACAACU |
| | | UACACAUAGUAUUACCUUAGGUUUACGGAUAAGUAGAGUGUAGGC |
| | | GUUUUUGAAACAGUUCAGCCAAUGCAAUCUUGUCUCGGCAUAAUC |
| | | ACUCUUUCUGCAUAUAAUAGUAGUAGUAGAUUUAUUCACAUCAAC |
| | | ACAGCGAAAAACUCCAGCAUCAAAGUACACCUAGAGACAGCCCUUA |
| | | AAAUAUAGUUUGCAGCUUUUAGAUGUACUUACACCAAAGAAGAUU |
| | | ACCGUCCUUACGAGAAAACAGAUACUCGGAUAUAGGAAUCAAGAC |
| | | AGCUCUGCACUGAAAACACACUCUCCUGUCACGACACCGCGCCACA |
| | | CCAGAGGCGUACGCGUGACUUCAUCGCAACGAUCCAUCGUGAUGUC |
| | | CCUCGCAGAACCUAAAAAGACCAAAAAAAAAUCUUGGACCACAGU |
| | | UGUCGAUUCUUGAAGACAAUAUUCUCGUGAGAACUUUGAGAUUCG |
| | | CACUUGAAACCUCUUAGGAUCCACAAAAACAACAACCUCUGUAUGG |
| | | AAAAUGCGCUAUUUUAUCUCAGCUUUUCUCCCAAACCUCGGUUUCU |
| | | UCCUAUUCUUAAGUUUUCCCUAGUAUAUUUGCCUCCUUAUAAGAA |
| | | AAGAAGCACAAGCUCGGUCGCACGGAUUAUUCCUUCUGCUAAUCU |
| | | AUUAUUUUGUUCCUUUUUUUUUUGUUUGCCUUCACCCCCUUCACUC |
| | | CCUGUAGCAACACAGAGUAGUAGACACAAUAAAUGAGAAGU |
| 83 | β2.7_s8_as | UCCCCAGAUCGCUGCUGCCCCGGCGUUCUCCAGAAGCCCCGGCGGG |
| | ATP8_core | CGAAUCGGCCGGCUGGUCGGUCGGCGCUCGGACGGAUGGGGAGAA |
| | | CGGCGGUGACUUAGCCGCCCGUGGCCGGGAGAAGAUGGAGGAGCC |
| | | GAGAUGACAACGGCAGUCGUGGAAGGGUCGCCAAGCCCCGGUCCUU |
| | | CUCUUCUGUCUGGUCGAAUCUCGUUUUCUUUUUUCAACCGCUCUUU |
| | | UUAUCACCUUUUUAUGUGAGUUUCUCUUCCGCGUCUCCCGGCCGUA |
| | | CCAUCCACCCAUGCAGCAUGCACGCGUGUAUGUAUGCAUCGUCUCU |
| | | CCUCCGUCCCGACUACCAUCAGCAGCACCACUACCGCCACCCCCAGC |
| | | GCCACCACCGCUGCCGUCGCCACCGCGUUAUCCGUUCCUCGUAGGC |
| | | UGGUCCUGGGGAACGGGUCGGCGGCCGGUCGGCUUCUGUUUUAUU |
| | | AUUUUUUUUUAUUUUUUAUCUUCUCCUUUCCUUAAUCUCGGAUUA |
| | | UCAUUUCCCUCUCCUACCUACCACGAAUCGCAGAUGAUAAACAAGA |
| | | GGGUAAAAAGAAAAAAGCUACAGACAUUUGGGUACCUCAGCUUUC |
| | | CGAUAACUCGAAGAAUUCAAAGUCGACGAUUCCCAACAAGAGAAA |
| | | ACAGAACAAAAACAAGGUCAUUUUUAUUUAUCCUCAUCGUCAACA |
| | | ACAACUACCGACAACAACGAAACACCACCAAGAAUGUCAAUCCGCA |
| | | AGGGUGUUCCUGCCCCCUCGACGCGCCUGUCGCGAUCCUCAUGGCG |
| | | AGGACCGCGAUCUCCGUAUAGGUAGAUGAAAUUAUCCCGUGUCCG |
| | | GUCCUGAUUCCCCGCAUGCCCUGCACAUCCUGACGCGUCGGUCAGC |
| | | AGCCAAACAAUCAUAGGAAAUGAACCAGAAGAACAAAAAGAUCAU |
| | | CUCUCUCGGUGUAUAGCAACACCAACAACAACCGCAUCGCAACAUC |
| | | UUCAUCCGCAAGACGGAAAGAAAACAACAAUAAUGAGAAUGAAAU |
| | | CACCACAACCAAGCCAGAUUUCACGUCCAUGAGUUUUUAUUAUAU |
| | | UAUUAUCAAAACGAAAAACAGAAAAACUGUCAUAGAUAAAUAUAA |
| | | AAAAAAAUAGAAACCACAAACGACUACUAGUACUCCAAUCUUAGA |
| | | UGUAUAUGCUCCUAGAUAAGAUUUAGUAUUACCAUAAUCAUCGAA |
| | | GAAUGAAAGACGACGAUGAUUCCUUACCGCUCCUGCCACCCGGUCU |
| | | GUAUGUAGAGAGAGAAGAGAGAAAACGGUGAAUCCAAGAUCCCCG |
| | | GGUCGGCGUCGGCAUGCCGCUGAUCGCAGUGGCCCCACCUCGGCAU |
| | | GCCGGCGCCGGGCGAGGAAUUGCUCAUGAAAAAAGUAUCUUUCUG |
| | | UAAAAAAAGAAAACAAUACAUGAUUAACCGAAAAGAAACCAACAA |
| | | AAAGAACCCGAGAUCAGUCGAUUUCGAUCACUACGAUAAACACAU |
| | | GGAAGAUUUCUUGAAAAAAGAAAAGAGAAAGAGACCACCUUCCCG |
| | | GCGGCGGACACGCUCCUCUCCGUCGCCGUUCUGCACCAUGAUUCGA |
| | | UCAAUAACAACAUCAUCAUCGGAGACCAUCUUUUAAUCAAUCAGC |
| | | GUUGCAGUAGUCGACUCCCUGGACACGAAGGAGUCAUCCAUUUUU |
| | | AUCCUCGCACUUCUUCGCUCUCAAAGCCGCCUUUAAAGUUGAAAUG |
| | | AAAGGAUGGAAACAUGGAAUACAGUUUUAAUUGCACGUAUCACCA |
| | | UUUUACUACAAAAAGAAAAAAAAACAACUUACACAUAGUAUUACC |
| | | UUAGGUUUACGGAUAAGUAGAGUGUAGGCGUUUUUGAAACAGUUC |
| | | AGCCAAUGCAAUCUUGUCUCGGCAUAAUCACUCUUUCUGCAUAUA |
| | | AUAGUAGUAGUAGAUUUAUUCACAUCAACACAGCGAAAAACUCCA |
| | | GCAUCAAAGUACACCUAGAGACAGCCCUUAAAAUAUAGUUUGCAG |
| | | CUUUUAGAUGUACUUACACCAAAGAAGAUUACCGUCCUUACGAGA |
| | | AAACAGAUACUCGGAUAUAGGAAUCAAGACAGCUCUGCACUGAAA |
| | | ACACACUCUCCUGUCACGACACCGCGCCACACCAGAGGCGUACGCG |
| | | UGACUUCAUCGCAACGAUCCAUCGUGAUGUCCCUCGCAGAACCUAA |
| | | AAAGACCAAAAAAAAAUCUUGGACCACAGUUGUCGAUUCUUGAAG |
| | | ACAAUAUUCUCGUGAGAACUUUGAGAUUCGCACUUGAAACCUCUU |
| | | AGGAUCCACAAAAACAACAACCUCUGUAUGGAAAAUGCGCUAUUU |
| | | UAUCUCAGCUUUUCUCCCAAACCUCGGUUUCUUCCUAUUCUUAAGU |
| | | UUUCCCUAGUAUAUUUGCCUCCUUAUAAGAAAAGAAGCACAAGCU |
| | | CGGUCGCACGGAUUAUUCCUUCUGCUAAUCUAUUAUUUUGUUCCU |
| | | UUUUUUUUUGUUUGCCUUCACCCCCUUCACUCCCUGUAGCAACACA |
| | | GAGUAGUAGACACAAUAAAUGAGAAGUUUUCGUUCAUUUUGGUUC |
| | | UCAGGGUUUGUUAUAAUUUUUUAUUUUUAUGGGCUUUGGUGAGGG |
| | | AGGUAGGUGGUAGUUUGUGUUUAAUAUUUUUAGUUGGGUGAUGA |
| 84 | ATP6_D3₄_ | AGUAGAAUUAGAAUUGUGAAGAUGAUAAGUGUAGAGGGAAGGUUA |
| | D2₄_(core) | AUGGUUGAUAUUGCUAGGGUGGCGCUUCCAAUUAGGUGCGUGAGU |
| | | AGGUGGCCUGCAGUAAUGUUGGUCAUUUUUAUUUAUCCUCAUCGU |
| | | CAACAACAACUACCGACAACAACGAAACACCACCAAGAAUGUCAAU |
| | | CCGCAAGGGUGUUCCUGCCCCCUCGACGCGCCUGUCGCGAUCCUCA |
| | | UGGCGAGGACCGCGAUCUCCGUAUAGGUAGAUGAAAUUAUCCCGU |
| | | GUCCGGUCCUGAUUCCCCGCAUGCCCUGCACAUCCUGACGCGUCGG |
| | | UCAGCAGCCAAACAAUCAUAGGAAAUGAACCGGUCAUUUUUAUUU |
| | | AUCCUCAUCGUCAACAACAACUACCGACAACAACGAAACACCACCA |
| | | AGAAUGUCAAUCCGCAAGGGUGUUCCUGCCCCCUCGACGCGCCUGU |
| | | CGCGAUCCUCAUGGCGAGGACCGCGAUCUCCGUAUAGGUAGAUGA |
| | | AAUUAUCCCGUGUCCGGUCCUGAUUCCCCGCAUGCCCUGCACAUCC |
| | | UGACGCGUCGGUCAGCAGCCAAACAAUCAUAGGAAAUGAACCGGU |
| | | CAUUUUUAUUUAUCCUCAUCGUCAACAACAACUACCGACAACAACG |
| | | AAACACCACCAAGAAUGUCAAUCCGCAAGGGUGUUCCUGCCCCCUC |
| | | GACGCGCCUGUCGCGAUCCUCAUGGCGAGGACCGCGAUCUCCGUAU |
| | | AGGUAGAUGAAAUUAUCCCGUGUCCGGUCCUGAUUCCCCGCAUGCC |
| | | CUGCACAUCCUGACGCGUCGGUCAGCAGCCAAACAAUCAUAGGAAA |
| | | UGAACCGGUCAUUUUUAUUUAUCCUCAUCGUCAACAACAACUACCG |
| | | ACAACAACGAAACACCACCAAGAAUGUCAAUCCGCAAGGGUGUUCC |
| | | UGCCCCCUCGACGCGCCUGUCGCGAUCCUCAUGGCGAGGACCGCGA |
| | | UCUCCGUAUAGGUAGAUGAAAUUAUCCCGUGUCCGGUCCUGAUUC |
| | | CCCGCAUGCCCUGCACAUCCUGACGCGUCGGUCAGCAGCCAAACAA |
| | | UCAUAGGAAAUGAACCUUUUAUUUUUUAUCUUCUCCUUUCCUUAA |
| | | UCUCGGAUUAUCAUUUCCCUCUCCUACCUACCACGAAUCGCAGAUG |
| | | AUAAACAAGAGGGUAAAAAGAAAAAUUUUAUUUUUUAUCUUCUCC |
| | | UUUCCUUAAUCUCGGAUUAUCAUUUCCCUCUCCUACCUACCACGAA |
| | | UCGCAGAUGAUAAACAAGAGGGUAAAAAGAAAAAUUUUAUUUUUU |
| | | AUCUUCUCCUUUCCUUAAUCUCGGAUUAUCAUUUCCCUCUCCUACC |
| | | UACCACGAAUCGCAGAUGAUAAACAAGAGGGUAAAAAGAAAAAUU |
| | | UUAUUUUUUAUCUUCUCCUUUCCUUAAUCUCGGAUUAUCAUUUCC |
| | | CUCUCCUACCUACCACGAAUCGCAGAUGAUAAACAAGAGGGUAAA |
| | | AAGAAAAA |
| 85 | in vitro | GGCGCUUCCGGAAGAAAGCGCCAGUAGAAUUAGAAUUGUGAAGAU |
| | transcribed | GAUAAGUGUAGAGGGAAGGUUAAUGGUUGAUAUUGCUAGGGGGGC |
| | ATP6_(D3)₄_ | GCUUCCAAUUAGGUGCAUGAGUAGGUGGCCUGCAGUAAUGUUAAA |
| | (D2)₄ | GCAAGGGCCUUAUUCAUGGGCUAGCCCGGCGGUCAUUUUUAUUUA |
| | RNA with | UCCUCAUCGUCAACAACAACUACCGACAACAACGAAACACCACCAA |
| | indicated 5′ | GAAUGUCAAUCCGCAAGGGUGUUCCUGCCCCCUCGACGCGCCUGUC |
| | end, 3′ end, | GCGAUCCUCAUGGCGAGGACCGCGAUCUCCGUAUAGGUAGAUGAA |
| | domains | AUUAUCCCGUGUCCGGUCCUGAUUCCCCGCAUGCCCUGCACAUCCU |
| | D3 and D2. | GACGCGUCGGUCAGCAGCCAAACAAUCAUAGGAAAUGAACCGCCGG |
| | and spacers | GAAGCGCCGAAUUCAUAUCGAUCGGUCAUUUUUAUUUAUCCUCAU |
| | | CGUCAACAACAACUACCGACAACAACGAAACACCACCAAGAAUGUC |
| | | AAUCCGCAAGGGUGUUCCUGCCCCCUCGACGCGCCUGUCGCGAUCC |
| | | UCAUGGCGAGGACCGCGAUCUCCGUAUAGGUAGAUGAAAUUAUCC |
| | | CGUGUCCGGUCCUGAUUCCCCGCAUGCCCUGCACAUCCUGACGCGU |
| | | CGGUCAGCAGCCAAACAAUCAUAGGAAAUGAACCGAUCGAAUAAA |
| | | GGUACCUGUGGGGUCAUUUUUAUUUAUCCUCAUCGUCAACAACAA |
| | | CUACCGACAACAACGAAACACCACCAAGAAUGUCAAUCCGCAAGGG |
| | | UGUUCCUGCCCCCUCGACGCGCCUGUCGCGAUCCUCAUGGCGAGGA |
| | | CCGCGAUCUCCGUAUAGGUAGAUGAAAUUAUCCCGUGUCCGGUCCU |
| | | GAUUCCCCGCAUGCCCUGCACAUCCUGACGCGUCGGUCAGCAGCCA |
| | | AACAAUCAUAGGAAAUGAACCCCACAUUUUACCGGUAAUACCGGG |
| | | GGGUCAUUUUUAUUUAUCCUCAUCGUCAACAACAACUACCGACAAC |
| | | AACGAAACACCACCAAGAAUGUCAAUCCGCAAGGGUGUUCCUGCCC |
| | | CCUCGACGCGCCUGUCGCGAUCCUCAUGGCGAGGACCGCGAUCUCC |
| | | GUAUAGGUAGAUGAAAUUAUCCCGUGUCCGGUCCUGAUUCCCCGC |
| | | AUGCCCUGCACAUCCUGACGCGUCGGUCAGCAGCCAAACAAUCAUA |
| | | GGAAAUGAACCCCCCGGAAGCUUUCCGGAAGAGCUAGCUUUUAUU |
| | | UUUUAUCUUCUCCUUUCCUUAAUCUCGGAUUAUCAUUUCCCUCUCC |
| | | UACCUACCACGAAUCGCAGAUGAUAAACAAGAGGGUAAAAAGAAA |
| | | AAAGCGCGAAUUCCGAUCUUUUAUUUUUUAUCUUCUCCUUUCCUU |
| | | AAUCUCGGAUUAUCAUUUCCCUCUCCUACCUACCACGAAUCGCAGA |
| | | UGAUAAACAAGAGGGUAAAAAGAAAAAGAUCGUAUCCGGUACCUG |
| | | UGGUUUUAUUUUUUAUCUUCUCCUUUCCUUAAUCUCGGAUUAUCA |
| | | UUUCCCUCUCCUACCUACCACGAAUCGCAGAUGAUAAACAAGAGGG |
| | | UAAAAAGAAAAACCACACCUCCACCGGUGGGCCGUUUUAUUUUUU |
| | | AUCUUCUCCUUUCCUUAAUCUCGGAUUAUCAUUUCCCUCUCCUACC |
| | | UACCACGAAUCGCAGAUGAUAAACAAGAGGGUAAAAAGAAAAACG |
| | | GCCCAAGCUUGACUCCUAUAGUGUCACCUAAAUGUCUAGAUACUA |
| | | AGGGAGUCUUGC |
| 86 | Multimer | GGUCAUUUUUAUUUAUCCUCAUCGUCAACAACAACUACCGACAACA |
| | (D3)₄_(D2)₄ | ACGAAACACCACCAAGAAUGUCAAUCCGCAAGGGUGUUCCUGCCCC |
| | RNA | CUCGACGCGCCUGUCGCGAUCCUCAUGGCGAGGACCGCGAUCUCCG |
| | including | UAUAGGUAGAUGAAAUUAUCCCGUGUCCGGUCCUGAUUCCCCGCA |
| | domains | UGCCCUGCACAUCCUGACGCGUCGGUCAGCAGCCAAACAAUCAUAG |
| | D3 and D2 | GAAAUGAACCGCCGGGAAGCGCCGAAUUCAUAUCGAUCGGUCAUU |
| | and spacers | UUUAUUUAUCCUCAUCGUCAACAACAACUACCGACAACAACGAAAC |
| | | ACCACCAAGAAUGUCAAUCCGCAAGGGUGUUCCUGCCCCCUCGACG |
| | | CGCCUGUCGCGAUCCUCAUGGCGAGGACCGCGAUCUCCGUAUAGGU |
| | | AGAUGAAAUUAUCCCGUGUCCGGUCCUGAUUCCCCGCAUGCCCUGC |
| | | ACAUCCUGACGCGUCGGUCAGCAGCCAAACAAUCAUAGGAAAUGA |
| | | ACCGAUCGAAUAAAGGUACCUGUGGGGUCAUUUUUAUUUAUCCUC |
| | | AUCGUCAACAACAACUACCGACAACAACGAAACACCACCAAGAAUG |
| | | UCAAUCCGCAAGGGUGUUCCUGCCCCCUCGACGCGCCUGUCGCGAU |
| | | CCUCAUGGCGAGGACCGCGAUCUCCGUAUAGGUAGAUGAAAUUAU |
| | | CCCGUGUCCGGUCCUGAUUCCCCGCAUGCCCUGCACAUCCUGACGC |
| | | GUCGGUCAGCAGCCAAACAAUCAUAGGAAAUGAACCCCACAUUUU |
| | | ACCGGUAAUACCGGGGGGUCAUUUUUAUUUAUCCUCAUCGUCAAC |
| | | AACAACUACCGACAACAACGAAACACCACCAAGAAUGUCAAUCCGC |
| | | AAGGGUGUUCCUGCCCCCUCGACGCGCCUGUCGCGAUCCUCAUGGC |
| | | GAGGACCGCGAUCUCCGUAUAGGUAGAUGAAAUUAUCCCGUGUCC |
| | | GGUCCUGAUUCCCCGCAUGCCCUGCACAUCCUGACGCGUCGGUCAG |
| | | CAGCCAAACAAUCAUAGGAAAUGAACCCCCCGGAAGCUUUCCGGAA |
| | | GAGCUAGCUUUUAUUUUUUAUCUUCUCCUUUCCUUAAUCUCGGAU |
| | | UAUCAUUUCCCUCUCCUACCUACCACGAAUCGCAGAUGAUAAACAA |
| | | GAGGGUAAAAAGAAAAAAGCGCGAAUUCCGAUCUUUUAUUUUUUA |
| | | UCUUCUCCUUUCCUUAAUCUCGGAUUAUCAUUUCCCUCUCCUACCU |
| | | ACCACGAAUCGCAGAUGAUAAACAAGAGGGUAAAAAGAAAAAGAU |
| | | CGUAUCCGGUACCUGUGGUUUUAUUUUUUAUCUUCUCCUUUCCUU |
| | | AAUCUCGGAUUAUCAUUUCCCUCUCCUACCUACCACGAAUCGCAGA |
| | | UGAUAAACAAGAGGGUAAAAAGAAAAACCACACCUCCACCGGUGG |
| | | GCCGUUUUAUUUUUUAUCUUCUCCUUUCCUUAAUCUCGGAUUAUC |
| | | AUUUCCCUCUCCUACCUACCACGAAUCGCAGAUGAUAAACAAGAGG |
| | | GUAAAAAGAAAAA |
| 87 | Multimer | GGUCAUUUUUAUUUAUCCUCAUCGUCAACAACAACUACCGACAACA |
| | (D3)₄_(D2)₄ | ACGAAACACCACCAAGAAUGUCAAUCCGCAAGGGUGUUCCUGCCCC |
| | RNA core | CUCGACGCGCCUGUCGCGAUCCUCAUGGCGAGGACCGCGAUCUCCG |
| | only | UAUAGGUAGAUGAAAUUAUCCCGUGUCCGGUCCUGAUUCCCCGCA |
| | including | UGCCCUGCACAUCCUGACGCGUCGGUCAGCAGCCAAACAAUCAUAG |
| | domains | GAAAUGAACCGGUCAUUUUUAUUUAUCCUCAUCGUCAACAACAAC |
| | D3 and D2 | UACCGACAACAACGAAACACCACCAAGAAUGUCAAUCCGCAAGGGU |
| | but without | GUUCCUGCCCCCUCGACGCGCCUGUCGCGAUCCUCAUGGCGAGGAC |
| | any spacers | CGCGAUCUCCGUAUAGGUAGAUGAAAUUAUCCCGUGUCCGGUCCU |
| | | GAUUCCCCGCAUGCCCUGCACAUCCUGACGCGUCGGUCAGCAGCCA |
| | | AACAAUCAUAGGAAAUGAACCGGUCAUUUUUAUUUAUCCUCAUCG |
| | | UCAACAACAACUACCGACAACAACGAAACACCACCAAGAAUGUCAA |
| | | UCCGCAAGGGUGUUCCUGCCCCCUCGACGCGCCUGUCGCGAUCCUC |
| | | AUGGCGAGGACCGCGAUCUCCGUAUAGGUAGAUGAAAUUAUCCCG |
| | | UGUCCGGUCCUGAUUCCCCGCAUGCCCUGCACAUCCUGACGCGUCG |
| | | GUCAGCAGCCAAACAAUCAUAGGAAAUGAACCGGUCAUUUUUAUU |
| | | UAUCCUCAUCGUCAACAACAACUACCGACAACAACGAAACACCACC |
| | | AAGAAUGUCAAUCCGCAAGGGUGUUCCUGCCCCCUCGACGCGCCUG |
| | | UCGCGAUCCUCAUGGCGAGGACCGCGAUCUCCGUAUAGGUAGAUG |
| | | AAAUUAUCCCGUGUCCGGUCCUGAUUCCCCGCAUGCCCUGCACAUC |
| | | CUGACGCGUCGGUCAGCAGCCAAACAAUCAUAGGAAAUGAACCUU |
| | | UUAUUUUUUAUCUUCUCCUUUCCUUAAUCUCGGAUUAUCAUUUCC |
| | | CUCUCCUACCUACCACGAAUCGCAGAUGAUAAACAAGAGGGUAAA |
| | | AAGAAAAAUUUUAUUUUUUAUCUUCUCCUUUCCUUAAUCUCGGAU |
| | | UAUCAUUUCCCUCUCCUACCUACCACGAAUCGCAGAUGAUAAACAA |
| | | GAGGGUAAAAAGAAAAAUUUUAUUUUUUAUCUUCUCCUUUCCUUA |
| | | AUCUCGGAUUAUCAUUUCCCUCUCCUACCUACCACGAAUCGCAGAU |
| | | GAUAAACAAGAGGGUAAAAAGAAAAAUUUUAUUUUUUAUCUUCUC |
| | | CUUUCCUUAAUCUCGGAUUAUCAUUUCCCUCUCCUACCUACCACGA |
| | | AUCGCAGAUGAUAAACAAGAGGGUAAAAAGAAAAA |

The foregoing sequences are further understood by reference to the following explanation of colour codes that are additionally set forth along with the colour-shaded sequences in the international publication no. WO 2017/192102 Al, dated Nov. 9, 2017, for international application no. PCT/SG2017/050238, filed May 8, 2017, of which this application is the U.S. national stage.

Colour codes for sequences 1 to 84
Domain 1; Domain 2; Domain 3; Domain 4
UCAGAUC: Last 7 bases of CMV promoter;
GGCGCU: last 6 bases of T7 promoter
GGAGUC: Last 6 bases of SP6 promoter
AUG: Genomic start codon;
UGA: Genomic stop codon UCCGGA (Sequence only:
UCCGGA):
BspEI restriction site;
GCUAGC:
NheI restriction site
ACCACCCCGGAGAACAGCUCCUC: spacer stabilizing GFP
structure
AUA: mitochondria specific start codon;
AGA: mitochondria specific stop codon
GAAUUC: EcoRI restriction site;
ACCGGU: AgeI restriction site
AAGCUU: HindIII restriction site;
UCUAGA: XbaI restriction site
UCCGGA: BspEI restriction site;
AAUAAA: PolyA cleavage site
CCGGC: S1a;
GCCGG: S1b
CGAUC: S2a;
GAUCG: S2b
UGUGG: S3a;
CCACA: S3b
CCGGGG: S4a;
CCCCGG: S4b
AAGCAAGGGUUAUUCCGAAUUGG: S6a;
CCAAUUCGGAAUAACCC: S6b
GGUUGGAUUGGGG: S8a;
CCCCAAACCAACCUCUACCGGAAGCGCCUUUU: S8b
Antisense AP8; Antisense ATP6
Colour codes for SEQ ID NOs: 85 to 87
ATP6 antisense RNA
4 Domains D3 (separated by spacers)
4 Domains D2 (separated by spacers)
Promoter-derived sequence
GGCGCUUCCGGAAGAAAGCGCC
ATP6-(D3x4_D2x4) spacer:
AAAGCAAGGGCCUUAUUCAUGGGCUAGCCCGGC
D3 spacer 1:
GCCGGGAAGCGCCGAAUUCAUAUCGAUC
D3 spacer 2:
GAUCGAAUAAAGGUACCUGUGG
D3 spacer 3:
CCACAUUUUACCGGUAAUACCGGGG
(CCACAUUUUACCGGUAAUACCGGGG)
D3x4-D2x4 spacer: CCCCGGAAGCUUUCCGGAAGAGCUAGC
(CCCCGGAAGCUUUCCGGAAGAGCUAGC)
D2 spacer 1:
AGCGCGAAUUCCGAUC (AGCGCGAAUUCCGAUC)
D2 spacer 2:
GAUCGUAUCCGGUACCUGUGG (GAUCGUAUCCGGUACCUGUGG)
D2 spacer 3:
CCACACCUCCACCGGUGGGCCG (CCACACCUCCACCGGUGGGCCG)
RNA 3′ end including polyadenylation signal:
CGGCCCAAGCUUGACUCCUAUAGUGUCACCUAAAUGUCUAGAUACUAAG
GGAGUCUUGC

1. A nucleic acid delivery construct comprising at least one sense or antisense RNA subdomain of the human cytomegalovirus β2.7 RNA, wherein each subdomain is capable of localization within the mitochondria.

2. The nucleic acid delivery construct of claim 1, wherein the RNA sequences from human cytomegalovirus β2.7 RNA has a sequence identity of 90%, 91%, 92%, 93%, 94%, 95%, 96%, 97%, 98% or 99% of one or more of the RNA sequences selected from the group consisting of β2.7 RNA (SEQ ID NO: 1 or SEQ ID NO: 2), domain 1 (D1; SEQ ID NO: 3 or SEQ ID NO: 7) of β2.7 RNA, domain 2 (D2; SEQ ID NO: 4 or SEQ ID NO: 8) of β2.7 RNA, domain 3 (D3; SEQ ID NO: 5 or SEQ ID NO: 9) β2.7 RNA, domain 4 (D4; SEQ ID NO: 6 or SEQ ID NO: 10) of β2.7 RNA and combinations thereof.

3. The nucleic acid delivery construct of claim 1, wherein the RNA sequences from human cytomegalovirus β2.7 RNA are selected from the group consisting of β2.7 RNA (SEQ ID NO: 1 or SEQ ID NO: 2), domain 1 (D1; SEQ ID NO: 3 or SEQ ID NO: 7) of β2.7 RNA, domain 2 (D2; SEQ ID NO: 4 or SEQ ID NO: 8) of β2.7 RNA, domain 3 (D3; SEQ ID NO: 5 or SEQ ID NO: 9) of β2.7 RNA, domain 4 (D4; SEQ ID NO: 6 or SEQ ID NO: 10) of β2.7 RNA and combinations thereof.

4. The nucleic acid delivery construct of claim 3, wherein the one, or two, or three, or four, or more RNA sequences from human cytomegalovirus β2.7 RNA comprise sequences selected from the group consisting of the full length sequence of β2.7 RNA (SEQ ID NO: 1 (sense)), the full length sequence of β2.7 RNA (SEQ ID NO: 2 (antisense)), domain 1 of β2.7 RNA (SEQ ID NO: 3 (sense)), domain 1 of β2.7 RNA (SEQ ID NO: 7 (antisense)), domain 2 of β2.7 RNA (SEQ ID NO: 4 (sense)), domain 2 of β2.7 RNA (SEQ ID NO: 8 (antisense)), domain 3 of β2.7 RNA (SEQ ID NO: 5 (sense)), domain 3 of β2.7 RNA (SEQ ID NO: 9 (antisense)), domain 4 of β2.7 RNA (SEQ ID NO: 6 (sense)), and domain 4 of β2.7 RNA (SEQ ID NO: 10 (antisense)).

5. The nucleic acid delivery construct of claim 3, wherein the RNA sequence from human cytomegalovirus β2.7 RNA comprises domain 2, domain 3 and domain 4 of β2.7 RNA (SEQ ID NO: 11 (sense) or SEQ ID NO: 39 (antisense)); or domain 1, domain 3 and domain 4 of β2.7 RNA (SEQ ID NO: 12 (sense) or SEQ ID NO: 40 (antisense)); or domain 1, domain 2 and domain 4 of β2.7 RNA (SEQ ID NO: 13 (sense) or SEQ ID NO: 41 (antisense)); or domain 1, domain 2 and domain 3 of β2.7 RNA (SEQ ID NO: 14 (sense) or SEQ ID NO: 42 (antisense)).

6. The nucleic acid delivery construct of claim 1, wherein the nucleic acid delivery construct comprises at least one spacer sequences.

7. The nucleic acid delivery construct of claim 6, wherein if the nucleic acid delivery construct comprises at least two spacer sequences, at least one spacer sequence is at the 5′ end and at least one another spacer sequence is at the 3′ end of the RNA sequence from human cytomegalovirus β2.7 RNA.

8. The nucleic acid delivery construct of any of claim 6, wherein the at least one spacer sequence is selected from the group consisting of S la (SEQ ID NO:26), S1b (SEQ ID NO:27), S2a (SEQ ID NO:28), S2b (SEQ ID NO:29), S3a (SEQ ID NO:30), S3b (SEQ ID NO:31), S4a (SEQ ID NO:32), S4b (SEQ ID NO:33), Sha (SEQ ID NO:34), S6b (SEQ ID NO:35), S8a (SEQ ID NO:36), S8b (SEQ ID NO:37) and Spacer F3A (SEQ ID NO: 38); and/or optionally, wherein the spacer sequence comprises a stop codon.

9. The nucleic acid delivery construct of claim 3, wherein each RNA sequence is selected from the group consisting of domain 1 (D1; SEQ ID NO: 3 or SEQ ID NO: 7), domain 2 (D2 SEQ ID NO: 4 or SEQ ID NO: 8), domain 3 (D3; SEQ ID NO: 5 or SEQ ID NO: 9) and domain 4 (D4; SEQ ID NO: 6 or SEQ ID NO: 10) of β2.7 RNA, according to formula I: 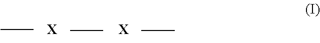
wherein each Xis independently selected from the group consisting of D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO:
6 or SEQ ID NO: 10) or combinations thereof, including duplicates, triplicates, quadruplicates, quintuplicates, sextuplicates, septuplicates, octuplicates, or longer repeats of single domains; and wherein each X is optionally preceded or followed or flanked by at least one or more spacer sequences as defined in claims 6 to 8.

10. The nucleic acid delivery construct of claim 3, wherein the RNA sequence from human cytomegalovirus β2.7 RNA is a tetramer of a RNA sequences, wherein each RNA sequence is selected from the group consisting of domain 1 (D1; SEQ ID NO: 3 or SEQ ID NO: 7), domain 2 (D2 SEQ ID NO: 4 or SEQ ID NO: 8), domain 3 (D3; SEQ ID NO: 5 or SEQ ID NO: 9) and domain 4 (D4; SEQ ID NO: 6 or SEQ ID NO: 10) of β2.7 RNA, according to formula II: 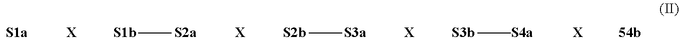
wherein each Xis independently selected from the group consisting of D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as defined in claim 8.

11. The nucleic acid delivery construct of claim 10, wherein each X is D2 (SEQ ID NO: 4 or SEQ ID NO: 8); or wherein each X is D3 (SEQ ID NO: 5 or SEQ ID NO: 9); or wherein the nucleic acid delivery construct comprises a nucleic acid sequence according to SEQ ID NO: 15 or SEQ ID NO: 51 or wherein the nucleic acid delivery construct comprises a nucleic acid sequence according to SEQ ID NO: 16 or SEQ ID NO: 52.

12. The nucleic acid delivery construct of claim 3, wherein the RNA sequence from human cytomegalovirus β2.7 RNA is an octamer of a RNA sequences selected from the group consisting of domain 1 (D1; SEQ ID NO: 3 or SEQ ID NO: 7), domain 2 (D2; SEQ ID NO: 4 or SEQ ID NO: 8), domain 3 (D3; SEQ ID NO: 5 or SEQ ID NO: 9) and domain 4 (D4; SEQ ID NO: 6 or SEQ ID NO: 10) of β2.7 RNA, according to formula III: 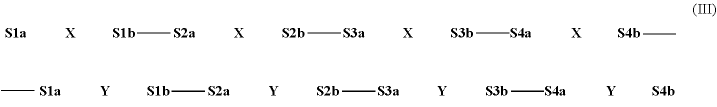
wherein X and Y are different from each other, wherein each X and each Y are independently selected from the group consisting of D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as defined in claim 8.

13. The nucleic acid delivery construct of claim 12, wherein X is D3 (SEQ ID NO: 5 or SEQ ID NO: 9) and Y is D2 (SEQ ID NO: 4 or SEQ ID NO: 8); or wherein X is D2 (SEQ ID NO: 4 or SEQ ID NO: 8) and Y is D3 (SEQ ID NO: 5 or SEQ ID NO: 9); or wherein the nucleic acid delivery construct comprises a nucleic acid sequence according to SEQ ID NO: 17 or SEQ ID NO: 53; or wherein the nucleic acid delivery construct comprises a nucleic acid sequence according to SEQ ID NO: 18.

14. The nucleic acid delivery construct of claim 3, wherein the RNA sequence from human cytomegalovirus β2.7 RNA is an octamer of a RNA sequences selected from the group consisting of domain 1 (D1; SEQ ID NO: 3 or SEQ ID NO: 7), domain 2 (D2; SEQ ID NO: 4 or SEQ ID NO: 8), domain 3 (D3; SEQ ID NO: 5 or SEQ ID NO: 9) and domain 4 (D4; SEQ ID NO: 6 or SEQ ID NO: 10) of β2.7 RNA, according to formula IV: 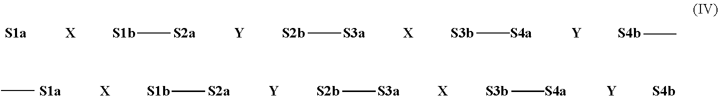
wherein X and Y are different from each other, wherein each X and each Y are independently selected from the group consisting of D1 (SEQ ID NO: 3 or SEQ ID NO: 7), D2 (SEQ ID NO: 4 or SEQ ID NO: 8), D3 (SEQ ID NO: 5 or SEQ ID NO: 9), D4 (SEQ ID NO: 6 or SEQ ID NO: 10) or combinations thereof, including duplicates, triplicates, quadruplicates, quintuplicates, sextuplicates, septuplicates, octuplicates, or longer repeats of single domains; and wherein the spacer sequences S1a, S1b, S2a, S2b, S3a, S3b S4a and S4b are as defined in claim 8.

15. The nucleic acid delivery construct of claim 14, wherein X is D3 (SEQ ID NO: 5 or SEQ ID NO: 9) and Y is D2 (SEQ ID NO: 4 or SEQ ID NO: 8); or wherein the nucleic acid delivery construct comprises a nucleic acid sequence according to SEQ ID NO: 19.

16. (canceled)

17. (canceled)

18. A vector, a recombinant cell, or a recombinant organism comprising the nucleic acid sequence of claim 1.

19. (canceled)

20. A nucleic acid sequence comprising at least one or more sense or antisense RNA sequences of the human cytomegalovirus β2.7 RNA selected from group consisting of domain 1 (D1; SEQ ID NO: 3 or 7), domain 2 (D2; SEQ ID NO: 4 or 8), domain 3 (D3; SEQ ID NO: 5 or 9) and domain 4 (D4; SEQ ID NO: 6 or 10).

21. A method of enhancing mitochondrial gene function, or suppressing defective mitochondrial gene function, or both (provided that in this case the mitochondrial genes are different from each other), the method comprising administering to a subject the nucleic acid delivery construct according to claim 1, wherein the mitochondrial gene functions are different from each other.

22. A method of treating a mitochondrial disorder, the method comprising administering to a subject the nucleic acid delivery construct according to claim 1.

23. (canceled)

24. The method according to claim 22, wherein the mitochondrial disorder is selected from the group consisting of maternally inherited diabetes mellitus, Leber's hereditary optic neuropathy (LHON), neuropathy, ataxia, retinitis pigmentosa, myoclonic epilepsy with ragged red fibres (MERRF), mitochondrial myopathy encephalopathy lactic acidosis and stroke like symptoms (MELAS), Parkinson's disease, chronic obstructive pulmonary disorder (COPD), Kearns-Sayre Syndrome (KSS), Pearson Syndrome and progressive opthalmoplegia (PEO).

25. (canceled)


SEQUENCE LISTING (87 sequences)

SEQ ID NO: 1 - RNA, 2512 nt, Artificial sequence
  Full length beta2.7 RNA (sense)
ggcgcuuccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcaagcu

SEQ ID NO: 2 - RNA, 2511 nt, Artificial sequence
  Full length beta2.7 RNA (antisense)
ggagucaagcuugcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucuggggaugugcuagcuccg

SEQ ID NO: 3 - RNA, 438 nt, Artificial sequence
  Sense domain 1 D1
ggcgcuuccggacagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucugaagcu

SEQ ID NO: 4 - RNA, 117 nt, Artificial sequence
  Sense domain 2 D2
ggcgcuuccggauuuuauuuuuuaucuucuccuuuccuuaaucucggauuaucauuucccucuccuaccuaccacgaaucgcagaugauaaacaagaggguaaaaagaaaaaaagcu

SEQ ID NO: 5 - RNA, 255 nt, Artificial sequence
  Sense domain 3 D3
ggcgcuuccggaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaacaagcu

SEQ ID NO: 6 - RNA, 384 nt, Artificial sequence
  Sense domain 4 D4
ggcgcuuccggacggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcaagcu

SEQ ID NO: 7 - RNA, 438 nt, Artificial sequence
  Antisense domain 1 D1AS
ggagucaagcuucagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuguccgg

SEQ ID NO: 8 - RNA, 117 nt, Artificial sequence
  Antisense domain 2 D2AS
ggagucaagcuuuuuuucuuuuuacccucuuguuuaucaucugcgauucgugguagguaggagagggaaaugauaauccgagauuaaggaaaggagaagauaaaaaauaaaauccgg

SEQ ID NO: 9 - RNA, 255 nt, Artificial sequence
  Antisense  domain 3 D3AS
ggagucaagcuuguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuccgg

SEQ ID NO: 10 - RNA, 384 nt, Artificial sequence
  Antisense domain 4 D4AS
ggagucaagcuugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccguccgg

SEQ ID NO: 11 - RNA, 2091 nt, Artificial sequence
  Sense domain 1 deletion mutant deltaD1
ggcgcuuccggaagagcuagcuccccagaucgcugcugccccggcguucucuuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcaagcu

SEQ ID NO: 12 - RNA, 2412 nt, Artificial sequence
  Sense domain 2 deletion mutant deltaD2
ggcgcuuccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcaagcu

SEQ ID NO: 13 - RNA, 2273 nt, Artificial sequence
  Sense domain 3 deletion mutant deltaD3
ggcgcuuccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcaagcu

SEQ ID NO: 14 - RNA, 2145 nt, Artificial sequence
  Sense domain 4 deletion mutant deltaD4
ggcgcuuccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccggguacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcaagcu

SEQ ID NO: 15 - RNA, 491 nt, Artificial sequence
  Multimer sequence D2x4
ggcgcuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcu

SEQ ID NO: 16 - RNA, 1068 nt, Artificial sequence
  Multimer sequence D3x4
ggcgcuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcu

SEQ ID NO: 17 - RNA, 1584 nt, Artificial sequence
  Multimer sequence D3x4_D2x4
ggcgcuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcuugacuccuauagugucaccuaaaugucuag

SEQ ID NO: 18 - RNA, 1569 nt, Artificial seqeunce
  Multimer sequence D2x4_D3x4
ggcgcuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcuuccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuugacuccuauagugucaccuaaaugucuag

SEQ ID NO: 19 - RNA, 1587 nt, Artificial sequence
  Multimer sequence (D3_D2)x4
ggcgcuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccggugggccguuuuauuuuuuaucuucuccuuuccuuaaucucggauuaucauuucccucuccuaccuaccacgaaucgcagaugauaaacaagaggguaaaaagaaaaacggcccaagcuuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccggugggccguuuuauuuuuuaucuucuccuuuccuuaaucucggauuaucauuucccucuccuaccuaccacgaaucgcagaugauaaacaagaggguaaaaagaaaaacggcccaagcuugacuccuauagugucaccuaaaugucuag

SEQ ID NO: 20 - RNA, 3251 nt, Artificial sequence
  gGFP_ beta2.7
ggagucucagauccgcuagcgcuaccggucgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaugagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcu

SEQ ID NO: 21 - RNA, 3275 nt, Artificial sequence
  gGFP_s_beta2.7
ggagucucagauccgcuagcgcuaccggucgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaugacaccaccccggagaacagcuccucgcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcu

SEQ ID NO: 22 - RNA, 3276 nt, Artificial sequence
  mtGFP_s_beta2.7
ggagucucagauccgcuagcgcuaccggucgccaccauagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaaagacaccaccccggagaacagcuccucgcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcu

SEQ ID NO: 23 - RNA, 786 nt, Artificial sequence
  mtGFP_s
ggagucucagauccgcuagcgcuaccggucgccaccauagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaagacaccaccccggagaacagcuccuc

SEQ ID NO: 24 - RNA, 2661 nt, Artificial sequence
  asATP6_s6_ beta2.7
ggagucuccggaauaagcgccaguagaauuagaauugugaagaugauaaguguagagggaagguuaaugguugauauugcuaggguggcgcuuccaauuaggugcgugaguagguggccugcaguaauguuaaagcaaggguuauuccgaauugggcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguccaauucggaauaacccaagcu

SEQ ID NO: 25 - RNA, 2657 nt, Artificial sequence
  beta2.7_s8_asATP8
ggcgcuuccggaauagguuggauugggggcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcuuccccaaaccaaccucuaccggaagcgccuuuuuuucguucauuuugguucucaggguuuguuauaauuuuuuauuuuuaugggcuuuggugagggagguaggugguaguuuguguuuaauauuuuuaguugggugauga

SEQ ID NO: 26 - RNA, 1757 nt, Artificial sequence
  ATP6_D34_D24
ggagucuccggaauaagcgccaguagaauuagaauugugaagaugauaaguguagagggaagguuaaugguugauauugcuaggguggcgcuuccaauuaggugcgugaguagguggccugcaguaauguuaaagcaaggguuauuccgaauugggcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccccaugaauaaggcccaagcuugacuccuauagugucaccuaaaugucuagauacuaagggagucuugc

SEQ ID NO: 27 - RNA, 5 nt, Artificial sequence
  S1a
ccggc

SEQ ID NO: 28 - RNA, 5 nt, Artificial sequence
  S1b
gccgg

SEQ ID NO: 29 - RNA, 5 nt, Artificial sequence
  S2a
cgauc

SEQ ID NO: 30 - RNA, 5 nt, Artificial sequence
  S2b
gaucg

SEQ ID NO: 31 - RNA, 5 nt, Artificial sequence
  S3a
ugugg

SEQ ID NO: 32 - RNA, 5 nt, Artificial sequence
  S3b
ccaca

SEQ ID NO: 33 - RNA, 6 nt, Artificial sequence
  S4a
ccgggg

SEQ ID NO: 34 - RNA, 6 nt, Artificial sequence
  S4b
ccccgg

SEQ ID NO: 35 - RNA, 23 nt, Artificial sequence
  S6a
aagcaaggguuauuccgaauugg

SEQ ID NO: 36 - RNA, 17 nt, Artificial sequence
  S6b
ccaauucggaauaaccc

SEQ ID NO: 37 - RNA, 13 nt, Artificial sequence
  S8a
gguuggauugggg

SEQ ID NO: 38 - RNA, 32 nt, Artificial sequence
  S8b
ccccaaaccaaccucuaccggaagcgccuuuu

SEQ ID NO: 39 - DNA, 24 bp, Artificial sequence
  Spacer Figure 3a
caccaccccggagaacagctcctc

SEQ ID NO: 40 - RNA, 2091 nt, Artificial sequence
  Antisense domain 1 deletion mutant deltaD1AS
ggagucaagcuugcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaagagaacgccggggcagcagcgaucuggggaugugcuagcuccgg

SEQ ID NO: 41 - RNA, 2412 nt, Artificial sequence
  Antisense domain 2 deletion mutant deltaD2AS
ggagucaagcuugcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucuggggaugugcuagcuccgg

SEQ ID NO: 42 - RNA, 2273 nt, Artificial sequence
  Antisense domain 3 deletion mutant deltaD3AS
ggagucaagcuugcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucuuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucuggggaugugcuagcuccgg

SEQ ID NO: 43 - RNA, 2145 nt, Artificial sequence
  Antisense domain 4 deletion mutant deltaD4AS
ggagucaagcuugcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaaguacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucuggggaugugcuagcuccgg

SEQ ID NO: 44 - RNA, 3358 nt, Artificial sequence
  gGFP_beta2.7_endo
ucagaucgcuagcgcuaccggucgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 45 - RNA, 3382 nt, Artificial sequence
  gGFP_s_beta2.7_endo
ucagaucgcuagcgcuaccggucgccaccauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaugacaccaccccggagaacagcuccucgcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 46 - RNA, 3382 nt, Artificial sequence
  mtGFP_ s_beta2.7_endo
ucagaucgcuagcgcuaccggucgccaccauagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaagacaccaccccggagaacagcuccucgcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 47 - RNA, 3500 nt, Artificial sequence
  mtGFP_NLS_s_ beta2.7_endo
ucagaucgcuagcgcuaccggucgccaccauagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaggccggacucagaucucgagcugauccaaaaaagaagagaaagguagauccaaaaaagaagagaaagguagauccaaaaaagaagagaaagguaaggauccaccggaucuagauaacuguccggaagacaccaccccggagaacagcuccucgcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 48 - RNA, 991 nt, Artificial sequence
  mtGFP_s_endo
ucagaucgcuagcgcuaccggucgccaccauagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguccggaagacaccaccccggagaacagcuccucggauccaccggaucuagauaacugaucauaaucagccauaccacauuuguagagguuuuacuugcuuuaaaaaaccucccacaccucccccugaaccugaaacauaaaaugaaugcaauuguuguuguuaacuuguuuauugcagcuuauaaugguuacaaauaaagcaauagcaucacaaauuucacaaauaaagcauuuuuuucacugca

SEQ ID NO: 49 - RNA, 2649 nt, Artificial sequence
  Full length beta2.7 RNA_endo (sense)
ucagaucuaauacgacucacuauaggcgcuuccggaagagcuagcuccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugcagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 50 - RNA, 264 nt, Artificial sequence
  beta2.7 RNA domain 2_endo (sense)
ucagaucuaauacgacucacuauaggcgcuuccggaagagcuagcuuuuauuuuuuaucuucuccuuuccuuaaucucggauuaucauuucccucuccuaccuaccacgaaucgcagaugauaaacaagaggguaaaaagaaaaaaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 51 - RNA, 403 nt, Artificial sequence
  beta2.7 RNA domain 3_endo (sense)
ucagaucuaauacgacucacuauaggcgcuuccggaagagcuagcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 52 - RNA, 629 nt, Artificial sequence
  Multimer sequence D2x4_endo
ucagaucuaauacgacucacuauaggcgcuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 53 - RNA, 1206 nt, Artificial sequence
  Multimer sequence D3x4_endo
ucagaucuaauacgacucacuauaggcgcuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 54 - RNA, 1692 nt, Artificial sequence
  Multimer sequence D3x4_D2x4_endo
ucagaucuaauacgacucacuauaggcgcuuccggaagagcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcuugacuccuauagugucaccuaaaugucuagaaacuuguuuauugcagcuuauaaugguuacaaauaaagcaauagcaucacaaauuucacaaauaaagcauuuuuuucacugca

SEQ ID NO: 55 - RNA, 2486 nt, Artificial sequence
  beta2.7_core
uccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugc

SEQ ID NO: 56 - RNA, 421 nt, Artificial sequence
  D1_core
cagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucug

SEQ ID NO: 57 - RNA, 100 nt, Artificial sequence
  D2_core
uuuuauuuuuuaucuucuccuuuccuuaaucucggauuaucauuucccucuccuaccuaccacgaaucgcagaugauaaacaagaggguaaaaagaaaaa

SEQ ID NO: 58 - RNA, 238 nt, Artificial sequence
  D3_core
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaac

SEQ ID NO: 59 - RNA, 367 nt, Artificial sequence
  D4_core
cggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgc

SEQ ID NO: 60 - RNA, 2065 nt, Artificial sequence
  deltaD1_core
uccccagaucgcugcugccccggcguucucuuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugc

SEQ ID NO: 61 - RNA, 2386 nt, Artificial sequence
  deltaD2_core
uccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugc

SEQ ID NO: 62 - RNA, 2247 nt, Artificial sequence
  deltaD3_core
uccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugc

SEQ ID NO: 63 - RNA, 2119 nt, Artificial sequence
  deltaD4_core
uccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccggguacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuugcaugc

SEQ ID NO: 64 - RNA, 2486 nt, Artificial sequence
  beta2.7 RNA_AS_core
gcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucugggga

SEQ ID NO: 65 - RNA, 421 nt, Artificial sequence
  D1AS_core
cagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucug

SEQ ID NO: 66 - RNA, 100 nt, Artificial sequence
  D2AS_core
uuuuucuuuuuacccucuuguuuaucaucugcgauucgugguagguaggagagggaaaugauaauccgagauuaaggaaaggagaagauaaaaaauaaaa

SEQ ID NO: 67 - RNA, 238 nt, Artificial sequence
  D3AS_core
guucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugacc

SEQ ID NO: 68 - RNA, 367 nt, Artificial sequence
  D4AS_core
gcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccg

SEQ ID NO: 69 - RNA, 2065 nt, Artificial sequence
  deltaD1AS_core
gcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaagagaacgccggggcagcagcgaucugggga

SEQ ID NO: 70 - RNA, 2386 nt, Artificial sequence
  deltaD2AS_core
gcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucugggga

SEQ ID NO: 71 - RNA, 2247 nt, Artificial sequence
  deltaD3AS_core
gcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaagugcgaggauaaaaauggaugacuccuucguguccagggagucgacuacugcaacgcugauugauuaaaagauggucuccgaugaugauguuguuauugaucgaaucauggugcagaacggcgacggagaggagcguguccgccgccgggaagguggucucuuucucuuuucuuuuuucaagaaaucuuccauguguuuaucguagugaucgaaaucgacugaucucggguucuuuuuguugguuucuuuucgguuaaucauguauuguuuucuuuuuuuacagaaagauacuuuuuucaugagcaauuccucgcccggcgccggcaugccgagguggggccacugcgaucagcggcaugccgacgccgacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucuuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucugggga

SEQ ID NO: 72 - RNA, 2119 nt, Artificial sequence
  deltaD4AS_core
gcaugcaaacuucucauuuauugugucuacuacucuguguugcuacagggagugaagggggugaaggcaaacaaaaaaaaaaggaacaaaauaauagauuagcagaaggaauaauccgugcgaccgagcuugugcuucuuuucuuauaaggaggcaaauauacuagggaaaacuuaagaauaggaagaaaccgagguuugggagaaaagcugagauaaaauagcgcauuuuccauacagagguuguuguuuuuguggauccuaagagguuucaagugcgaaucucaaaguucucacgagaauauugucuucaagaaucgacaacugugguccaagauuuuuuuuuggucuuuuuagguucugcgagggacaucacgauggaucguugcgaugaagucacgcguacgccucugguguggcgcggugucgugacaggagaguguguuuucagugcagagcugucuugauuccuauauccgaguaucuguuuucucguaaggacgguaaucuucuuugguguaaguacaucuaaaagcugcaaacuauauuuuaagggcugucucuagguguacuuugaugcuggaguuuuucgcuguguugaugugaauaaaucuacuacuacuauuauaugcagaaagagugauuaugccgagacaagauugcauuggcugaacuguuucaaaaacgccuacacucuacuuauccguaaaccuaagguaauacuauguguaaguuguuuuuuuuucuuuuuguaguaaaauggugauacgugcaauuaaaacuguauuccauguuuccauccuuucauuucaacuuuaaaggcggcuuugagagcgaagaaguacccggggaucuuggauucaccguuuucucucuucucucucuacauacagaccggguggcaggagcgguaaggaaucaucgucgucuuucauucuucgaugauuaugguaauacuaaaucuuaucuaggagcauauacaucuaagauuggaguacuaguagucguuugugguuucuauuuuuuuuuauauuuaucuaugacaguuuuucuguuuuucguuuugauaauaauauaauaaaaacucauggacgugaaaucuggcuugguuguggugauuucauucucauuauuguuguuuucuuuccgucuugcggaugaagauguugcgaugcgguuguuguugguguugcuauacaccgagagagaugaucuuuuuguucuucugguucauuuccuaugauuguuuggcugcugaccgacgcgucaggaugugcagggcaugcggggaaucaggaccggacacgggauaauuucaucuaccuauacggagaucgcgguccucgccaugaggaucgcgacaggcgcgucgagggggcaggaacacccuugcggauugacauucuuggugguguuucguuguugucgguaguuguuguugacgaugaggauaaauaaaaaugaccuuguuuuuguucuguuuucucuuguugggaaucgucgacuuugaauucuucgaguuaucggaaagcugagguacccaaaugucuguagcuuuuuucuuuuuacccucuuguuuaucaucugcgauucgugguagguaggagagggaaaugauaauccgagauuaaggaaaggagaagauaaaaaauaaaaaaaaauaauaaaacagaagccgaccggccgccgacccguuccccaggaccagccuacgaggaacggauaacgcgguggcgacggcagcggugguggcgcuggggguggcgguaguggugcugcugaugguagucgggacggaggagagacgaugcauacauacacgcgugcaugcugcauggguggaugguacggccgggagacgcggaagagaaacucacauaaaaaggugauaaaaagagcgguugaaaaaagaaaacgagauucgaccagacagaagagaaggaccggggcuuggcgacccuuccacgacugccguugucaucucggcuccuccaucuucucccggccacgggcggcuaagucaccgccguucuccccauccguccgagcgccgaccgaccagccggccgauucgcccgccggggcuucuggagaacgccggggcagcagcgaucugggga

SEQ ID NO: 73 - RNA, 400 nt, Artificial sequence
  Multimer sequence D2x4_core
uuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaa

SEQ ID NO: 74 - RNA, 954 nt, Artificial sequence
  Multimer sequence D3x4_core
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaacc

SEQ ID NO: 75 - RNA, 1360 nt, Artificial sequence
  Multimer sequence D3x4_D2x4_core
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaacggccc

SEQ ID NO: 76 - RNA, 1326 nt, Artificial sequence
  Multimer sequence D2x4_D3X4_core
uuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcuuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaacc

SEQ ID NO: 77 - RNA, 1356 nt, Artificial sequence
  Multimer sequence (D3_D2)x4_core
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaa

SEQ ID NO: 78 - RNA, 3198 nt, Artificial sequence
  gGFP_beta2.7_core
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaagu

SEQ ID NO: 79 - RNA, 3198 nt, Artificial sequence; identical to SEQ ID NO: 78
  gGFP_s_beta2.7_core

SEQ ID NO: 80 - RNA, 3198 nt, Artificial sequence
  mtGFP_s_beta2.7_core
auagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagagauccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaagu

SEQ ID NO: 81 - RNA, 720 nt, Artificial sequence
  mtGFP_s_core
auagugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagaga

SEQ ID NO: 82 - RNA, 2589 nt, Artificial sequence
  asATP6_s6_ beta2.7_core
aguagaauuagaauugugaagaugauaaguguagagggaagguuaaugguugauauugcuaggguggcgcuuccaauuaggugcgugaguagguggccugcaguaauguuauccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaagu

SEQ ID NO: 83 - RNA, 2585 nt, Artificial sequence
  beta2.7_s8_asATP8_core
uccccagaucgcugcugccccggcguucuccagaagccccggcgggcgaaucggccggcuggucggucggcgcucggacggauggggagaacggcggugacuuagccgcccguggccgggagaagauggaggagccgagaugacaacggcagucguggaagggucgccaagccccgguccuucucuucugucuggucgaaucucguuuucuuuuuucaaccgcucuuuuuaucaccuuuuuaugugaguuucucuuccgcgucucccggccguaccauccacccaugcagcaugcacgcguguauguaugcaucgucucuccuccgucccgacuaccaucagcagcaccacuaccgccacccccagcgccaccaccgcugccgucgccaccgcguuauccguuccucguaggcugguccuggggaacgggucggcggccggucggcuucuguuuuauuauuuuuuuuuauuuuuuaucuucuccuuuccuuaaucucggauuaucauuucccucuccuaccuaccacgaaucgcagaugauaaacaagaggguaaaaagaaaaaagcuacagacauuuggguaccucagcuuuccgauaacucgaagaauucaaagucgacgauucccaacaagagaaaacagaacaaaaacaaggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccagaagaacaaaaagaucaucucucucgguguauagcaacaccaacaacaaccgcaucgcaacaucuucauccgcaagacggaaagaaaacaacaauaaugagaaugaaaucaccacaaccaagccagauuucacguccaugaguuuuuauuauauuauuaucaaaacgaaaaacagaaaaacugucauagauaaauauaaaaaaaaauagaaaccacaaacgacuacuaguacuccaaucuuagauguauaugcuccuagauaagauuuaguauuaccauaaucaucgaagaaugaaagacgacgaugauuccuuaccgcuccugccacccggucuguauguagagagagaagagagaaaacggugaauccaagauccccgggucggcgucggcaugccgcugaucgcaguggccccaccucggcaugccggcgccgggcgaggaauugcucaugaaaaaaguaucuuucuguaaaaaaagaaaacaauacaugauuaaccgaaaagaaaccaacaaaaagaacccgagaucagucgauuucgaucacuacgauaaacacauggaagauuucuugaaaaaagaaaagagaaagagaccaccuucccggcggcggacacgcuccucuccgucgccguucugcaccaugauucgaucaauaacaacaucaucaucggagaccaucuuuuaaucaaucagcguugcaguagucgacucccuggacacgaaggagucauccauuuuuauccucgcacuucuucgcucucaaagccgccuuuaaaguugaaaugaaaggauggaaacauggaauacaguuuuaauugcacguaucaccauuuuacuacaaaaagaaaaaaaaacaacuuacacauaguauuaccuuagguuuacggauaaguagaguguaggcguuuuugaaacaguucagccaaugcaaucuugucucggcauaaucacucuuucugcauauaauaguaguaguagauuuauucacaucaacacagcgaaaaacuccagcaucaaaguacaccuagagacagcccuuaaaauauaguuugcagcuuuuagauguacuuacaccaaagaagauuaccguccuuacgagaaaacagauacucggauauaggaaucaagacagcucugcacugaaaacacacucuccugucacgacaccgcgccacaccagaggcguacgcgugacuucaucgcaacgauccaucgugaugucccucgcagaaccuaaaaagaccaaaaaaaaaucuuggaccacaguugucgauucuugaagacaauauucucgugagaacuuugagauucgcacuugaaaccucuuaggauccacaaaaacaacaaccucuguauggaaaaugcgcuauuuuaucucagcuuuucucccaaaccucgguuucuuccuauucuuaaguuuucccuaguauauuugccuccuuauaagaaaagaagcacaagcucggucgcacggauuauuccuucugcuaaucuauuauuuuguuccuuuuuuuuuuguuugccuucacccccuucacucccuguagcaacacagaguaguagacacaauaaaugagaaguuuucguucauuuugguucucaggguuuguuauaauuuuuuauuuuuaugggcuuuggugagggagguaggugguaguuuguguuuaauauuuuuaguugggugauga

SEQ ID NO: 84 - RNA, 1466 nt, Artificial sequence
  ATP6_D34_D24 (core)
aguagaauuagaauugugaagaugauaaguguagagggaagguuaaugguugauauugcuaggguggcgcuuccaauuaggugcgugaguagguggccugcaguaauguuggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaa

SEQ ID NO: 85 - RNA, 1741 nt, Artificial sequence
  Sequence of the in vitro transcribed ATP6_(D3)4_(D2)4 RNA
ggcgcuuccggaagaaagcgccaguagaauuagaauugugaagaugauaaguguagagggaagguuaaugguugauauugcuaggggggcgcuuccaauuaggugcaugaguagguggccugcaguaauguuaaagcaagggccuuauucaugggcuagcccggcggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaacggcccaagcuugacuccuauagugucaccuaaaugucuagauacuaagggagucuugc

SEQ ID NO: 86 - RNA, 1517 nt, Artificial sequence
  Multimer (D3)4_(D2)4 RNA including domains D3 and D2 and spacers
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgccgggaagcgccgaauucauaucgaucggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccgaucgaauaaagguaccuguggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccacauuuuaccgguaauaccggggggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccccccggaagcuuuccggaagagcuagcuuuuauuuuuuaucuucuccuuuccuuaaucucggauuaucauuucccucuccuaccuaccacgaaucgcagaugauaaacaagaggguaaaaagaaaaaagcgcgaauuccgaucuuuuauuuuuuaucuucuccuuuccuuaaucucggauuaucauuucccucuccuaccuaccacgaaucgcagaugauaaacaagaggguaaaaagaaaaagaucguauccgguaccugugguuuuauuuuuuaucuucuccuuuccuuaaucucggauuaucauuucccucuccuaccuaccacgaaucgcagaugauaaacaagaggguaaaaagaaaaaccacaccuccaccggugggccguuuuauuuuuuaucuucuccuuuccuuaaucucggauuaucauuucccucuccuaccuaccacgaaucgcagaugauaaacaagaggguaaaaagaaaaa

SEQ ID NO: 87 - RNA, 1356 nt, Artificial sequence
  Multimer (D3)4_(D2)4 RNA core only including domains D3 and D2 but without any spacers
ggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccggucauuuuuauuuauccucaucgucaacaacaacuaccgacaacaacgaaacaccaccaagaaugucaauccgcaaggguguuccugcccccucgacgcgccugucgcgauccucauggcgaggaccgcgaucuccguauagguagaugaaauuaucccguguccgguccugauuccccgcaugcccugcacauccugacgcgucggucagcagccaaacaaucauaggaaaugaaccuuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaauuuuauuuuuuaucuucuccuuuccuuaaucucggauuaucauuucccucuccuaccuaccacgaaucgcagaugauaaacaagaggguaaaaagaaaaa